SEQUENCE LISTING (297 sequences)

SEQ ID NO: 1 - RNA, 20 nt, Artificial Sequence
  synthetic construct
aaccacaucuuccucaacua

SEQ ID NO: 2 - RNA, 22 nt, Artificial Sequence
  synthetic construct
uaguugaggaagaugugguugg

SEQ ID NO: 3 - RNA, 20 nt, Artificial Sequence
  synthetic construct
accacaucuuccucaacuua

SEQ ID NO: 4 - RNA, 22 nt, Artificial Sequence
  synthetic construct
uaaguugaggaagauguggugg

SEQ ID NO: 5 - RNA, 20 nt, Artificial Sequence
  synthetic construct
ccacaucuuccucaacuuua

SEQ ID NO: 6 - RNA, 22 nt, Artificial Sequence
  synthetic construct
uaaaguugaggaagaugugggg

SEQ ID NO: 7 - RNA, 20 nt, Artificial Sequence
  synthetic construct
cacaucuuccucaacuuuga

SEQ ID NO: 8 - RNA, 22 nt, Artificial Sequence
  synthetic construct
ucaaaguugaggaagauguggg

SEQ ID NO: 9 - RNA, 20 nt, Artificial Sequence
  synthetic construct
acaucuuccucaacuuugua

SEQ ID NO: 10 - RNA, 22 nt, Artificial Sequence
  synthetic construct
uacaaaguugaggaagaugugg

SEQ ID NO: 11 - RNA, 20 nt, Artificial Sequence
  synthetic construct
caucuuccucaacuuuguga

SEQ ID NO: 12 - RNA, 22 nt, Artificial Sequence
  synthetic construct
ucacaaaguugaggaagauggg

SEQ ID NO: 13 - RNA, 20 nt, Artificial Sequence
  synthetic construct
aucuuccucaacuuugugca

SEQ ID NO: 14 - RNA, 22 nt, Artificial Sequence
  synthetic construct
ugcacaaaguugaggaagaugg

SEQ ID NO: 15 - RNA, 20 nt, Artificial Sequence
  synthetic construct
ucuuccucaacuuugugcca

SEQ ID NO: 16 - RNA, 22 nt, Artificial Sequence
  synthetic construct
uggcacaaaguugaggaagagg

SEQ ID NO: 17 - RNA, 20 nt, Artificial Sequence
  synthetic construct
cuuccucaacuuugugccca

SEQ ID NO: 18 - RNA, 22 nt, Artificial Sequence
  synthetic construct
ugggcacaaaguugaggaaggg

SEQ ID NO: 19 - RNA, 20 nt, Artificial Sequence
  synthetic construct
uuccucaacuuugugcccaa

SEQ ID NO: 20 - RNA, 22 nt, Artificial Sequence
  synthetic construct
uugggcacaaaguugaggaagg

SEQ ID NO: 21 - RNA, 20 nt, Artificial Sequence
  synthetic construct
uccucaacuuugugcccaca

SEQ ID NO: 22 - RNA, 22 nt, Artificial Sequence
  synthetic construct
ugugggcacaaaguugaggagg

SEQ ID NO: 23 - RNA, 20 nt, Artificial Sequence
  synthetic construct
uugucaucggcaaugacaua

SEQ ID NO: 24 - RNA, 22 nt, Artificial Sequence
  synthetic construct
uaugucauugccgaugacaagg

SEQ ID NO: 25 - RNA, 20 nt, Artificial Sequence
  synthetic construct
guuaugugagugcugggaca

SEQ ID NO: 26 - RNA, 22 nt, Artificial Sequence
  synthetic construct
ugucccagcacucacauaacgg

SEQ ID NO: 27 - RNA, 20 nt, Artificial Sequence
  synthetic construct
uuucaaacauggugguggca

SEQ ID NO: 28 - RNA, 22 nt, Artificial Sequence
  synthetic construct
ugccaccaccauguuugaaagg

SEQ ID NO: 29 - RNA, 20 nt, Artificial Sequence
  synthetic construct
aacauggugguggcuuugaa

SEQ ID NO: 30 - RNA, 22 nt, Artificial Sequence
  synthetic construct
uucaaagccaccaccauguugg

SEQ ID NO: 31 - RNA, 20 nt, Artificial Sequence
  synthetic construct
auggugguggcuuugaagga

SEQ ID NO: 32 - RNA, 22 nt, Artificial Sequence
  synthetic construct
uccuucaaagccaccaccaugg

SEQ ID NO: 33 - RNA, 20 nt, Artificial Sequence
  synthetic construct
accuguguguuugagaagga

SEQ ID NO: 34 - RNA, 22 nt, Artificial Sequence
  synthetic construct
uccuucucaaacacacaggugg

SEQ ID NO: 35 - RNA, 20 nt, Artificial Sequence
  synthetic construct
ccuguguguuugagaaggaa

SEQ ID NO: 36 - RNA, 22 nt, Artificial Sequence
  synthetic construct
uuccuucucaaacacacagggg

SEQ ID NO: 37 - RNA, 20 nt, Artificial Sequence
  synthetic construct
caguaugcuagcaacaucaa

SEQ ID NO: 38 - RNA, 22 nt, Artificial Sequence
  synthetic construct
uugauguugcuagcauacuggg

SEQ ID NO: 39 - RNA, 20 nt, Artificial Sequence
  synthetic construct
aguaugcuagcaacaucaca

SEQ ID NO: 40 - RNA, 22 nt, Artificial Sequence
  synthetic construct
ugugauguugcuagcauacugg

SEQ ID NO: 41 - RNA, 20 nt, Artificial Sequence
  synthetic construct
gugguggaauuccaguucaa

SEQ ID NO: 42 - RNA, 22 nt, Artificial Sequence
  synthetic construct
uugaacuggaauuccaccacgg

SEQ ID NO: 43 - RNA, 20 nt, Artificial Sequence
  synthetic construct
guggaauuccaguucaugca

SEQ ID NO: 44 - RNA, 22 nt, Artificial Sequence
  synthetic construct
ugcaugaacuggaauuccacgg

SEQ ID NO: 45 - RNA, 20 nt, Artificial Sequence
  synthetic construct
uggaauuccaguucaugcua

SEQ ID NO: 46 - RNA, 22 nt, Artificial Sequence
  synthetic construct
uagcaugaacuggaauuccagg

SEQ ID NO: 47 - RNA, 20 nt, Artificial Sequence
  synthetic construct
ggaauuccaguucaugcuga

SEQ ID NO: 48 - RNA, 22 nt, Artificial Sequence
  synthetic construct
ucagcaugaacuggaauuccgg

SEQ ID NO: 49 - RNA, 20 nt, Artificial Sequence
  synthetic construct
gaauuccaguucaugcugca

SEQ ID NO: 50 - RNA, 22 nt, Artificial Sequence
  synthetic construct
ugcagcaugaacuggaauucgg

SEQ ID NO: 51 - RNA, 20 nt, Artificial Sequence
  synthetic construct
aauuccaguucaugcugcca

SEQ ID NO: 52 - RNA, 22 nt, Artificial Sequence
  synthetic construct
uggcagcaugaacuggaauugg

SEQ ID NO: 53 - RNA, 20 nt, Artificial Sequence
  synthetic construct
uaaccacaucuuccucaaca

SEQ ID NO: 54 - RNA, 22 nt, Artificial Sequence
  synthetic construct
uguugaggaagaugugguuagg

SEQ ID NO: 55 - RNA, 20 nt, Artificial Sequence
  synthetic construct
aaccacaucuuccucaacua

SEQ ID NO: 56 - RNA, 22 nt, Artificial Sequence
  synthetic construct
uaguugaggaagaugugguugg

SEQ ID NO: 57 - RNA, 20 nt, Artificial Sequence
  synthetic construct
accacaucuuccucaacuua

SEQ ID NO: 58 - RNA, 22 nt, Artificial Sequence
  synthetic construct
uaaguugaggaagauguggugg

SEQ ID NO: 59 - RNA, 20 nt, Artificial Sequence
  synthetic construct
uacaaggaagugacugacua

SEQ ID NO: 60 - RNA, 22 nt, Artificial Sequence
  synthetic construct
uagucagucacuuccuuguagg

SEQ ID NO: 61 - RNA, 20 nt, Artificial Sequence
  synthetic construct
caaggaagugacugacucca

SEQ ID NO: 62 - RNA, 22 nt, Artificial Sequence
  synthetic construct
uggagucagucacuuccuuggg

SEQ ID NO: 63 - RNA, 20 nt, Artificial Sequence
  synthetic construct
aggaagugacugacuccaga

SEQ ID NO: 64 - RNA, 22 nt, Artificial Sequence
  synthetic construct
ucuggagucagucacuuccugg

SEQ ID NO: 65 - RNA, 20 nt, Artificial Sequence
  synthetic construct
gaugacuuugaagggguuua

SEQ ID NO: 66 - RNA, 22 nt, Artificial Sequence
  synthetic construct
uaaaccccuucaaagucaucgg

SEQ ID NO: 67 - RNA, 20 nt, Artificial Sequence
  synthetic construct
uggauucugaagccaagaua

SEQ ID NO: 68 - RNA, 22 nt, Artificial Sequence
  synthetic construct
uaucuuggcuucagaauccagg

SEQ ID NO: 69 - RNA, 20 nt, Artificial Sequence
  synthetic construct
ggauucugaagccaagauaa

SEQ ID NO: 70 - RNA, 22 nt, Artificial Sequence
  synthetic construct
uuaucuuggcuucagaauccgg

SEQ ID NO: 71 - RNA, 20 nt, Artificial Sequence
  synthetic construct
gauucugaagccaagauaaa

SEQ ID NO: 72 - RNA, 22 nt, Artificial Sequence
  synthetic construct
uuuaucuuggcuucagaaucgg

SEQ ID NO: 73 - RNA, 20 nt, Artificial Sequence
  synthetic construct
auucugaagccaagauaaua

SEQ ID NO: 74 - RNA, 22 nt, Artificial Sequence
  synthetic construct
uauuaucuuggcuucagaaugg

SEQ ID NO: 75 - RNA, 20 nt, Artificial Sequence
  synthetic construct
aggccaucaaggacuucaaa

SEQ ID NO: 76 - RNA, 22 nt, Artificial Sequence
  synthetic construct
uuugaaguccuugauggccugg

SEQ ID NO: 77 - RNA, 20 nt, Artificial Sequence
  synthetic construct
caucaaggacuucaaccgga

SEQ ID NO: 78 - RNA, 22 nt, Artificial Sequence
  synthetic construct
uccgguugaaguccuugauggg

SEQ ID NO: 79 - RNA, 20 nt, Artificial Sequence
  synthetic construct
ucaaggacuucaaccgggaa

SEQ ID NO: 80 - RNA, 22 nt, Artificial Sequence
  synthetic construct
uucccgguugaaguccuugagg

SEQ ID NO: 81 - RNA, 20 nt, Artificial Sequence
  synthetic construct
auggucuuugccaacuggaa

SEQ ID NO: 82 - RNA, 22 nt, Artificial Sequence
  synthetic construct
uuccaguuggcaaagaccaugg

SEQ ID NO: 83 - RNA, 20 nt, Artificial Sequence
  synthetic construct
accaagugcugaaguuugga

SEQ ID NO: 84 - RNA, 22 nt, Artificial Sequence
  synthetic construct
uccaaacuucagcacuuggugg

SEQ ID NO: 85 - RNA, 20 nt, Artificial Sequence
  synthetic construct
uguggguuauuuaaaagaaa

SEQ ID NO: 86 - RNA, 22 nt, Artificial Sequence
  synthetic construct
uuucuuuuaaauaacccacagg

SEQ ID NO: 87 - RNA, 20 nt, Artificial Sequence
  synthetic construct
guuauuuaaaagaaauuaua

SEQ ID NO: 88 - RNA, 22 nt, Artificial Sequence
  synthetic construct
uauaauuucuuuuaaauaacgg

SEQ ID NO: 89 - RNA, 20 nt, Artificial Sequence
  synthetic construct
accaggaacuauaucuuuga

SEQ ID NO: 90 - RNA, 22 nt, Artificial Sequence
  synthetic construct
ucaaagauauaguuccuggugg

SEQ ID NO: 91 - RNA, 20 nt, Artificial Sequence
  synthetic construct
aggaacuauaucuuuggaua

SEQ ID NO: 92 - RNA, 22 nt, Artificial Sequence
  synthetic construct
uauccaaagauauaguuccugg

SEQ ID NO: 93 - RNA, 20 nt, Artificial Sequence
  synthetic construct
uggugaagacacacaaccua

SEQ ID NO: 94 - RNA, 22 nt, Artificial Sequence
  synthetic construct
uagguugugugucuucaccagg

SEQ ID NO: 95 - RNA, 20 nt, Artificial Sequence
  synthetic construct
caggaacuauaucuuuggaa

SEQ ID NO: 96 - RNA, 22 nt, Artificial Sequence
  synthetic construct
uuccaaagauauaguuccuggg

SEQ ID NO: 97 - RNA, 20 nt, Artificial Sequence
  synthetic construct
ccaggaacuauaucuuugga

SEQ ID NO: 98 - RNA, 22 nt, Artificial Sequence
  synthetic construct
uccaaagauauaguuccugggg

SEQ ID NO: 99 - RNA, 20 nt, Artificial Sequence
  synthetic construct
cugaccaccaggaacuauaa

SEQ ID NO: 100 - RNA, 22 nt, Artificial Sequence
  synthetic construct
uuauaguuccugguggucaggg

SEQ ID NO: 101 - RNA, 20 nt, Artificial Sequence
  synthetic construct
guccagaagaaguuccagaa

SEQ ID NO: 102 - RNA, 22 nt, Artificial Sequence
  synthetic construct
uucuggaacuucuucuggacgg

SEQ ID NO: 103 - RNA, 20 nt, Artificial Sequence
  synthetic construct
agaaugagguauacaagcaa

SEQ ID NO: 104 - RNA, 22 nt, Artificial Sequence
  synthetic construct
uugcuuguauaccucauucugg

SEQ ID NO: 105 - RNA, 20 nt, Artificial Sequence
  synthetic construct
cuugagugcauuuuguucua

SEQ ID NO: 106 - RNA, 22 nt, Artificial Sequence
  synthetic construct
uagaacaaaaugcacucaaggg

SEQ ID NO: 107 - RNA, 20 nt, Artificial Sequence
  synthetic construct
cauugcaauguuagauguua

SEQ ID NO: 108 - RNA, 22 nt, Artificial Sequence
  synthetic construct
uaacaucuaacauugcaauggg

SEQ ID NO: 109 - RNA, 20 nt, Artificial Sequence
  synthetic construct
agguauacaagcaggugaua

SEQ ID NO: 110 - RNA, 22 nt, Artificial Sequence
  synthetic construct
uaucaccugcuuguauaccugg

SEQ ID NO: 111 - RNA, 20 nt, Artificial Sequence
  synthetic construct
ggugaagcucuuugacaaua

SEQ ID NO: 112 - RNA, 22 nt, Artificial Sequence
  synthetic construct
uauugucaaagagcuucaccgg

SEQ ID NO: 113 - RNA, 20 nt, Artificial Sequence
  synthetic construct
auagacuacuugcucuccaa

SEQ ID NO: 114 - RNA, 22 nt, Artificial Sequence
  synthetic construct
uuggagagcaaguagucuaugg

SEQ ID NO: 115 - RNA, 20 nt, Artificial Sequence
  synthetic construct
cuguaaauuuggaagcguca

SEQ ID NO: 116 - RNA, 22 nt, Artificial Sequence
  synthetic construct
ugacgcuuccaaauuuacaggg

SEQ ID NO: 117 - RNA, 20 nt, Artificial Sequence
  synthetic construct
aagaaguuccagaaauacaa

SEQ ID NO: 118 - RNA, 22 nt, Artificial Sequence
  synthetic construct
uuguauuucuggaacuucuugg

SEQ ID NO: 119 - RNA, 20 nt, Artificial Sequence
  synthetic construct
auagacuauuugcuuucaaa

SEQ ID NO: 120 - RNA, 22 nt, Artificial Sequence
  synthetic construct
uuugaaagcaaauagucuaugg

SEQ ID NO: 121 - RNA, 20 nt, Artificial Sequence
  synthetic construct
agaaugaaguguacaagcaa

SEQ ID NO: 122 - RNA, 22 nt, Artificial Sequence
  synthetic construct
uugcuuguacacuucauucugg

SEQ ID NO: 123 - RNA, 20 nt, Artificial Sequence
  synthetic construct
aaguguacaagcaggugaua

SEQ ID NO: 124 - RNA, 22 nt, Artificial Sequence
  synthetic construct
uaucaccugcuuguacacuugg

SEQ ID NO: 125 - RNA, 20 nt, Artificial Sequence
  synthetic construct
cauagacuauuugcuuucaa

SEQ ID NO: 126 - RNA, 22 nt, Artificial Sequence
  synthetic construct
uugaaagcaaauagucuauggg

SEQ ID NO: 127 - RNA, 20 nt, Artificial Sequence
  synthetic construct
acaccauagacuauuugcua

SEQ ID NO: 128 - RNA, 22 nt, Artificial Sequence
  synthetic construct
uagcaaauagucuauggugugg

SEQ ID NO: 129 - RNA, 20 nt, Artificial Sequence
  synthetic construct
uuggagagaaugaaguguaa

SEQ ID NO: 130 - RNA, 22 nt, Artificial Sequence
  synthetic construct
uuacacuucauucucuccaagg

SEQ ID NO: 131 - RNA, 20 nt, Artificial Sequence
  synthetic construct
augaaguguacaagcaggua

SEQ ID NO: 132 - RNA, 22 nt, Artificial Sequence
  synthetic construct
uaccugcuuguacacuucaugg

SEQ ID NO: 133 - RNA, 20 nt, Artificial Sequence
  synthetic construct
caccauagacuauuugcuua

SEQ ID NO: 134 - RNA, 22 nt, Artificial Sequence
  synthetic construct
uaagcaaauagucuaugguggg

SEQ ID NO: 135 - RNA, 20 nt, Artificial Sequence
  synthetic construct
ccauagacuauuugcuuuca

SEQ ID NO: 136 - RNA, 22 nt, Artificial Sequence
  synthetic construct
ugaaagcaaauagucuaugggg

SEQ ID NO: 137 - RNA, 20 nt, Artificial Sequence
  synthetic construct
cucauguacauauucugcaa

SEQ ID NO: 138 - RNA, 22 nt, Artificial Sequence
  synthetic construct
uugcagaauauguacaugaggg

SEQ ID NO: 139 - RNA, 20 nt, Artificial Sequence
  synthetic construct
gacaccauagacuauuugca

SEQ ID NO: 140 - RNA, 22 nt, Artificial Sequence
  synthetic construct
ugcaaauagucuauggugucgg

SEQ ID NO: 141 - RNA, 20 nt, Artificial Sequence
  synthetic construct
gaagaaguuccagaaauaca

SEQ ID NO: 142 - RNA, 22 nt, Artificial Sequence
  synthetic construct
uguauuucuggaacuucuucgg

SEQ ID NO: 143 - RNA, 20 nt, Artificial Sequence
  synthetic construct
accauagacuauuugcuuua

SEQ ID NO: 144 - RNA, 22 nt, Artificial Sequence
  synthetic construct
uaaagcaaauagucuauggugg

SEQ ID NO: 145 - RNA, 20 nt, Artificial Sequence
  synthetic construct
auccucauguacauauucua

SEQ ID NO: 146 - RNA, 22 nt, Artificial Sequence
  synthetic construct
uagaauauguacaugaggaugg

SEQ ID NO: 147 - RNA, 20 nt, Artificial Sequence
  synthetic construct
gagaaugaaguguacaagca

SEQ ID NO: 148 - RNA, 22 nt, Artificial Sequence
  synthetic construct
ugcuuguacacuucauucucgg

SEQ ID NO: 149 - DNA, 2251 bp, Mus musculus
ccgcgcttcgctggctttctgctcatctagggtggcagcggctacctacctcagctctcgccctgctgccgccacggcctgggcgctgtccctcagctcccggagctcagcgcgaagccctggccccggcggccggggcatgggtcaggggcgcggcgtgaggcggctttctgcacggccgtgacgtgcattggcttcagcatgaagaccctcatcgccgcctactccggggtcctgcggggtgagcgtcgggcggaagctgcccgcagcgaaaacaagaataaaggatctgccctgtcacgcgaggggtctgggcgatggggcactggctccagcatcctctcagccctccaagacatcttctctgtcacctggctcaacagatctaaggtggaaaaacagctgcaggtcatctcagtactacaatgggtcctatccttcctggtgctaggagtggcctgcagtgtcatcctcatgtacaccttctgcacagactgctggctgatagctgtgctctacttcacctggctggcatttgactggaacacgcccaagaaaggtggcaggagatcgcagtgggtgcgaaactgggccgtgtggcgctacttccgagactactttcccatccagctggtgaagacacacaacctgctgaccaccaggaactatatctttggataccacccccatggcatcatgggcctgggtgccttctgtaacttcagcacagaggctactgaagtcagcaagaagtttcctggcataaggccctatttggctacgttggctggtaacttccggatgcctgtgcttcgcgagtacctgatgtctggaggcatctgccctgtcaaccgagacaccatagactacttgctctccaagaatgggagtggcaatgctatcatcatcgtggtgggaggtgcagctgagtccctgagctccatgcctggcaagaacgcagtcaccctgaagaaccgcaaaggctttgtgaagctggccctgcgccatggagctgatctggttcccacttattcctttggagagaatgaggtatacaagcaggtgatctttgaggagggttcctggggccgatgggtccagaagaagttccagaagtatattggtttcgccccctgcatcttccatggccgaggcctcttctcctctgacacctgggggctggtgccctactccaagcccatcaccaccgtcgtgggggagcccatcactgtccccaagctggagcacccgacccagaaagacatcgacctgtaccatgccatgtacatggaggccctggtgaagctctttgacaatcacaagaccaaatttggccttccagagactgaggtgctggaggtgaactgacccagccctcgcgtgccagctcctgggagggacgactgcagatccttttctaccgagttcttgagtgcattttgttctgtaaatttggaagcgtcatgggtgtctgtgggttatttaaaagaaattataatttgttaaaccattgcaatgttagatgttttttaagaagggaagagtcagtattttaagctcacttctagtgtgtcctgctcaaggtggaggctgatatttatgggccttggtggtttcttacccaccccttctagcgttccccagacgacagacacttggccctggctagctgggcaagggcagtccttagtgactccagggattcttgagaggcagaggccatgtcccacccgtggctgcaggtcgggttcctcgtaccaaggggaggctgagggcacagctggccccacttggggagggtagataacatctggactgcccggcttgggtctctgctcctcaccctagccctcttctccaatctgagcctaccctggcctcctgtctcctggctagggacacggctgtcccacaggtgccgtcttgggttatctcgctgctgttggctggtttcactctggaggttggcaccatggacacagctcagcgttgctctggcgcatatcctcctgagccacaccccaagtctggtgtgaggaagggcttctcttctcttcacagaggtgcctggcttcctgtgcagcacactgggtccaggacaggaggccccccccccaaaccaagcctcacgtgtgtgcctttatgaggcgttgggagaaagctaccctcctgtgtattctgttttctccatgagattgttgtgccatgtcacacttttgtatattcctagactaataaatggaaacaagaa

SEQ ID NO: 150 - DNA, 10013 bp, Homo sapiens
gccgcctccgcccctcggccgtggaggcccccgccgggtgctgagcgcccgccaccgccctcttggccttttcccggtccccgccgcgcgcctgctgctgtccccgtgcccgaggactgggaggatggggctgagccgcttgcctgacttttgatccgaccagtaatcactttgcccgtgtggcgcgtggcccgttgcctgaggcttcctggcgcgtggggctgtttggtcccctccggagtgcggtgaggcgggccgagccgggactgcctgggtttggggataacgttcccatctccacccctgttgcagcaagggaaattgaggctgagggaactgggcccagggacggcgagccgtggcgcctctccagtccgggccccgaagggctgctcgtggatgaaccagactgattttaaggggtgaagagggtgcgtttcaatcagatgctcctggaacgtcgaaattgtcttctttggaaagaaccatcccctctttgggcttcagaggcccaaattgaggcgcaatgatgagaggatgtggtggtctactctgatgtcaatcttgagggctaggtctttttggaagtggatatctactcagacagtaagaattataagagctgtaagagctcattttggaggaataatggatgaaccatctcccttggcccaacctctggagctgaaccagcactctcgattcataataggttctgtgtctgaagataactcagaggatgagatcagcaacctggtgaagttggacctactggaggagaaggagggctccttgtcacctgcttctgttggctcagatacactctctgatttggggatctctagcctacaggatggcttggccttgcacataaggtccagcatgtctggcttgcacctagtaaagcagggccgagacagaaagaaaatagattctcaacgagatttcactgtggcttctccagcagaatttgttactcgctttgggggaaataaagtgattgagaaggttcttattgctaacaatggcattgcagcagtgaaatgcatgcggtctatccgtaggtggtcttatgaaatgtttcgaaatgaacgtgcaattagattcgttgtcatggtcacacctgaagaccttaaagccaatgcagaatacattaagatggcagatcactatgtgccagtgcctggaggaccaaacaacaacaactatgcaaatgtggaattaattcttgatattgctaaaaggatcccagtacaagcagtgtgggctggctggggtcatgcttctgagaatcccaaactaccggaacttctcttgaaaaatggcattgccttcatgggtcctccaagccaggccatgtgggctttaggggataagattgcatcttccatagtggctcaaactgcaggtatcccaactcttccctggagcggcagtggtcttcgtgtggactggcaggaaaatgatttttcaaaacgtatcttaaatgttccccaggagctatatgaaaaaggttatgtgaaagatgtggatgatgggctacaggcagctgaggaagttggatatccagtaatgatcaaggcctcagagggaggaggagggaagggaattagaaaagtcaacaatgcagatgacttccctaatctcttcagacaggttcaagctgaagttcctggatctcccatatttgtgatgagactagccaaacaatctcgtcatctggaggtgcagatcttagcggaccaatatggcaatgctatctctttgtttggtcgtgattgctctgtacaacgcaggcatcagaagattattgaagaagcacctgctactattgctactccagcagtatttgaacacatggaacagtgtgcggtgaaacttgccaaaatggtgggttatgtgagtgctgggactgtggaatacctgtacagccaggatggcagcttctactttctggaattgaatcctcggctgcaggtagagcacccttgtacagagatggtggctgatgtcaatctccctgcagcacagctccagattgccatggggattcctctatatagaatcaaggatatccgtatgatgtatggggtatctccctggggtgattctcccattgattttgaagattctgcacacgttccttgtccaaggggccatgttattgctgctcggatcactagtgaaaatccagatgagggttttaagcccagctcaggaacagttcaggagctaaatttccgcagcaataagaatgtttggggatatttcagtgttgctgctgcagggggacttcatgaatttgctgattctcagtttggtcactgcttttcttggggagaaaacagagaagaggcaatttcaaacatggtggtggctttgaaggagctgtctattcggggtgactttcgaactacagttgaatacctgatcaaattgttagagactgaaagctttcagatgaacagaattgatactggctggctggacagactgatagcagaaaaagtacaggctgagcgacctgacaccatgttgggggttgtgtgtggtgccctccacgtggcagatgtgagcctgcggaatagcgtctctaacttccttcactccttagaaaggggtcaagtccttcctgctcatacacttctgaatacagtagatgttgaacttatctatgagggagtcaagtatgtacttaaggtgactcgacagtcccccaactcctatgtggtgatcatgaatggctcatgtgtagaagtagatgtacatcggctgagtgacggtggactgctcttgtcctatgatggcagcagttatactacgtatatgaaagaggaagtggatagatatcgcatcacaattggcaataaaacctgtgtgtttgagaaggaaaatgacccatcggtgatgcgctcaccttctgctgggaagttaatccagtacattgtagaagatggaggtcatgtgtttgccggccagtgctatgctgagattgaggtaatgaagatggtaatgaccttaacagctgtggagtctggctgtatccattacgtcaagcgacctggagcagctcttgaccctggctgtgtactagccaaaatgcaactggacaaccccagcaaggttcagcaggctgaacttcacacaggtagtctgccacggatccagagcacggcactcagaggcgagaaactccatcgagtgttccattatgtcctggataatctggtcaatgtaatgaatggatactgccttccagatcctttctttagcagcaaggtaaaagactgggtagagcgattgatgaaaaccctcagagatccctccctgcctctcctagaattgcaagatattatgaccagtgtgtctggccgcattccccccaatgtggagaagtctatcaagaaggaaatggctcagtatgctagcaacatcacatcagtcctctgtcagtttcccagccagcagattgcaaacatcctagatagccatgcagctacattgaaccggaaatctgaacgggaagtcttctttatgaatactcagagcattgttcagctggtacagaggtaccgaagtggcatccgaggccacatgaaggctgtggtgatggatctgctccggcagtacctgcgagtagagacacaattccagaatggtcactatgacaaatgtgtattcgccctccgagaagagaataaaagtgacatgaacactgtactgaactacatcttctctcacgctcaagtcaccaagaagaatcttctggtcacaatgcttattgatcagttgtgtggccgggaccctactctcactgatgagctgctgaatattctcacagagctaactcaactcagtaagaccaccaatgccaaagtagcacttcgagcacgccaggttcttattgcctcccatttgccatcatatgagcttcgccataaccaagtagagtctatcttcctatcagctattgacatgtatggacatcaattttgcattgagaacctgcagaaactcatcctatcagaaacatctatttttgatgtcctaccaaacttcttctatcacagcaaccaagtagtgaggatggcagctctggaggtgtatgttcgaagggcttatattgcctatgaacttaacagcgtacaacaccgccagcttaaggacaacacctgtgtggtggaattccagttcatgctgcccacatctcatccaaacagagggaacatccctacgctaaacagaatgtccttctcctccaacctcaaccactatggcatgacccatgtagctagtgtcagcgatgtactgttggacaactcattcactccaccttgtcagcggatgggcggaatggtctcttttcggacttttgaagattttgtcaggatctttgatgaagtgatgggctgcttctctgactccccaccccagagtcccacattccctgaggcaggtcacacgtctctttatgatgaggataaggttcccagggatgaaccaattcacattctcaatgtggctatcaagactgactgtgatattgaggatgacaggctggcagctatgttcagagaatttacccagcaaaataaagctaccctggttgaccatgggatccggcgccttactttcctggttgcacaaaaggatttcagaaagcaggtcaactatgaggtggatcggagatttcatagagaattccctaaattttttacattccgagcaagggataagtttgaggaggatcgtatctatcgtcatctggagcctgctctggctttccagttagagctgaaccggatgagaaattttgacctcactgccattccatgtgctaatcacaagatgcacctgtatctcggggcagccaaggtggaagtgggcacagaagtgacagactacaggttctttgttcgtgcaatcatcaggcattctgatctggtcaccaaggaagcttcttttgaatatctgcaaaatgaaggggagcggctactcctggaagccatggatgagttggaagttgcttttaacaatacaaatgtccgcactgactgtaaccacatcttcctcaactttgtgcccacggttatcatggacccatcaaagattgaggaatccgtgcggagcatggtaatgcggtatggaagtcgcctgtggaaattgcgcgtcctccaggcagaactgaaaatcaacattcgcctgacgccaactggaaaagcaattcccatccgcctcttcctgacaaacgagtctggctattacttggatatcagcctatacaaggaagtgactgactccaggacagcacagatcatgtttcaggcatatggagacaaacagggaccactgcatggaatgttaatcaatactccatatgtgaccaaagacctgctgcaatcaaagaggttccaggcacaatccttagggacaacatacatatatgatatcccagagatgtttcggcagtccctgatcaaactctgggagtctatgtccactcaagcatttcttccatctccccctctgccttctgacatgctgacttacactgaactggtactggatgatcaaggtcagctggtccacatgaacaggcttccaggaggaaatgagattggcatggtagcttggaaaatgacctttaaaagtcctgaatatccagaaggccgagatatcattgttattggcaatgacatcacataccgaattgggtcctttgggcctcaagaggatttgttatttctcagagcttccgaacttgctagggcagaaggtattccacgcatctatgtatcagccaacagtggagcaagaatcggactggcagaagaaattcgccatatgtttcatgtggcctgggtagatcctgaggatccttacaagggatacaggtatttatatctgactcctcaagattataagagagtcagtgctctcaactctgtccattgtgaacacgtggaagatgaaggagaatccaggtacaagataacagatattattgggaaagaagagggaattggacccgagaaccttcgaggttctggaatgattgctggagaatcctcattggcctataatgagatcattaccatcagcctggtgacgtgccgggccattgggattggggcttaccttgtccggctgggacagagaaccatccaggttgagaattctcacttaattctaacaggagctggagccctcaacaaagtcctcgggcgggaagtgtacacctccaataaccagctggggggcatccagattatgcacaacaatggggtgacccactgcactgtgtgtgatgactttgaaggggttttcactgtcctgcactggctgtcttacatgcccaagagcgtgcacagttcagttcctcttctgaactcaaaggatcctatagacagaatcatcgagtttgttcccacaaagaccccatacgatcctcgatggatgctagcaggccgtcctcacccaacccaaaaaggtcagtggttgagtggcttttttgactatggatctttctcagagattatgcagccctgggcacagactgtggtggttggtagagccaggctaggaggaatacctgtgggagttgttgctgtagaaacccgaacagtagaactaagtatcccagctgatccagcaaacctggattctgaagccaagataatccagcaggctggccaggtttggttcccagattctgcgtttaagacgtatcaggccatcaaggacttcaaccgggaagggctgcctctgatggtctttgccaactggagaggcttctctggtggaatgaaagatatgtacgaccaagtgctgaagtttggtgcttacattgtggatggcttgagggagtgctgccagcctgtgctggtttacattcctccccaggctgagctgcggggtggctcctgggtggtgattgactcctccatcaacccccggcacatggagatgtatgctgaccgagaaagcaggggatctgttctggagccagaagggacagtagaaatcaaattccgcagaaaggatctggtgaaaaccatgcgtcgggtggacccagtctacatccacttggctgagcgattggggaccccagagctaagcacagctgagcggaaggagttggagaacaagttgaaggagcgggaggaattcctaattcccatttaccatcaggtagccgtgcagtttgctgacttgcacgacacaccaggccggatgcaggagaagggtgttattagcgatatcctggattggaaaacatcccgtaccttcttctactggcggctgaggcgtcttctgctggaggacctggtcaagaagaaaatccacaatgccaaccctgagctgactgatggccagattcaagccatgttaaggcgctggtttgtggaagtggaaggaacagtgaaggcttatgtttgggacaataataaggatctggcggagtggctagagaaacagctgacagaggaggatggtgttcactcggtaatagaggaaaacatcaaatgcatcagcagagactacgtcctcaagcaaatccgcagcttggtccaggccaatccagaggttgccatggattccatcatccatatgacgcagcacatatcacccactcagcgagcagaagtcatacggatcctctccacaatggattccccttccacgtaggaagagcttcctgcctgtccctgccctgtctctggagaaaagggctagagctgccttttacaactgtaaccactgtaatgagaaggcacaggagacccagcactggagtcaaatggcattttacttcctctcgtttcaggttatgcatgacatcctgggatgtaagatcacagaatccccctccagcccaccagtcacacctaccccattcagtatttattaccctggccaggcctagtcctccactccctgcacaggactgagaaggcaatgaaaggtacaaacatgtaccatgaggtcttactaaccaaagtagggctgcccctcctgtcctgacagccccttggcctcccagcatggggaagcgtgaggagttgcccagcagtgagcagcccccctcactcctggccccatgagccgcagccacaggcagcagaggagggctaaggagaggaggaagcctcaagtccattgtttattaccccgactcttagcccagcacacagtaggcactggagaggaatgattcccagtttaaccacactacggtaccttttatgaagaaaaattagagcataaaatctactacaagctccataggaactcaaagatgagggcaaaactgtgagccaagaagcagagaaagaaaatagaaccagttattcttgatttaggggacctcaaccttgggttcagtctctgaggacaaagggaaaggtagttgttggcctgcctctcgcctgcacgtcactgctggactagctgtcgcatgtggctgggagctgcaaggccagtgcttgaggggccccagcagttccacaggtggtgaagcctgagttggcagaggaggagccagaagagaactgccctttctgcactggtggaaactagttatttatgccatgtggagagccagtgagatagatagatagtctgtttgttttgaggacttggaaagttgttcctatgaagcctggagcttggatggttttgagaggttaatggtgcctccacactcactcttccctagttccaggattactgtcctagcagctaacggttctactctcttccccagagtgtagacaggcagcaggtctccccacagctctgaaaggaccctggtgacagctacaccctcagcaccaggagctggccttcctgatgagggaggcttccaggaaacacagaatccacatgaccttaagattatttacaactcagtcatggtgctgctgtcctccaggcttactggcccctcctgactggcatcaggggcttcctcaggtggtggagagagtttactttcaacaactagtttattcaagaaaagaacttactgattcctctgttcctaaagcaagagtggcaggtgatcagggctggtgtagcatccggttcctttagtgcagctaactgcatttgtcactgatgaccaaggaggaaatcactaagacatttgagaagcagtggtatgaacgttcttggacaagccacagttctgagccttaaccctgtagtttgcacacaagaacgagctccacctccccttcttcaggaggaatctgtgcggatagattggctggacttttcaatggttctgggttgcaggtgggcactgtatggctgggtatggagcggacagcccccaggagtcagagcctcagcccggctgccctggtggaaggtacaggtgttcagcaccttcagaaaagggcataaagtggtgggggacaattctcagtccaggaaaatgcattgaccattgctggctatttgcttacctagtaagaattggattcatttttgaccagattattcttctatgcttttttgcaataaatcaaatcccacatatctacaagtggtatgaagtcctgcaccccccaggaggcctgtccaggcatgtcttcagaggcagggtgggttacactcatttacctcccctctccccaccaaattatgacacaaacgagtatgtttcctctctagaaccctgtaatgcctcctcccccatccccagagctccttactgtaggtcttaccctggacaaggattttttcaagttggaggcacagaacatgagcaatctgacattcccacagcccctcaaacatgcaaggctactaaggcaggaggagtataaatgatggatattgaccaagacctgcttggacggagaccgccatattatctgttctcttcgttcacaaaacagccttcacttgtctcagaatttgatggacacatactgtgatgagcaggagcttcagatgcactctttacacattttgttgaaataaacctctacatttgtagaaga

SEQ ID NO: 151 - DNA, 9368 bp, Homo sapiens
agagtaagcagctagcaggcttagattcaggccctcagcaaacaaggaacctggaaaatgtaaccctgaatgcacggtggggaggacatggcaagagaaaagcggcaggaataaagtgattttctgaatggtcttgcttctttgtctatcttgtctgattttctcctgtctgaccttttcctggttaaaaatctgggggaaaatgacggactccaagccgatcaccaagagtaaatcagaagcaaacctcatcccgagccaggagccctttccagcctctgataactcaggggagacaccgcagagaaatggggagggccacactctgcccaagacacccagccaggccgagccagcctcccacaaaggccccaaagatgccggtcggcggagaaactccctaccaccctcccaccagaagcccccaagaaaccccctttcttccagtgacgcagcaccctccccagagcttcaagccaacgggactgggacacaaggtctggaggccacagataccaatggcctgtcctcctcagccaggccccagggccagcaagctggctccccctccaaagaagacaagaagcaggcaaacatcaagaggcagctgatgaccaacttcatcctgggctcttttgatgactactcctccgacgaggactctgttgctggctcatctcgtgagtctacccggaagggcagccgggccagcttgggggccctgtccctggaggcttatctgaccacaggtgaagctgagacccgcgtccccactatgaggccgagcatgtcgggactccacctggtgaagaggggacgggaacacaagaagctggacctgcacagagactttaccgtggcttctcccgctgagtttgtcacacgctttgggggggatcgggtcatcgagaaggtgcttattgccaacaacgggattgccgccgtgaagtgcatgcgctccatccgcaggtgggcctatgagatgttccgcaacgagcgggccatccggtttgttgtgatggtgacccccgaggaccttaaggccaacgcagagtacatcaagatggcggatcattacgtccccgtcccaggagggcccaataacaacaactatgccaacgtggagctgattgtggacattgccaagagaatccccgtgcaggcggtgtgggctggctggggccatgcttcagaaaaccctaaacttccggagctgctgtgcaagaatggagttgctttcttaggccctcccagtgaggccatgtgggccttaggagataagatcgcctccaccgttgtcgcccagacgctacaggtcccaaccctgccctggagtggaagcggcctgacagtggagtggacagaagatgatctgcagcagggaaaaagaatcagtgtcccagaagatgtttatgacaagggttgcgtgaaagacgtagatgagggcttggaggcagcagaaagaattggttttccattgatgatcaaagcttctgaaggtggcggagggaagggaatccggaaggctgagagtgcggaggacttcccgatccttttcagacaagtacagagtgagatcccaggctcgcccatctttctcatgaagctggcccagcacgcccgtcacctggaagttcagatcctcgctgaccagtatgggaatgctgtgtctctgtttggtcgcgactgctccatccagcggcggcatcagaagatcgttgaggaagcaccggccaccatcgccccgctggccatattcgagttcatggagcagtgtgccatccgcctggccaagaccgtgggctatgtgagtgcagggacagtggaatacctctatagtcaggatggcagcttccacttcttggagctgaatcctcgcttgcaggtggaacatccctgcacagaaatgattgctgatgttaatctgccggccgcccagctacagatcgccatgggcgtgccactgcaccggctgaaggatatccggcttctgtatggagagtcaccatggggagtgactcccatttcttttgaaaccccctcaaaccctcccctcgcccgaggccacgtcattgccgccagaatcaccagcgaaaacccagacgagggttttaagccgagctccgggactgtccaggaactgaatttccggagcagcaagaacgtgtggggttacttcagcgtggccgctactggaggcctgcacgagtttgcggattcccaatttgggcactgcttctcctggggagagaaccgggaagaggccatttcgaacatggtggtggctttgaaggaactgtccatccgaggcgactttaggactaccgtggaatacctcattaacctcctggagaccgagagcttccagaacaacgacatcgacaccgggtggttggactacctcattgctgagaaagtgcaggcggagaaaccggatatcatgcttggggtggtatgcggggccttgaacgtggccgatgcgatgttcagaacgtgcatgacagatttcttacactccctggaaaggggccaggtcctcccagcggattcactactgaacctcgtagatgtggaattaatttacggaggtgttaagtacattctcaaggtggcccggcagtctctgaccatgttcgttctcatcatgaatggctgccacatcgagattgatgcccaccggctgaatgatggggggctcctgctctcctacaatgggaacagctacaccacctacatgaaggaagaggttgacagttaccgaattaccatcggcaataagacgtgtgtgtttgagaaggagaacgatcctacagtcctgagatccccctcggctgggaagctgacacagtacacagtggaggatgggggccacgttgaggctgggagcagctacgctgagatggaggtgatgaagatgatcatgaccctgaacgttcaggaaagaggccgggtgaagtacatcaagcgtccaggtgccgtgctggaagcaggctgcgtggtggccaggctggagctcgatgacccttctaaagtccacccggctgaaccgttcacaggagaactccctgcccagcagacactgcccatcctcggagagaaactgcaccaggtcttccacagcgtcctggaaaacctcaccaacgtcatgagtggcttttgtctgccagagcccgtttttagcataaagctgaaggagtgggtgcagaagctcatgatgaccctccggcacccgtcactgccgctgctggagctgcaggagatcatgaccagcgtggcaggccgcatccccgcccctgtggagaagtctgtccgcagggtgatggcccagtatgccagcaacatcacctcggtgctgtgccagttccccagccagcagatagccaccatcctggactgccatgcagccaccctgcagcggaaggctgatcgagaggtcttcttcatcaacacccagagcatcgtgcagttggtccagagataccgcagcgggatccgcggctatatgaaaacagtggtgttggatctcctgagaagatacttgcgtgttgagcaccattttcagcaagcccactacgacaagtgtgtgataaacctcagggagcagttcaagccagacatgtcccaggtgctggactgcatcttctcccacgcacaggtggccaagaagaaccagctggtgatcatgttgatcgatgagctgtgtggcccagacccttccctgtcggacgagctgatctccatcctcaacgagctcactcagctgagcaaaagcgagcactgcaaagtggccctcagagcccggcagatcctgattgcctcccacctcccctcctacgagctgcggcataaccaggtggagtccattttcctgtctgccattgacatgtacggccaccagttctgccccgagaacctcaagaaattaatactttcggaaacaaccatcttcgacgtcctgcctactttcttctatcacgcaaacaaagtcgtgtgcatggcgtccttggaggtttacgtgcggaggggctacatcgcctatgagttaaacagcctgcagcaccggcagctcccggacggcacctgcgtggtagaattccagttcatgctgccgtcctcccacccaaaccggatgaccgtgcccatcagcatcaccaaccctgacctgctgaggcacagcacagagctcttcatggacagcggcttctccccactgtgccagcgcatgggagccatggtagccttcaggagattcgaggacttcaccagaaattttgatgaagtcatctcttgcttcgccaacgtgcccaaagacacccccctcttcagcgaggcccgcacctccctatactccgaggatgactgcaagagcctcagagaagagcccatccacattctgaatgtgtccatccagtgtgcagaccacctggaggatgaggcactggtgccgattttacggacattcgtacagtccaagaaaaatatccttgtggattatggactccgacgaatcacattcttgattgcccaagagaaagaatttcccaagtttttcacattcagagcaagagatgagtttgcagaagatcgcatttaccgtcacttggaacctgccctggccttccagctggaacttaaccggatgcgtaacttcgatctgaccgccgtgccctgtgccaaccacaagatgcacctttacctgggtgctgccaaggtgaaggaaggtgtggaagtgacggaccataggttcttcatccgcgccatcatcaggcactctgacctgatcacaaaggaagcctccttcgaatacctgcagaacgagggtgagcggctgctcctggaggccatggacgagctggaggtggcgttcaataacaccagcgtgcgcaccgactgcaaccacatcttcctcaacttcgtgcccactgtcatcatggaccccttcaagatcgaggagtccgtgcgctacatggttatgcgctacggcagccggctgtggaaactccgtgtgctacaggctgaggtcaagatcaacatccgccagaccaccaccggcagtgccgttcccatccgcctgttcatcaccaatgagtcgggctactacctggacatcagcctctacaaagaagtgactgactccagatctggaaatatcatgtttcactccttcggcaacaagcaagggccccagcacgggatgctgatcaatactccctacgtcaccaaggatctgctccaggccaagcgattccaggcccagaccctgggaaccacctacatctatgacttcccggaaatgttcaggcaggctctctttaaactgtggggctccccagacaagtatcccaaagacatcctgacatacactgaattagtgttggactctcagggccagctggtggagatgaaccgacttcctggtggaaatgaggtgggcatggtggccttcaaaatgaggtttaagacccaggagtacccggaaggacgggatgtgatcgtcatcggcaatgacatcacctttcgcattggatcctttggccctggagaggaccttctgtacctgcgggcatccgagatggcccgggcagagggcattcccaaaatttacgtggcagccaacagtggcgcccgtattggcatggcagaggagatcaaacacatgttccacgtggcttgggtggacccagaagacccccacaaaggatttaaatacctgtacctgactccccaagactacaccagaatcagctccctgaactccgtccactgtaaacacatcgaggaaggaggagagtccagatacatgatcacggatatcatcgggaaggatgatggcttgggcgtggagaatctgaggggctcaggcatgattgctggggagtcctctctggcttacgaagagatcgtcaccattagcttggtgacctgccgagccattgggattggggcctacttggtgaggctgggccagcgagtgatccaggtggagaattcccacatcatcctcacaggagcaagtgctctcaacaaggtcctgggaagagaggtctacacatccaacaaccagctgggtggcgttcagatcatgcattacaatggtgtctcccacatcaccgtgccagatgactttgagggggtttataccatcctggagtggctgtcctatatgccaaaggataatcacagccctgtccctatcatcacacccactgaccccattgacagagaaattgaattcctcccatccagagctccctacgacccccggtggatgcttgcaggaaggcctcacccaactctgaagggaacgtggcagagcggattctttgaccacggcagtttcaaggaaatcatggcaccctgggcgcagaccgtggtgacaggacgagcaaggcttggggggattcccgtgggagtgattgctgtggagacacggactgtggaggtggcagtccctgcagaccctgccaacctggattctgaggccaagataattcagcaggcaggacaggtgtggttcccagactcagcctacaaaaccgcccaggccgtcaaggacttcaaccgggagaagttgcccctgatgatctttgccaactggagggggttctccggtggcatgaaagacatgtatgaccaggtgctgaagtttggagcctacatcgtggacggccttagacaatacaaacagcccatcctgatctatatcccgccctatgcggagctccggggaggctcctgggtggtcatagatgccaccatcaacccgctgtgcatagaaatgtatgcagacaaagagagcaggggtggtgttctggaaccagaggggacagtggagattaagttccgaaagaaagatctgataaagtccatgagaaggatcgatccagcttacaagaagctcatggaacagctaggggaacctgatctctccgacaaggaccgaaaggacctggagggccggctaaaggctcgcgaggacctgctgctccccatctaccaccaggtggcggtgcagttcgccgacttccatgacacacccggccggatgctggagaagggcgtcatatctgacatcctggagtggaagaccgcacgcaccttcctgtattggcgtctgcgccgcctcctcctggaggaccaggtcaagcaggagatcctgcaggccagcggggagctgagtcacgtgcatatccagtccatgctgcgtcgctggttcgtggagacggagggggctgtcaaggcctacttgtgggacaacaaccaggtggttgtgcagtggctggaacagcactggcaggcaggggatggcccgcgctccaccatccgtgagaacatcacgtacctgaagcacgactctgtcctcaagaccatccgaggcctggttgaagaaaaccccgaggtggccgtggactgtgtgatatacctgagccagcacatcagcccagctgagcgggcgcaggtcgttcacctgctgtctaccatggacagcccggcctccacctgaccgtggcccgcccagccactcccgggaccacggcaaaaggaaccacccagacccaccacccgtacaccctcagcagaccctgaagacttgcttttaaacaaagaaaatcctgggcacttctgcagggctgctggttccgagctgacacccgtcttaacaaaaggcccaggagtgcctcttccaaacaaaaacagcctcctctccatagctgggaagtttattttgttttgtctctgaagacagcagttttattgcatcactaaatctaatcaagctaaaacatccctgtttccttttgcaaaacagtgcctggcatgtgggatccaggcgttctttaggatccttggataccacatcgtgaaatcttttatttttttactctgagaccagcaccagatgtaagtaagcatctcatatatttcagccaaataaatgggccaagggaaaaaaatatatatatatagacaggactagagaaaacctatttttgtaatgatgtttctttggatactgtctagtcacccagaaaaatgtatggatgaatttttttttttttttttgagacaaagtctcactgtgtcatgcaggctggagtgcagtggcatgatctcactgcaacctccatctcctgtctcagcctcctagataactgggattacaggtgcccaccaccatgcccggctaatttttgtatttttggtagagacagagtttcaccaggttggtcaggctggtctcaaactcttgacttcaggtaatccacccaccttggcctcccaaagtgctgggattacaggcatgagccaccatcttcagccagatgatttttttattgagagagtgaaatgctattttgttccccaaatggcgctagtgaatcactaggagggtcccactgataggccatgtttagcactggttgccagggattctctttttgagagagggaaagcaaaatgaatggaagtacccagctggaggtttcagggcttctggaggatgctctcgcatagctcgaggtcctctgcccacctcttctctccaaggaaaatgaggactgccccttccccctgcaggattggcccccagcctgcgcatgcaccctcctcttgcccaagtggggagcacagaggcggagaggaatcccttaccacacccacggcccagcttgctcacgagtgtcacctctgtgacggtcaccactgctcccttggagggccacttgagttactgttgcttcctcgcctgctggcttgatgagcaccgatggtgggatctgaccccgaggggcagagctgtcggtgactgaggactggactgtggtgaccatgccgatttgctcagggagaacgttgcaatgcacccagcagctcctggctctgcaggcggcacagcctggggccctgtgatcctctggtttcttccattggggcggagtcggggatggagggagctggccacaacccactgctctgatgggtggtttgtccaaggatgctgaatgtaatgcctggtcaatgtggaagcccatgaggttgcccagggaagcctccaaaagctgggatgcttgagggtatccaagttgaaaaagacaaaatctgaccatcagccagtgacagtcctggcaaatgaaggtggggcggggcagtgaggggtgggagaaggtgaatgattcattattccaccccgaggtttgctggggtgaggggaagaatcgatgctgctttgggaactgaaggtttttctgttgggaaggccctcttggttttggagagaaagacaagttatgagtagctgctaccctggaacggtgggcagagagcctactaggaaatgtgcagaataaactattttttgaaggaaa

SEQ ID NO: 152 - DNA, 9816 bp, Macaca fascicularis
gcccctcggctgtggaggcccccagcgggtgctgagcgccagccgctgcccccttggccttttcccggccaccgcccggcgcccgtcgctgtccctgtgcctgaggactgggaggatggggctaagccgcttgcctgacttttgatccgccaagtaatcactttgtccgtgtggcgcgtggcccgttgccggaggcttcccagcgcgtggggccgtttggtcccctccggagtgcggtgaggcgggccaagccgggactgcctgggtttggggataacgttcccacccccacccctgttgcagcaagggaaattgaggctgagggaactgggcccagggacggcgagccgtggctgcctctccagtccgggccctgaagggctgctcgtggatgaaccagactgatttttaaggggtgaagagggtgcgtttcaatcagatgcttctggaacgtcgaaattgtcttctttggaaagaaccatcccctctttgggcttcagaggcccaaattgaggcgcgatgatgagaggatgtggtggtctactctgatgtcaatcttgagggctagctctaagagctcattttggaggaataatggatgaaccatctcccttggccaaacctctggagctgaaccagcactctcgattcataattggttctgtgtctgaagataactcagaggatgagatcagcaacctggtgaagctggacctactggaggagaaggagggttctttgtcacctgcttctgttggctcagatacactctctgatttggggatctctagcctacaggatggcctggccttgcacataaggtccagcatgtctggcttgcacctagtaaagcagggccgagacagaaagaaaatagattctcaacgagatttcactgtggcttctccagcagaatttgttactcgctttgggggaaataaagtgattgagaaggttcttattgctaacaatggcattgcagcagtgaaatgcatgcggtctatccgtaggtggtcttatgaaatgtttcgaaatgaacgtgcaattcgattcgttgtcatggtcacacctgaagaccttaaagccaatgcagaatacattaagatggcagatcactacgtgccggtgcctggaggaccaaacaacaacaactatgcaaatgtggaattaattcttgatattgctaaaaggatcccagtacaagcagtgtgggctggctggggtcatgcttctgagaatcccaaactaccggaacttctcttgaaaaatggcattgccttcatgggtcctccaagccaggccatgtgggctttaggggataagattgcatcttccatagtggctcaaactgcaggtatcccaactcttccctggagtggcagtggtcttcgtatggactggcaggaaaatgatttttcaaaacgtatcttaaatgttccccaggagctatatgaaaaaggttatgtgaaagatgtggatgacgggctacaggcagctgaggaagttggatatccagtaatgatcaaggcctcagaaggaggaggagggaagggaattagaaaagtcaacaatgcagatgacttccctaacctcttcagacaggttcaagctgaagtccctggatctcccatatttgtgatgagactagccaaacaatctcgtcatctggaggtgcagatcttagcggaccaatatggcaatgccatctctttgtttggtcgtgattgctctgtacaacgcaggcatcagaagattattgaagaggcaccagctactattgctactccagcagtatttgaacacatggaacagtgtgcggtgaaacttgccaaaatggtgggttatgtgagtgctgggactgtggaatacctgtacagccaggacggcagcttctactttctggaattgaatcctcggctacaggtagagcacccttgtacagagatggtggctgatgtcaatctccctgcagctcagctccagattgccatggggattcctctatatagaatcaaggatatccgtatgatgtacggggtatctccctggggtgattctcccattgattttgaagattctgcacatgttccttgcccaaggggccatgttattgctgctcggatcactagtgaaaatccagatgagggttttaagcccagttcaggaacagttcaggaactaaatttccgcagcaataagaatgtttggggatacttcagtgttgctgctgcagggggacttcatgaatttgctgattctcagtttggtcactgcttttcttggggagaaaacagagaggaggcaatttcaaacatggtggtggctttgaaggagctgtctattcggggtgactttcgaactacagttgaatacctgatcaaattgttagagactgaaagctttcagatgaacagaattgatactggctggctggacagactgatagcagaaaaagtacaggctgagcgacctgacaccatgttgggggttgtatgtggggccctccacgtggcagacgtgagcctgcggaatagcgtctctaacttccttcactccttagaaaggggtcaagtccttcctgctcatacactgttgaatacagtagatgttgaacttatctatgagggagtcaagtatgtacttaaggtgactcgacagtcccccaactcctatgtggtgatcatgaatggctcatgtgtagaagtagatgtacatcggctgagtgacggtggactgctcttgtcctatgatggcagcagttatactacgtacatgaaggaggaagtggatagatatcgcatcacaattggcaataaaacctgtgtgtttgagaaggaaaatgacccatcagtgatgcgctcaccttctgctgggaagttaatccagtacattgtagaagatggaggtcatgtgtttgctggccagtgctatgctgagattgaggtgatgaagatggtaatgaccttaacagctgtggagtctggctgtatccattacgtcaagcgacctggagcagctcttgaccctggctgtgtactagccagaatgcaactggacaaccccagcaaggttcagcaggctgagcttcacacaggtagtctgccacggatccagagcacggcactcagaggcgagaaactgcatcgagtgttccattatgtcctggataatctggtcaacgtaatgaatggatactgccttccagatcctttctttagcagcaaggtaaaagactgggtagagcgattgatgaaaaccctcagagatccctccctgcctctcctagaattgcaagatatcatgactagcgtgtctggccgcattccccccaatgtggagaagtctatcaagaaggaaatggctcagtatgctagcaacatcacatcagtcctctgtcagtttcccagtcagcagattgccaacatcctagatagccatgcagctacattgaaccggaaatctgaacgggaggtcttctttatgaatactcagagcattgttcagctggtacagaggtaccgaagtggcatccgaggccacatgaaggctgtggtgatggatctgctccggcagtacctgcgagtagagacacaattccagaatggtcactatgacaaatgtgtattcgccctccgggaagagaataaaagtgacatgaacactgtactgaactacatcttctctcacgctcaagtcaccaagaagaatcttctggtcacaatgcttatcgatcagttgtgtggccgggaccctactctcactgatgagctgctgaatattctcacagagctaactcaactcagtaagactaccaatgctaaagtagcacttcgagcacgccaggttcttattgcctcccatttgccatcatacgagcttcgccataaccaagtagagtctatcttcctatcagctattgacatgtatggacatcaattttgcattgagaacctgcagaaactcatcctatcagaaacatctatttttgatgtcctaccaaacttcttctatcacagcaaccaagtagtgaggatggcagctctggaggtgtatgttcgaagggcttatattgcctacgaacttaacagcgtacaacaccgccagcttaaggacaacacctgtgtggtggaattccagttcatgctgcccacatctcatccaaacagagggaacatccctacgctaaacagaatgtccttctcctccaacctcaaccactatggcatgacccatgtagctagtgtcagcgatgtgctgttggacaactcattcactccaccttgtcagcggatgggcggaatggtctcttttcggactttcgaagattttgtcaggatctttgatgaagtgatgagctgcttctctgactccccaccccagagtcccacattccctgaggcaggtcacacgtctctttatgatgaagataaggttcctagggatgaaccaattcacattctcaatgtggctatcaagaccgactgtgatattgaggatgacaggctggcagctatgttcagagaatttacccagcaaaataaagctaccctggttgaccatgggatccggcgccttactttcctggttgcacaaaaggatttcagaaaacaggtcaactatgaggtggatcggagatttcatagagaattccctaaattttttacattccgagcaagggataagtttgaggaggatcgtatctatcgtcatctggagcctgctctggctttccagttagagctgaaccggatgagaaattttgacctcactgccattccatgtgctaatcacaagatgcacctgtatctcggggcagccaaggtggaagtgggcacagaagtgacagactacaggttctttgttcgtgcaatcatcaggcattctgatctggtcaccaaggaagcttcttttgaatatctgcaaaatgaaggggagcggctactcctggaagccatggatgagttggaagttgcttttaacaatacaaatgtccgcactgactgtaaccacatcttcctcaactttgtgcccacggttatcatggacccatcaaagattgaggaatctgtgcggagcatggtaatgcggtatggaagtcgcctgtggaaattgcgcgtcctccaggcagaactgaaaatcaacattcgcctgacgccaactggaaaagcaattcccatccgcctcttcctgacaaacgagtctggctattacttggatatcagcctatacaaggaagtgactgactccaggacagcacagatcatgtttcaggcatatggagacaaacagggaccacttcatggaatgttaatcaatactccttatgtgaccaaagacctgctgcaatcaaagaggttccaggcacaatccttagggacaacatacatatatgatatcccagagatgtttcggcagtccctgatcaaactctgggagtctatgtccactcaagcatttcttccatctccccctctgccttctgacatgctgacttacaccgaactggtattggatgatcaaggtcagctggtccacatgaacaggcttccaggaggaaatgagattggcatggtagcttggaaaatgacctttaaaagtcctgaatatccagaaggccgagatatcattgttattggcaatgacatcacataccgaattgggtcctttgggcctcaagaggattggttatttctcagagcttctgaacttgccagggcagaaggtattccacgcatctatgtatcagccaacagtggagcaagaatcggactggcagaagaaattcgtcatatgtttcatgtggcctgggtagatcctgaggatccttacaagggatacaggtatttatatctgacccctcaagattataagagagtcagtgctctcaactctgtccattgtgaacacgtggaggatgaaggagaatccaggtacaagataacagatattattgggaaagaagagggaattggacctgagaaccttcgaggttctggaatgattgctggagaatcctcattggcctataatgagatcattaccatcagcctggtgacgtgccgggccattgggattggggcttaccttgtccggctgggacagagaaccatccaggttgagaattctcacttaattctaacaggagctggagccctcaacaaagtcctcgggcgggaagtgtacacctccaataaccagctcgggggcatccagatcatgcacaacaatggggtgacccactgcactgtgtgtgatgactttgaaggggttttcactgtcctgcactggctgtcttacatgcccaagagcgtgcacagttcagttcctcttctgaactcaaaggatcctatagacagaatcatcgagtttgttcccacaaaggccccatatgatcctcgatggatgctagcaggccgtcctcatccaacccaaaaaggtcaatggttgagtggcttttttgactatggatctttctcagagattatgcagccctgggcacagactgtggtggttggtagagccaggctaggaggaatacctgtgggagttgttgctgtagaaacccgaacagtagagctaagtatcccagctgatccagcaaacttggattctgaagccaagataatccagcaggctggccaggtttggttcccagattctgcgtttaagacgtatcaggccatcaaggacttcaaccgggaagggctgcctctgatggtctttgccaactggagaggcttctctggtggaatgaaagatatgtacgaccaagtgctgaagtttggtgcttacattgtggacggcttgagggagtgctcccagcctgtgctggtttacattcctccccaggctgagctgcggggcggctcctgggtagtgattgactcctccatcaatccccggcacatggagatgtatgctgaccgagaaagcaggggatctgttctggagccagaagggacagtagaaatcaaattccgcagaaaggatctggtgaaaaccatgcgtcgggtggacccagtctacatccacttggctgagcgattggggaccccggagctaagcacagctgaacggaaggagttggagaacaagttgaaggagcgggaggaattcctaattcccatttaccatcaggtagccgtgcagtttgctgacttgcacgacacaccaggccggatgcaggagaagggtgttattagcgatatcctggattggaagacatcccgtaccttcttctactggcggctgaggcgtctcctgctggaggacctggtcaagaagaaaatccacaatgccaaccctgagctgactgacggccagattcaagccatgttaaggcgctggtttgtggaagtggaaggtacagtgaagcttggtccaggccaatccagaggtcgccatggattccatcatccatatgacccagcacatatcacctactcagcgagcagaagttgtacggatcctctccacaatggactccccttccacgtaggaaaagcttcctgcctgtccctgccctgtctctggagaaaagggctagagctgccttttacaactgcagccactgtaatgagaaggcacaggagacccagcactggagtcaaagtggcattttacttcctcccaagtgtttcaggttacgcatgacatcctgggatgtaagatcacagaacccctccagcccaccagtcacacccaccccattcagtatttattaccccagccaggcctagtcctccactccctgcacaggactgagaaggcaatgaaaggtacaaacatgtaccatgaggtcttactacccaaagcagggctgcccctcctgtcctgacagccccttggcccccagcatggggaagcgtgaggagttggcgttgccaggcagcagcccctctcacccctggccccatgagccgcagccacaggcagcagaggagggctaaggagaggaggaagcctcaagtccattgtttattaccctgactcttagcccagcacacagtaggcactggagaggaatgatgcccagtttaaccacaccatggtaccttttatgacgaaaaattagagcataaaatctatcacaagctccataggaacgcaaagatgagggcaaaactgtgagccaagaagcagagaaagaaaatagacccagttattcttgatttaggggacttcaaccttgggttcagtctctgaggacagagggaaaggtagttggcctacctctcacctgcatgttactgctggactagctgttgcatgtggctgggagctgcaaggccccagtgcgtgaggggccccagcagttccataggcggtgaagcctgagttggcagaggaggagccagagagaactgctctttctgcactggtggaaactagttacttatgccatgtggagggccagccacatagtctgttttgaggacttagaaagttgttcctatgaagcctggagcttggatggttttgaggggttaatggtgcctccacagtcactcttccctagttccaggattactgtcctagcagctaacgtttctactgtcttccccagaatgtagacaggcagcagatgtagcagctctccccacagttctgaaaggaccctggtgacagccacgccctcagcaccaggagctggccttcccgatgagggaggcttccaggaaacacagaatccacacgaccttaagattctttacaactcagtcatggtgctgctgtcctccaggcttactggcccctcctgaccggcatcgggggcttcctcaggtggtagagagagtttactttcaacaactactttattcaagaaaagaacttactgattcccctgttcctaaagcaagagtggcaggtgatcagggctggtgtagcatccagttcctttagtgcagctaattgcatttgtcactgacgaccaaggaggaaatcactaaggcgtttgagaagcagtggtatgaacgctcttggacgagctacagttctgagccttaaccctgtagttcgcacacaagagcggagctccacctccccttcttcgggaggaatctgtgcagatggattggctgaacttaatggttctgggttgcaggtgggtactgtatggctgggtatggagcggactgcccccaggagtcagagcctcagcctggctgccctggtggaaggtgtgggtgttcagcaccttcagagaagggcataaagtggtgggggacaattctcagtccaggaaaatgcattgaccattgctggctatttgcttgcctagtaagaattggattcatttttgaccagattattcttctatgcttttttgcaataaatcaaatcccacatatctacaagtggtatgaagtcctgcaccccccaggaggcctgtccaggcatctcttcagaggcagggtgggttacactcatttacctcccctctccccaccaaattatgacacaaatgagtatgtttcctctctagaaccctgtaatgcctcctccccggtccccagagctcctcactgtcggtctcaccctggacaaggattttttagtcagagccacagaacatgggcactctgacattcccacagcccctcgcacatgcgaggctaccaaggcaggagagtataaatgatggctattgaccaagacctgcctggacggggactgccatattatctgttctcttcgttcacaaaacagccttcatttgtctcagaattcaatggacgcataccgtgatgagcaggagcttcagatgcactctttacacattttgttgaaataaacctctacatttgtagaaga

SEQ ID NO: 153 - DNA, 9967 bp, Macaca fascicularis
tttaaaccgttttgcacagatcccgtgctcagcgctctcgccagtttctccacttgctgtttagtaaggccatccttgttggcaggaagtggggccactgaactgctgttcccccttccctgttgcctgagcacaatgtgctcctatgtggtctccgctttcctgctgcctagttggctgggctgctccagctgggccttagaggcgacctttgtcccacatgccagccctccccacgttgcagaacactgtgtaactgcagtgtgtcctgaatttcctcttctctgctcaaccctagcaagagccagtcgaccctgaatgagaatgtgggaatggagctgtttttttcttttatccccacgtcacaagaaattggaaacattccattaagtggcgtgattcgagagagtttcgccagatcttttcaggggagagaagccaggtgacctgatagccatcatcaccttcccattcacggttttcaccgggggcttctcaaataaccaacacctgtaggcagttaattttcagcatgtcacaggagggcccaaatgactttgtgcctaatgcttacgacaacatggactttagattcctcgtggtttcaatagcgaagaattatgaaataaagcgagttatgggattaaaaagcagatagcagaactcccccaggagtagccagggctgcgcaagtggtgtggcgtgggatctttcttgtctttgccctgggttgttgctgctttgaaggttaagtgctccagggatatttttaaacagcaggagctggttaatctaaaagtgacatttgcagttcccacagtaagcagctagcaggcttagattcaggccctcagcaaacacggaacctggaaaatgtaaccctgaatgcacggttgggaggacatggcaagagaaaagcagcaggaagaaagtgattttctgaatggtctttcttctttgtctatcttgtctgattttctcctgtctgaccttttcctggttaaaaatctgggggaaaatgacggactcccagccaatcaccaagagtacatcagaagcaaacttcatcctgagccaggagccctttccagcctctgacaactcaggggagacaccgcagagaaatggggaggaccactctctgcccaagactcccagccaggccgagccagcctcccacaaaggccccaaagatgctggtcggcggagaaactccctgccaccctcccaccagaagcccccaaaaaaccccctttcttccagtgacgcagcaccctccccagagctccaagccaacgggaccgggacacaaggtctggaggccccagataccaatggcctgtcttcctcagccaggccccagggccagcaagctggctccccctccaaagaagacagcaagcaggcaaacatcaagaggcagctgatgaccaacttcatcctgggctcttttgatgactactcctccgacgaggactctgttgccggctcatctcgtgagtctaccaggaagggcagccgggccagcttgggggccctgtccctggaggctgctctgaccgcaggtgaagctgagacccgtgtccccactatgaggccgagcatgtcaggactccacctggtgaagaggggacgggaacacaagaagctggacctgcacagagactttacagtggcttctcccgctgagtttgtcacacgctttggcggggatcgggtcatcgagaaggtgcttattgccaacaacgggatcgccgccgtgaagtgcatgcgctccatccgcaggtgggcctatgagatgttccgcaacgagcgggccatccggtttgttgtgatggtgacccctgaggaccttaaggccaacgcagagtacatcaagatggcggatcagtacgtccccgtcccaggagggcctaataacaacaactatgccaacgtggagctgattgtggacattgccaagagaatccctgtgcaggcggtgtgggctggctggggccatgcttccgaaaaccctaaacttccggagctgctgtgcaagaatggagttgctttcttaggccctcccagtgaggccatgtgggccttaggagataagatcgcctccaccattgtcgcccagacgctgcaggtcccaaccctgccctggagtggaagcggcctgacagtggagtggacagaagatgatctgcagcagggaaaaataatcagtgtcccagaagatgtttatgaccagggttgtgtgaaagacgtagatgagggcttggaggcagcagaaaaaattggttttccattgatgatcaaagcttctgaaggtggcggagggaagggaatccggaaggctgagagtgcggaggacttcccgatccttttcagacaagtacagagtgagatcccaggctcgcccatctttctcatgaagctggcccagcacgcccgtcacctggaagttcagatcctcgccgaccagtatgggaatgctgtgtctctgtttggtcgcgactgctccatccagcggcggcatcagaagattgttgaggaagcaccggccaccattgccccgctggccatattcgagttcatggagcagtgtgccgtccgcctggccaagaccgtgggctatgtgagtgcagggacagtggaatacctctacagccaggacggcagcttccacttcttggagctgaatcctcgtttgcaggtggaacatccctgcacggaaatgatcgctgacgtcaatctgccggctgcccagctacagatcgccatgggcgtgccactgcaccggctgaaggatatccggcttctgtacggagagtcgccatggggagtgactcccatttctttcgaaaccccgtcaaaccctcccctcgcccgaggtcacgtcattgccgccagaatcaccagtgaaaacccagatgaggggtttaagccgagctccgggactgtccaggaactgaatttccgcagcagcaagaacgtgtggggttacttcagtgtggctgctactggaggcctgcatgagtttgcggattcccagtttgggcactgcttctcctggggagagaaccgggaagaggccatttcgtcagtatctctctcccttccttcttttctttccttccttccttccttccttccttccttccttccttccacccaggcggtgacaaggggcctgtccccacaggtggcccggcagtctctgaccatgttcgttctcatcatgaatggctgccacatcgagattgatgcccaccggctgaacgatggggggctcctgctctcctacaatgggaacagctacaccacctacatgaaggaagaggttgacagttaccgaattaccatcggcaataagacatgtgtgtttgagaaggagaatgatcctacggtcctgagatccccctcggctgggaagctgacacagtacacagtggaggacgggggccacgttgaggctgggagcagctacgctgagatggaggtgatgaagatgatcatgactctgaacgtgcaggaaagtggccgggtgaagtacatcaagcgtccaggggccgtgctggaagcaggctgcgtggtggccaggctggagcttgatgacccttctaaggtccacccggctgaaccgttcacaggagaactccctgcccagcagacactgcccatccttggagagaaactgcaccaggtcttccacagcgtcctggaaaacctcaccaacgtcatgagtggcttttgtctgccggagcccatttttagcataaagttgaaggagtgggtgcagaagctcatgatgaccctccggcatccgtcactgccgctgctggagctgcaggagatcatgaccagcgtggcaggccgcatcccggcccccgtggagaagtcagtccgcagggtgatggcccagtatgccagcaacatcacctcggtgctgtgccagttccccagccagcagatagccaccatcctggactgccacgcggccaccctgcagcggaaggctgaccgagaggtcttcttcctcaacacccagagcatcgtgcagttggtccagagataccgcagcgggatccgcggctatatgaaaacagtggtgttggatctcctgagaagatacttgcatgtcgagcaccattttcagcaagcccattacgacaagtgtgtgataaacctcagggagcagttcaagccagacatgtcccaggtgctggactgcatcttctcccacgcacaggtggccaagaagaaccagctggtgatcatgctgatcgatgagctgtgtggcccagacccttccctatcggacgagctgacctccatccttaatgagctcactcagctgagcaaaagcgagcactgcaaagtggccctcagagcccggcagatcctgattgcctcccacctcccctcctatgagctgcggcacaaccaggtggagtccattttcctgtctgccattgacatgtacggccatcagttctgccccgagaacctcaagaagttaataatttcagaaacaaccatcttcgacgtcctgcctgctttcttctatcacgctaacaaagtcgtgtgcatggcatccttggaggtttacgtgcggaggggctacatcgcctatgagttaaacagcctgcagcaccggcagctcccggacggcacctgcgtggtagaattccagttcatgctgccctcctcccacccaaaccggatgaccgtgcccatcagcatcaccaaccctgacctgctgaggcacagcacagagctcttcatggacagcggcttctccccactgtgccagcgcatgggggccatggtagccttcaggagattcgaggacttcaccagaaattttgatgaagtcatctcttgctttgccaacgtgcccaaagacacccccctcttcagcgaggcccgcacctccctgtactccgaggatgactgcaagagcctcagagaagaccccatccacattctgaatgtgtccatccagtgtgcagaccacctggaggatgaggcactggtgccgattttacggacgttcgtacagtccaagaaaaatatccttgtggattacggactccgacgaatcacattcctgattgcccaagagaaagaatttcccaagtttttcacattcagagcaagagatgagtttgcagaagatcgcatttaccgtcacttggagcctgccctggccttccaactggagctcagccggatgcgtaacttcgatctgaccgccgtgccctgtgccaaccacaagatgcacctttacctgggggctgccaaggtgaaggaaggggcggaagtgacggaccataggttcttcatccgcgccatcatcaggcactcagacctgattacaaaggaagcctccttcgagtacctgcagaacgagggtgagcggctgctcctggaggccatggacgagctggaggtggcgttcaacaacaccagcgtgcgcaccgactgcaaccacatcttcctcaacttcgtgcccactgtcatcatggaccccttgaagatcgaggagtccgtgcgctccatggttatgcgctacggcagccggttgtggaaactccgcgtgctgcaggctgaagtcaagatcaacatacgccagaccaccaccggcagcgctgttcccatccgcctgttcatcaccaatgagtcgggctactacctggacatcagcctctacaaagaagtgactgactccagatccggaaatatcatgtttcactcctttggcaacaagcaagggccccagcacgggatgctgatcaatactccctacgtcaccaaggatctgctccaggccaagcgattccaggcccagtccctgggaactacctacatctatgacttcccagaaatattcagacaggctctctttaaactatggggctccccagacaagtatcccaaagacatcctgacatacactgaattagtgttggactctcagggccagctggtggagatgaaccgacttcctggtggaaatgaggtgggcatggtggccttcaaaatgaggtttaagacccaggagtaccctgaaggacgggacatgatcgtcatcggcaacgatatcacctttcgcatcggatcctttggccctggggaggaccttctatacctgcgggcatctgagatggcccgggcagagggcattcccaaaatctacgtggcagccaacagtggcgcccgtattggcatggcagaggagatcaaacacatgttccacgtggcttgggtggacccagaagacccccacaaaggatttaaatacctgtacctgactccccaagactacaccagaatcagctccctgaactctgtccattgtaaacacatcgaggaaggaggagaatccagatacatgatcacggatatcatcgggaaagatgatggcttgggcgtggagaatctgaggggctcaggcatgattgctggggagtcctctctggcttacgaagagatcgtcaccattagcttggtgacctgccgagcccttgggatcggggcctacttggtgaggctgggccagcgagtgatccaggtggagaactctcacatcatcctcacgggagcaagtgctctcaacaaggtcctgggaagagaggtctacacatccaacaaccagctgggtggcgttcagatcatgcattacaatggcgtttcccacatcaccgtgccagatgactttgagggggtttataccattctggagtggctgtcctatatgccaaaggataatcacagccctgtccctatcatcacacccactgaccccattgacagagaaattgaattcctcccatccagagctccctacgacccccggtggatgcttgcaggaaggcctcacccaacgctgaagggaacgtggcagagcggattctttgaccatggcagtttcaaggaaatcatggcaccctgggcgcagaccgtggtgacaggacgagcaaggcttggggggattcccgtgggagtgattgctgtggagacgcggactgtggaggtggtggtccctgcagaccctgccaacctggattctgaggccaagataattcagcaggcagggcaggtgtggttcccagactcagcctacaaaaccgcccaggccatcaaggacttcaaccgggagaagttgcccctgatgatctttgccaactggagggggttctccggtggcatgaaagacatgtatgatcaggtgctgaagtttggagcctacatcgtggatggcctcagacaatacaaacagcccatcctgatctatatcccaccctatgcggagctccggggaggctcctgggtggtcatagatgccaccatcaacccgctgtgcatagaaatgtatgcggacaaagagagcagggggggtgttctggagccagaggggacagtggagattaagttccgaaagaaagatctgataaagtccatgagaaggatcgatccagcttacaagaagctcatggaacagctaggggaacctgatctctcagacaaggaccgcagggacctggagggccggctgaaggctcgcgaggacctgctgctccccatctaccaccaggtggcggtgcagttcgccgacttccacgacacgcccggccggatgctggagaagggcgtcatatctgacatcctggagtggaagaccgcgcgcaccttcctgtattggcgtctgcgccgcctcctcctggaggaccaggtcaagcaggagatcctgcgggccagcggggagctgagtcacgtgcacatccagtccatgctgcgccgctggttcgtggagacggagggggctgtcaaggcctacctgtgggacaacaaccaggtggttgtgcagtggctagaacagcactggcaggtggaggacggcccgcgctccaccatccgcgagaacatcacgtacctgaagcacgactctgtcctcaagaccatccgaggcctggttcaagaaaaccccgaggtggccgtggactgtgtgatatacctgagccagcacatcagcccagctgagcgggcacaggtcatccacctgctgtctaccatggacagcccggcctccacctgaccccggcctgccccgccattcccgggaccacagcaggaggaaccacccacacccaccatcggtacgccctcagcagaccttgaagacctgcttttaaaaaaagaaaatcctgggcacttctgcagggctgctggttccgagctgacacctgtctcaataaaaggcccaggagtgcctctcccaaacacaaacaacctcctctgcatagctgggaagtttcttttgttttgtctctgaagacagcatttttattgcatcactaaatctaatcaagctaaaacatccctgtttccttttgcaaaacagtgcctggcctgtgggatccaggcgttcttcaggcttcttggatatcatatcatgaaatcttttatttttttactctgagaccagcactagatgtaagcatctcatatatttcagccaaataaatgggccaaggaaaaaaaatatatatatatagacaggactagagaaaaacctatttttgtaatgatgtttctttggatactgtctagtcacccaggaaaatgtatggatgaaatttttttttttttttttttgagacagagtcttactctgtcacctaggctggaatggggtagcatgatctcactgcaaccttcatctcccaggttcaagctgttctcctgtctcagcctcctagatagctgggattacaggtgcccatcaccatgtccagctaattcttgtatttttagtagagacacagtttcaccatgtaggtcaggctggtcttgaacccttgacttcaggtaatccacccaccttggccccccaaagtgctgggattacaggcgtgagccaccatctttggccagatgattttttattgcaagaatgaaacggcattttgttccccaaatggccctagtgaatcactaggagggctccactggtaggccatgtttagcactggttgccagggattctctttttgagagagggaaagcaaaatgaatggaagtgcctggccagaggtttcagggcttctggaggatcctctcgcatagctcgaggtcctctgcctgcctcttccctccaaggaaaatgaggactgccccttccccctgcaggattggcccccagcctgtgcaggcacccttctcttgcccaagtggggagcacagaggcggagaggaatcccttgccacacccatggcccagcttgctcacaagtgtcacgtctgtgacggtcaccactgctcccttggagggccacttgagtcactgttgctcccttgcctgctggcttgatgagcacagatggtgggatctgaccgaggggcagagctgtcggtgactaaggactggactgtggtgaccataccgatttgctcagggaggatgttgcaatgcatccagcagctcctggctctgcaggcggcacagcccggggccctgcaatcctctggtttctcccattggggtggagtgggggagtggagggagttggccacaacccactgctgtgatgggcggtttgtccaaggatgctgtatatgatgcctggtcaatgtggaagcccatgaagtcacccagggaagcctccaaaagctgggatgcttgagggtatccaagttgaaaaagacaaaatctgaccattagccagtgacggccctggcaaatgaaggtgtggtggggcagtgagggatgggagaaggtgaattattccttattccaccccgaggtttgctggggtgaggggaagaattgatgctgctttgggaactgaaggtttttctgttgggaaggccctcttggttttggagagaaaggcacgttatgagtagctgctaccctggaacggtgggcagagagcctaccaggaaatgtgcagaataaactattttttgaaggaaa

SEQ ID NO: 154 - DNA, 9054 bp, Mus musculus
ctctgagagcttattttgaaagaataatggatgaaccatctccgttggccaaaactctggagctaaaccagcactcccgattcataattgggtctgtgtctgaagacaactcagaagatgagatcagtaacctggtgaagctggacctagaagagaaggagggctccctgtcaccagcctccgtcagctcagatacactttctgatttggggatctctggcttacaggatggtttggcctttcacatgagatccagcatgtctggcttgcacctagtaaaacaaggtcgagacagaaagaaaatagactcacaacgagatttcactgtggcttctccagcagaatttgttactcgttttgggggaaataaagtaattgagaaggttcttatcgccaacaatggtattgcagcagtgaaatgcatgcgatctatccgtcggtggtcttatgaaatgtttcgaaatgaacgtgcaatccgatttgttgtcatggttacacctgaagaccttaaagccaatgcagaatacattaagatggcagaccactatgttccagtgcctggaggacccaacaacaacaattacgcaaatgtggagttgattcttgatattgctaaaaggatacctgtacaagcagtgtgggctggctggggtcatgcctctgagaacccgaaactcccagaactgctcttaaaaaatggcattgctttcatgggccctccaagccaggccatgtgggctttgggggataagattgcatcttctattgtggctcaaactgcaggtatcccaactcttccctggagtggcagtggtcttcgagtggattggcaagaaaatgatttttcgaaacgtatcttaaatgttccacaggatctgtatgagaaaggctatgtgaaggatgtggatgatggtctgaaggcagctgaggaagttggctatccagtgatgatcaaggcctcagagggaggaggagggaaagggatcagaaaagttaacaatgcagatgacttccctaacctcttcagacaggttcaagctgaagttcctggatcacctatatttgtaatgagactagcaaaacaatctcgacatctggaggtccagattctggcagatcagtatggcaatgctatttctttgtttggtcgtgactgctctgtgcagcgcaggcatcagaagatcattgaagaagctcctgctgcgattgctaccccagcagtatttgaacacatggaacagtgtgcagtgaaacttgccaaaatggttggttatgtgagtgctgggactgtggaatacttgtacagccaggatggaagcttctactttttggaactgaaccctcggctacaggttgaacatccttgtacagagatggtggctgatgtcaatcttcctgcagcacagctccagattgccatggggatccctctatttaggatcaaggatattcgtatgatgtatggggtatctccttggggagatgctcccattgattttgaaaattctgctcatgttccttgcccaaggggccacgtgattgctgctcggatcaccagtgaaaacccagatgaggggtttaagcccagctctggaacagttcaggaacttaattttcgtagcaataagaacgtttggggttatttcagtgttgctgctgctggaggacttcatgaatttgctgattctcagttcgggcactgcttttcctggggagaaaacagggaggaagcaatctcaaatatggtggtggcactgaaggagctgtctattcggggtgactttcgaactacagtggaatacctcatcaaactgctggagacagaaagctttcagcttaacagaatcgacactggctggctggacagactgatcgcagagaaagtgcaggcagagcgacctgacaccatgttgggagttgtgtgtggggctctccatgtagcagatgtgagcctgaggaacagcatctctaacttccttcactccttagagaggggtcaagtccttcctgctcacacacttctgaacacagtagatgttgaacttatctatgaaggaatcaaatatgtacttaaggtgactcggcagtctcccaactcctacgtagtgataatgaatggctcgtgtgtggaagtggatgtgcatcggctgagtgatggtggcctgctcttgtcttatgacggcagcagttacaccacatacatgaaggaagaggtggacagatatcgaatcacaattggcaataaaacctgtgtgtttgagaaggaaaatgacccatctgtaatgcgctcaccgtctgctgggaagttaatccagtatattgtggaagatggcggacatgtgtttgctggccagtgctatgctgagattgaggtaatgaagatggtgatgactttaacagctgtagaatctggctgcatccattatgtcaaacgacctggagcagcacttgaccctggctgtgtgatagccaaaatgcaactggacaaccccagtaaagttcaacaggctgagcttcacacgggcagtctaccacagatccagagcacagctctcagaggcgagaagctccatcgagttttccactatgtcctggataacctggtcaatgtgatgaatggatactgccttccagaccctttcttcagcagcagggtaaaagactgggtagaaagattgatgaagactctgagagacccctccttgcctctgctagagctgcaggatatcatgaccagtgtctctggccggatccccctcaatgtggagaagtctattaagaaggaaatggctcagtatgctagcaacatcacatcagtcctgtgtcagtttcccagccagcagattgccaacatcctagatagtcatgcagctacactgaaccggaaatctgagcgggaagtcttcttcatgaacacccagagcattgtccagctggtgcagaggtaccgaagtggcatccgtggccacatgaaggctgtggtgatggatctgctgcggcagtacctgcgagtagagacacagtttcagaacggccactacgacaaatgtgtattcgcccttcgggaagagaacaaaagcgacatgaacaccgtactgaactacatcttctcccacgctcaggtcaccaaaaagaatctcctggtgacaatgcttattgatcagttatgtggccgggaccctacacttactgatgagctgctaaatatcctcacagagctaacccaactcagcaagaccaccaacgctaaagtggcgctgcgcgctcgtcaggttcttattgcttcccatttgccatcatatgagcttcgccataaccaagtagagtctatcttcctatcagccattgacatgtatggacaccagttttgcattgagaacctgcagaaactcatcctctcggaaacatctattttcgatgtcctcccaaactttttttaccacagcaaccaggtggtgaggatggcagctctggaggtgtatgttcgaagggcttacattgcctatgaactcaacagcgtacaacaccgccagcttaaggacaacacctgtgtggtggaatttcagttcatgctgcccacatcccatccaaacagagggaacatccccacgctaaacagaatgtcctttgcctccaacctcaaccactatggcatgactcatgtagctagtgtcagcgatgttctgttggacaacgccttcacgccaccttgtcagcggatgggcggaatggtctctttccggacctttgaagattttgtcaggatctttgatgaaataatgggctgcttctgtgactccccaccccaaagccccacattcccagagtctggtcatacttcgctctatgatgaagacaaggtccccagggatgaaccaatacatattctgaatgtggctatcaagactgatggcgatattgaggatgacaggcttgcagctatgttcagagagttcacccagcagaataaagctactttggttgagcatggcatccggcgacttacgttcctagttgcacaaaaggatttcagaaaacaagtcaactgtgaggtggatcagagatttcatagagaattccccaaatttttcacattccgagcaagggataagtttgaggaggaccgcatttatcgacacctggagcctgctctggctttccagttagagttgaaccggatgagaaattttgaccttactgccatcccatgtgctaatcacaagatgcacctgtaccttggggctgctaaggtggaagtaggcacagaagtgactgactacaggttctttgttcgtgcgatcatcaggcactctgatctggtcacaaaggaagcttctttcgaatatctacaaaatgaaggggaacgactgctcctggaagctatggatgaattggaagttgcttttaataatacaaatgtccgcactgactgtaaccacatcttcctcaactttgtgcccacggtcatcatggacccatcaaagattgaagaatctgtgcggagcatggtaatgcgctatggaagtcggctatggaaattgcgggtcctccaggcagaactgaaaatcaacattcgcctgacaacaactggaaaagcaattcccatccgcctcttcctgacaaacgagtctggctactacttggacatcagcctgtataaggaagtgactgactccaggacagcacagatcatgtttcaggcatatggagacaagcagggaccactgcatggaatgttaattaatactccatatgtgaccaaagaccttcttcaatcaaagaggttccaggcacagtccttaggaacaacatatatatatgatatcccagagatgtttcggcagtcactcatcaaactctgggagtccatgtccacccaagcatttcttccttcgcctcctttgccttccgacatcctgacgtatactgaactggtgttggatgatcaaggccagctggtccatatgaacagacttccaggaggaaatgagattggcatggtagcctggaaaatgagccttaaaagccctgaatatccagatggccgagatatcattgtcatcggcaatgacattacatatcggatcggttcctttgggcctcaggaggatttgctgtttctcagagcttctgaacttgccagagcagaaggcatcccacgcatctacgtggcagcgaacagtggagctagaattggacttgcagaagaaatacgccatatgttccatgtggcctgggtagatcctgaagatccctacaagggatacaagtatttatatctgacaccccaggattataagagagtcagtgccctcaattctgtccactgtgaacatgtggaggatgaaggggaatccaggtacaagataacagatattatcgggaaagaagaaggacttggagcagagaaccttcggggttctggaatgattgctggggaatcctcattggcttacgatgaggtcatcaccatcagcctggttacatgccgggccattggtattggggcttaccttgtccggctgggacaaagaaccatccaggttgagaattctcacttaattctgacaggagcaggtgccctcaacaaagtccttggtcgggaagtatacacctccaacaaccagcttgggggcatccagattatgcacaacaatggggttacccactccactgtttgtgatgactttgagggagtcttcacagtcttacactggctgtcatacatgccgaagagcgtacacagttcagttcctctcctgaattccaaggatcctatagatagaatcatcgagtttgttcccacaaaggccccatatgatcctcggtggatgctagcaggccgtcctcacccaacccagaaaggccaatggttgagtgggttttttgactatggatctttctcagaaatcatgcagccctgggcacagaccgtggtagttggcagagccaggttagggggaatacccgtgggagtagttgctgtagaaacccgaacagtggaactcagtatcccagctgatcctgcgaacctggattctgaagccaagataatccagcaggccggccaggtttggttcccagactctgcatttaagacctatcaagctatcaaggactttaaccgtgaagggctacctctaatggtctttgccaactggagaggtttctctggtgggatgaaagatatgtatgaccaagtgctcaagtttggcgcttacattgtggatggcttgcgggaatgttcccagcctgtaatggtttacatcccgccccaggctgagcttcggggtggttcttgggttgtgatcgaccccaccatcaaccctcggcacatggagatgtacgctgaccgagaaagcaggggatctgttctggagccagaagggacagtagaaatcaaattccgtaaaaaggatctggtgaaaaccatgcgtcgggtagatccagtttacatccgcttggctgagcgattgggcaccccagagctaagccccactgagcggaaggagctggagagcaagttgaaggagcgggaggagttcctaattcccatttaccatcaggtagctgtgcagtttgctgacttgcacgacacaccaggccggatgcaggagaagggtgtcattaacgatatcttggattggaaaacatcccgcaccttcttctactggaggctgaggcgcctcctgctggaggacctggtcaagaagaaaatccacaatgccaaccctgagctgactgacggccagatccaggccatgttgagacgctggtttgtagaagttgaaggcacagtgaaggcttacgtctgggacaataataaggacctggtggagtggctggagaagcaactgacagaggaagatggcgtccgctctgtgatagaggagaacatcaaatacatcagcagggactatgtcctgaagcagatccgcagcttggtccaggccaacccagaagtcgccatggactccatcgtccacatgacccagcacatctcacccacccagcgagcagaagttgtaaggatcctctccactatggattccccttccacgtaggaggagcttcccgcccacccctgccctgtctctggagaagagagtcgggctgcctctcccatctgagaccactgtaatgagaaggcaccggaggcctgagactggatcagtggcatttgcttcccttgagtgtttcaggctctgcatgacatcctgggctataggatcacacagcccagtcacacatacccgattcagtatttattagcccagctatgatgacagtcctcttcccatgcacaggactgagaaggcaatgaaaggtacttgcctgtaccatgaggtcttactagactaaagcaggactgccctccgtcctgcctgccccagcatagggtcgtgtgaacagtgtccaagtgtctgcagcccctgccccatgagccacagccaacaggggagggctggggctgccaggagacgcaagtccatcgatcactaccccacattgcaaccaagcacatgccaggcactggagagagacagtcccatctaaccacagcaagataactgagaaatgtcagagcagaaaatctgtcgcaagcccgtgagaacacagagataaaggcaaagccatgatctaacaggcaggctcaaaaccagatcatgtcattgttcctggtcaaacacactcaaccacctcagtggcgtctcagtatccctggggagacggtcaccctgccaccaccttatcactatgtaatcacatagtgactatacctaggagctaaaggcctgtgcataagggccctagcctctgcgggtggatggagacaggcaggcagtgcaggagcctgaggtggctaagaggaggctcttttctgttggtgataggggaagagtacttcaaaacaggagaagtctcttctggggacaggttgtcactatggggccatgcctattcctggcagaaggtatggcgggtcacccacctgatgacacctgtgctttcagcaccagaaactggccttcttgatgttaggaggcttccagggaacaagaattcacctgcccttaagataccttacagttcagtctcggtgctggtgtttccagacttctgggccctatgtggccagcaacacggcttcctcaggggtggcctgaaaagtttaactacccatttgtccaagaaagcagctgatggttcagcatttgtgttcataaagcaagaatgaagatgatcataactcgtataacatccagtttcaactttatgactaatacatctgtcgctgaagatcgagaaggaaaaagatacctgccacagaaaccattcaagtgagtacttcagtctgagccttaacctggattccacgaaaagagctgacctccccttcaggagcacctcaagcagatattccagaggaccttctcaatggtgccacattgcaagtggacatccaagggactatagatgcaagttgccccacccccacaagccctggtggaaggaacaggagggccagagctggatcaggtggggagcatccttagtccagaaggaagctccctggcccctggttccctgcttacctggtaggtaaggatgggatttatctctggcctccacttttgctacagcacgtccaatcccacacccccggtggtatgaaagctgctttcctggagaggtggagtgggctgggcttgtataagtcccttttccctgctgccccatccccgggaccgggccgtctcaatacggagaagtcagagccacggcacatgggcagtgtgatggtaccacagcccattacacatgcagggttactgaggaggagggtgaatgatgtattgacccagactggcttgaactgagattgccatattatctgtccacttgttcacaaagcagccttcacacgtgtcagtgagagttcgatggacacacacgccagcgagcaggggcttcagtccaatgcactcttctcacgttttgttgaaataaacctccacatttgtagaagaaaaaaaaaaaaaaaaaaaaaaa

SEQ ID NO: 155 - DNA, 8497 bp, Mus musculus
atggtcttgcttctctttctgacttgcctggttttctcctgcctgaccttttcctggctaaaaatctgggggaagatgacagactcgaagccgctcaccaacagtaaggtggaagccaatctcctttcaagcgaggagtccctttcagcctcagagctgtcaggggaacagctgcaggaacatggcgaccacagctgtctgtcctacagaggtcccagagacgccagtcagcagaggaactccctgccaagctcatgccagaggcctccgaggaacccactgtcttccaatgacacctggccctccccagaactccaaaccaactggacagccgccccgggcccagaggttccagatgctaatgggttgtccttcccagccaggcccccgagccagaggacagtatccccctccagagaggacaggaagcaggcacacatcaagagacagctgatgaccagctttatcctgggctccctggatgacaactcctctgatgaggaccctagtgccggctccttccagaactcctcccggaagagcagcagggctagcctgggcactctgtcccaggaggctgcattgaacacaagtgatcctgaatctcacgcgcctactatgaggcccagcatgtctggactccatctggtgaagagaggccgagaacacaagaaactggacctgcacagagatttcaccgtcgcgtccccagccgagtttgtcactcggtttggaggcaacagggtcatagagaaggtgctcatcgccaacaatggcatcgctgcggtcaagtgtatgcgctccatccgccgctgggcctacgagatgttccgtaatgaacgtgccatccggttcgtggtcatggtgacgcccgaggatctgaaggccaacgcagagtacatcaagatggccgatcagtacgtccctgtcccaggaggacccaataacaacaactatgccaatgttgagctgatcatagacattgccaagagaatccctgtacaggccgtgtgggctggctggggccacgcttcggaaaaccccaaacttccagagctgctgtgtaaacacgagattgctttcctaggtccccccagtgaggccatgtgggccctgggagacaagatcgcctccaccatcgtagcgcagacactgcaaatcccaaccttaccctggagtggaagcggtctcacagtggagtggacggaggacagccggcaccagggcaaatgcatcagtgtccccgaagacgtttacgagcagggctgtgtgaaggatgtggacgaaggcttgcaggcagcagaaaaaataggatttcctttgatgatcaaagcctccgaaggcggaggcgggaaaggtatccgcaaggctgagagtgcagaggacttcccgatgcttttccgacaagtgcagagtgagattccgggctcgccgatctttctaatgaagctggcccagaacgcacggcacctggaggtccaggtcttggcagatcagtatggaaacgcggtgtcactgttcggacgggactgttccattcagaggcggcaccagaagatcattgaggaggctccggccaccatcgccgcgcctgccgtgtttgagttcatggaacagtgtgctgtcctcctggccaagatggtgggttatgtgagcgcggggactgtggagtacttatacagccaggatggcagcttccacttcctggagctgaacccacgcctgcaggtggagcatccgtgcactgaaatgatcgcagatgtcaacctgcctgctgcgcagttgcagatcgccatgggcgtgcccctgcaccgactgaaggacatacggcttctgtacggagagtccccctggggagtgacccccattccttttgaaacccccttgagccctcccattgcccgaggccacgtcattgcagccaggatcaccagcgaaaacccagatgagggctttaagccaagctcggggacagtgcaggagctgaacttccgcagcaacaagaacgtgtggggttacttcagcgtggccgctgctgggggcttgcacgagttcgccgattcccagtttgggcactgcttctcctggggcgagaaccgtgaagaggccatttcgaacatggtagtggctttgaaggaactgtccatccggggtgacttccggaccactgtggagtatctcgttaaccttctggagaccgagagcttccagaacaacgatatcgacacggggtggctggaccacctcatcgctcagcgggtgcaggcagagaagccggacatcatgcttggggtggtgtgtggggccttgaatgtggcagacgcgatgttcagaacctgcatgacggaattcttgcactccctggaaaggggtcaggtcctccctgctgattctctgctgaacattgtggacgttgaattgatttacggcggcatcaagtatgctctcaaggtagcccgacagtccctgactatgtttgtcctcatcatgaacggctgccacatcgagatcgatgcccaccggctgaacgatggggggctgctcctgtcctacaatggcagcagttacactacgtacatgaaggaagaggttgacagttaccggatcactatcggcaataagacttgcgtgtttgaaaaggaaaacgaccccactgtcctgagatccccctcggctgggaagctgatgcagtatacggtggaggacggagaccacgtggaagctgggagcagctacgcggagatggaggtgatgaagatgatcatgaccctgaacgtgcaggagagcggccgggtgaagtacatcaagcggccaggggttatactggaggctggctgcgtggtggcgaggctagaactcgatgacccttcaaaagtgcacgcggcccagccgtttactggggagctccctgcccagcagactctgcccatcctcggggagaagctacaccaagtgttccacggcgtcttggaaaatctgaccaatgtcatgagtggctactgcctgcccgagcccttcttcagcatgaagctgaaggactgggtccagaagctcatgatgacgctccggcatccctccctacctctgctggagctacaggagatcatgaccagtgtggccggccgcatccccgccccggtggagaaggcagtccgcagggtgatggcccagtacgccagtaacatcacctccgtgctatgccagttccccagccagcagatagccaccatcctggactgccatgccgccaccctgcagcgtaaggcggaccgagaggtcttcttcatgaacacacagagcatcgtgcagctggtccagagataccgcagcgggacccgtggctacatgaaggctgtggtgctggacctcctgaggaaatatctaaacgtggagcaccattttcagcaagcccactatgacaagtgtgtgatcaacctgagggagcagttcaagccggacatgacccaggtgctggactgcatcttctcacactcccaggtggccaagaagaaccagctggtgaccatgctgatagatgagctgtgcggcccagaccccaccctgtcagacgagctgacctccatcctctgtgagctcacgcagctgagcagaagcgagcactgcaaggttgccctcagagccagacaggtcctgatcgcctcccacctcccctcctacgagctgcggcacaaccaggtggagtccatcttcctgtccgccatcgacatgtacggccaccagttctgcccagaaaatctcaagaaactaatactttcagaaacgaccatattcgacgtcctgcccactttcttctatcacgagaacaaggttgtctgtatggcgtccctggaggtttacgtgcggagaggctacatcgcctacgagctaaacagcctgcagcaccgggagctcccagatggcacctgtgtggtggagttccagttcatgctgccctcttcccaccccaaccggatggccgtgcccatcagtgtctccaaccccgacctgctgaggcacagcacagaactcttcatggacagtggcttctccccactgtgccagcggatgggggccatggtggccttcaggagatttgaagaattcaccaggaactttgatgaagtcatctcctgctttgccaacgtgcagacagacactcttctcttcagtaaggcgtgcacttccctgtactcagaggaagacagcaagagcctccgagaggaacccatccacatcctgaatgtggccatccagtgtgccgaccacatggaggacgaggcgctggtgccagtcttccgtgcctttgtacagtccaagaagcacatccttgtggattacggactccgaagaatcacattccttgttgcccaagagagagaattccccaagttcttcacgttcagagcgagggatgagtttgcagaagaccggatttatcgccacttggagcctgccctggccttccagctggagctgagccggatgcgcaactttgacctgaccgctgtgccctgtgccaaccacaagatgcatctttacctgggagccgctaaggtgaaggaagggttggaggtgaccgaccacaggttctttatccgagccattatccggcactcagacctgatcaccaaggaagcctccttcgagtacctgcagaacgaaggtgagcggctgctgctggaagccatggacgagctggaggtggcgttcaacaacaccagtgtgcgcactgactgcaaccacatcttcctcaactttgtgcccacagtcatcatggacccgctcaagatcgaggagtcggtgcgtgacatggtcatgcgctatggcagccgtctgtggaagctccgagtgctgcaggccgaggttaagatcaacatccgtcagacaacctcagatagtgccatccccatccgcctcttcatcaccaacgagtctggctactacctggacatcagcctctatagagaagtgacggactccagatccggaaatatcatgtttcattcctttggcaacaagcaaggtagcctgcatgggatgctgatcaatacaccctatgtcaccaaggacctgctccaggctaagcgattccaggcacagtccctcgggaccacctatgtgtacgacttcccagagatgttcaggcaggctctctttaaactgtggggctccccagagaagtaccccaaagatatcctgacatacacagagctggtgttggactctcaaggtcagctggtggagatgaaccggcttcctggttgtaacgaggtgggcatggtggccttcaaaatgaggttcaagaccccggagtatccagaaggccgggacgccgttgtcatcggcaatgacatcaccttccaaatcggctctttcggcataggggaggacttcctgtacctgcgagcgtctgagatggcccggacagagggcatcccccaaatctacctggcagctaacagcggggcccgtatgggcctggctgaggagatcaaacagatattccaagtggcttgggtggacccagaggatccccacaaaggatttagatacctgtacctgacgccccaagactacacccagatcagttcccagaactcagtgcactgcaagcacatcgaagatgaaggcgaatccagatacgtcatcgtggatgtcatcgggaaggatgccaacctgggtgtggagaacctgaggggctcgggcatgattgcaggagaagcttccctggcttatgagaaaacggtcaccatcagcatggtgacctgccgcgcccttggaatcggggcttacctggtgaggcttggccagcgggtgatccaggtggaaaactcccacatcatcctcaccggagccggtgctctcaacaaggtcctgggaagagaggtctacacgtccaacaaccagctaggtggcgtgcagatcatgcataccaacggggtctcccacgtcaccgtgccagatgacttcgagggggtctgcaccattctggaatggctgtcatttataccgaaggacaatcgcagcccggtccccatcaccactccttctgaccccatcgacagggaaattgaattcaccccaaccaaagctccctacgaccccaggtggatgcttgcaggcaggcctcacccaactctgaaggggacctggcagagtggattcttcgaccatggcagtttcaaggaaatcatggcaccctgggcccagactgtggtgacgggacgagcaaggctggggggcatccctgtgggagtgattgctgtggagactcggactgtggaggtggctgtccccgctgaccctgccaacttggattctgaggccaagatcatccagcaggcgggccaggtgtggttcccggactcggcctacaagacggcgcaggtcatcagggacttcaacaaggagcgcctgcctctcatgatcttcgccaactggaggggcttctccggaggcatgaaggacatgtatgagcagatgctgaagtttggcgcctacatcgtggatggtctccgcctgtacgagcagcccattctcatctacatccctccctgtgcggagctccgtgggggctcctgggttgtcctggactccaccatcaaccccctgtgcatagagatgtatgcagacaaagagagcagggggggtgttctggagccggagggcacggtggagattaagttccggaagaaagatctggtgaaaaccataaggaggatagacccagtgtgtaagaagctcgtgggacagctggggaaagcccagcttcccgacaaggaccggaaggagctggagggccagctgaaggcccgggaggaactgctgcttcccatctaccaccaggtggcagtgcagttcgcagacctgcacgacaccccgggccacatgctggagaaaggaatcatttctgatgtgctggaatggaagactgcccgcaccttcttctactggaggctgcgccggctgctgctggaggcccaggtgaagcaggagatactccgagccagccccgagctgaaccacgagcacacacagtccatgctgcgacgctggttcgtggagactgagggcgctgtcaaggcctacctgtgggacagcaaccaggtggtggtccagtggctggaacagcactggtcagccaaggacggcttgcgctccaccatccgtgaaaacatcaattatctgaagcgggactctgtcctcaagaccatccaaagtctggtccaagaacatccagaggtcatcatggactgcgtggcctacctgagccagcacctcacgccagccgagcggatacaggtggctcagctgctgtctaccacggagagcccagcttcctcctgatgagcaactctggccatccccaggaccctggacggtgggatggccatgcggcaggccttcgagggacacgggcttattttgggaaaactcacggagccactccaaatgtagctgcggttcctgaccagcgacttctggacagaaggcccctgggcccctgctaaggcgggcccagcctctcctcacctggggacttcagggtttgtcccagtggataattctgccactgcctctctgaggccaactgaaccaggcacaccgagttccttgtggtttttgcctgaggtggggatccggtgtctgatcacaagacacttgttctaccacgttggtttgcactcagctccttgtgtccctcagccgagtaaatgggccaaagagaacgccagatgagacccaagaaaacgtctttttgtgatgaggcttattagacacttagcaagagcaaaaatgatttccttttttgttgttgttgttgtttttcatttaagagaataaagtgacattttattctccaagtggccctagtgagtcaggacaaccatctgtcccattagagaggttctccagagataaaggacaaacagacaggacacctagcattcagcatggctggaggagagttactgcctcggttcccaggctctgtaagccctctcttcccaagtggagcaaggactggacctccgtgcctcctgcagattggccacccacagggtactgagtgccctactgcctgagtgcagagaggctgtccctcacccctcccagaacctctggccaatatacttcctcaccttgtgccttgataccacggcaggagccaacagcgctggaccagtgacccagggctgtgcctcgaacactctggatccgcttacagagactttcccagcactgccaggcaccgcagcatcaggacccgaatgagatgctccggccaggggtggggtggggatgttggggtagtggagaaccccactctgtcgctgcccttcttgggggataaagggttcttctgggcaaggtcctcttgatgtggaggaaagcccagaccgacactggctgccaccctgaccacttgggtgcgctgaagaggctgctgaaagagctgcaaaataaactattttttttgaagaaaaaaaaaaaa

SEQ ID NO: 156 - DNA, 2407 bp, Homo sapiens
gccgcggctgccgcctctgctggggtctaggctgtttctctcgcgccaccactggccgccggccgcagctccaggtgtcctagccgcccagcctcgacgccgtcccgggacccctgtgctctgcgcgaagccctggccccgggggccggggcatgggccaggggcgcggggtgaagcggcttcccgcggggccgtgactgggcgggcttcagccatgaagaccctcatagccgcctactccggggtcctgcgcggcgagcgtcaggccgaggctgaccggagccagcgctctcacggaggacctgcgctgtcgcgcgaggggtctgggagatggggcactggatccagcatcctctccgccctccaggacctcttctctgtcacctggctcaataggtccaaggtggaaaagcagctacaggtcatctcagtgctccagtgggtcctgtccttccttgtactgggagtggcctgcagtgccatcctcatgtacatattctgcactgattgctggctcatcgctgtgctctacttcacttggctggtgtttgactggaacacacccaagaaaggtggcaggaggtcacagtgggtccgaaactgggctgtgtggcgctactttcgagactactttcccatccagctggtgaagacacacaacctgctgaccaccaggaactatatctttggataccacccccatggtatcatgggcctgggtgccttctgcaacttcagcacagaggccacagaagtgagcaagaagttcccaggcatacggccttacctggctacactggcaggcaacttccgaatgcctgtgttgagggagtacctgatgtctggaggtatctgccctgtcagccgggacaccatagactatttgctttcaaagaatgggagtggcaatgctatcatcatcgtggtcgggggtgcggctgagtctctgagctccatgcctggcaagaatgcagtcaccctgcggaaccgcaagggctttgtgaaactggccctgcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactgagccagccttcggggccaattccctggaggaaccagctgcaaatcacttttttgctctgtaaatttggaagtgtcatgggtgtctgtgggttatttaaaagaaattataacaattttgctaaaccattacaatgttaggtcttttttaagaaggaaaaagtcagtatttcaagttctttcacttccagcttgccctgttctaggtggtggctaaatctgggcctaatctgggtggctcagctaacctctcttcttcccttcctgaagtgacaaaggaaactcagtcttcttggggaagaaggattgccattagtgacttggaccagttagatgattcactttttgcccctagggatgagaggcgaaagccacttctcatacaagcccctttattgccactaccccacgctcgtctagtcctgaaactgcaggaccagtttctctgccaaggggaggagttggagagcacagttgccccgttgtgtgagggcagtagtaggcatctggaatgctccagtttgatctcccttctgccacccctacctcacccctagtcactcatatcggagcctggactggcctccaggatgaggatgggggtggcaatgacaccctgcaggggaaaggactgccccccatgcaccattgcagggaggatgccgccaccatgagctaggtggagtaactggtttttcttgggtggctgatgacatggatgcagcacagactcagccttggcctggagcacatgcttactggtggcctcagtttaccttccccagatcctagattctggatgtgaggaagagatccctcttcagaaggggcctggccttctgagcagcagattagttccaaagcaggtggcccccgaacccaagcctcacttttctgtgccttcctgagggggttgggccggggaggaaacccaaccctctcctgtgtgttctgttatctcttgatgagatcattgcaccatgtcagacttttgtatatgccttgaaaataaatgaaagtgagaatcctc

SEQ ID NO: 157 - DNA, 2336 bp, Homo sapiens
tgccccgttgtgaggtgataaagtgttgcgctccgggacgccagcgccgcggctgccgcctctgctggggtctaggctgtttctctcgcgccaccactggccgccggccgcagctccaggtgtcctagccgcccagcctcgacgccgtcccgggacccctgtgctctgcgcgaagccctggccccgggggccggggcatgggccaggggcgcggggtgaagcggcttcccgcggggccgtgactgggcgggcttcagccatgaagaccctcatagccgcctactccggggtcctgcgcggcgagcgtcaggccgaggctgaccggagccagcgctctcacggaggacctgcgctgtcgcgcgaggggtctgggagatggggagtggcctgcagtgccatcctcatgtacatattctgcactgattgctggctcatcgctgtgctctacttcacttggctggtgtttgactggaacacacccaagaaaggtggcaggaggtcacagtgggtccgaaactgggctgtgtggcgctactttcgagactactttcccatccagctggtgaagacacacaacctgctgaccaccaggaactatatctttggataccacccccatggtatcatgggcctgggtgccttctgcaacttcagcacagaggccacagaagtgagcaagaagttcccaggcatacggccttacctggctacactggcaggcaacttccgaatgcctgtgttgagggagtacctgatgtctggaggtatctgccctgtcagccgggacaccatagactatttgctttcaaagaatgggagtggcaatgctatcatcatcgtggtcgggggtgcggctgagtctctgagctccatgcctggcaagaatgcagtcaccctgcggaaccgcaagggctttgtgaaactggccctgcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactgagccagccttcggggccaattccctggaggaaccagctgcaaatcacttttttgctctgtaaatttggaagtgtcatgggtgtctgtgggttatttaaaagaaattataacaattttgctaaaccattacaatgttaggtcttttttaagaaggaaaaagtcagtatttcaagttctttcacttccagcttgccctgttctaggtggtggctaaatctgggcctaatctgggtggctcagctaacctctcttcttcccttcctgaagtgacaaaggaaactcagtcttcttggggaagaaggattgccattagtgacttggaccagttagatgattcactttttgcccctagggatgagaggcgaaagccacttctcatacaagcccctttattgccactaccccacgctcgtctagtcctgaaactgcaggaccagtttctctgccaaggggaggagttggagagcacagttgccccgttgtgtgagggcagtagtaggcatctggaatgctccagtttgatctcccttctgccacccctacctcacccctagtcactcatatcggagcctggactggcctccaggatgaggatgggggtggcaatgacaccctgcaggggaaaggactgccccccatgcaccattgcagggaggatgccgccaccatgagctaggtggagtaactggtttttcttgggtggctgatgacatggatgcagcacagactcagccttggcctggagcacatgcttactggtggcctcagtttaccttccccagatcctagattctggatgtgaggaagagatccctcttcagaaggggcctggccttctgagcagcagattagttccaaagcaggtggcccccgaacccaagcctcacttttctgtgccttcctgagggggttgggccggggaggaaacccaaccctctcctgtgtgttctgttatctcttgatgagatcattgcaccatgtcagacttttgtatatgccttgaaaataaatgaaagtgagaatcctctaaaaaaaaaaaa

SEQ ID NO: 158 - DNA, 2470 bp, Macaca fascicularis
gcccagttgtgaggtgataaagtgccgcgctcccggacgccagcaccgccgtcgcagtcgctgctgcgccctaggttgtttctcccgcgccgcctctggccgactggccgaccggccgcagctccaggtgtcctagccacccaacctcggcgccgtcccggggcccccgtgctctgcgcgaagccctggccccggcggccggggcatgggccaggggctcggggtgaggcggcttcccgcggggccgtgactgggcgggcttcatattcagccatgaagaccctcatagccgcctactccggggtcctgcgcggagagcgtcaggccgaggctgaccggagccagcgctctcacggaggacctgcgctgtcgcgcgaggggtctgggagatggggcactggatccagcatcctctccgccctccaggacctcttctccgtcacctggctcaataggtccaaggtggaaaagcagctacaggtcatctcagtgctccagtgggtcctgtccttccttgtactgggagtggcctgcagtgccatcctcatgtacatattctgcaccgattgctggctcatcgctgtgctctacttcacttggctggtgtttgactggaacacacccaagaagggtggcaggaggtcacagtgggtccgaaactgggctgtgtggcgctactttcgagactactttcccatccagctggtgaagacacacaacctgctgaccaccaggaactatatctttggataccacccccatggcatcatgggcctgggtgccttctgcaacttcagcacagaggccacagaagtgagcaagaagttcccaggcatacggccttacctggctacactggcgggcaacttccgattgcctgtgttgagggagtacctgatgtctggaggtatctgccctgtcaaccgggacaccatagactatttgctttcaaagaatgggagtggcaatgctatcatcatcgtggtggggggtgcggctgagtctctgagctccatgcctggcaagaatgcagtcaccctgcggaaccgcaagggctttgtgaaactggccctgcgccatggagccgacctggttcccatgtactcctttggagagaatgaagtgtacaagcaggtgatcttcgaggagggctcctggggccgatgggtccagaagaagttccagaaatacattggtttcgccccatgcatcttccatggccgaggcctcttctcctccgacacctgggggctggtgccctactccaagcccatcaccactgttgtgggagagcccatcaccatccccaagctggagcacccaacccagcaagatatcgacctgtaccacaccatgtacatggaggccctggtgaagctcttcgacaagcacaagaccaagttcggcctcctggagactgaggtcctggaggtgaactgagccaggcttcggggccaactccttggaggaaccagttgcaaattgcttttatgctctgtaaatttggaagcgtcatgggtgtctgtgggttatttaaaagaaattataataattttgctaaaccattacaatgttaggtcttttttaagaaggaaaaagtcagtaattcaagctcttttacttccagtttgccctgttctaggtggtggctaaatctgggcctaatctgggtggctcagctaacctctattcttcccttcctgaagtgacagaggaaactcagtcttggcctggggaagaaggatcgccattagtgacttgggccagttagatgattcactttttgcccccagggatgagaggcaaaagccacttctcatacaagcccctctattgccactaccccacacttgtctagtcctgaaactgcaggaccagtttctctgccaaggggaggagttggagagcacggttgacctgttgtgtgaggacagtagtaggcatctggaatgctcctgtttgatctcccttctgtcacccactcctcacccccagtcactcacatcagagcctggactggccttcaggatgaggatgggggtggcaatgacaccgtgcaggggaaaggactgccccccatgcaccattgcagagaggatgccgccaccatgagctaggtggagtaactggtttttcttgggtggctgatgacatggatgcagcacagactcagccttggcctggagcacatacttgctggtggcctcagtttaccttccccacatcacagattctggatgtgaggaagaggtccctcttcagaaggggcctggccttctgagcagcagattagttccaaagcaggtggcccccgaacccaagcctcacttttctgtgccttcctgagggggttgggccagggaggaaacccaaccctctcctgtgtattctgttatctcttgatgagatcatagcaccatgtcagacttttgtatatgcctagaaaataaatgaaagtgagaatcctcta

SEQ ID NO: 159 - RNA, 16 nt, Artificial Sequence
  synthetic construct
gcagccgaaaggcugc

SEQ ID NO: 160 - RNA, 36 nt, Artificial Sequence
  synthetic construct
guuaugugagugcugggacagcagccgaaaggcugc

SEQ ID NO: 161 - RNA, 36 nt, Artificial Sequence
  synthetic construct
uuucaaacauggugguggcagcagccgaaaggcugc

SEQ ID NO: 162 - RNA, 36 nt, Artificial Sequence
  synthetic construct
aacauggugguggcuuugaagcagccgaaaggcugc

SEQ ID NO: 163 - RNA, 36 nt, Artificial Sequence
  synthetic construct
auggugguggcuuugaaggagcagccgaaaggcugc

SEQ ID NO: 164 - RNA, 36 nt, Artificial Sequence
  synthetic construct
accuguguguuugagaaggagcagccgaaaggcugc

SEQ ID NO: 165 - RNA, 36 nt, Artificial Sequence
  synthetic construct
ccuguguguuugagaaggaagcagccgaaaggcugc

SEQ ID NO: 166 - RNA, 36 nt, Artificial Sequence
  synthetic construct
caguaugcuagcaacaucaagcagccgaaaggcugc

SEQ ID NO: 167 - RNA, 36 nt, Artificial Sequence
  synthetic construct
aguaugcuagcaacaucacagcagccgaaaggcugc

SEQ ID NO: 168 - RNA, 36 nt, Artificial Sequence
  synthetic construct
gugguggaauuccaguucaagcagccgaaaggcugc

SEQ ID NO: 169 - RNA, 36 nt, Artificial Sequence
  synthetic construct
guggaauuccaguucaugcagcagccgaaaggcugc

SEQ ID NO: 170 - RNA, 36 nt, Artificial Sequence
  synthetic construct
uggaauuccaguucaugcuagcagccgaaaggcugc

SEQ ID NO: 171 - RNA, 36 nt, Artificial Sequence
  synthetic construct
ggaauuccaguucaugcugagcagccgaaaggcugc

SEQ ID NO: 172 - RNA, 36 nt, Artificial Sequence
  synthetic construct
gaauuccaguucaugcugcagcagccgaaaggcugc

SEQ ID NO: 173 - RNA, 36 nt, Artificial Sequence
  synthetic construct
aauuccaguucaugcugccagcagccgaaaggcugc

SEQ ID NO: 174 - RNA, 36 nt, Artificial Sequence
  synthetic construct
uaaccacaucuuccucaacagcagccgaaaggcugc

SEQ ID NO: 175 - RNA, 36 nt, Artificial Sequence
  synthetic construct
aaccacaucuuccucaacuagcagccgaaaggcugc

SEQ ID NO: 176 - RNA, 36 nt, Artificial Sequence
  synthetic construct
accacaucuuccucaacuuagcagccgaaaggcugc

SEQ ID NO: 177 - RNA, 36 nt, Artificial Sequence
  synthetic construct
uacaaggaagugacugacuagcagccgaaaggcugc

SEQ ID NO: 178 - RNA, 36 nt, Artificial Sequence
  synthetic construct
caaggaagugacugacuccagcagccgaaaggcugc

SEQ ID NO: 179 - RNA, 36 nt, Artificial Sequence
  synthetic construct
aggaagugacugacuccagagcagccgaaaggcugc

SEQ ID NO: 180 - RNA, 36 nt, Artificial Sequence
  synthetic construct
gaugacuuugaagggguuuagcagccgaaaggcugc

SEQ ID NO: 181 - RNA, 36 nt, Artificial Sequence
  synthetic construct
uggauucugaagccaagauagcagccgaaaggcugc

SEQ ID NO: 182 - RNA, 36 nt, Artificial Sequence
  synthetic construct
ggauucugaagccaagauaagcagccgaaaggcugc

SEQ ID NO: 183 - RNA, 36 nt, Artificial Sequence
  synthetic construct
gauucugaagccaagauaaagcagccgaaaggcugc

SEQ ID NO: 184 - RNA, 36 nt, Artificial Sequence
  synthetic construct
auucugaagccaagauaauagcagccgaaaggcugc

SEQ ID NO: 185 - RNA, 36 nt, Artificial Sequence
  synthetic construct
aggccaucaaggacuucaaagcagccgaaaggcugc

SEQ ID NO: 186 - RNA, 36 nt, Artificial Sequence
  synthetic construct
caucaaggacuucaaccggagcagccgaaaggcugc

SEQ ID NO: 187 - RNA, 36 nt, Artificial Sequence
  synthetic construct
ucaaggacuucaaccgggaagcagccgaaaggcugc

SEQ ID NO: 188 - RNA, 36 nt, Artificial Sequence
  synthetic construct
auggucuuugccaacuggaagcagccgaaaggcugc

SEQ ID NO: 189 - RNA, 36 nt, Artificial Sequence
  synthetic construct
accaagugcugaaguuuggagcagccgaaaggcugc

SEQ ID NO: 190 - RNA, 36 nt, Artificial Sequence
  synthetic construct
aagaaguuccagaaauacaagcagccgaaaggcugc

SEQ ID NO: 191 - RNA, 36 nt, Artificial Sequence
  synthetic construct
auagacuauuugcuuucaaagcagccgaaaggcugc

SEQ ID NO: 192 - RNA, 36 nt, Artificial Sequence
  synthetic construct
agaaugaaguguacaagcaagcagccgaaaggcugc

SEQ ID NO: 193 - RNA, 36 nt, Artificial Sequence
  synthetic construct
aaguguacaagcaggugauagcagccgaaaggcugc

SEQ ID NO: 194 - RNA, 36 nt, Artificial Sequence
  synthetic construct
cauagacuauuugcuuucaagcagccgaaaggcugc

SEQ ID NO: 195 - RNA, 36 nt, Artificial Sequence
  synthetic construct
acaccauagacuauuugcuagcagccgaaaggcugc

SEQ ID NO: 196 - RNA, 36 nt, Artificial Sequence
  synthetic construct
uuggagagaaugaaguguaagcagccgaaaggcugc

SEQ ID NO: 197 - RNA, 36 nt, Artificial Sequence
  synthetic construct
augaaguguacaagcagguagcagccgaaaggcugc

SEQ ID NO: 198 - RNA, 36 nt, Artificial Sequence
  synthetic construct
caccauagacuauuugcuuagcagccgaaaggcugc

SEQ ID NO: 199 - RNA, 36 nt, Artificial Sequence
  synthetic construct
ccauagacuauuugcuuucagcagccgaaaggcugc

SEQ ID NO: 200 - RNA, 36 nt, Artificial Sequence
  synthetic construct
cucauguacauauucugcaagcagccgaaaggcugc

SEQ ID NO: 201 - RNA, 36 nt, Artificial Sequence
  synthetic construct
gacaccauagacuauuugcagcagccgaaaggcugc

SEQ ID NO: 202 - RNA, 36 nt, Artificial Sequence
  synthetic construct
gaagaaguuccagaaauacagcagccgaaaggcugc

SEQ ID NO: 203 - RNA, 36 nt, Artificial Sequence
  synthetic construct
accauagacuauuugcuuuagcagccgaaaggcugc

SEQ ID NO: 204 - RNA, 36 nt, Artificial Sequence
  synthetic construct
auccucauguacauauucuagcagccgaaaggcugc

SEQ ID NO: 205 - RNA, 36 nt, Artificial Sequence
  synthetic construct
gagaaugaaguguacaagcagcagccgaaaggcugc

SEQ ID NO: 206 - RNA, 36 nt, Artificial Sequence; identical to SEQ ID NO: 160
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 207 - RNA, 36 nt, Artificial Sequence; identical to SEQ ID NO: 161
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 208 - RNA, 36 nt, Artificial Sequence; identical to SEQ ID NO: 162
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 209 - RNA, 36 nt, Artificial Sequence; identical to SEQ ID NO: 163
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 210 - RNA, 36 nt, Artificial Sequence; identical to SEQ ID NO: 164
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 211 - RNA, 36 nt, Artificial Sequence; identical to SEQ ID NO: 165
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 212 - RNA, 36 nt, Artificial Sequence; identical to SEQ ID NO: 166
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 213 - RNA, 36 nt, Artificial Sequence; identical to SEQ ID NO: 167
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 214 - RNA, 36 nt, Artificial Sequence; identical to SEQ ID NO: 168
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 215 - RNA, 36 nt, Artificial Sequence; identical to SEQ ID NO: 169
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 216 - RNA, 36 nt, Artificial Sequence; identical to SEQ ID NO: 170
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 217 - RNA, 36 nt, Artificial Sequence; identical to SEQ ID NO: 171
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 218 - RNA, 36 nt, Artificial Sequence; identical to SEQ ID NO: 172
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 219 - RNA, 36 nt, Artificial Sequence; identical to SEQ ID NO: 173
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 220 - RNA, 36 nt, Artificial Sequence; identical to SEQ ID NO: 174
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 221 - RNA, 36 nt, Artificial Sequence; identical to SEQ ID NO: 175
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 222 - RNA, 36 nt, Artificial Sequence; identical to SEQ ID NO: 176
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 223 - RNA, 36 nt, Artificial Sequence; identical to SEQ ID NO: 177
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 224 - RNA, 36 nt, Artificial Sequence; identical to SEQ ID NO: 178
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 225 - RNA, 36 nt, Artificial Sequence; identical to SEQ ID NO: 179
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 226 - RNA, 36 nt, Artificial Sequence; identical to SEQ ID NO: 180
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 227 - RNA, 36 nt, Artificial Sequence; identical to SEQ ID NO: 181
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 228 - RNA, 36 nt, Artificial Sequence; identical to SEQ ID NO: 182
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 229 - RNA, 36 nt, Artificial Sequence; identical to SEQ ID NO: 183
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 230 - RNA, 36 nt, Artificial Sequence; identical to SEQ ID NO: 184
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 231 - RNA, 36 nt, Artificial Sequence; identical to SEQ ID NO: 185
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 232 - RNA, 36 nt, Artificial Sequence; identical to SEQ ID NO: 186
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 233 - RNA, 36 nt, Artificial Sequence; identical to SEQ ID NO: 187
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 234 - RNA, 36 nt, Artificial Sequence; identical to SEQ ID NO: 188
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 235 - RNA, 36 nt, Artificial Sequence; identical to SEQ ID NO: 189
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 236 - RNA, 36 nt, Artificial Sequence; identical to SEQ ID NO: 190
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 237 - RNA, 36 nt, Artificial Sequence; identical to SEQ ID NO: 191
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 238 - RNA, 36 nt, Artificial Sequence; identical to SEQ ID NO: 192
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 239 - RNA, 36 nt, Artificial Sequence; identical to SEQ ID NO: 193
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 240 - RNA, 36 nt, Artificial Sequence; identical to SEQ ID NO: 194
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 241 - RNA, 36 nt, Artificial Sequence; identical to SEQ ID NO: 195
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 242 - RNA, 36 nt, Artificial Sequence; identical to SEQ ID NO: 196
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 243 - RNA, 36 nt, Artificial Sequence; identical to SEQ ID NO: 197
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 244 - RNA, 36 nt, Artificial Sequence; identical to SEQ ID NO: 198
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 245 - RNA, 36 nt, Artificial Sequence; identical to SEQ ID NO: 199
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 246 - RNA, 36 nt, Artificial Sequence; identical to SEQ ID NO: 200
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 247 - RNA, 36 nt, Artificial Sequence; identical to SEQ ID NO: 201
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 248 - RNA, 36 nt, Artificial Sequence; identical to SEQ ID NO: 202
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 249 - RNA, 36 nt, Artificial Sequence; identical to SEQ ID NO: 203
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 250 - RNA, 36 nt, Artificial Sequence; identical to SEQ ID NO: 204
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 251 - RNA, 36 nt, Artificial Sequence; identical to SEQ ID NO: 205
  synthetic construct
  misc_feature (1)..(2): linked via phosphorothioate
  modified_base (1)..(1): 2'-O-methyl modified
  modified_base (2)..(2): 2'-O-methyl modified
  modified_base (3)..(3): 2'-O-methyl modified
  modified_base (4)..(4): 2'-O-methyl modified
  modified_base (5)..(5): 2'-O-methyl modified
  modified_base (6)..(6): 2'-O-methyl modified
  modified_base (7)..(7): 2'-O-methyl modified
  modified_base (8)..(8): 2'-fluoro modified
  modified_base (9)..(9): 2'-fluoro modified
  modified_base (10)..(10): 2'-fluoro modified
  modified_base (11)..(11): 2'-fluoro modified
  modified_base (12)..(12): 2'-O-methyl modified
  modified_base (13)..(13): 2'-O-methyl modified
  modified_base (14)..(14): 2'-O-methyl modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  modified_base (20)..(20): 2'-O-methyl modified
  modified_base (21)..(21): 2'-O-methyl modified
  modified_base (22)..(22): 2'-O-methyl modified
  modified_base (23)..(23): 2'-O-methyl modified
  modified_base (24)..(24): 2'-O-methyl modified
  modified_base (25)..(25): 2'-O-methyl modified
  modified_base (26)..(26): 2'-O-methyl modified
  modified_base (27)..(27): 2'-O-methyl modified
  modified_base (28)..(28): GalNAc-conjugated
  modified_base (29)..(29): GalNAc-conjugated
  modified_base (30)..(30): GalNAc-conjugated
  modified_base (31)..(31): 2'-O-methyl modified
  modified_base (32)..(32): 2'-O-methyl modified
  modified_base (33)..(33): 2'-O-methyl modified
  modified_base (34)..(34): 2'-O-methyl modified
  modified_base (35)..(35): 2'-O-methyl modified
  modified_base (36)..(36): 2'-O-methyl modified

SEQ ID NO: 252 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugucccagcacucacauaacgg

SEQ ID NO: 253 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugccaccaccauguuugaaagg

SEQ ID NO: 254 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uucaaagccaccaccauguugg

SEQ ID NO: 255 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uccuucaaagccaccaccaugg

SEQ ID NO: 256 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uccuucucaaacacacaggugg

SEQ ID NO: 257 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuccuucucaaacacacagggg

SEQ ID NO: 258 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uugauguugcuagcauacuggg

SEQ ID NO: 259 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugugauguugcuagcauacugg

SEQ ID NO: 260 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uugaacuggaauuccaccacgg

SEQ ID NO: 261 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugcaugaacuggaauuccacgg

SEQ ID NO: 262 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uagcaugaacuggaauuccagg

SEQ ID NO: 263 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ucagcaugaacuggaauuccgg

SEQ ID NO: 264 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugcagcaugaacuggaauucgg

SEQ ID NO: 265 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uggcagcaugaacuggaauugg

SEQ ID NO: 266 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uguugaggaagaugugguuagg

SEQ ID NO: 267 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaguugaggaagaugugguugg

SEQ ID NO: 268 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaaguugaggaagauguggugg

SEQ ID NO: 269 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uagucagucacuuccuuguagg

SEQ ID NO: 270 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uggagucagucacuuccuuggg

SEQ ID NO: 271 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ucuggagucagucacuuccugg

SEQ ID NO: 272 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaaaccccuucaaagucaucgg

SEQ ID NO: 273 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaucuuggcuucagaauccagg

SEQ ID NO: 274 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuaucuuggcuucagaauccgg

SEQ ID NO: 275 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuuaucuuggcuucagaaucgg

SEQ ID NO: 276 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uauuaucuuggcuucagaaugg

SEQ ID NO: 277 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuugaaguccuugauggccugg

SEQ ID NO: 278 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uccgguugaaguccuugauggg

SEQ ID NO: 279 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uucccgguugaaguccuugagg

SEQ ID NO: 280 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuccaguuggcaaagaccaugg

SEQ ID NO: 281 - RNA, 22 nt, Artificial Sequence
  synthetic construct
  misc_feature (1)..(3): linked via phosphorothioate
  modified_base (1)..(1): 4'-phosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-O-methyl modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uccaaacuucagcacuuggugg

SEQ ID NO: 282 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuguauuucuggaacuucuugg

SEQ ID NO: 283 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuugaaagcaaauagucuaugg

SEQ ID NO: 284 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uugcuuguacacuucauucugg

SEQ ID NO: 285 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaucaccugcuuguacacuugg

SEQ ID NO: 286 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uugaaagcaaauagucuauggg

SEQ ID NO: 287 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uagcaaauagucuauggugugg

SEQ ID NO: 288 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uuacacuucauucucuccaagg

SEQ ID NO: 289 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaccugcuuguacacuucaugg

SEQ ID NO: 290 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaagcaaauagucuaugguggg

SEQ ID NO: 291 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugaaagcaaauagucuaugggg

SEQ ID NO: 292 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uugcagaauauguacaugaggg

SEQ ID NO: 293 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugcaaauagucuauggugucgg

SEQ ID NO: 294 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uguauuucuggaacuucuucgg

SEQ ID NO: 295 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uaaagcaaauagucuauggugg

SEQ ID NO: 296 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
uagaauauguacaugaggaugg

SEQ ID NO: 297 - RNA, 22 nt, Artificial Sequence
  synthetic construct
  misc_feature (1)..(3): linked via phosphorothioate
  modified_base (1)..(1): 4'-monomoethylphosphonate-2'-O-methyl modified
  modified_base (1)..(1): 2'-O-methyl modified
  modified_base (2)..(2): 2'-fluoro modified
  modified_base (3)..(3): 2'-fluoro modified
  modified_base (4)..(4): 2'-fluoro modified
  modified_base (5)..(5): 2'-fluoro modified
  modified_base (6)..(6): 2'-O-methyl modified
  modified_base (7)..(7): 2'-fluoro modified
  modified_base (8)..(8): 2'-O-methyl modified
  modified_base (9)..(9): 2'-O-methyl modified
  modified_base (10)..(10): 2'-fluoro modified
  modified_base (11)..(11): 2'-O-methyl modified
  modified_base (12)..(12): 2'-O-methyl modified
  modified_base (13)..(13): 2'-O-methyl modified
  modified_base (14)..(14): 2'-fluoro modified
  modified_base (15)..(15): 2'-O-methyl modified
  modified_base (16)..(16): 2'-O-methyl modified
  modified_base (17)..(17): 2'-O-methyl modified
  modified_base (18)..(18): 2'-O-methyl modified
  modified_base (19)..(19): 2'-O-methyl modified
  misc_feature (20)..(22): linked via phosphorothioate
  modified_base (20)..(20): 2'-O-methyl modified
  modified_base (21)..(21): 2'-O-methyl modified
  modified_base (22)..(22): 2'-O-methyl modified
ugcuuguacacuucauucucgg

(19) United States
(12) Patent Application Publication
(10) Pub. No.: US 2023/0340487 A1
(43) Pub. Date: Oct. 26, 2023

(54) COMPOSITIONS AND METHODS FOR THE TREATMENT OF METABOLIC SYNDROME

(71) Applicant: Dicerna Pharmaceuticals, Inc., Lexington, MA (US)

(72) Inventors: Marc ABRAMS, Natick, MA (US); Elisabeth WONDIMU, Lexington, MA (US); Soumik BASURAY, Woburn, MA (US)

(21) Appl. No.: 18/019,536

(22) PCT Filed: Aug. 4, 2021
(86) PCT No.: PCT/US2021/044544

§ 371 Date: Feb. 3, 2023

Related U.S. Application Data
(60) Provisional application No. 63/082,762, filed on Sep. 24, 2020.
(60) Provisional application No. 63/061,045, filed on Aug. 4, 2020.

(51) Int. Cl. C12N 15/113; A61P 1/16

(52) U.S. Cl. CPC C12N 15/1137; A61P 1/16; C12N 2310/11; C12N 2310/315; C12N 2310/321; C12N 2310/322; C12N 2310/351; C12N 2310/531

(57) ABSTRACT

Disclosed are methods for treating, preventing and alleviating obesity, fatty liver syndrome, diabetes, liver fibrosis, NASH or other more metabolic syndrome conditions or complications comprising administering an effective amount of oligonucleotides designed to prevent, limit or modulate the expression of mRNA molecules, preferably ACC and/or DGAT2.

### CROSS-REFERENCE TO RELATED APPLICATIONS

This application claims priority to, and the benefit of, U.S. Provisional Application No. 63/061,045 filed on Aug. 4, 2020 and U.S. Provisional Application No. 63/082,762 filed on Sep. 24, 2020. The contents of each of the aforementioned patent applications are incorporated herein by reference in their entireties.

### TECHNICAL FIELD

The disclosure relates to the use of oligonucleotides to inhibit and control Metabolic Syndrome. In certain embodiments, the method comprises administering to the subject a therapeutically effective amount of one or more RNAi oligonucleotides that inhibit Acetyl-coA carboxylase (ACC) expression in a subject and/or that inhibit Diacylglycerol O-acyltransferase 2 (DGAT2) expression in a subject.

### BACKGROUND

Metabolic Syndrome, or metabolic disease, is a cluster of associated medical conditions and associated pathologies. Typically, the syndrome is associated with at least three of five of the following medical conditions: abdominal obesity, elevated blood pressure, elevated fasting plasma glucose, high serum triglycerides, and low levels of high-density lipoprotein (HDL) levels. An individual with Metabolic Syndrome is at higher risk of developing cardiovascular disease and diabetes. Presently, a third of the U.S. population is thought to have Metabolic Syndrome and one or more of the listed pathologies. Despite treatment advances, there remains a high unmet medical need for therapies to treat cardiovascular and metabolic diseases.

### BRIEF SUMMARY OF THE DISCLOSURE

The disclosure provides a method of treating, reverting, and/or preventing Metabolic Syndrome in a subject in need thereof. The disclosure further provides RNAi oligonucleotide molecules that can limit, control or eliminate the expression of key genes associated with Metabolic Syndrome. Such RNAi oligonucleotide molecules are a variety of double-stranded RNAi oligonucleotides where one can target Acetyl-CoA Carboxylase (ACC or ACAC) alone another can target Diacylglycerol O-acyltransferase 2 (DGAT2) alone or a combination of such molecules can be used to target ACC and DGAT2 simultaneously or in sequence.

The disclosure is based, at least in part, on the discovery that oligonucleotides (e.g., double-stranded oligonucleotides, e.g., RNAi oligonucleotides) reduce ACC or DGAT2 expression. Accordingly, target sequences within ACC or DGAT2 mRNA were identified and oligonucleotides that bind to these target sequences and inhibit ACC or DGAT2 mRNA expression were generated. As demonstrated herein, the oligonucleotides inhibited murine, monkey and/or human ACC or DGAT2 expression in vivo. Without being bound by theory, the oligonucleotides targeting ACC described herein are useful for treating a disease, disorder or condition associated with ACC expression, and the oligonucleotides targeting DGAT2 described herein are useful for treating a disease, disorder or condition associated with DGAT2 expression. Further, as demonstrated herein, a combination of inhibitors (e.g., oligonucleotides) of ACC and DGAT2 act synergistically to reduce expression of ACC. When evaluated in a mouse model of NASH, the combination of ACC and DGAT2 inhibitors reduced serum cholesterol, ALT levels, liver steatosis, triglyceride levels, and other markers of liver inflammation (e.g., IL-6 and IL-12b) significantly. Without being bound by theory, a combination of inhibitors targeting ACC and DGAT2, such as the oligonucleotides described herein, are useful for treating a disease, disorder or condition having pathologies such as liver fibrosis and/or pathologies associated with Metabolic Syndrome.

In certain embodiments, the method comprises administering to the subject a therapeutically effective amount of a composition that inhibits ACC expression or activity and/or a composition that inhibits DGAT2 expression or activity in the subject. Such RNAi oligonucleotide molecules can be used to treat a subject having Metabolic Syndrome and associated pathologies and may thereby therapeutically benefit a subject suffering from liver disease (e.g., fatty liver, steatohepatitis), dyslipidemia (e.g., hyperlipidemia, high LDL cholesterol, low HDL cholesterol, hypertriglyceridemia, postprandial hypertriglyceridemia), disorders of glycemic control (e.g., insulin resistance, diabetes), cardiovascular disease (e.g., hypertension, endothelial cell dysfunction), kidney disease (e.g., acute kidney disorder, tubular dysfunction, proinflammatory changes to the proximal tubules), metabolic syndrome, adipocyte dysfunction, visceral adipose deposition, obesity, hyperuricemia, gout, eating disorders, and excessive sugar craving.

Accordingly, in one aspect, the present disclosure provides RNAi oligonucleotide molecules each capable of inhibiting expression of either ACC or DGAT2 or both when two oligonucleotides of the disclosure are used together, either in sequence or simultaneously. Such molecules can be used alone or in combination and can each vary in dosage. In some aspects, each such RNAi oligonucleotide molecule is comprised of a sense strand and an antisense strand forming a double-stranded region. In some embodiments, the antisense strand comprises a region of complementarity to ACC or DGAT2. In some embodiments, an ACC targeting oligonucleotide comprises a sense strand that comprises at least 15 contiguous nucleotides differing by no more than 3 nucleotides from anyone of the nucleotide sequences of SEQ ID NOs: 1, 29, 31, 43 and 55 and an antisense strand comprises at least 15 contiguous nucleotides differing by no more than 3 nucleotides from the nucleotide sequences of SEQ ID NOs: 2, 30, 32, 44 and 56. In some embodiments, a DGAT2 targeting oligonucleotide comprises a sense strand that comprises at least 15 contiguous nucleotides differing by no more than 3 nucleotides from anyone of the nucleotide sequences of SEQ ID NOs: 105, 107, 111, 126, 129 and 137 and an antisense strand comprises at least 15 contiguous nucleotides differing by no more than 3 nucleotides from the nucleotide sequences of SEQ ID NOs: 106, 108, 112, 125, 130 and 138. In some embodiments, a DGAT2 targeting oligonucleotide comprises a sense strand that comprises at least 15 contiguous nucleotides differing by no more than 3 nucleotides from anyone of the nucleotide sequences of SEQ ID NOs: 105, 107, 111, 117, 119, 125, 129 and 137 and an antisense strand comprises at least 15 contiguous nucleotides differing by no more than 3 nucleotides from the nucleotide sequences of SEQ ID NOs: 106, 108, 112, 118, 120, 126, 130 and 138. In some embodiments, a DGAT2 targeting oligonucleotide comprises a sense strand that comprises at least 15 contiguous nucleotides differing by no more than 3 nucleotides from anyone of the nucleotide sequences of SEQ ID NOs: 126, 129 and 137 and an antisense strand comprises at least 15 contiguous nucleotides differing by no more than 3 nucleotides from the nucleotide sequences of SEQ ID NOs: 125, 130 and 138.

In some embodiments, the antisense strand is 19 to 27 nucleotides in length or 21 to 27 nucleotides in length. In some embodiments, the antisense strand is 22 nucleotides in length.

In some embodiments, the sense strand is 19 to 40 nucleotides in length. In some embodiments, the sense strand is 36 nucleotides in length.

In some embodiments, the oligonucleotide has a duplex region of at least 19 nucleotides in length or at least 21 nucleotides in length. In some embodiments, the duplex region is 20 nucleotides in length.

In some embodiments, the region of complementarity to either ACC or DGAT2 is at least 19 contiguous nucleotides in length or at least 21 contiguous nucleotides in length.

In some embodiments, the oligonucleotide comprises on the sense strand at its 3′ end a stem-loop set forth as: S1-L-S2, wherein S1 is complementary to S2, and wherein L forms a loop between S1 and S2 of 3 to 5 nucleotides in length.

In some embodiments, an oligonucleotide for reducing either ACC or DGAT2 for treating, reverting, and/or preventing Metabolic Syndrome comprises an antisense strand and a sense strand, wherein the antisense strand is 21 to 27 nucleotides in length and has a region of complementarity to ACC or DGAT2, wherein the sense strand comprises at its 3′ end a stem-loop set forth as: S1-L-S2, wherein S1 is complementary to S2, and wherein L forms a loop between S1 and S2 of 3 to 5 nucleotides in length, and wherein the antisense strand and the sense strand form a duplex structure of at least 19 nucleotides in length but are not covalently linked.

In some embodiments, the loop L is a tetraloop. In some embodiments, L is 4 nucleotides in length. In some embodiments, L comprises a sequence GAAA.

In some embodiments, the oligonucleotide comprises an antisense strand which is 27 nucleotides in length and a sense strand which is 25 nucleotides in length. In some embodiments, the oligonucleotide comprises an antisense strand which is 22 nucleotides in length and a sense strand which is 36 nucleotides in length.

In some embodiments, the duplex region oligonucleotide of the present disclosure comprises a 3′-overhang sequence on the antisense strand. In some embodiments, the 3′-overhang sequence on the antisense strand is 2 nucleotides in length. In some embodiments, the 3′-overhang sequence comprises purine nucleotides only. In some embodiments, the 3′-overhang sequence is selected from AA, GG, AG and GA. In some embodiments, the 3′-overhang sequence is GG.

In some embodiments, the oligonucleotide comprises an antisense strand and a sense strand that are each in a range of 21 to 23 nucleotides in length. In some embodiments, the oligonucleotide comprises a duplex structure in a range of 19 to 21 nucleotides in length. In some such embodiments, the oligonucleotide comprises a 3′-overhang sequence of one or more nucleotides in length, wherein the 3′-overhang sequence is present on the antisense strand, the sense strand, or the antisense strand and sense strand. In some embodiments, the 3′-overhang sequence of 2 nucleotides in length, wherein the 3′-overhang sequence is on the antisense strand, and wherein the sense strand is 21 nucleotides in length and the antisense strand is 23 nucleotides in length, such that the sense strand and antisense strand form a duplex of 21 nucleotides in length.

In some embodiments, the oligonucleotide comprises at least one modified nucleotide. In some embodiments, the modified nucleotide comprises a 2′-modification. In some embodiments, all of the nucleotides of the oligonucleotide are modified, for example with a 2′-modification.

In some embodiments, the oligonucleotide comprises at least one modified internucleotide linkage, preferably a phosphorothioate linkage.

In some embodiments, the 4′-carbon of the sugar of the 5′-nucleotide of the antisense strand comprises a phosphate analog, for example, an oxymethylphosphonate, vinylphosphonate or malonyl phosphonate. In some embodiments, the phosphate analog is 4′-oxymethylphosphonate.

In some embodiments, at least one nucleotide of the oligonucleotide is conjugated to one or more targeting ligands, such as a carbohydrate, amino sugar, cholesterol, polypeptide or lipid. In some embodiments, the targeting ligand comprises a N-acetylgalactosamine (GalNAc) moiety. In some embodiments, the (GalNAc) moiety comprises a monovalent GalNAc moiety, a bivalent GalNAc moiety, a trivalent GalNAc moiety, or a tetravalent GalNAc moiety.

In some embodiments, the targeting ligand is conjugated to one or more nucleotides of L of the stem loop. In some embodiments, up to 4 nucleotides of L of the stem-loop are each conjugated to a monovalent GalNAc moiety. In some embodiments, 3 nucleotides of L of the stem-loop are each conjugated to a monovalent GalNAc moiety.

In some embodiments, the oligonucleotides of the present disclosure are RNAi oligonucleotides.

In some embodiments, the disclosure of the present disclosure is a pharmaceutical composition comprising one or more oligonucleotides and a pharmaceutically acceptable carrier, delivery agent or excipient.

In some embodiments, the present disclosure provides a method of delivering an oligonucleotide to a subject, the method comprising administering a pharmaceutical composition to a subject.

In another aspect, the present disclosure provides a method of reducing ACC or DGAT2 expression in a cell, a population of cells or a subject by administering an oligonucleotide of the disclosure. In some embodiments, a method of reducing ACC or DGAT2 expression in a cell, a population of cells or a subject comprises the step of contacting the cell or the population of cells or administering to the subject an effective amount of an oligonucleotide or oligonucleotides described herein, or a pharmaceutical composition thereof. In some embodiments, the method for reducing ACC or DGAT2 expression comprises reducing an amount or a level of ACC or DGAT2 mRNA, an amount or a level of ACC or DGAT2 protein, or both.

In some embodiments the present disclosure provides a pharmaceutical product for use as a therapeutic agent. In some embodiments a therapeutic agent is administered as a monotherapy and is an inhibitor of ACC or DGAT2 expression. In some embodiments, the present disclosure provides a pharmaceutical product comprising at least a first and second therapeutic agent, wherein the first therapeutic agent is an inhibitor of ACC or DGAT2. In some embodiments a therapeutic agent is administered prior to, or intermittently with, administration of a second therapeutic agent. In some embodiments, a first therapeutic agent is administered concurrently or simultaneously with a second therapeutic agent, wherein the first therapeutic agent is an inhibitor of ACC expression and wherein the second therapeutic agent is an inhibitor of DGAT2 expression. In some embodiments a first therapeutic agent and a second therapeutic agent are administered sequentially, in either order, wherein the first therapeutic agent is an inhibitor of ACC expression and wherein the second therapeutic agent is an inhibitor of DGAT2 expression.

In some embodiments the first therapeutic agent is an oligonucleotide and is an inhibitor of ACC expression and the second therapeutic agent is an inhibitor of DGAT2 expression. In some embodiments the first therapeutic agent is an inhibitor of ACC expression, and the second therapeutic agent is an oligonucleotide and is an inhibitor of DGAT2 expression. In some embodiments both therapeutic agents are oligonucleotides.

In some embodiments the first therapeutic agent is an oligonucleotide and is an inhibitor of ACC expression and the second therapeutic agent is an oligonucleotide and is an inhibitor of DGAT2 expression and both therapeutic agents are delivered preferentially to the liver. In some embodiments the first therapeutic agent is an oligonucleotide and is an inhibitor of ACC expression and the second therapeutic agent is an oligonucleotide and is an inhibitor of DGAT2 expression and both therapeutic agents are delivered preferentially to adipose tissue. In some embodiments the first therapeutic agent is an oligonucleotide and is an inhibitor of ACC expression and is preferentially delivered to the liver and the second therapeutic agent is an oligonucleotide and is an inhibitor of DGAT2 expression and is preferentially delivered to adipose tissue. In some embodiments the first therapeutic agent is an oligonucleotide and is an inhibitor of ACC expression and is preferentially delivered to adipose tissue and the second therapeutic agent is an oligonucleotide and is an inhibitor of DGAT2 expression and is preferentially delivered to the liver.

In some embodiments the present disclosure provides a combination product comprising: (i) an ACC inhibiting oligonucleotide and, (ii) a DGAT inhibiting oligonucleotide. In some embodiments, the combination product is a product wherein component (i) is an oligonucleotide comprising an antisense strand of 15 to 30 nucleotides in length and a sense strand of 15 to 40 nucleotides in length, and is an inhibitor of ACC expression, and wherein component (ii) is an oligonucleotide comprising an antisense strand of 15 to 30 nucleotides in length and a sense strand of 15 to 40 nucleotides in length and is an inhibitor of DGAT2 expression. In some embodiments, the combination product is a composition comprising components (i) and (ii) and a pharmaceutically acceptable salt thereof.

In some embodiments, components of the combination product (i) and (ii) are formulated in an injectable suspension, a gel, an oil, a pill, a tablet, a suppository, a powder, a capsule, an aerosol, an ointment, a cream, a patch, or means of galenic forms for a prolonged and/or slow release.

In some embodiments, a subject for treatment with the oligonucleotide of the disclosure has a disease, disorder or condition associated with ACC or DGAT2 expression. In some embodiments, a method for treating a subject having a disease, disorder or condition associated with ACC or DGAT2 expression, comprises administering to the subject in need thereof a therapeutically effective amount of one or more of the oligonucleotides described herein, or a pharmaceutical composition thereof, thereby treating the subject. In some embodiments, a subject for treatment has received or is receiving an inhibitor of ACC, and the disclosure provides a method of administering an inhibitor of DGAT2. In some embodiments, a subject for treatment has received or is receiving an inhibitor of DGAT2, and the disclosure provides a method of administering an inhibitor of ACC.

In some embodiments, the disease, disorder or condition associated with ACAC and/or DGAT2 expression is selected from the group consisting of metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g., small intestine) fibrosis, colon fibrosis, stomach fibrosis, lung fibrosis, lung fibrosis consecutive to chronic inflammatory airway diseases, such as COPD, asthma, emphysema, smoker’s lung, tuberculosis, pulmonary fibrosis, idiopathic pulmonary fibrosis (IPF).

In a further aspect, the present disclosure provides use of any of the oligonucleotides of the present disclosure, or the pharmaceutical composition thereof, in the manufacture of a medicament for the treatment of a disease, disorder or condition associated with ACAC or DGAT2 expression.

In some embodiments, the oligonucleotide of the disclosure, or the pharmaceutical composition of the disclosure, is for use, or adaptable for use, in the treatment of a disease, disorder or condition associated with ACC or DGAT2 expression.

In a further aspect, the oligonucleotide of the present disclosure is provided in the form of a kit for treating a disease, disorder or condition associated with ACC or DGAT2 expression. In some embodiments, the kit comprises an oligonucleotide described herein, and a pharmaceutically acceptable carrier. In some embodiments, the kit further includes a package insert comprising instructions for administration of the oligonucleotide to a subject having a disease, disorder or condition associated with ACC or DGAT2 expression.

In some aspects, the disclosure provides a method for reducing liver fibrosis, comprising providing a patient an siRNA specific for a) ACC and b) an siRNA specific for DGAT2.

In some embodiments of the use or kits, the disease, disorder or condition associated with ACC or DGAT2 expression is selected from the group consisting of liver disease (e.g., fatty liver, steatohepatitis), dyslipidemia (e.g., hyperlipidemia, high LDL cholesterol, low HDL cholesterol, hypertriglyceridemia, postprandial hypertriglyceridemia), disorders of glycemic control (e.g., insulin resistance, diabetes), cardiovascular disease (e.g., hypertension, endothelial cell dysfunction), kidney disease (e.g., acute kidney disorder, tubular dysfunction, proinflammatory changes to the proximal tubules), metabolic syndrome, adipocyte dysfunction, visceral adipose deposition, obesity, hyperuricemia, gout, eating disorders, and excessive sugar.

### BRIEF DESCRIPTION OF THE DRAWINGS

FIGS. 1A-1C provide schematics depicting the structure and chemical modification patterns of generic N-Acetylgalactosamine (GalNAc)-conjugated oligonucleotides. Nucleotide positions 27-30 (not numbered) show a tetraloop structure. 2′-OMe = 2′-O-methyl; 2′-F = 2′-fluoro.

FIG. 2 provides a graph depicting the percent (%) of endogenous mouse ACACA and ACACB mRNA remaining in liver samples from mice treated with the indicated GalNAc conjugated ACAC oligonucleotides relative to mice treated with phosphate buffered saline.

FIGS. 3A-3B provide graphs depicting the percent (%) of endogenous mouse ACACA and ACACB mRNA remaining (FIG. 3A) or log % expression (FIG. 3B) in liver samples from mice treated with a GalNAc-conjugated ACAC oligonucleotide (GalXC-ACAC-5083) relative to untreated mice or mice treated with phosphate buffered saline (PBS).

FIG. 4 provides a graph depicting the percent (%) of endogenous mouse ACC½ (total ACC) protein remaining, as measured by western blot in liver samples from mice treated with a GalNAc-conjugated ACAC oligonucleotide (GalXC-ACAC-5083) relative to untreated mice or mice treated with phosphate buffered saline (PBS).

FIG. 5A provides images depicting the level of liver steatosis as measured by Sirius red staining in liver samples from mice treated with a GalNAc-conjugated ACAC oligonucleotide (GalXC-ACAC-5083) relative to mice treated with phosphate buffered saline (PBS).

FIG. 5B provides a graph depicting the liver fibrosis score in liver samples from mice treated with a GalNAc-conjugated ACAC oligonucleotide (GalXC-ACAC-5083) relative to untreated mice or mice treated with phosphate buffered saline (PBS). **** = p<0.0001

FIG. 5C provides a graph depicting the percent (%) of Col1a1 mRNA remaining in liver samples from mice treated with the indicated GalNAc-conjugated ACAC oligonucleotide relative to untreated mice or mice treated with phosphate buffered saline (PBS). *=p<0.05

FIG. 5D provides a graph depicting the percent (%) of Vim mRNA remaining in liver samples from mice treated with a GalNAc-conjugated ACAC oligonucleotide (GalXC-ACAC-5083) relative to untreated mice or mice treated with phosphate buffered saline (PBS). **=p<0.01

FIG. 6 provides a graph depicting the percent (%) of ACACA and ACACB mRNA remaining in liver samples from a hydrodynamic injection (HDI) model in CD-1 mice. Three (3) days after subcutaneous dosing of 3 mg/kg of ACAC oligonucleotides conjugated to GalNAc, and formulated in phosphate buffered saline (PBS), plasmids encoding human ACACA and ACACB mRNA were injected into the mice via HDI and the percent (%) of human ACACA and ACACB mRNA was measured 1 day later in liver samples from the mice relative to mice treated with PBS.

FIGS. 7A-7B provide graphs depicting the percent (%) of ACACA (FIG. 7A) and ACACB (FIG. 7B) mRNA remaining in liver samples from a hydrodynamic injection (HDI) model in CD-1 mice. Three (3) days after subcutaneous dosing of 0.3, 1, or 3 mg/kg of ACAC oligonucleotides conjugated to GalNAc, and formulated in phosphate buffered saline (PBS), plasmids encoding human ACACA and ACACB mRNA was injected into the mice via HDI and the percent (%) of human ACACA and ACACB mRNA was measured 1 day later in liver samples from the mice relative to mice treated with PBS.

FIGS. 8A-8D provide graphs depicting the percent (%) of monkey ACACA (FIGS. 8A and 8C) and ACACB (FIGS. 8B and 8D) mRNA remaining in liver samples from non-human primates (NHPs) treated with the indicated GalNAc-conjugated ACAC oligonucleotides relative to NHPs treated with phosphate buffered saline (PBS).

FIG. 9 provides a graph depicting the percent (%) of ACC½ (total ACC) protein, as measured by western blot in liver samples from NHPs treated with the indicated GalNAc-conjugated ACAC oligonucleotide relative to NHPs treated with phosphate buffered saline (PBS).

FIG. 10 provides a graph depicting the percent (%) of endogenous mouse DGAT2 mRNA knockdown in liver samples from mice treated with the indicated GalNAc-conjugated DGAT2 oligonucleotides relative to mice treated with phosphate buffered saline (PBS).

FIG. 11 provides a graph depicting the percent (%) of endogenous mouse DGAT2 mRNA knockdown in liver samples from mice treated with the indicated GalNAc conjugated DGAT2 oligonucleotides at varying doses (0.3, 1 and 3 mg/kg) relative to mice treated with phosphate buffered saline (PBS).

FIG. 12 provides a graph depicting the percent (%) of DGAT2 mRNA knockdown in liver samples from a hydrodynamic injection (HDI) model in CD-1 mice. Three (3) days after subcutaneous dosing of 3 mg/kg of DGAT2 oligonucleotides conjugated to GalNAc, and formulated in phosphate buffered saline (PBS), a plasmid encoding human DGAT2 mRNA was injected into the mice via HDI and the percent (%) of human DGAT2 mRNA was measured 1 day later in liver samples from the mice relative to mice treated with PBS.

FIG. 13 provides a graph depicting the percent (%) of DGAT2 mRNA knockdown in liver samples from a hydrodynamic injection (HDI) model in CD-1 mice. Three (3) days after subcutaneous dosing of 0.3, 1, or 3 mg/kg of DGAT2 oligonucleotides conjugated to GalNAc, and formulated in phosphate buffered saline (PBS), a plasmid encoding human DGAT2 mRNA was injected into the mice via HDI and the percent (%) of human DGAT2 mRNA was measured 1 day later in liver samples from the mice relative to mice treated with PBS.

FIGS. 14A-14G provide graphs depicting the percent (%) of DGAT2 mRNA knockdown in liver samples from non-human primates (NHPs) treated with the indicated GalNAc-conjugated DGAT2 oligonucleotides relative to NHPs treated with phosphate buffered saline.

FIG. 15A provides a graph depicting the percent (%) of endogenous mouse DGAT2 mRNA knockdown in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 15B provides a graph depicting the percent (%) of endogenous mouse DGAT1 mRNA knockdown in liver samples from mice fed a GAN-NASH diet and treated with the indicated GalNAc-conjugated ACAC and/or DGAT2 oligonucleotides relative to mice treated with phosphate buffered saline (PBS).

FIGS. 16A-16B provide graphs depicting the percent (%) of endogenous mouse ACACA (FIG. 16A) and ACACB (FIG. 16B) mRNA knockdown in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 17 provides a graph depicting the level of serum cholesterol in samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 18 provides a graph depicting the level of serum ALT in samples from mice fed a GAN-NASH diet treated with GalNAc-conjugated ACAC (GalXC-ACAC-4458) and/or DGAT2 (GalXC-ACAC-1463) oligonucleotides relative to mice treated with phosphate buffered saline (PBS).

FIGS. 19A-19B provide a graph (FIG. 19A) and an image (FIG. 19B) depicting the level of liver steatosis as measured by % lipid area in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 20 provides a graph depicting the percent (%) of Srebp1 mRNA in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIGS. 21A-21B provide a graph (FIG. 21A) and images (FIG. 21B) depicting the level of macrophage infiltration as measured by F4/80 staining in liver samples from mice fed a GAN-NASH diet and treated with the indicated GalNAc-conjugated ACAC (GalXC-ACAC-4458) and/or DGAT2 (GalXC-ACAC-1463) oligonucleotides relative to mice treated with phosphate buffered saline (PBS).

FIGS. 22A-22B provide a graph (FIG. 22A) and images (FIG. 22B) depicting the level of macrophage pathogenesis as measured by an assay to identify crown-like structures (CLS) in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 23 provides a graph depicting the percent (%) of IL6 mRNA in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 24 provides a graph depicting the percent (%) of IL12b mRNA in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 25 provides a graph depicting the percent (%) of Col1a1 mRNA in liver samples from mice fed a GAN-NASH diet and treated with GalNAc-conjugated ACAC (GalXC-ACAC-4458) and/or DGAT2 (GalXC-ACAC-1463) oligonucleotides relative to mice treated with phosphate buffered saline (PBS).

FIG. 26 provides a graph depicting the percent (%) of Timp1 mRNA in liver samples from mice fed a GAN-NASH diet and treated with GalNAc-conjugated ACAC(GalXC-ACAC-4458) and/or DGAT2 (GalXC-ACAC-1463) oligonucleotides relative to mice treated with phosphate buffered saline (PBS).

### DETAILED DESCRIPTION

### I. Definitions

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a condition in the subject).

As used herein, the term “ACC” or “ACAC” refers to acetyl-Coenzyme A carboxylase (acetyl-CoA), a biotin-dependent enzyme that catalyzes the irreversible carboxylation of acetyl-CoA to produce malonyl CoA through its two catalytic activities, biotin carboxylase and carboxyltransferase. The term ACC, as used in this application refers to both isoforms of the ACC protein together; ACC1 and ACC2. ‘ACC’ may also refer to as both genes which encode the proteins, ACACA and ACACB, respectively. Inhibition of ACC can refer to inhibition of both ACC protein isoforms, inhibition of both ACC genes at the transcriptional level, inhibition of ACC enzymatic activity, or all of these.

As used herein, the term “antisense oligonucleotide” encompasses a nucleic acid-based molecule which has a sequence complementary to all or part of the target mRNA (e.g., ACC or DGAT2), in particular seed sequence thereby capable of forming a duplex with a mRNA. Thus, the term “antisense oligonucleotide”, as used herein, may be referred to as “complementary nucleic acid-based inhibitor”.

As used herein, “attenuate,” “attenuating,” “attenuation” and the like refers to reducing or effectively halting. As a non-limiting example, one or more of the treatments herein may reduce or effectively halt the onset or progression of dyslipidemia/hypertriglyceridemia/hyperlipidemia in a subject. This attenuation may be exemplified by, for example, a decrease in one or more aspects (e.g., symptoms, tissue characteristics, and cellular, inflammatory or immunological activity, etc.,) of dyslipidemia/hypertriglyceridemia/hyperlipidemia, no detectable progression (worsening) of one or more aspects of dyslipidemia/hypertriglyceridemia/hyperlipidemia, or no detectable aspects of dyslipidemia/hypertriglyceridemia/hyperlipidemia in a subject when they might otherwise be expected.

As used herein, “combination product”, “combination therapy”, “polytherapy” and the like refer to a therapy used for the treatment of a disease or disorder using more than one therapeutic agent or more than one medicament or modality. The therapeutic agents comprising a combination product may be dosed concurrently, intermittently or in any sequence. A combination product may comprise, for example, two oligonucleotides or an oligonucleotide combined with an antibody or small-molecule drug. For such therapies the dosages of each agent used may vary to optimize and/or enhance patient outcome.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “DGAT2” is used to refer to Diacylglycerol O-acyltransferase 2. DGAT2 is one of two enzymes which catalyze the final reaction in the synthesis of triglycerides in which diacylglycerol is covalently bound to long chain fatty acyl-CoA molecules. As used in this application, DGAT2 can refer to either the DGAT2 protein or the DGAT2 gene. Inhibition of DGAT2 can refer to inhibition of DGAT2 protein, inhibition of the DGAT2 gene at the transcription level, inhibition of the DGAT2 activity, or all of these.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded RNA” or “dsRNA” refers to an RNA oligonucleotide that is substantially in a duplex form. In some embodiments, the complementary base-pairing of duplex region(s) of a dsRNA oligonucleotide is formed between antiparallel sequences of nucleotides of covalently separate nucleic acid strands. In some embodiments, complementary base-pairing of duplex region(s) of a dsRNA formed between antiparallel sequences of nucleotides of nucleic acid strands that are covalently linked. In some embodiments, complementary base-pairing of duplex region(s) of a dsRNA is formed from single nucleic acid strand that is folded (e.g., via a hairpin) to provide complementary antiparallel sequences of nucleotides that base pair together. In some embodiments, a dsRNA comprises two covalently separate nucleic acid strands that are fully duplexed with one another. However, in some embodiments, a dsRNA comprises two covalently separate nucleic acid strands that are partially duplexed (e.g., having overhangs at one or both ends). In some embodiments, a dsRNA comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “hepatocyte” or “hepatocytes” refers to cells of the parenchymal tissues of the liver. These cells make up about 70%-85% of the liver’s mass and manufacture serum albumin, FBN and the prothrombin group of clotting factors (except for Factors 3 and 4). Markers for hepatocyte lineage cells include, but are not limited to, transthyretin (Ttr), glutamine synthetase (Glul), hepatocyte nuclear factor 1a (Hnf1a) and hepatocyte nuclear factor 4a (Hnf4a). Markers for mature hepatocytes may include, but are not limited to, cytochrome P450 (Cyp3a11), fumarylacetoacetate hydrolase (Fah), glucose 6-phosphate (G6p), albumin (Alb) and OC2-2F8. See, e.g., Huch et al. (2013) NATURE 494:247-50.

As used herein, a “hepatotoxic agent” refers to a chemical compound, virus or other substance that is itself toxic to the liver or can be processed to form a metabolite that is toxic to the liver. Hepatotoxic agents may include, but are not limited to, carbon tetrachloride (CCl₄), acetaminophen (paracetamol), vinyl chloride, arsenic, chloroform, nonsteroidal antiinflammatory drugs (such as aspirin and phenylbutazone).

As used herein, “labile linker” refers to a linker that can be cleaved (e.g., by acidic pH). A “fairly stable linker” refers to a linker that cannot be cleaved.

As used herein, “liver inflammation” or “hepatitis” refers to a physical condition in which the liver becomes swollen, dysfunctional and/or painful, especially as a result of injury or infection, as may be caused by exposure to a hepatotoxic agent. Symptoms may include jaundice (yellowing of the skin or eyes), fatigue, weakness, nausea, vomiting, appetite reduction and weight loss. Liver inflammation, if left untreated, may progress to fibrosis, cirrhosis, liver failure or liver cancer.

As used herein, “liver fibrosis” “Liver Fibrosis” or “fibrosis of the liver” refers to an excessive accumulation in the liver of extracellular matrix proteins, which could include collagens (I, III, and IV), FBN, undulin, elastin, laminin, hyaluronan and proteoglycans resulting from inflammation and liver cell death. Liver fibrosis, if left untreated, may progress to cirrhosis, liver failure or liver cancer.

As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “Metabolic syndrome’ or “metabolic liver disease” refers to a disorder characterized by a cluster of associated medical conditions and associated pathologies including, but not limited to the following medical conditions: abdominal obesity, elevated blood pressure, elevated fasting plasma glucose, high serum triglycerides, liver fibrosis, and low levels of high-density lipoprotein (HDL) levels. As used herein, the term metabolic syndrome or metabolic liver disease may encompass a wide array of direct and indirect manifestations, diseases and pathologies associated with metabolic syndrome and metabolic liver disease, with an expanded list of conditions used throughout the document.

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of a RNAi oligonucleotide that is characterized by separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

As used herein, “oligonucleotide” refers to a short nucleic acid (e.g., less than about 100 nucleotides in length). An oligonucleotide may be single-stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a double-stranded (dsRNA) is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a dsRNA. In certain embodiments, the overhang is a 3′ or 5′ overhang on the antisense strand or sense strand of a dsRNA.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., U.S. Provisional Pat. Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al. (2015) Nucleic Acids Res. 43:2993-3011).

As used herein, “reduced expression” of a gene (e.g., ACC and/or DGAT2) refers to a decrease in the amount or level of RNA transcript (e.g., ACC and/or DGAT2 mRNA) or protein encoded by the gene and/or a decrease in the amount or level of activity of the gene in a cell, a population of cells, a sample or a subject, when compared to an appropriate reference (e.g., a reference cell, population of cells, sample or subject). For example, the act of contacting a cell with an oligonucleotide herein (e.g., an oligonucleotide comprising an antisense strand having a nucleotide sequence that is complementary to a nucleotide sequence comprising ACC and/or DGAT2 mRNA) may result in a decrease in the amount or level of ACC and/or DGAT2 mRNA, protein and/or activity (e.g., via degradation of ACC and/or DGAT2 mRNA by the RNAi pathway) when compared to a cell that is not treated with the dsRNA. Similarly, and as used herein, “reducing expression” refers to an act that results in reduced expression of a gene (e.g., ACC and/or DGAT2). As used herein, “reduction of ACC and/or DGAT2 expression” refers to a decrease in the amount or level of ACC and/or DGAT2 mRNA, ACC and/or DGAT2 protein and/or ACC and/or DGAT2 activity in a cell, a population of cells, a sample or a subject when compared to an appropriate reference (e.g., a reference cell, population of cells, sample, or subject).

As used herein, “region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence (e.g., ACC or DGAT2).

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a dsRNA having a sense strand (passenger) and antisense strand (guide), in which the antisense strand or part of the antisense strand is used by the Argonaute 2 (Ago2) endonuclease in the cleavage of a target mRNA or (b) a ss oligonucleotide having a single antisense strand, where that antisense strand (or part of that antisense strand) is used by the Ago2 endonuclease in the cleavage of a target mRNA.

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraloop may stabilize a bp in an adjacent stem duplex by stacking interactions. In addition, interactions among the nucleotides in a tetraloop include, but are not limited to, non-Watson-Crick base pairing, stacking interactions, hydrogen bonding and contact interactions (Cheong et al. (1990) Nature 346:680-82; Heus & Pardi (1991) SCIENCE 253:191-94). In some embodiments, a tetraloop comprises or consists of 3 to 6 nucleotides and is typically 4 to 5 nucleotides. In certain embodiments, a tetraloop comprises or consists of 3, 4, 5 or 6 nucleotides, which may or may not be modified (e.g., which may or may not be conjugated to a targeting moiety). In one embodiment, a tetraloop consists of 4 nucleotides. Any nucleotide may be used in the tetraloop and standard IUPAC-IUB symbols for such nucleotides may be used as described in Cornish-Bowden (1985) NUCLEIC ACIDS RES. 13:3021-30. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al. (1990) PROC. NATL. ACAD. SCI. USA 87:8467-8471; Antao et al. (1991) NUCLEIC ACIDS RES. 19:5901-5905). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, e.g., Nakano et al. (2002) BIOCHEM. 41:4281-14292; Shinji et al. (2000) NIPPON KAGAKKAI KOEN YOKOSHU 78:731. In some embodiments, the tetraloop is contained within a nicked tetraloop structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### II. Oligonucleotide Inhibitors of ACC and/or DGAT2 Expression

The disclosure provides, inter alia, oligonucleotides that inhibit ACC and/or DGAT2 expression. In some embodiments, an oligonucleotide that inhibits ACC and/or DGAT2 expression herein is targeted to an ACC and/or DGAT2 mRNA. In some embodiments, the oligonucleotides reduce ACC and/or DGAT2 expression.

Acetyl-CoA Carboxylase (ACC), is a biotin-dependent enzyme that catalyzes the irreversible carboxylation of acetyl-CoA to produce malonyl-CoA through two of its catalytic activities, biotin carboxylase (BC) and carboxyltransferase (CT). ACC performs a third function as a biotin carboxyl carrier protein. Malonyl-CoA produced through the catalytic activities of ACC serves as the substrate for the biosynthesis of fatty acids. ACC acts as a key switch regulator in the transition from fatty acid synthesis to fatty acid oxidation. (Esler & Bense, CELL MOL GASTROENTEROL HEPATOL. (2019) 8(2): 247-67). Regulation of fatty acid synthesis plays an important role in the energy metabolism of fatty acids humans. For this reason, regulators of fatty acid synthesis such as ACC are considered an attractive target to regulate the human diseases associated with Metabolic Syndrome including obesity, diabetes, and cardiovascular complications (Wakil & Abu-Elheiga, (2009) J LIPID RES. 50: S138-S143). Several attempts have been made to modulate the activity of ACC at the transcriptional level or by small molecule modulators for use in a wide variety of indications, from cancer to diabetes and even as agricultural herbicides (Luo et al. (2012) RECENT PAT ANTICANCER DRUG DISCOV. 7(2):168-84). To date, this effort has not produced viable therapeutics capable of long-term improvements for patients. RNAi usage according to the current disclosure offers a different modality of approach and oligonucleotide configuration to assist in this endeavor.

From a genomic perspective, the human genome contains the genes for two different ACC proteins; ACACA and ACACB which are associated with the two main protein isoforms, ACC1 and ACC2, respectively. Due to discordance of the gene and protein nomenclature with this target, leading to inconsistent usage and labelling in the prior art, this application will refer to the target generally as ACC. The term ACC will be inclusive of both genes and their mRNA products (ACACA and ACACB), as well as their respective protein products (ACC1 and ACC2). At times, when differences between mRNA or protein isoforms are highlighted, the application will refer specifically to each isoform.

Inhibition of ACC, due to its role as a modulator of lipid metabolism pathways, holds promise as a therapeutic for treating Metabolic Syndrome and associated disorders. Despite decades as an identified target, attractive for therapeutic development across a broad range of indications, ACC remains difficult to meaningfully regulate. Studies conducted in humans using a small molecule inhibitor of ACC (ACC1/ACC2) revealed decreased lipogenesis, increased ketones, and reduced liver triglycerides after administration to subjects with NAFLD for one month (Horton et al. (2017) CELL METAB., 26(2): 394-406). However, these same patients also experienced a 200% increase in serum triglyceride levels.

Diacylglycerol O-acyltransferase 2, (DGAT2) is one of two enzymes which catalyze the final reaction in the synthesis of triglycerides in which diacylglycerol is covalently bound to long chain fatty acyl-CoA molecules. Increased serum triglycerides are a hallmark of Metabolic Syndrome and modulation of serum triglyceride levels at the transcriptional level or through other means is key to the control of related manifestations of the disorder. In addition, studies using mice fed a diet resulting in a phenotype which mimics non-alcoholic fatty liver disease (NAFLD) show that the specific knockdown of DGAT2 in liver tissue can lead to a decrease in lower levels of liver steatosis in these animals without increasing inflammation or fibrosis (Walther, et al. HEPATOLOGY (2019) 70(6): 1972-85). However, DGAT2 is associated with a narrower range of function, and possibly activity, than some other gene targets involved in fatty acid metabolism. There is also another enzyme, DGAT1, which shares redundant and overlapping functions with DGAT2 (Chitraju et al. (2019) J LIPID RES. 60(6): 1112-20). However, those previously in the field have had limited success in finding a small molecule compound that can reliably lower this synthesis without widespread effects on other related genes involved in fatty acid metabolism.

Accordingly, the present disclosure provides RNAi therapeutics targeting ACC and DGAT, alone or in combination.

### I. ACC and/or DGAT2 Target Sequences

In some embodiments, the oligonucleotide herein (e.g., an RNAi oligonucleotide) is targeted to a target sequence comprising an ACC and/or DGAT2 mRNA. In some embodiments, an oligonucleotide described herein corresponds to a target sequence within an ACC and/or DGAT2 mRNA sequence. In some embodiments, the oligonucleotide, or a portion, fragment or strand thereof (e.g., an antisense strand or a guide strand of a dsRNA) binds or anneals to a target sequence comprising an ACC and/or DGAT2 mRNA, thereby inhibiting ACC and/or DGAT2 expression.

In some embodiments, the oligonucleotide is targeted to an ACC and/or DGAT2 target sequence for the purpose of inhibiting ACC and/or DGAT2 expression in vivo. In some embodiments, the amount or extent of inhibition of ACC and/or DGAT2 expression by an oligonucleotide targeted to an ACC and/or DGAT2 target sequence correlates with the potency of the oligonucleotide. In some embodiments, the amount or extent of inhibition of ACC and/or DGAT2 expression by an oligonucleotide targeted to an ACC and/or DGAT2 target sequence correlates with the amount or extent of therapeutic benefit in a subject or patient having a disease, disorder or condition associated with the expression of ACC and/or DGAT2 treated with the oligonucleotide.

Through examination of the nucleotide sequence of mRNAs encoding ACC and/or DGAT2, including mRNAs of multiple different species (e.g., human, cynomolgus monkey, mouse, and rat; see, e.g., Example 1) and as a result of in vitro and in vivo testing (see, e.g., Example 2, Example 3, and Examples 4-10), it has been discovered that certain nucleotide sequences of ACC and/or DGAT2 mRNA are more amenable than others to oligonucleotide-based inhibition and are thus useful as target sequences for the oligonucleotides herein. In some embodiments, a sense strand of an oligonucleotide (e.g., a dsRNA) described herein comprises an ACC and/or DGAT2 target sequence. In some embodiments, a portion or region of the sense strand of a dsRNA described herein comprises an ACC and/or DGAT2 target sequence. In some embodiments, an ACC and/or DGAT2 target sequence comprises, or consists of, a sequence of any one of SEQ ID Nos: 149, 150, 151, 152, 153, 154, 155, 156, 157 and 158. In some embodiments, the ACC target sequence comprises, or consists of, a sequence of any one of SEQ ID NOs: 150, 151, 152, 153, 154, and 155. In some embodiments, the ACC target sequence comprises, or consists of, a sequence of any one of SEQ ID NOs: 150 and 151. In some embodiments, the DGAT2 target sequence comprises, or consists of, a sequence of any one of SEQ ID NOs: 149, 156, 157, and 158. In some embodiments, the DGAT2 target sequence comprises, or consists of, a sequence of any one of SEQ ID NOs: 156 and 157.

### Ii. ACC and/or DGAT2 Targeting Sequences

In some embodiments, the oligonucleotides herein (e.g., RNAi oligonucleotides) have regions of complementarity to ACC and/or DGAT2 mRNA (e.g., within a target sequence of ACC and/or DGAT2 mRNA) for purposes of targeting the mRNA in cells and inhibiting its expression. In some embodiments, the oligonucleotides herein comprise an ACC and/or DGAT2 targeting sequence (e.g., an antisense strand or a guide strand of a dsRNA) having a region of complementarity that binds or anneals to an ACC and/or DGAT2 target sequence by complementary (Watson-Crick) base pairing. The targeting sequence or region of complementarity is generally of a suitable length and base content to enable binding or annealing of the oligonucleotide (or a strand thereof) to an ACC and/or DGAT2 mRNA for purposes of inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29 or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length.

In some embodiments, an oligonucleotide herein comprises a targeting sequence or a region of complementarity (e.g., an antisense strand or a guide strand of a double-stranded oligonucleotide) that is fully complementary to an ACC and/or DGAT2 target sequence. In some embodiments, the targeting sequence or region of complementarity is partially complementary to an ACC and/or DGAT2 target sequence. In some embodiments, the oligonucleotide comprises a targeting sequence or region of complementarity that is fully complementary to a sequence of ACC or DGAT2.

In some embodiments, the oligonucleotide comprises a targeting sequence or region of complementarity complementary to a sequence of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, and 83, and the targeting sequence or region of complementarity is 18 nucleotides in length. In some embodiments, an oligonucleotide comprises a target sequence or region of complementarity complementary to a sequence of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, and 83, and the targeting sequence or region of complementarity is 19 nucleotides in length. In some embodiments, an oligonucleotide comprises a target sequence or region of complementarity complementary to a sequence of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, and 83, and the targeting sequence or region of complementarity is 20 nucleotides in length. In some embodiments, the oligonucleotide comprises a targeting sequence or region of complementarity complementary to a sequence selected from SEQ ID Nos: 29, 31 and 43, and the targeting sequence or region of complementarity is 18 nucleotides in length. In some embodiments, the oligonucleotide comprises a targeting sequence or region of complementarity complementary to a sequence selected from SEQ ID Nos: 29, 31 and 43, and the targeting sequence or region of complementarity is 19 nucleotides in length. In some embodiments, the oligonucleotide comprises a targeting sequence or region of complementarity complementary to a sequence selected from SEQ ID Nos: 29, 31 and 43, and the targeting sequence or region of complementarity is 20 nucleotides in length. In some embodiments, an oligonucleotide comprises a target sequence or region of complementarity complementary to a sequence of any one of SEQ ID NOs: 150 and 151, and the targeting sequence or region of complementarity is 21 nucleotides in length. In some embodiments, an oligonucleotide comprises a target sequence or region of complementarity complementary to a sequence of any one of SEQ ID NOs: 150 and 151, and the targeting sequence or region of complementarity is 22 nucleotides in length. In some embodiments, an oligonucleotide comprises a target sequence or region of complementarity complementary to a sequence of any one of SEQ ID NOs: 150 and 151, and the targeting sequence or region of complementarity is 23 nucleotides in length. In some embodiments, an oligonucleotide comprises a target sequence or region of complementarity complementary to a sequence of any one of SEQ ID NOs: 150 and 151, and the targeting sequence or region of complementarity is 24 nucleotides in length.

In some embodiments, the oligonucleotide comprises a targeting sequence or region of complementarity complementary to a sequence of any one of SEQ ID NOs: 117, 119, 121, 123, 125, 127, 129, 131, 133, 135, 137, 139, 141, 143, 145, 147, and the targeting sequence or region of complementarity is 18 nucleotides in length. In some embodiments, an oligonucleotide comprises a target sequence or region of complementarity complementary to a sequence of any one of SEQ ID NOs: 117, 119, 121, 123, 125, 127, 129, 131, 133, 135, 137, 139, 141, 143, 145, 147, and the targeting sequence or region of complementarity is 19 nucleotides in length. In some embodiments, an oligonucleotide comprises a target sequence or region of complementarity complementary to a sequence of any one of SEQ ID NOs: 117, 119, 121, 123, 125, 127, 129, 131, 133, 135, 137, 139, 141, 143, 145, 147, and the targeting sequence or region of complementarity is 20 nucleotides in length. In some embodiments, the oligonucleotide comprises a targeting sequence or region of complementarity complementary to a sequence selected from SEQ ID Nos: 117, 119, 125, 129, 137 and 143, and the targeting sequence or region of complementarity is 18 nucleotides in length. In some embodiments, the oligonucleotide comprises a targeting sequence or region of complementarity complementary to a sequence selected from SEQ ID Nos: 117, 119, 125, 129, 137 and 143, and the targeting sequence or region of complementarity is 19 nucleotides in length. In some embodiments, the oligonucleotide comprises a targeting sequence or region of complementarity complementary to a sequence selected from SEQ ID Nos: 117, 119, 125, 129, 137 and 143, and the targeting sequence or region of complementarity is 20 nucleotides in length. In some embodiments, an oligonucleotide comprises a target sequence or region of complementarity complementary to a sequence of any one of SEQ ID NOs: 156 and 157, and the targeting sequence or region of complementarity is 21 nucleotides in length. In some embodiments, an oligonucleotide comprises a target sequence or region of complementarity complementary to a sequence of any one of SEQ ID NOs: 156 and 157 and the targeting sequence or region of complementarity is 22 nucleotides in length. In some embodiments, an oligonucleotide comprises a target sequence or region of complementarity complementary to a sequence of any one of SEQ ID NOs: 156 and 157, and the targeting sequence or region of complementarity is 23 nucleotides in length. In some embodiments, an oligonucleotide comprises a target sequence or region of complementarity complementary to a sequence of any one of SEQ ID NOs: 156 and 157, and the targeting sequence or region of complementarity is 24 nucleotides in length. In some embodiments, the oligonucleotide comprises a targeting sequence or region of complementarity that is partially complementary to a sequence of ACC or DGAT2.

In some embodiments, the oligonucleotide comprises a targeting sequence or region of complementarity that is fully complementary to a sequence of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, and 83. In some embodiments, the oligonucleotide comprises a targeting sequence or region of complementarity that is fully complementary to the sequence set forth in SEQ ID NO: 29, 31, and 43. In some embodiments, the oligonucleotide comprises a targeting sequence or region of complementarity that is partially complementary to a sequence of any one of SEQ ID NOs: 117, 119, 121, 123, 125, 127, 129, 131, 133, 135, 137, 139, 141, 143, 145, 147. In some embodiments, the oligonucleotide comprises a targeting sequence or region of complementarity that is partially complementary to the sequence set forth in SEQ ID NO: 117, 119, 125, 129, 137, and 143.

In some embodiments, the oligonucleotide herein (e.g. an RNAi oligonucleotide) comprises a targeting sequence or region of complementarity that is complementary to a contiguous sequence of nucleotides comprising an ACC and/or DGAT2 mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20 or 18 to 19 nucleotides in length). In some embodiments, the oligonucleotide comprises a targeting sequence or region of complementarity that is complementary to a contiguous sequence of nucleotides comprising an ACC and/or DGAT2 mRNA, wherein the contiguous sequence of nucleotides is 10, 11, 12, 13, 14, 15, 16, 17, 18, 19 or 20 nucleotides in length. In some embodiments, the oligonucleotide comprises a targeting sequence or region of complementarity that is complementary to a contiguous sequence of nucleotides comprising an ACC and/or DGAT2 mRNA, wherein the contiguous sequence of nucleotides is 19 nucleotides in length. In some embodiments, the oligonucleotide comprises a targeting sequence or region of complementarity that is complementary to a contiguous sequence of nucleotides comprising an ACC and/or DGAT2 mRNA, wherein the contiguous sequence of nucleotides is 20 nucleotides in length.

In some embodiments, a targeting sequence or region of complementarity of an oligonucleotide that is complementary to contiguous nucleotides of ACC or DGAT2 target sequence spans the entire length of an antisense strand. In some embodiments, a region of complementarity of an oligonucleotide that is complementary to contiguous nucleotides of ACC or DGAT2 target sequence spans a portion of the entire length of an antisense strand. In some embodiments, an oligonucleotide herein comprises a region of complementarity (e.g., on an antisense strand of a dsRNA) that is at least partially (e.g., fully) complementary to a contiguous stretch of nucleotides spanning nucleotides 1-20 of a target sequence of ACC or DGAT2.

In some embodiments, an oligonucleotide herein comprises a targeting sequence or region of complementarity having one or more base pair (bp) mismatches with the corresponding ACC and/or DGAT2 target sequence. In some embodiments, the targeting sequence or region of complementarity may have up to about 1, up to about 2, up to about 3, up to about 4, up to about 5, etc. mismatches with the corresponding ACC and/or DGAT2 target sequence provided that the ability of the targeting sequence or region of complementarity to bind or anneal to the ACC and/or DGAT2 mRNA under appropriate hybridization conditions and/or the ability of the oligonucleotide to inhibit ACC and/or DGAT2 expression is maintained. Alternatively, the targeting sequence or region of complementarity may have no more than 1, no more than 2, no more than 3, no more than 4, or no more than 5 mismatches with the corresponding ACC and/or DGAT2 target sequence provided that the ability of the targeting sequence or region of complementarity to bind or anneal to the ACC and/or DGAT2 mRNA under appropriate hybridization conditions and/or the ability of the oligonucleotide to inhibit ACC and/or DGAT2 expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or where in the mismatches are interspersed throughout the targeting sequence or region of complementarity. In some embodiments, the oligonucleotide comprises a targeting sequence or region of complementarity having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

In some embodiments, the oligonucleotide comprises a targeting sequence or a region of complementary that is complementary to a contiguous sequence of nucleotides of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, and 83, wherein the targeting sequence or region of complementarity may have up to about 1, up to about 2, up to about 3, up to about 4, up to about 5, etc. mismatches with the corresponding ACC target sequence. In some embodiments, the oligonucleotide comprises a targeting sequence or a region of complementary that is complementary to a contiguous sequence of nucleotides of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, and 83, wherein the targeting sequence or region of complementarity may have no more than 1, no more than 2, no more than 3, no more than 4, or no more than 5 mismatches with the corresponding ACC target sequence. In some embodiments, the oligonucleotide comprises a targeting sequence or a region of complementary that is complementary to a contiguous sequence of nucleotides of any one of SEQ ID NOs: 29, 31, 43, wherein the targeting sequence or region of complementarity may have up to about 1, up to about 2, up to about 3, up to about 4, up to about 5, etc. mismatches with the corresponding ACC target sequence. In some embodiments, the oligonucleotide comprises a targeting sequence or a region of complementary that is complementary to a contiguous sequence of nucleotides of any one of SEQ ID NOs: 29, 31, 43, wherein the targeting sequence or region of complementarity may have no more than 1, no more than 2, no more than 3, no more than 4, or no more than 5 mismatches with the corresponding ACC target sequence.

In some embodiments, the oligonucleotide comprises a targeting sequence or a region of complementary that is complementary to a contiguous sequence of nucleotides of any one of SEQ ID NOs: 117, 119, 121, 123, 125, 127, 129, 131, 133, 135, 137, 139, 141, 143, 145, 147, wherein the targeting sequence or region of complementarity may have up to about 1, up to about 2, up to about 3, up to about 4, up to about 5, etc. mismatches with the corresponding DGAT2 target sequence. In some embodiments, the oligonucleotide comprises a targeting sequence or a region of complementary that is complementary to a contiguous sequence of nucleotides of any one of SEQ ID NOs: 117, 119, 121, 123, 125, 127, 129, 131, 133, 135, 137, 139, 141, 143, 145, 147, wherein the targeting sequence or region of complementarity may have no more than 1, no more than 2, no more than 3, no more than 4, or no more than 5 mismatches with the corresponding DGAT2 target sequence. In some embodiments, the oligonucleotide comprises a targeting sequence or a region of complementary that is complementary to a contiguous sequence of nucleotides of any one of SEQ ID NOs: 117, 119, 125, 129, 137, and 143, wherein the targeting sequence or region of complementarity may have up to about 1, up to about 2, up to about 3, up to about 4, up to about 5, etc. mismatches with the corresponding DGAT2 target sequence. In some embodiments, the oligonucleotide comprises a targeting sequence or a region of complementary that is complementary to a contiguous sequence of nucleotides of any one of SEQ ID NOs: 117, 119, 125, 129, 137, and 143, wherein the targeting sequence or region of complementarity may have no more than 1, no more than 2, no more than 3, no more than 4, or no more than 5 mismatches with the corresponding DGAT2 target sequence.

### Iii. Types of Oligonucleotides

A variety of oligonucleotide types and/or structures are useful for targeting ACC and/or DGAT2 in the methods herein including, but not limited to, RNAi oligonucleotides, antisense oligonucleotides, miRNAs, etc. Any of the oligonucleotide types described herein or elsewhere are contemplated for use as a framework to incorporate an ACC and/or DGAT2 targeting sequence herein.

In some embodiments, the oligonucleotides herein inhibit ACC and/or DGAT2 expression by engaging with RNA interference (RNAi) pathways upstream or downstream of Dicer involvement. For example, RNAi oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended dsRNAs where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as Intl. Patent Application Publication No. WO 2010/033225). Such structures may include ss extensions (on one or both sides of the molecule) as well as ds extensions.

In some embodiments, the oligonucleotides herein engage with the RNAi pathway downstream of the involvement of Dicer (e.g., Dicer cleavage). In some embodiments, the oligonucleotides described herein are Dicer substrates. In some embodiments, the oligonucleotide has an overhang (e.g., of 1, 2, or 3 nucleotides in length) in the 3′ end of the sense strand. In some embodiments, the oligonucleotide (e.g., siRNA) comprises a 21-nucleotide guide strand that is antisense to a target RNA and a complementary passenger strand, in which both strands anneal to form a 19-bp duplex and 2 nucleotide overhangs at either or both 3′ ends. Longer oligonucleotide designs also are available including oligonucleotides having a guide strand of 23 nucleotides and a passenger strand of 21 nucleotides, where there is a blunt end on the right side of the molecule (3′ end of passenger strand/5′ end of guide strand) and a two nucleotide 3′-guide strand overhang on the left side of the molecule (5′ end of the passenger strand/3′ end of the guide strand). In such molecules, there is a 21 bp duplex region. See, e.g., U.S. Pat. Nos. 9,012,138; 9,012,621 and 9,193,753.

In some embodiments, the oligonucleotides herein comprise sense and antisense strands that are both in the range of about 17 to 26 (e.g., 17 to 26, 20 to 25 or 21-23) nucleotides in length. In some embodiments, the oligonucleotides described herein comprise an antisense strand of 19-30 nucleotides in length and a sense strand of 19-50 nucleotides in length, wherein the antisense and sense strands are separate strands which form an asymmetric duplex region having an overhang of 1-4 nucleotides at the 3′ terminus of the antisense strand. In some embodiments, an oligonucleotide herein comprises a sense and antisense strand that are both in the range of about 19-22 nucleotides in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′-guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY. Blackburn (ed.), Royal Society of Chemistry, 2006), shRNAs (e.g., having 19 bp or shorter stems; see, e.g., Moore et al. (2010) METHODS MOL. BIOL. 629:141-158), blunt siRNAs (e.g., of 19 bps in length; see, e.g., Kraynack & Baker (2006) RNA 12:163-176), asymmetrical siRNAs (aiRNA; see, e.g., Sun et al. (2008) NAT. BIOTECHNOL. 26:1379-1382), asymmetric shorter-duplex siRNA (see, e.g., Chang et al. (2009) MOL. THER. 17:725-32), fork siRNAs (see, e.g., Hohjoh (2004) FEBS LETT. 557:193-198), ss siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) NUCLEIC ACIDS RES. 35:5886-97). Further non-limiting examples of an oligonucleotide structures that may be used in some embodiments to reduce or inhibit the expression of ACC and/or DGAT2 are microRNA (miRNA), short hairpin RNA (shRNA) and short siRNA (see, e.g., Hamilton et al. (2002) EMBO J. 21:4671-79; see also, U.S. Pat. Application Publication No. 2009/0099115).

Still, in some embodiments, an oligonucleotide for reducing or inhibiting ACC and/or DGAT2 expression herein is ss. Such structures may include but are not limited to ss RNAi molecules. Recent efforts have demonstrated the activity of ss RNAi molecules (see, e.g., Matsui et al. (2016) MOL. THER. 24:946-55). However, in some embodiments, oligonucleotides herein are antisense oligonucleotides (ASOs). An antisense oligonucleotide is a ss oligonucleotide that has a nucleobase sequence which, when written in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) ANNU. REV. PHARMACOL. 57:81-105).

In some embodiments, the antisense oligonucleotide shares a region of complementarity with ACC and/or DGAT2 mRNA. In some embodiments, the antisense oligonucleotide targets various areas of the human ACACB gene identified as NM_001093. In some embodiments, the antisense oligonucleotide targets various areas of the human ACACB gene identified as NM_198834. In some embodiments, the antisense oligonucleotide targets various areas of the human DGAT2 gene identified as NM_001253891.1. In some embodiments, the antisense oligonucleotide targets various areas of the human DGAT2 gene identified as NM_032564.5. In some embodiments, the antisense oligonucleotide is 15-50 nucleotides in length. In some embodiments, the antisense oligonucleotide is 15-25 nucleotides in length. In some embodiments, the antisense oligonucleotide is 22 nucleotides in length. In some embodiments, the antisense oligonucleotide is complementary to any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, and 83. In some embodiments, the antisense oligonucleotide is complementary to any one of SEQ ID NOs: 29, 31 and 43. In some embodiments, the antisense oligonucleotide is complementary to any one of SEQ ID NOs: 117, 119, 121, 123, 125, 127, 129, 131, 133, 135, 137, 139, 141, 143, 145, 147. In some embodiments, the antisense oligonucleotide is complementary to any one of SEQ ID NOs: 117, 119, 125, 129, 137 and 143. In some embodiments, the antisense oligonucleotide is at least 15 contiguous nucleotides in length. In some embodiments, the antisense oligonucleotide is at least 19 contiguous nucleotides in length. In some embodiments, the antisense oligonucleotide is at least 20 contiguous nucleotides in length. In some embodiments, the antisense oligonucleotide differs by 1, 2, or 3 nucleotides from the target sequence.

### IV Double-Stranded Oligonucleotides

The disclosure provides dsRNAs for targeting ACC and/or DGAT2 and inhibiting ACC and/or DGAT2 expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and antisense strand are covalently linked. In some embodiments, the sense strand and antisense strand form a duplex region, wherein the sense strand and antisense strand, or a portion thereof, binds with one another in a complementary fashion (e.g., by Watson-Crick base pairing).

In some embodiments, the sense strand has a first region (R1) and a second region (R2), wherein R2 comprises a first subregion (S1), a tetraloop or triloop (L), and a second subregion (S2), wherein L is located between S1 and S2, and wherein S1 and S2 form a second duplex (D2). D2 may have various length. In some embodiments, D2 is about 1-6 bp in length. In some embodiments, D2 is 2-6, 3-6, 4-6, 5-6, 1-5, 2-5, 3-5 or 4-5 bp in length. In some embodiments, D2 is 1, 2, 3, 4, 5 or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20 or at least 21) nucleotides in length. In some embodiments, D1 is in the range of about 12 to 30 nucleotides in length (e.g., 12 to 30, 12 to 27, 15 to 22, 18 to 22, 18 to 25, 18 to 27, 18 to 30 or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29 or 30 nucleotides in length. In some embodiments, D1 is 20 nucleotides in length. In some embodiments, D1 comprising sense strand and antisense strand does not span the entire length of the sense strand and/or antisense strand. In some embodiments, D1 comprising the sense strand and antisense strand spans the entire length of either the sense strand or antisense strand or both. In certain embodiments, D1 comprising the sense strand and antisense strand spans the entire length of both the sense strand and the antisense strand.

In some embodiments, a dsRNA herein comprises a sense strand having a sequence of any one of SEQ ID NOs: 1, 29, 31, 43, 55, 105, 107, 111, 125, 129, and 137 and an antisense strand comprising a complementary sequence selected from SEQ ID NOs: 2, 30, 32, 44, 56, 106, 108, 11, 126, 130 and 138, as is arranged Tables 1, 3, 4, 6, 8 and 9.

In some embodiments, an oligonucleotide provided herein (e.g., an RNAi oligonucleotide) comprises a sense strand and an antisense strand comprising nucleotide sequences selected from:
(a) SEQ ID NOs: 25 and 26, respectively;
(b) SEQ ID NOs: 27 and 28, respectively;
(c) SEQ ID NOs: 29 and 30, respectively;
(d) SEQ ID NOs: 31 and 32, respectively;
(e) SEQ ID NOs: 33 and 34, respectively;
(f) SEQ ID NOs: 35 and 36, respectively;
(g) SEQ ID NOs: 37 and 38, respectively;
(h) SEQ ID NOs: 39 and 40, respectively;
(i) SEQ ID NOs: 41 and 42, respectively;
(j) SEQ ID NOs: 43 and 44, respectively;
(k) SEQ ID NOs: 45 and 46, respectively;
(l) SEQ ID NOs: 47 and 48, respectively;
(m) SEQ ID NOs: 49 and 50, respectively;
(n) SEQ ID NOs: 51 and 52, respectively;
(o) SEQ ID NOs: 53 and 54, respectively;
(p) SEQ ID NOs: 55 and 56, respectively;
(q) SEQ ID NOs: 57 and 58, respectively;
(r) SEQ ID NOs: 59 and 60, respectively;
(s) SEQ ID NOs: 61 and 62, respectively;
(t) SEQ ID NOs: 63 and 64, respectively;
(u) SEQ ID NOs: 65 and 66, respectively;
(v) SEQ ID NOs: 67 and 68, respectively;
(w) SEQ ID NOs: 69 and 70, respectively;
(x) SEQ ID NOs: 71 and 72, respectively;
(y) SEQ ID NOs: 73 and 74, respectively;
(z) SEQ ID NOs: 75 and 76, respectively;
(aa) SEQ ID NOs: 77 and 78, respectively;
(bb) SEQ ID NOs: 79 and 80, respectively;
(cc) SEQ ID NOs: 81 and 82, respectively; and
(dd) SEQ ID NOs: 83 and 84, respectively.

In some embodiments, the sense strand comprises the sequence of SEQ ID NO: 43 and the antisense strand comprises the sequence of SEQ ID NO: 44.

In some embodiments, an oligonucleotide provided herein (e.g., an RNAi oligonucleotide) comprises a sense strand and an antisense strand comprising nucleotide sequences selected from:
(a) SEQ ID NOs: 117 and 118, respectively;
(b) SEQ ID NOs: 119 and 120, respectively;
(c) SEQ ID NOs: 121 and 122, respectively;
(d) SEQ ID NOs: 123 and 124, respectively;
(e) SEQ ID NOs: 125 and 126, respectively;
(f) SEQ ID NOs: 127 and 128, respectively;
(g) SEQ ID NOs: 129 and 130, respectively;
(h) SEQ ID NOs: 131 and 132, respectively;
(i) SEQ ID NOs: 133 and 134, respectively;
(j) SEQ ID NOs: 135 and 136, respectively;
(k) SEQ ID NOs: 137 and 138, respectively;
(l) SEQ ID NOs: 139 and 140, respectively;
(m) SEQ ID NOs: 141 and 142, respectively;
(n) SEQ ID NOs: 143 and 144, respectively;
(o) SEQ ID NOs: 145 and 146, respectively; and,
(p) SEQ ID NOs: 147 and 148, respectively;

In some embodiments, the sense strand comprises the sequence of SEQ ID NO: 119 and the antisense strand comprises the sequence of SEQ ID NO: 120. In some embodiments, the sense strand comprises the sequence of SEQ ID NO: 129 and the antisense strand comprises the sequence of SEQ ID NO: 130.

It should be appreciated that, in some embodiments, sequences presented in the Sequence Listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a double-stranded RNA (dsRNA) herein comprises a 25-nucleotide sense strand and a 27-nucleotide antisense strand that when acted upon by a Dicer enzyme results in an antisense strand that is incorporated into the mature RISC. In some embodiments, the sense strand of the dsRNA is longer than 27 nucleotides (e.g., 28, 29, 30, 31, 32, 33, 34, 35, 36, 37, 38, 39 or 40 nucleotides). In some embodiments, the sense strand of the dsRNA is longer than 25 nucleotides (e.g., 26, 27, 28, 29 or 30 nucleotides). In some embodiments, the sense strand of the oligonucleotide comprises a nucleotide sequence selected from SEQ ID NOs: 160-189, wherein the nucleotide sequence is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide comprises a nucleotide sequence selected from SEQ ID NOs: 160-189, wherein the nucleotide sequence is longer than 25 nucleotides (e.g., 26, 27, 28, 29 or 30 nucleotides).

In some embodiments, the sense strand of the oligonucleotide comprises a nucleotide sequence selected from SEQ ID NOs: 190-205, wherein the nucleotide sequence is longer than 27 nucleotides (e.g., 28, 29, 30, 31, 32, 33, 34, 35, 36, 37, 38, 39, 40, 41, 42, 43, 44, 45, 46, 47, 48, 49 or 50 nucleotides). In some embodiments, the sense strand of the oligonucleotide comprises a nucleotide sequence selected from SEQ ID NOs: 190-205, wherein the nucleotide sequence is longer than 25 nucleotides (e.g., 26, 27, 28, 29 or 30 nucleotides).

In some embodiments, oligonucleotides herein have one 5′ end that is thermodynamically less stable when compared to the other 5′ end. In some embodiments, an asymmetry oligonucleotide is provided that includes a blunt end at the 3′ end of a sense strand and a 3′-overhang at the 3′ end of an antisense strand. In some embodiments, the 3′-overhang on the antisense strand is about 1-8 nucleotides in length (e.g., 1, 2, 3, 4, 5, 6, 7 or 8 nucleotides in length). Typically, an oligonucleotide for RNAi has a two-nucleotide overhang on the 3′ end of the antisense (guide) strand. However, other overhangs are possible. In some embodiments, an overhang is a 3′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. In some embodiments, the oligonucleotide comprises a targeting sequence or a region of complementary that is complementary to a contiguous sequence of nucleotides of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, 83, 117, 119, 121, 123, 125, 127, 129, 131, 133, 135, 137, 139, 141, 143, 145, 147, and a 5′-overhang comprising a length of between 1 and 6 nucleotides. In some embodiments, the oligonucleotide comprises a sense strand comprising a nucleotide sequence selected from SEQ ID NOs: 160-189, and 190-205 wherein the oligonucleotide comprises a 5′-overhang comprising a length of between 1 and 6 nucleotides. In some embodiments, the oligonucleotide comprises an antisense strand comprising a nucleotide sequence selected from SEQ ID NOs: 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 118, 120, 122, 124, 126, 128, 130, 132, 134, 136, 138, 140, 142, 144, 146, and 148, wherein the oligonucleotide comprises a 5′-overhang comprising a length of between 1 and 6 nucleotides. In some embodiments, the oligonucleotide comprises a sense strand comprising a nucleotide sequence selected from SEQ ID NOs: 160-189, and 190-205 and antisense strand comprising a nucleotide sequence selected from SEQ ID NOs: 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 118, 120, 122, 124, 126, 128, 130, 132, 134, 136, 138, 140, 142, 144, 146, and 148, wherein the oligonucleotide comprises a 5′-overhang comprising a length of between 1 and 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., ACC or DGAT2 mRNA). In some embodiments, the two terminal nucleotides on the 3′ end of the antisense strand are not complementary with the target. In some embodiments, the two (2) terminal nucleotides on the 3′ end of the antisense strand of an oligonucleotide herein are unpaired. In some embodiments, the two (2) terminal nucleotides on the 3′ end of the antisense strand of an oligonucleotide herein comprise an unpaired GG. In some embodiments, the two (2) terminal nucleotides on the 3′ end of an antisense strand of an oligonucleotide herein are not complementary to the target mRNA. In some embodiments, two (2) terminal nucleotides on each 3′ end of an oligonucleotide are GG. In some embodiments, one or both of the two (2) terminal GG nucleotides on each 3′ end of an oligonucleotide herein is not complementary with the target mRNA. In some embodiments, two terminal nucleotides on each 3′ end of an oligonucleotide in the nicked tetraloop structure are GG. Typically, one or both of the two terminal GG nucleotides on each 3′ end of an oligonucleotide is not complementary with the target. In some embodiments, the oligonucleotide comprises a targeting sequence or a region of complementary that is complementary to a contiguous sequence of nucleotides of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, 83, 117, 119, 121, 123, 125, 127, 129, 131, 133, 135, 137, 139, 141, 143, 145, 147, wherein the two (2) terminal nucleotides on the 3′ end of the antisense strand of the oligonucleotide herein comprises an unpaired GG. In some embodiments, the oligonucleotide comprises an antisense strand comprising a nucleotide sequence selected from SEQ ID NOs: 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 118, 120, 122, 124, 126, 128, 130, 132, 134, 136, 138, 140, 142, 144, 146, and 148, wherein the two (2) terminal nucleotides on the 3′ end of the antisense strand of the oligonucleotide comprises an unpaired GG. In some embodiments, the oligonucleotide comprises a sense strand comprising a nucleotide sequence selected from SEQ ID NOs: 160-189 and 190-205, and antisense strand comprising a nucleotide sequence selected from SEQ ID NOs: 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 118, 120, 122, 124, 126, 128, 130, 132, 134, 136, 138, 140, 142, 144, 146, and 148 wherein the two (2) terminal nucleotides on the 3′ end of the antisense strand of the oligonucleotide comprises an unpaired GG.

In some embodiments, there is one or more (e.g., 1, 2, 3, 4 or 5) mismatch between a sense and antisense strand. If there is more than one mismatch between a sense and antisense strand, they may be positioned consecutively (e.g., 2, 3 or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches or destabilization of segments at the 3′ end of the sense strand of the oligonucleotide improved the potency of synthetic duplexes in RNAi, possibly through facilitating processing by Dicer.

In some embodiments, the sense and antisense strands of an oligonucleotide herein comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(l) SEQ ID NOs: 171 and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and
(dd) SEQ ID NOs: 189 and 84, respectively,
wherein there is one or more (e.g., 1, 2, 3, 4 or 5) mismatch(s) between the sense and antisense strands.

In some embodiments, the sense and antisense strands of an oligonucleotide herein comprise nucleotides sequences selected from the group consisting of:
(a) SEQ ID NOs: 162 and 30, respectively;
(b) SEQ ID NOs: 163 and 32, respectively; and
(c) SEQ ID NOs: 169 and 44, respectively,
wherein there is one or more (e.g., 1, 2, 3, 4 or 5) mismatch(s) between the sense and antisense strands.

In some embodiments, the sense and antisense strands of an oligonucleotide herein comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and
(p) SEQ ID NOs: 205 and 148, respectively,
wherein there is one or more (e.g., 1, 2, 3, 4 or 5) mismatch(s) between the sense and antisense strands.

In some embodiments, the sense and antisense strands of an oligonucleotide herein comprise nucleotides sequences selected from the group consisting of:
(a) SEQ ID NOs: 200 and 138, respectively;
(b) SEQ ID NOs: 203 and 144, respectively;
(c) SEQ ID NOs: 194 and 126, respectively;
(d) SEQ ID NOs: 191 and 120, respectively;
(e) SEQ ID NOs: 196 and 130, respectively; and
(f) SEQ ID NOs: 190 and 118, respectively,
wherein there is one or more (e.g., 1, 2, 3, 4 or 5) mismatch(s) between the sense and antisense strands.

### α. Antisense Strands

In some embodiments, an oligonucleotide disclosed herein (e.g., and RNAi oligonucleotide) for targeting ACC and/or DGAT2 comprises an antisense strand comprising or consisting of a sequence as set forth in any one of SEQ ID NOs: 2, 30, 32, 44, 56, 106, 108, 112, 126, 130 and 138. In some embodiments, an oligonucleotide comprises an antisense strand comprising or consisting of at least about 12 (e.g., at least 12, at least 13, at least 14, at least 15, at least 16, at least 17, at least 18, at least 19, at least 20, at least 21, at least 22 or at least 23) contiguous nucleotides of a sequence as set forth in any one of SEQ ID NOs: 2, 30, 32, 44, 56, 106, 108, 112, 126, 130 and 138. In some embodiments, an oligonucleotide comprises an antisense strand comprising or consisting of a sequence selected from SEQ ID Nos: 2, 30, 32, 44 and 56. In some embodiments, an oligonucleotide comprises an antisense strand comprising or consisting of a sequence selected from SEQ ID Nos: 30, 32, and 44. In some embodiments, an oligonucleotide comprises an antisense strand comprising or consisting of a sequences selected from SEQ ID Nos: 106, 108, 112, 118, 120, 126, 130 and 128. In some embodiments, an oligonucleotide comprises an antisense strand comprising or consisting of a sequences selected from SEQ ID Nos: 118, 120, 126, 130 and 128.

In some embodiments, an oligonucleotide herein (e.g., an RNAi oligonucleotide) comprises an antisense strand of up to about 50 nucleotides in length (e.g., up to 50, up to 40, up to 35, up to 30, up to 27, up to 25, up to 21, up to 19, up to 17 or up to 12 nucleotides in length). In some embodiments, a dsRNA comprises an antisense strand of up to about 40 nucleotides in length (e.g., up to 40, up to 35, up to 30, up to 27, up to 25, up to 21, up to 19, up to 17 or up to 12 nucleotides in length). In some embodiments, an oligonucleotide may have an antisense strand of at least about 12 nucleotides in length (e.g., at least 12, at least 15, at least 19, at least 21, at least 22, at least 25, at least 27, at least 30, at least 35 or at least 38 nucleotides in length). In some embodiments, an oligonucleotide may have an antisense strand in a range of about 12 to about 40 (e.g., 12 to 40, 12 to 36, 12 to 32, 12 to 28, 15 to 40, 15 to 36, 15 to 32, 15 to 28, 17 to 22, 17 to 25, 19 to 27, 19 to 30, 20 to 40, 22 to 40, 25 to 40 or 32 to 40) nucleotides in length. In some embodiments, an oligonucleotide may have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, an oligonucleotide comprises antisense strand of 15 to 30 nucleotides in length. In some embodiments, an antisense strand of any one of the oligonucleotides disclosed herein is of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length. In some embodiments, an oligonucleotide comprises an antisense strand of 22 nucleotides in length.

In some embodiments, an antisense strand of an oligonucleotide may be referred to as a “guide strand.” For example, if an antisense strand can engage with RNA-induced silencing complex (RISC) and bind to an Argonaute protein such as Ago2, or engage with or bind to one or more similar factors, and direct silencing of a target gene, it may be referred to as a guide strand. In some embodiments, a sense strand complementary to a guide strand may be referred to as a “passenger strand.”

### B. Sense Strands

In some embodiments, an oligonucleotide herein (e.g., an RNAi oligonucleotide) for targeting ACC and/or DGAT2 comprises or consists of a sense strand sequence as set forth in in any one of SEQ ID NOs: 1, 29, 31, 43, 55, 105, 107, 111, 125, 129 and 137. In some embodiments, an oligonucleotide has a sense strand that comprises or consists of at least about 12 (e.g., at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 1, 29, 31, 43, 55, 105, 107, 111, 125, 129 and 137. In some embodiments, an oligonucleotide herein comprises a sense strand comprised of at least about 12 (e.g. at least 13, at least 14, at least 15, at least 16, at least 17, at least 18, at least 19, at least 20, at least 21, at least 22 or at least 23) contiguous nucleotides of a sequence as set forth in in any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, 83, 117, 119, 121, 123, 125, 127, 129, 131, 133, 135, 137, 139, 141, 143, 145, and 147. In some embodiments, an oligonucleotide comprises a sense strand comprising or consisting of a sequence selected from SEQ ID Nos: 29, 31, 43 and 55. In some embodiments, an oligonucleotide comprises a sense strand comprising or consisting of a sequence selected from SEQ ID Nos: 162, 163, 169 and 175. In some embodiments, an oligonucleotide comprises a sense strand comprising or consisting of a sequence selected from SEQ ID Nos: 117, 119, 125, 129, 137 and 143. In some embodiments, an oligonucleotide comprises a sense strand comprising or consisting of a sequence selected from SEQ ID Nos: 190, 191, 194, 196, 200 and 203.

In some embodiments, an oligonucleotide comprises a sense strand (or passenger strand) of up to about 40 nucleotides in length (e.g., up to 40, up to 36, up to 30, up to 27, up to 25, up to 21, up to 19, up to 17 or up to 12 nucleotides in length). In some embodiments, an oligonucleotide may have a sense strand of at least about 12 nucleotides in length (e.g., at least 12, at least 15, at least 19, at least 21, at least 25, at least 27, at least 30, at least 36 or at least 38 nucleotides in length). In some embodiments, an oligonucleotide may have a sense strand in a range of about 12 to about 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40 or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments, an oligonucleotide disclosed herein for targeting ACC mRNA and inhibiting ACC expression comprises a sense strand sequence as set forth in any one of SEQ ID NOs: 160-189. In some embodiments, an oligonucleotide herein has a sense strand comprised of least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 160-189. In some embodiments, an oligonucleotide disclosed herein for targeting ACC mRNA and inhibiting ACC expression comprises a sense strand sequence as set forth in any one of SEQ ID NOs: 162, 163, and 169. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 162, 163, and 169. In some embodiments, an oligonucleotide disclosed herein for targeting ACC mRNA and inhibiting ACC expression comprises a sense strand sequence as set forth in any one of SEQ ID NOs: 29, 31, 43. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 29, 31, and 43.

In some embodiments, an oligonucleotide disclosed herein for targeting DGAT2 mRNA and inhibiting DGAT2 expression comprises a sense strand sequence as set forth in any one of SEQ ID NOs: 190-205. In some embodiments, an oligonucleotide herein has a sense strand comprised of least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 190-205. In some embodiments, an oligonucleotide disclosed herein for targeting DGAT2 mRNA and inhibiting DGAT2 expression comprises a sense strand sequence as set forth in any one of SEQ ID NOs: 200, 203, 194, 191, 196, and 190. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 200, 203, 194, 191, 196, and 190. In some embodiments, an oligonucleotide disclosed herein for targeting DGAT2 mRNA and inhibiting DGAT2 expression comprises a sense strand sequence as set forth in any one of SEQ ID NOs: 137, 143, 119, 125, 129, and 117. In some embodiments, an oligonucleotide herein has a sense strand that comprise at least about 12 (e.g., at least 13, at least 14, at least 15, at least 16, at least 17, at least 18, at least 19, at least 20, at least 21, at least 22 or at least 23) contiguous nucleotides of a sequence as set forth in any one of SEQ ID NOs: 137, 143, 119, 125, 129, and 117.

In some embodiments, an oligonucleotide provided herein (e.g., an RNAi oligonucleotide) comprises a sense strand comprising a stem-loop structure at the 3′ end of the sense strand. In some embodiments, the stem-loop is formed by intrastrand base pairing. In some embodiments, a sense strand comprises a stem-loop structure at its 5′ end. In some embodiments, the stem of the stem-loop comprises a duplex of 2, 3, 4, 5, 6, 7, 8, 9, 10, 11, 12, 13 or 14 nucleotides in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides the oligonucleotide protection against degradation (e.g., enzymatic degradation), facilitates or improves targeting and/or delivery to a target cell, tissue, or organ (e.g., the liver), or both. For example, in some embodiments, the loop of a stem-loop is comprised of nucleotides comprising one or more modifications that facilitate, improve, or increase targeting to a target mRNA (e.g., an ACC and/or DGAT2 mRNA), inhibition of target gene expression (e.g., ACC and/or DGAT2 expression), and/or delivery, uptake, and/or penetrance into a target cell, tissue, or organ (e.g., the liver), or a combination thereof. In some embodiments, the stem-loop itself or modification(s) to the stem-loop do not affect or do not substantially affect the inherent gene expression inhibition activity of the oligonucleotide, but facilitates, improves, or increases stability (e.g., provides protection against degradation) and/or delivery, uptake, and/or penetrance of the oligonucleotide to a target cell, tissue, or organ (e.g., the liver). In certain embodiments, an oligonucleotide herein comprises a sense strand comprising (e.g., at its 3′ end) a stem-loop set forth as: S1-L-S2, in which S1 is complementary to S2, and in which L forms a single-stranded loop of linked nucleotides between S1 and S2 of up to about 10 nucleotides in length (e.g., 3, 4, 5, 6, 7, 8, 9 or 10 nucleotides in length). In some embodiments, the loop (L) is 3 nucleotides in length. In some embodiments, the loop (L) is 4 nucleotides in length. In some embodiments, the loop (L) is 5 nucleotides in length. In some embodiments, the loop (L) is 6 nucleotides in length. In some embodiments, the loop (L) is 7 nucleotides in length. In some embodiments, the loop (L) is 8 nucleotides in length. In some embodiments, the loop (L) is 9 nucleotides in length. In some embodiments, the loop (L) is 10 nucleotides in length.

In some embodiments, the tetraloop comprises the sequence 5′-GAAA-3′. In some embodiments, the stem loop comprises the sequence 5′-GCAGCCGAAAGGCUGC-3′ (SEQ ID NO: 159).

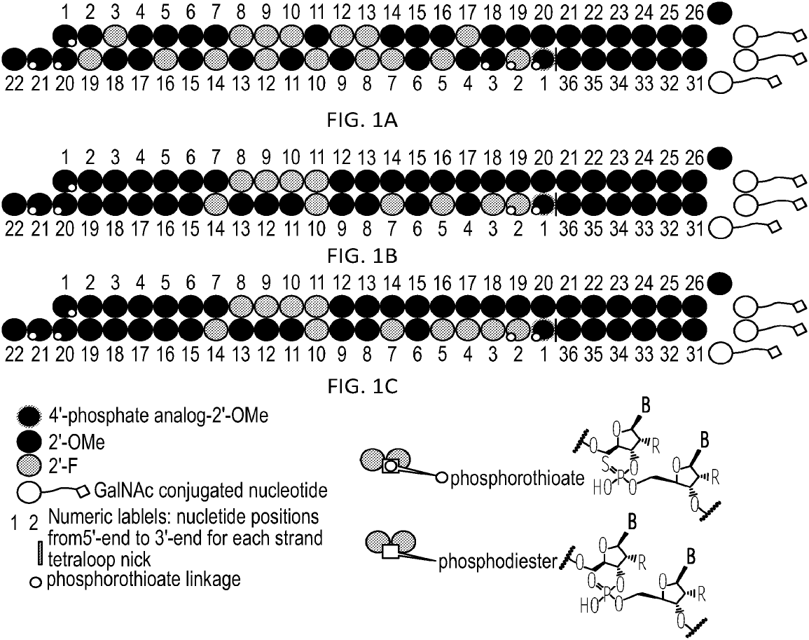

In some embodiments, a sense strand comprises a stem-loop structure at its 3′ end. In some embodiments, a sense strand comprises a stem-loop structure at its 5′ end. In some embodiments, a stem is a duplex of 2, 3, 4, 5, 6, 7, 8, 9, 10, 11, 12, 13 or 14 bp in length. In some embodiments, a stem-loop provides the molecule protection against degradation (e.g., enzymatic degradation) and facilitates targeting characteristics for delivery to a target cell. For example, in some embodiments, a loop provides added nucleotides on which modification can be made without substantially affecting the gene expression inhibition activity of an oligonucleotide. In certain embodiments, an oligonucleotide is herein in which the sense strand comprises (e.g., at its 3′ end) a stem-loop set forth as: S1-L-S2, in which S1 is complementary to S2, and in which L forms a loop between S1 and S2 of up to about 10 nucleotides in length (e.g., 3, 4, 5, 6, 7, 8, 9 or 10 nucleotides in length). In some embodiments, the oligonucleotide comprises a targeting sequence or a region of complementary that is complementary to a contiguous sequence of nucleotides of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, 83, 117, 119, 121, 123, 125, 127, 129, 131, 133, 135, 137, 139, 141, 143, 145, and 147, and the oligonucleotide comprises a sense strand comprising (e.g., at its 3′ end) a stem-loop set forth as: S1-L-S2, in which S1 is complementary to S2, and in which L forms a single-stranded loop between S1 and S2 of 4 nucleotides in length. FIGS. 1A-1C depict non-limiting examples of such an oligonucleotide.

In some embodiments, a loop (L) of a stem-loop having the structure S1-L-S2 as described herein is a triloop. In some embodiments, the oligonucleotide comprises a targeting sequence or a region of complementary that is complementary to a contiguous sequence of nucleotides of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, 83, 117, 119, 121, 123, 125, 127, 129, 131, 133, 135, 137, 139, 141, 143, 145, and 147 and a triloop. In some embodiments, the triloop comprises ribonucleotides, deoxyribonucleotides, modified nucleotides, ligands (e.g., delivery ligands), and combinations thereof.

In some embodiments, a loop (L) of a stem-loop having the structure S1-L-S2 as described above is a tetraloop. In some embodiments, an oligonucleotide herein comprises a targeting sequence or a region of complementary that is complementary to a contiguous sequence of nucleotides of any one of SEQ ID NOs: 25, 27, 29, 31, 33, 35, 37, 39, 41, 43, 45, 47, 49, 51, 53, 55, 57, 59, 61, 63, 65, 67, 69, 71, 73,75, 77, 79, 81, 83, 117, 119, 121, 123, 125, 127, 129, 131, 133, 135, 137, 139, 141, 143, 145, and 147 and a tetraloop. In some embodiments, the tetraloop comprises ribonucleotides, deoxyribonucleotides, modified nucleotides, ligands (e.g., delivery ligands), and combinations thereof.

In some embodiments, a loop (F) of a stem-loop is a tetraloop (e.g., within a nicked tetraloop structure). A tetraloop may contain ribonucleotides, deoxyribonucleotides, modified nucleotides and combinations thereof. Typically, a tetraloop has 4 to 5 nucleotides.

### C. Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 29, 21, 22, 23, 24, 25, 26, 27, 28, 29 or 30 nucleotides in length. In some embodiments, a duplex formed between a sense and antisense strand is 12 nucleotides in length. In some embodiments, a duplex formed between a sense and antisense strand is 13 nucleotides in length. In some embodiments, a duplex formed between a sense and antisense strand is 14 nucleotides in length. In some embodiments, a duplex formed between a sense and antisense strand is 15 nucleotides in length. In some embodiments, a duplex formed between a sense and antisense strand is 16 nucleotides in length. In some embodiments, a duplex formed between a sense and antisense strand is 17 nucleotides in length. In some embodiments, a duplex formed between a sense and antisense strand is 18 nucleotides in length. In some embodiments, a duplex formed between a sense and antisense strand is 19 nucleotides in length. In some embodiments, a duplex formed between a sense and antisense strand is 20 nucleotides in length. In some embodiments, a duplex formed between a sense and antisense strand is 21 nucleotides in length. In some embodiments, a duplex formed between a sense and antisense strand is 22 nucleotides in length. In some embodiments, a duplex formed between a sense and antisense strand is 23 nucleotides in length. In some embodiments, a duplex formed between a sense and antisense strand is 24 nucleotides in length. In some embodiments, a duplex formed between a sense and antisense strand is 25 nucleotides in length. In some embodiments, a duplex formed between a sense and antisense strand is 26 nucleotides in length. In some embodiments, a duplex formed between a sense and antisense strand is 27 nucleotides in length. In some embodiments, a duplex formed between a sense and antisense strand is 28 nucleotides in length. In some embodiments, a duplex formed between a sense and antisense strand is 29 nucleotides in length. In some embodiments, a duplex formed between a sense and antisense strand is 30 nucleotides in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

In some embodiments, the sense and antisense strands of an oligonucleotide comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(l) SEQ ID NOs: 171 and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and
(dd) SEQ ID NOs: 189 and 84, respectively,
wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length).

In some embodiments, a duplex between a sense and antisense strand spans the entire length of either the sense or antisense strands. In some embodiments, a duplex between a sense and antisense strand spans the entire length of both the sense strand and the antisense strand. In some embodiments, the sense and antisense strands of an oligonucleotide comprise nucleotides sequences selected from the group consisting of:
(a) SEQ ID NOs: 162 and 30, respectively;
(b) SEQ ID NOs: 163 and 32, respectively; and
(c) SEQ ID NOs: 169 and 44, respectively,
wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length).

In some embodiments, a duplex between a sense and antisense strand spans the entire length of either the sense or antisense strands. In some embodiments, a duplex between a sense and antisense strand spans the entire length of both the sense strand and the antisense strand. In some embodiments, the sense and antisense strands of an oligonucleotide comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and
(p) SEQ ID NOs: 205 and 148, respectively,
wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length).

In some embodiments, a duplex between a sense and antisense strand spans the entire length of either the sense or antisense strands. In some embodiments, a duplex between a sense and antisense strand spans the entire length of both the sense strand and the antisense strand. In some embodiments, the sense and antisense strands of an oligonucleotide comprise nucleotides sequences selected from the group consisting of:
(a) SEQ ID NOs: 200 and 138, respectively;
(b) SEQ ID NOs: 203 and 144, respectively;
(c) SEQ ID NOs: 194 and 126, respectively;
(d) SEQ ID NOs: 191 and 120, respectively;
(e) SEQ ID NOs: 196 and 130, respectively; and
(f) SEQ ID NOs: 190 and 118, respectively,
wherein a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length).

### V. Oligonucleotide Modifications

### A. Sugar Modifications

In some embodiments, an oligonucleotide described herein (e.g., an RNAi oligonucleotide) comprises a modified sugar. In some embodiments, a modified sugar (also referred herein to a sugar analog) includes a modified deoxyribose or ribose moiety in which, for example, one or more modifications occur at the 2′, 3′, 4′ and/or 5′ carbon position of the sugar. In some embodiments, a modified sugar may also include non-natural alternative carbon structures such as those present in locked nucleic acids (“LNA”; see, e.g., Koshkin et al. (1998) TETRAHEDON 54:3607-3630), unlocked nucleic acids (“UNA”; see, e.g., Snead et al. (2013) MOL. THER-NUCL. ACIDS 2:e103) and bridged nucleic acids (“BNA”; see, e.g., Imanishi & Obika (2002) CHEM COMMUN. (CAMB) 21:1653-1659).

In some embodiments, a nucleotide modification in a sugar comprises a 2′-modification. In some embodiments, a 2′-modification may be 2′—O—propargyl, 2′—O—propylamin, 2′-amino, 2′-ethyl, 2′-fluoro (2′—F), 2′-aminoethyl (EA), 2′—O—methyl (2′—OMe), 2′—O—methoxyethyl (2′-MOE), 2′—O—[2-(methylamino)-2-oxoethyl] (2′-O-NMA) or 2′-deoxy-2′-fluoro-β-d-arabinonucleic acid (2′-FANA). In some embodiments, the modification is 2′—F, 2′—OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′—F or 2′—OMe, 2′-MOE, and 2′-deoxy-2′-fluoro-β-d-arabinonucleic acid). In some embodiments, the modified nucleotide comprises a 2′-modification (e.g., a 2′—F or 2′—OMe).

In some embodiments, the disclosure provides oligonucleotides having different modification patterns. In some embodiments, an oligonucleotide herein comprises a sense strand having a modification pattern as set forth in the Examples and Sequence Listing and an antisense strand having a modification pattern as set forth in the Examples and Sequence Listing.

In some embodiments, an oligonucleotide disclosed herein (e.g., an RNAi oligonucleotide) comprises an antisense strand having nucleotides that are modified with 2′—F. In some embodiments, an oligonucleotide herein comprises an antisense strand comprising nucleotides that are modified with 2′—F and 2′—OMe. In some embodiments, an oligonucleotide disclosed herein comprises a sense strand having nucleotides that are modified with 2′—F. In some embodiments, an oligonucleotide disclosed herein comprises a sense strand comprises nucleotides that are modified with 2′—F and 2′—OMe.

In some embodiments, an oligonucleotide described herein comprises a sense strand with about 10-15%, 10%, 11%, 12%, 13%, 14% or 15% of the nucleotides of the sense strand comprising a 2′-fluoro modification. In some embodiments, about 11% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, about 32% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, about 19% of the nucleotides in the dsRNAi oligonucleotide comprise a 2′-fluoro modification.

In some embodiments, one or more of positions 8, 9, 10 or 11 of the sense strand is modified with a 2′—F group. In some embodiments, one or more of positions 3, 8, 9, 10, 12, 13 and 17 of the sense strand is modified with a 2′—F group. In some embodiments, one or more of positions 2, 3, 4, 5, 7, 10 and 14 of the antisense strand is modified with a 2′—F group. In some embodiments, one or more of positions 2, 3, 5, 7, 10 and 14 of the antisense strand is modified with a 2′—F group. In some embodiments, one or more of positions 2, 5, 7, 8, 10, 12, 14, 16 and 19 of the antisense strand is modified with a 2′—F group. In some embodiments, one or more of positions 2, 3, 4, 5, 7, 8, 10, 14, 16 and 19 of the antisense strand is modified with a 2′—F group. In some embodiments, the sugar moiety at each of nucleotides at positions 1-7 and 12-20 in the sense strand is modified with a 2′—OMe. In some embodiments, the sugar moiety at each of nucleotides at positions 1-7, 12-27 and 31-36 in the sense strand is modified with a 2′—OMe. In some embodiments, the sugar moiety at each of nucleotides at positions 1-2, 4-7, 11, 14-16 and 18-20 in the sense strand is modified with a 2′—OMe. In some embodiments, the sugar moiety at each of nucleotides at positions 1-2, 4-7, 11, 14-16 and 18-27 and 31-36 in the sense strand is modified with a 2′—OMe. In some embodiments, the sugar moiety at each of nucleotides at positions 3, 4, 6, 9, 11, 13, 15, 17, 18 and 20-22 in the antisense strand is modified with a 2′—OMe. In some embodiments, the sugar moiety at each of nucleotides at positions 4, 6, 8, 9, 11, 12, 13, and 15-22 in the antisense strand is modified with a 2′—OMe. In some embodiments, the sugar moiety at each of nucleotides at positions 6, 8, 9, 11-13, and 15-22 in the antisense strand is modified with a 2′—OMe.

The disclosure provides oligonucleotides having different modification patterns. In some embodiments, the modified oligonucleotides comprise a sense strand sequence having a modification pattern as set forth in FIGS. 1A-1C and an antisense strand having a modification pattern as set forth in FIGS. 1A-1C. In some embodiments, for these oligonucleotides, one or more of positions 8, 9, 10 or 11 of the sense strand is modified with a 2′—F group. In other embodiments, for these oligonucleotides, the sugar moiety at each of nucleotides at positions 1-7 and 12-20 in the sense strand is modified with a 2′—OMe.

In some embodiments, the sense and antisense strands of an oligonucleotide comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(l) SEQ ID NOs: 171 and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and,
(dd) SEQ ID NOs: 189 and 84, respectively;
wherein one or more of positions 8, 9, 10 or 11 of the sense strand is modified with a 2′-F group.

In some embodiments, the sense and antisense strands of an oligonucleotide comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and,
(p) SEQ ID NOs: 205 and 148, respectively;
wherein one or more of positions 8, 9, 10 or 11 of the sense strand is modified with a 2′—F group.

In some embodiments, an oligonucleotide provided herein comprises an antisense strand having the sugar moiety of each of the nucleotides at positions 2, 5, 7, 8, 10, 12, 14, 16 and 19 of the antisense strand modified with 2′—F and the sugar moiety of each of the remaining nucleotides of the antisense strand modified with a modification selected from the group consisting of 2′—O—propargyl, 2′—O—propylamin, 2′-amino, 2′-ethyl, 2′-aminoethyl (EA), 2′—O—methyl (2′—OMe), 2′—O—methoxyethyl (2′-MOE), 2′—O—[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2, 3, 5, 7, 10, and 14 of the antisense strand modified with 2′—F and the sugar moiety of each of the remaining nucleotides of the antisense strand modified with a modification selected from the group consisting of 2′—O—propargyl, 2′—O—propylamin, 2′-amino, 2′-ethyl, 2′-aminoethyl (EA), 2′—O—methyl (2′—OMe), 2′—O—methoxyethyl (2′-MOE), 2′—O—[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety of each of the nucleotides at positions 2-5, 7, 10, and 14 of the antisense strand modified with 2′—F and the sugar moiety of each of the remaining nucleotides of the antisense strand modified with a modification selected from the group consisting of 2′—O—propargyl, 2′—O—propylamin, 2′-amino, 2′-ethyl, 2′-aminoethyl (EA), 2′—O—methyl (2′—OMe), 2′—O—methoxyethyl (2′-MOE), 2′—O—[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′—F.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′—OMe.

In some embodiments, an oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′—O—propargyl, 2′—O—propylamin, 2′-amino, 2′-ethyl, 2′-aminoethyl (EA), 2′—O—methyl (2′—OMe), 2′—O—methoxyethyl (2′-MOE), 2′—O—[2-(methylamino)-2-oxoethyl] (2′-O-NMA), and 2′-deoxy-2′-fluoro-β-d-arabinonucleic acid (2′-FANA).

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′—F.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′—OMe.

In some embodiments, an oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′—O—propargyl, 2′—O—propylamin, 2′-amino, 2′-ethyl, 2′-aminoethyl (EA), 2′—O—methyl (2′—OMe), 2′—O—methoxyethyl (2′-MOE), 2′—O—[2-(methylamino)-2-oxoethyl] (2′-O-NMA), and 2′-deoxy-2′-fluoro-β-d-arabinonucleic acid (2′-FANA).

### B. 5′ Terminal Phosphates

In some embodiments, an oligonucleotide described herein (e.g., an RNAi oligonucleotide) comprises a sense strand and an antisense strand, wherein the antisense strand comprises a 5′-terminal phosphate. In some embodiments, 5′-terminal phosphate groups of oligonucleotides enhance the interaction with Ago2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate or malonyl phosphonate. In certain embodiments, the 1′ end of an oligonucleotide strand is attached to chemical moiety that mimics the electrostatic and steric properties of a natural 5′-phosphate group (“phosphate mimic”).

In some embodiments, the sense and antisense strands of an oligonucleotide comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(l) SEQ ID NOs: 171 and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and,
(dd) SEQ ID NOs: 189 and 84, respectively;
wherein the oligonucleotide comprises a 5′-terminal phosphate, optionally a 5′-terminal phosphate analog.

In some embodiments, the sense and antisense strands of an oligonucleotide comprise nucleotides sequences selected from the group consisting of:
(a) SEQ ID NOs: 162 and 30, respectively;
(b) SEQ ID NOs: 163 and 32, respectively; and,
(c) SEQ ID NOs: 169 and 44, respectively;
wherein the oligonucleotide comprises a 5′-terminal phosphate, optionally a 5′-terminal phosphate analog.

In some embodiments, the sense and antisense strands of an oligonucleotide comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and,
(p) SEQ ID NOs: 205 and 148, respectively;
wherein the oligonucleotide comprises a 5′-terminal phosphate, optionally a 5′-terminal phosphate analog.

In some embodiments, the sense and antisense strands of an oligonucleotide comprise nucleotides sequences selected from the group consisting of:
(a) SEQ ID NOs: 200 and 138, respectively;
(b) SEQ ID NOs: 203 and 144, respectively;
(c) SEQ ID NOs: 194 and 126, respectively;
(d) SEQ ID NOs: 191 and 120, respectively;
(e) SEQ ID NOs: 196 and 130, respectively; and,
(f) SEQ ID NOs: 190 and 118, respectively;
wherein the oligonucleotide comprises a 5′-terminal phosphate, optionally a 5′-terminal phosphate analog.

In some embodiments, an oligonucleotide has a phosphate analog at a 4′-carbon position of the sugar (referred to as a “4′-phosphate analog”). See, e.g., Intl. Patent Application Publication No. WO 2018/045317. In some embodiments, an oligonucleotide herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si (CH₃)₃ or a protecting group. In certain embodiments, the alkyl group is CH₂CH₃. More typically, R is independently selected from H, CH₃ or CH₂CH₃. In some embodiments, the 4′-phosphate analog is 4′-oxymethylphoshonate. In some embodiments, the modified nucleotide having the 4′-phosphonate analog is a uridine. In some embodiments, the modified nucleotide is 4′—O—monomethylphosphonate-2′—O—methyl uridine.

In some embodiments, an oligonucleotide provided herein comprises an antisense strand comprising a 4′-phosphate analog at the 5′-terminal nucleotide, wherein 5′-terminal nucleotide comprises the following structure:

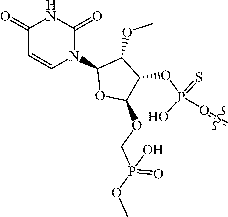

4′-monomethylphosphonate-2′—O—methyluridine phosphorothioate [MePhosphonate-4O-mUs].

### C. Modified Intranucleoside Linkages

In some embodiments, an oligonucleotide may comprise a modified internucleoside linkage. In some embodiments, phosphate modifications or substitutions may result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, the oligonucleotide described herein has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand.

In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(l) SEQ ID NOs: 171 and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and,
(dd) SEQ ID NOs: 189 and 84, respectively,
wherein the oligonucleotide comprises a modified internucleotide linkage.

In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 162 and 30, respectively;
(b) SEQ ID NOs: 163 and 32, respectively; and
(c) SEQ ID NOs: 169 and 44, respectively,
wherein the oligonucleotide comprises a modified internucleotide linkage.

In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and
(p) SEQ ID NOs: 205 and 148, respectively,
wherein the oligonucleotide comprises a modified internucleotide linkage.

In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the sense and antisense strands of an oligonucleotide comprise nucleotides sequences selected from the group consisting of:
(a) SEQ ID NOs: 200 and 138, respectively;
(b) SEQ ID NOs: 203 and 144, respectively;
(c) SEQ ID NOs: 194 and 126, respectively;
(d) SEQ ID NOs: 191 and 120, respectively;
(e) SEQ ID NOs: 196 and 130, respectively; and,
(f) SEQ ID NOs: 190 and 118, respectively,
wherein the oligonucleotide comprises a modified internucleotide linkage.

### D. Base Modifications

In some embodiments, oligonucleotides herein (e.g., an RNAi oligonucleotide) have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., U.S. Pat Application Publication No. 2008/0274462. In some embodiments, a modified nucleotide comprises a universal base. However, in certain embodiments, a modified nucleotide does not contain a nucleobase (abasic).

In some embodiments, the sense and antisense strands of an oligonucleotide comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(l) SEQ ID NOs: 171 and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and,
(dd) SEQ ID NOs: 189 and 84, respectively,
wherein the oligonucleotide comprises one or more modified nucleobases.

In some embodiments, the sense and antisense strands of an oligonucleotide comprise nucleotides sequences selected from the group consisting of:
(a) SEQ ID NOs: 162 and 30, respectively;
(b) SEQ ID NOs: 163 and 32, respectively; and,
(c) SEQ ID NOs: 169 and 44, respectively,
wherein the oligonucleotide comprises one or more modified nucleobases.

In some embodiments, the sense and antisense strands of an oligonucleotide comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and,
(p) SEQ ID NOs: 205 and 148, respectively,
wherein the oligonucleotide comprises one or more modified nucleobases.

In some embodiments, the sense and antisense strands of an oligonucleotide comprise nucleotides sequences selected from the group consisting of:
(a) SEQ ID NOs: 200 and 138, respectively;
(b) SEQ ID NOs: 203 and 144, respectively;
(c) SEQ ID NOs: 194 and 126, respectively;
(d) SEQ ID NOs: 191 and 120, respectively;
(e) SEQ ID NOs: 196 and 130, respectively; and,
(f) SEQ ID NOs: 190 and 118, respectively;
wherein the oligonucleotide comprises one or more modified nucleobases.

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, U.S. Pat. Application Publication No. 2007/0254362; Van Aerschot et al. (1995) NUCLEIC ACIDS RES. 23:4363-4370; Loakes et al. (1995) NUCLEIC ACIDS RES. 23:2361-2366; and Loakes & Brown (1994) NUCLEIC ACIDS RES. 22:4039-4043).

### E. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See U.S. Pat Application Publication No. 2011/0294869, Intl. Patent Application Publication Nos. WO 2014/088920 and WO 2015/188197, and Meade et al. (2014) NAT. BIOTECHNOL. 32:1256-1263. This reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (see, Dellinger et al. (2003) J. AM. CHEM. SOC. 125:940-50).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione-sensitive moieties, it is possible to introduce sterically larger chemical groups into the oligonucleotide of interest when compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′terminal nucleotide of the oligonucleotide. In some embodiments, the glutathione-sensitive moiety is located at the 3′-carbon of sugar, particularly when the modified nucleotide is the 3′terminal nucleotide of the oligonucleotide. In some embodiments, the glutathione-sensitive moiety comprises a sulfonyl group. See, e.g., U.S. Provisional Pat. Application No. 62/378,635, entitled Compositions Comprising Reversibly Modified Oligonucleotides and Uses Thereof, which was filed on Aug. 23, 2016.

### VI. Targeting Ligands

In some embodiments, it is desirable to target the oligonucleotides of the disclosure to one or more cells or one or more organs. Such a strategy can help to avoid undesirable effects in other organs or avoid undue loss of the oligonucleotide to cells, tissue or organs that would not benefit from the oligonucleotide. Targeting of oligonucleotides to one or more cells or one or more organs can be achieved through a variety of approaches. Conjugation of oligonucleotides to tissue or cell specific antibodies, small molecules or targeting ligands can facilitate delivery to and modify accumulation of the oligonucleotide in one or more target cells or tissues (Chernolovskaya et al. (2019) FRONT PHARMACOL. 10:444). For example, conjugation of an oligonucleotide to a saturated fatty acid (e.g,. C22) may facilitate delivery to cells or tissues like adipose tissue which uptake such ligands more readily than conventional oligonucleotide ligands. Accordingly, in some embodiments, oligonucleotides disclosed herein are modified to facilitate targeting and/or delivery of a tissue, cell or organ (e.g., to facilitate delivery of the oligonucleotide to the liver). In certain embodiments, oligonucleotides disclosed herein are modified to facilitate delivery of the oligonucleotide to the hepatocytes of the liver. In certain embodiments, oligonucleotides disclosed herein are modified to facilitate delivery of the oligonucleotide to the adipocytes of adipose tissue. In some embodiments, an oligonucleotide comprises at least one nucleotide (e.g., 1, 2, 3, 4, 5, 6 or more nucleotides) conjugated to one or more targeting ligand(s).

In some embodiments, the sense and antisense strands of an oligonucleotide comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(l) SEQ ID NOs: 171 and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and,
(dd) SEQ ID NOs: 189 and 84, respectively,
wherein the oligonucleotide comprises a targeting ligand conjugated to at least one nucleotide.

In some embodiments, the sense and antisense strands of an oligonucleotide comprise nucleotides sequences selected from the group consisting of:
(a) SEQ ID NOs: 162 and 30, respectively;
(b) SEQ ID NOs: 163 and 32, respectively; and,
(c) SEQ ID NOs: 169 and 44, respectively,
wherein the oligonucleotide comprises a targeting ligand conjugated to at least one nucleotide.

In some embodiments, the sense and antisense strands of an oligonucleotide comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and,
(p) SEQ ID NOs: 205 and 148, respectively,
wherein the oligonucleotide comprises a targeting ligand conjugated to at least one nucleotide.

In some embodiments, the sense and antisense strands of an oligonucleotide comprise nucleotides sequences selected from the group consisting of:
(a) SEQ ID NOs: 200 and 138, respectively;
(b) SEQ ID NOs: 203 and 144, respectively;
(c) SEQ ID NOs: 194 and 126, respectively;
(d) SEQ ID NOs: 191 and 120, respectively;
(e) SEQ ID NOs: 196 and 130, respectively; and,
(f) SEQ ID NOs: 190 and 118, respectively,
wherein the oligonucleotide comprises a targeting ligand conjugated to at least one nucleotide.

In some embodiments, the targeting ligand comprises a carbohydrate, amino sugar, cholesterol, peptide, polypeptide, protein or part of a protein (e.g., an antibody or antibody fragment), or lipid. In some embodiments, the targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush, and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand. In some embodiments, an oligonucleotide (e.g., a dsRNA) provided by the disclosure comprises a stem-loop at the 3′ end of the sense strand, wherein the loop of the stem-loop comprises a triloop or a tetraloop, and wherein the 3 or 4 nucleotides comprising the triloop or tetraloop, respectfully, are individually conjugated to a targeting ligand.

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotides of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties. In some embodiments, a bivalent, trivalent or tetravalent GalNAc moiety is conjugated to an oligonucleotide via a branched linker. In some embodiments, a monovalent GalNAc moiety is conjugated to a first nucleotide and a bivalent, trivalent, or tetravalent GalNAc moiety is conjugated to a second nucleotide via a branched linker.

In some embodiments, 1 or more (e.g., 1, 2, 3, 4, 5 or 6) nucleotides of an oligonucleotide are each conjugated to a GalNAc moiety. In some embodiments, 2 to 4 nucleotides of a tetraloop are each conjugated to a separate GalNAc. In some embodiments, 1 to 3 nucleotides of a triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush, and the oligonucleotide resembles a toothbrush. In some embodiments, GalNAc moieties are conjugated to a nucleotide of the sense strand. For example, 4 GalNAc moieties can be conjugated to nucleotides in the tetraloop of the sense strand where each GalNAc moiety is conjugated to 1 nucleotide.

In some embodiments, the tetraloop is any combination of adenine and guanine nucleotides.

In some embodiments, the tetraloop (L) has a monovalent GalNAc moiety attached to any one or more guanine nucleotides of the tetraloop via any linker described herein, as depicted below (X=heteroatom):

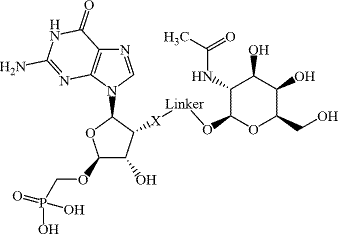

In some embodiments, the tetraloop (L) has a monovalent GalNAc attached to any one or more adenine nucleotides of the tetraloop via any linker described herein, as depicted below (X=heteroatom):

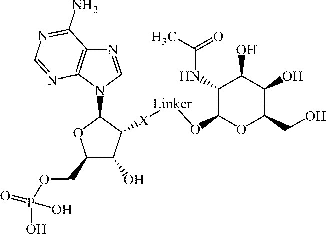

In some embodiments, an oligonucleotide herein comprises a monovalent GalNAc attached to a guanine nucleotide referred to as [ademG-GalNAc] or 2′-aminodiethoxymethanol-Guanine-GalNAc, as depicted below:

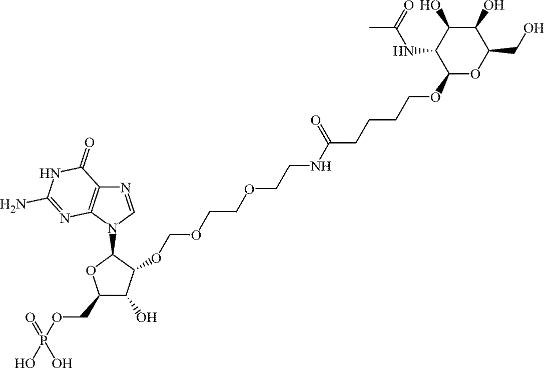 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below:

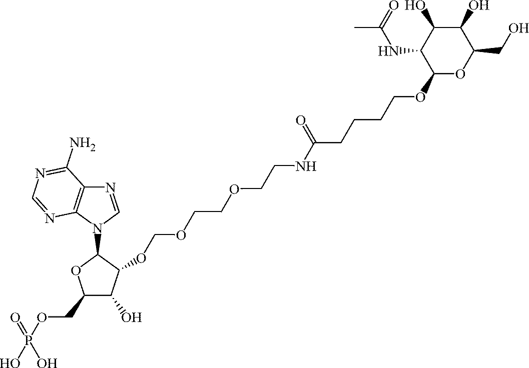

An example of such conjugation is shown below for a loop comprising from 5′ to 3′ the nucleotide sequence GAAA (L = linker, X = heteroatom) stem attachment points are shown. Such a loop may be present, for example, at positions 27-30 of the sense strand as shown in FIGS. 1A-1C. In the chemical formula,

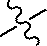

is used to describe an attachment point to the oligonucleotide strand.

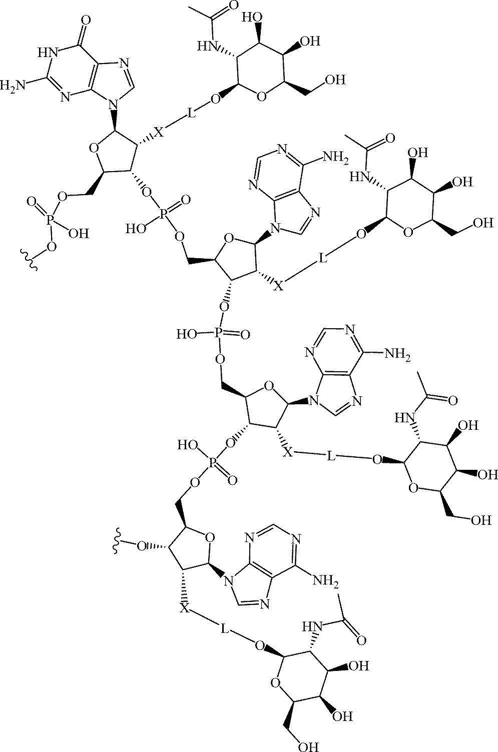

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. An example is shown below for a loop comprising from 5′ to 3′ the nucleotides GAAA, in which GalNAc moieties are attached to nucleotides of the loop using an acetal linker. Such a loop may be present, for example, at positions 27-30 of the any one of the sense strand as shown in FIGS. 1A-1C. In the chemical formula,

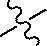

is anattachment point to the oligonucleotide strand.

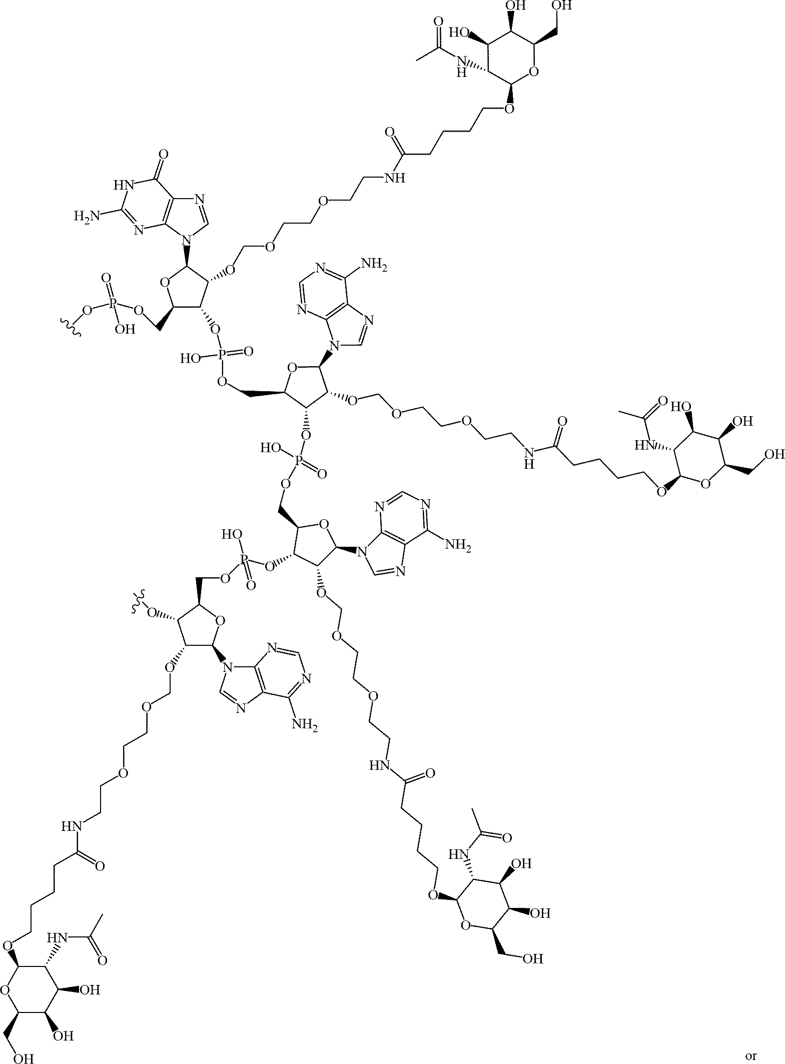

or

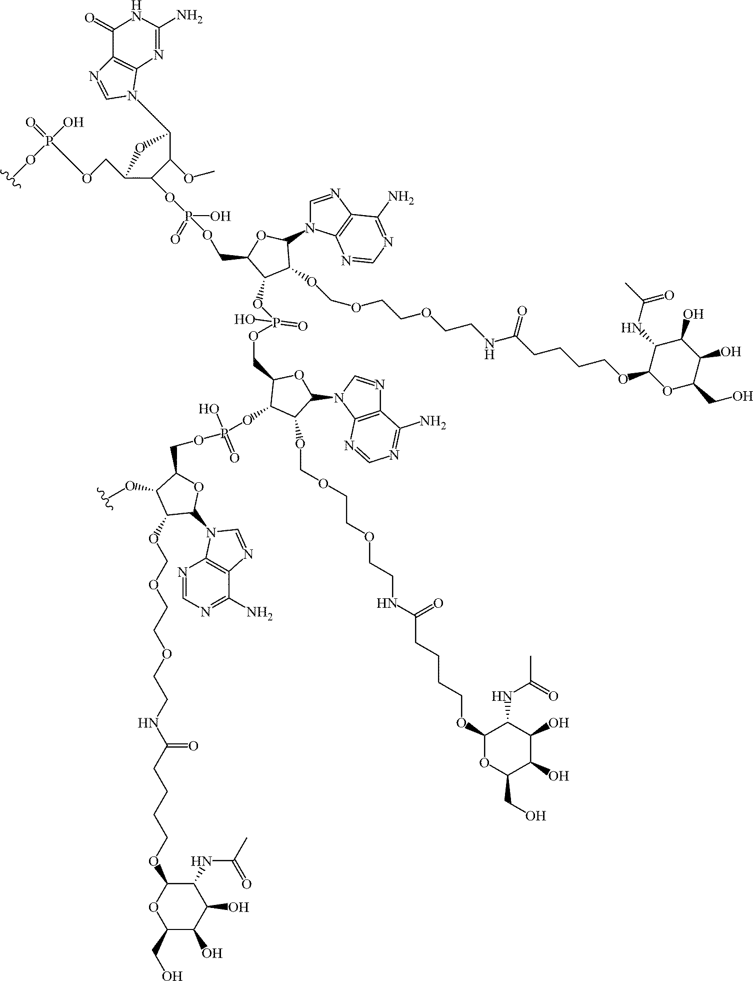

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a dsRNA. In some embodiments, the oligonucleotides herein do not have a GalNAc conjugated thereto.

In some embodiments, the sense and antisense strands of an oligonucleotide comprise nucleotides sequences selected from the group consisting of:
(a) SEQ ID NOs: 160 and 26, respectively;
(b) SEQ ID NOs: 161 and 28, respectively;
(c) SEQ ID NOs: 162 and 30, respectively;
(d) SEQ ID NOs: 163 and 32, respectively;
(e) SEQ ID NOs: 164 and 34, respectively;
(f) SEQ ID NOs: 165 and 36, respectively;
(g) SEQ ID NOs: 166 and 38, respectively;
(h) SEQ ID NOs: 167 and 40, respectively;
(i) SEQ ID NOs: 168 and 42, respectively;
(j) SEQ ID NOs: 169 and 44, respectively;
(k) SEQ ID NOs: 170 and 46, respectively;
(1) SEQ ID NOs: 17and 48, respectively;
(m) SEQ ID NOs: 172 and 50, respectively;
(n) SEQ ID NOs: 173 and 52, respectively;
(o) SEQ ID NOs: 174 and 54, respectively;
(p) SEQ ID NOs: 175 and 56, respectively;
(q) SEQ ID NOs: 176 and 58, respectively;
(r) SEQ ID NOs: 177 and 60, respectively;
(s) SEQ ID NOs: 178 and 62, respectively;
(t) SEQ ID NOs: 179 and 64, respectively;
(u) SEQ ID NOs: 180 and 66, respectively;
(v) SEQ ID NOs: 181 and 68, respectively;
(w) SEQ ID NOs: 182 and 70, respectively;
(x) SEQ ID NOs: 183 and 72, respectively;
(y) SEQ ID NOs: 184 and 74, respectively;
(z) SEQ ID NOs: 185 and 76, respectively;
(aa) SEQ ID NOs: 186 and 78, respectively;
(bb) SEQ ID NOs: 187 and 80, respectively;
(cc) SEQ ID NOs: 188 and 82, respectively; and,
(dd) SEQ ID NOs: 189 and 84, respectively,
wherein the oligonucleotide comprises at least one GalNAc moiety conjugated to a nucleotide.

In some embodiments, the sense and antisense strands of an oligonucleotide comprise nucleotides sequences selected from the group consisting of:
(a) SEQ ID NOs: 162 and 30, respectively;
(b) SEQ ID NOs: 163 and 32, respectively; and,
(c) SEQ ID NOs: 169 and 44, respectively,
wherein the oligonucleotide comprises at least one GalNAc moiety conjugated to a nucleotide.

In some embodiments, the sense and antisense strands of an oligonucleotide comprise nucleotides sequences selected from the group consisting of:
(a) SEQ ID NOs: 190 and 118, respectively;
(b) SEQ ID NOs: 191 and 120, respectively;
(c) SEQ ID NOs: 192 and 122, respectively;
(d) SEQ ID NOs: 193 and 124, respectively;
(e) SEQ ID NOs: 194 and 126, respectively;
(f) SEQ ID NOs: 195 and 128, respectively;
(g) SEQ ID NOs: 196 and 130, respectively;
(h) SEQ ID NOs: 197 and 132, respectively;
(i) SEQ ID NOs: 198 and 134, respectively;
(j) SEQ ID NOs: 199 and 136, respectively;
(k) SEQ ID NOs: 200 and 138, respectively;
(l) SEQ ID NOs: 201 and 140, respectively;
(m) SEQ ID NOs: 202 and 142, respectively;
(n) SEQ ID NOs: 203 and 144, respectively;
(o) SEQ ID NOs: 204 and 146, respectively; and,
(p) SEQ ID NOs: 205 and 148, respectively,
wherein the oligonucleotide comprises at least one GalNAc moiety conjugated to a nucleotide.

In some embodiments, the sense and antisense strands of an oligonucleotide comprise nucleotides sequences selected from the group consisting of:
(a) SEQ ID NOs: 200 and 138, respectively;
(b) SEQ ID NOs: 203 and 144, respectively;
(c) SEQ ID NOs: 194 and 126, respectively;
(d) SEQ ID NOs: 191 and 120, respectively;
(e) SEQ ID NOs: 196 and 130, respectively; and
(f) SEQ ID NOs: 190 and 118, respectively,
wherein the oligonucleotide comprises at least one GalNAc moiety conjugated to a nucleotide.

### VII. Exemplary Modified Oligonucleotides

In some embodiments, an oligonucleotide targeting ACC comprises a sense strand and an antisense strand as set forth in Table 3, wherein the oligonucleotide comprises a stem loop structure having a double-stranded stem of about 2-6 base pairs and a loop of 3-4 nucleotides, and wherein the sense and antisense strands comprise the modification pattern set forth in FIG. 1B. In some embodiments, an oligonucleotide targeting ACC comprises a sense strand and an antisense strand as set forth in Table 3, wherein the oligonucleotide comprises a stem loop structure having a double-stranded stem of about 2-6 base pairs and a loop of 3-4 nucleotides, wherein the sense and antisense strands comprise the modification pattern set forth in FIG. 1B, and wherein antisense strand is modified with an oxymethylphosphonate at the 4′ carbon of the 5′ terminal nucleotide. In some embodiments, the oligonucleotide comprises a stem loop comprising the nucleotide sequence of SEQ ID NO: 159. In some embodiments, the oligonucleotide comprises a double-stranded stem of 6 base pairs and a stem loop of 4 nucleotides comprising one, two, three or four GalNAc conjugated nucleotides. In some embodiments, the GalNAc conjugated nucleotide is a monovalent GalNAc conjugated to an adenine nucleotide, referred to as [ademA-GalNAc] or 2′-aminodiethoxymethanol-Adenine-GalNAc, as depicted below:

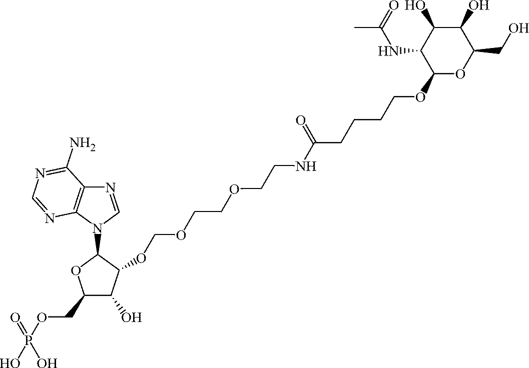 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some embodiments, the stem loop comprises a double-stranded stem of 6 base pairs and a loop comprising the nucleotide sequence GAAA, wherein each adenine nucleotide is ademA-GalNAc.

In some embodiments, an oligonucleotide targeting ACC comprises a sense strand and an antisense strand as set forth in Table 4, wherein the sense and antisense strands comprise the modification pattern set forth in FIG. 1B. In some embodiments, an oligonucleotide targeting ACC comprises a sense strand and an antisense strand as set forth in Table 4, wherein the sense and antisense strands comprise the modification pattern set forth in FIG. 1B, and wherein antisense strand is modified with an oxymethylphosphonate at the 4′ carbon of the 5′ terminal nucleotide. In some embodiments, the oligonucleotide comprises a stem loop comprising the nucleotide sequence of SEQ ID NO: 159. In some embodiments, the oligonucleotide comprises a double-stranded stem of 6 base pairs and a stem loop of 4 nucleotides comprising one, two, three or four GalNAc conjugated nucleotides. In some embodiments, the GalNAc conjugated nucleotide is a monovalent GalNAc conjugated to an adenine nucleotide, referred to as [ademA-GalNAc] or 2′-aminodiethoxymethanol-Adenine-GalNAc, as depicted below:

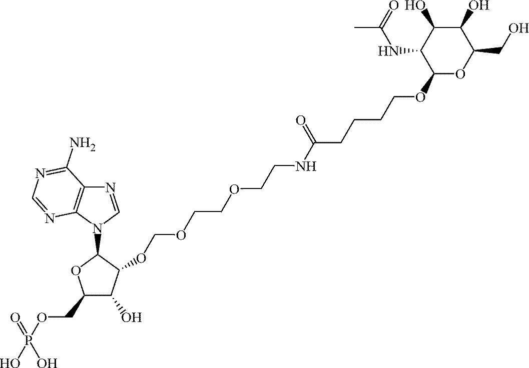 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some embodiments, the stem loop comprises a double-stranded stem of 6 base pairs and a loop comprising the nucleotide sequence GAAA, wherein each adenine nucleotide is ademA-GalNAc.

In some embodiments, an oligonucleotide targeting DGAT comprises a sense strand and an antisense strand as set forth in Table 8, wherein the oligonucleotide comprises a stem loop structure having a double-stranded stem of about 2-6 base pairs and a loop of 3-4 nucleotides, and wherein the sense and antisense strands comprise the modification pattern set forth in FIG. 1C. In some embodiments, an oligonucleotide targeting DGAT comprises a sense strand and an antisense strand as set forth in Table 8, wherein the oligonucleotide comprises a stem loop structure having a double-stranded stem of about 2-6 base pairs and a loop of 3-4 nucleotides, wherein the sense and antisense strands comprise the modification pattern set forth in FIG. 1C, and wherein antisense strand is modified with an oxymethylphosphonate at the 4′ carbon of the 5′ terminal nucleotide. In some embodiments, the oligonucleotide comprises a stem loop comprising the nucleotide sequence of SEQ ID NO: 159. In some embodiments, the oligonucleotide comprises a double-stranded stem of 6 base pairs and a stem loop of 4 nucleotides comprising one, two, three or four GalNAc conjugated nucleotides. In some embodiments, the GalNAc conjugated nucleotide is a monovalent GalNAc conjugated to an adenine nucleotide, referred to as [ademA-GalNAc] or 2′-aminodiethoxymethanol-Adenine-GalNAc, as depicted below:

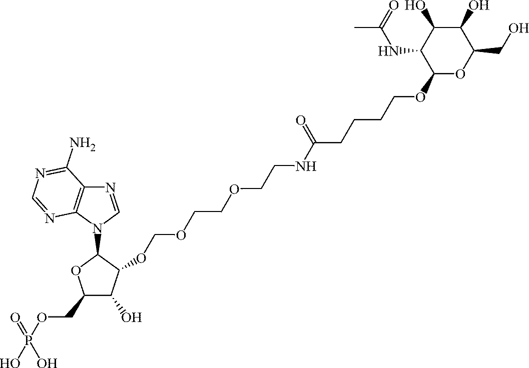 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some embodiments, the stem loop comprises a double-stranded stem of 6 base pairs and a loop comprising the nucleotide sequence GAAA, wherein each adenine nucleotide is ademA-GalNAc.

In some embodiments, an oligonucleotide targeting DGAT comprises a sense strand and an antisense strand as set forth in Table 9, wherein the sense and antisense strands comprise the modification pattern set forth in FIG. 1C. In some embodiments, an oligonucleotide targeting DGAT comprises a sense strand and an antisense strand as set forth in Table 9, wherein the sense and antisense strands comprise the modification pattern set forth in FIG. 1C, and wherein antisense strand is modified with an oxymethylphosphonate at the 4′ carbon of the 5′ terminal nucleotide. In some embodiments, the oligonucleotide comprises a stem loop comprising the nucleotide sequence of SEQ ID NO: 159. In some embodiments, the oligonucleotide comprises a double-stranded stem of 6 base pairs and a stem loop of 4 nucleotides comprising one, two, three or four GalNAc conjugated nucleotides. In some embodiments, the GalNAc conjugated nucleotide is a monovalent GalNAc conjugated to an adenine nucleotide, referred to as [ademA-GalNAc] or 2′-aminodiethoxymethanol-Adenine-GalNAc, as depicted below:

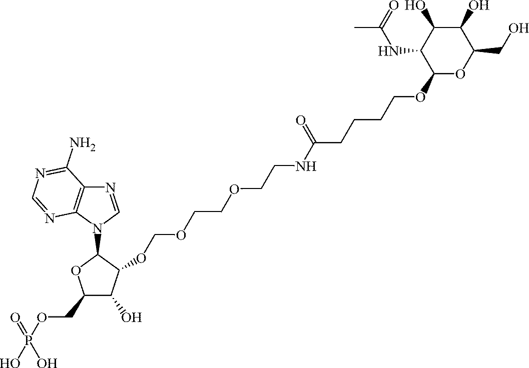 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some embodiments, the stem loop comprises a double-stranded stem of 6 base pairs and a loop comprising the nucleotide sequence GAAA, wherein each adenine nucleotide is ademA-GalNAc.

In some embodiments, an oligonucleotide provided herein (e.g., and RNAi oligonucleotide) for reducing ACC expression comprises:
a sense strand comprising a 2′—F modified nucleotide at positions 3, 8-10, 12, 13, and 17, a 2′—OMe modified nucleotide at positions 1-2, 4-7, 11, 14-16, 18-27, and 31-36, a GalNAc-conjugated nucleotide at position 28, 29 and 30; and a phosphorothioate linkage between positions 1 and 2;
an antisense strand comprising a 2′—F modified nucleotide at positions 2, 5, 7-8, 10, 12, 14, 16, and 19, a 2′—OMe at positions 1, 3-4, 6, 9, 11, 13, 15, 17-18, and 20-22, a phosphorothioate linkage between positions 1 and 2, positions 2 and 3, positions 3 and 4, positions 20 and 21, and positions 21 and 22,, and a 5′-terminal nucleotide at position 1 comprising a 4′-phosphate analog, optionally wherein the 5′-terminal nucleotide comprises 5′-methoxyphosphonate-4′-oxy-2′—O—methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
   (a) SEQ ID NOs: 160 and 26, respectively;
   (b) SEQ ID NOs: 161 and 28, respectively;
   (c) SEQ ID NOs: 162 and 30, respectively;
   (d) SEQ ID NOs: 163 and 32, respectively;
   (e) SEQ ID NOs: 164 and 34, respectively;
   (f) SEQ ID NOs: 165 and 36, respectively;
   (g) SEQ ID NOs: 166 and 38, respectively;
   (h) SEQ ID NOs: 167 and 40, respectively;
   (i) SEQ ID NOs: 168 and 42, respectively;
   (j) SEQ ID NOs: 169 and 44, respectively;
   (k) SEQ ID NOs: 170 and 46, respectively;
   (1) SEQ ID NOs: 17and 48, respectively;
   (m) SEQ ID NOs: 172 and 50, respectively;
   (n) SEQ ID NOs: 173 and 52, respectively;
   (o) SEQ ID NOs: 174 and 54, respectively;
   (p) SEQ ID NOs: 175 and 56, respectively;
   (q) SEQ ID NOs: 176 and 58, respectively;
   (r) SEQ ID NOs: 177 and 60, respectively;
   (s) SEQ ID NOs: 178 and 62, respectively;
   (t) SEQ ID NOs: 179 and 64, respectively;
   (u) SEQ ID NOs: 180 and 66, respectively;
   (v) SEQ ID NOs: 181 and 68, respectively;
   (w) SEQ ID NOs: 182 and 70, respectively;
   (x) SEQ ID NOs: 183 and 72, respectively;
   (y) SEQ ID NOs: 184 and 74, respectively;
   (z) SEQ ID NOs: 185 and 76, respectively;
   (aa) SEQ ID NOs: 186 and 78, respectively;
   (bb) SEQ ID NOs: 187 and 80, respectively;
   (cc) SEQ ID NOs: 188 and 82, respectively; and
   (dd) SEQ ID NOs: 189 and 84, respectively.

In some embodiments, an oligonucleotide provided herein (e.g., and RNAi oligonucleotide) for reducing DGAT2 expression comprises:
a sense strand comprising a 2′—F modified nucleotide at positions 3, 8-10, 12, 13, and 17, a 2′—OMe modified nucleotide at positions 1-2, 4-7, 11, 14-16, 18-27, and 31-36, a GalNAc-conjugated nucleotide at position 28, 29 and 30; and a phosphorothioate linkage between positions 1 and 2;
an antisense strand comprising a 2′—F modified nucleotide at positions 2, 5, 7-8, 10, 12, 14, 16, and 19, a 2′—OMe at positions 1, 3-4, 6, 9, 11, 13, 15, 17-18, and 20-22, a phosphorothioate linkage between positions 1 and 2, positions 2 and 3, positions 3 and 4, positions 20 and 21, and positions 21 and 22,, and a 5′-terminal nucleotide at position 1 comprising a 4′-phosphate analog, optionally wherein the 5′-terminal nucleotide comprises 5′-methoxyphosphonate-4′-oxy-2′—O—methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
   (a) SEQ ID NOs: 190 and 118, respectively;
   (b) SEQ ID NOs: 191 and 120, respectively;
   (c) SEQ ID NOs: 192 and 122, respectively;
   (d) SEQ ID NOs: 193 and 124, respectively;
   (e) SEQ ID NOs: 194 and 126, respectively;
   (f) SEQ ID NOs: 195 and 128, respectively;
   (g) SEQ ID NOs: 196 and 130, respectively;
   (h) SEQ ID NOs: 197 and 132, respectively;
   (i) SEQ ID NOs: 198 and 134, respectively;
   (j) SEQ ID NOs: 199 and 136, respectively;
   (k) SEQ ID NOs: 200 and 138, respectively;
   (l) SEQ ID NOs: 201 and 140, respectively;
   (m) SEQ ID NOs: 202 and 142, respectively;
   (n) SEQ ID NOs: 203 and 144, respectively;
   (o) SEQ ID NOs: 204 and 146, respectively; and
   (p) SEQ ID NOs: 205 and 148, respectively.

In some embodiments, an oligonucleotide provided herein (e.g., and RNAi oligonucleotide) for reducing ACC expression comprises:
a sense strand comprising a 2′—F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′—F modified nucleotide at positions 2-3, 5, 7, 10, and 14, a 2′—OMe at positions 1, 4, 6, 8-9, 11-13, and 15-22, a phosphorothioate linkage between positions 1 and 2, positions 2 and 3, positions 20 and 21, and positions 21 and 22, and a 5′terminal nucleotide at position 1 comprising a 4′-phosphate analog, optionally wherein the 5′terminal nucleotide comprises 5′-methoxyphosphonate-4′-oxy-2′—O—methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
   (a) SEQ ID NOs: 160 and 26, respectively;
   (b) SEQ ID NOs: 161 and 28, respectively;
   (c) SEQ ID NOs: 162 and 30, respectively;
   (d) SEQ ID NOs: 163 and 32, respectively;
   (e) SEQ ID NOs: 164 and 34, respectively;
   (f) SEQ ID NOs: 165 and 36, respectively;
   (g) SEQ ID NOs: 166 and 38, respectively;
   (h) SEQ ID NOs: 167 and 40, respectively;
   (i) SEQ ID NOs: 168 and 42, respectively;
   (j) SEQ ID NOs: 169 and 44, respectively;
   (k) SEQ ID NOs: 170 and 46, respectively;
   (1) SEQ ID NOs: 17and 48, respectively;
   (m) SEQ ID NOs: 172 and 50, respectively;
   (n) SEQ ID NOs: 173 and 52, respectively;
   (o) SEQ ID NOs: 174 and 54, respectively;
   (p) SEQ ID NOs: 175 and 56, respectively;
   (q) SEQ ID NOs: 176 and 58, respectively;
   (r) SEQ ID NOs: 177 and 60, respectively;
   (s) SEQ ID NOs: 178 and 62, respectively;
   (t) SEQ ID NOs: 179 and 64, respectively;
   (u) SEQ ID NOs: 180 and 66, respectively;
   (v) SEQ ID NOs: 181 and 68, respectively;
   (w) SEQ ID NOs: 182 and 70, respectively;
   (x) SEQ ID NOs: 183 and 72, respectively;
   (y) SEQ ID NOs: 184 and 74, respectively;
   (z) SEQ ID NOs: 185 and 76, respectively;
   (aa) SEQ ID NOs: 186 and 78, respectively;
   (bb) SEQ ID NOs: 187 and 80, respectively;
   (cc) SEQ ID NOs: 188 and 82, respectively; and,
   (dd) SEQ ID NOs: 189 and 84, respectively.

In some embodiments, an oligonucleotide provided herein (e.g., and RNAi oligonucleotide) for reducing DGAT2 expression comprises:
a sense strand comprising a 2′—F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′—F modified nucleotide at positions 2-3, 5, 7, 10, and 14, a 2′—OMe at positions 1, 4, 6, 8-9, 11-13, and 15-22, a phosphorothioate linkage between positions 1 and 2, positions 2 and 3, positions 20 and 21, and positions 21 and 22, and a 5′terminal nucleotide at position 1 comprising a 4′-phosphate analog, optionally wherein the 5′terminal nucleotide comprises 5′-methoxyphosphonate-4′-oxy-2′—O—methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
   (a) SEQ ID NOs: 190 and 118, respectively;
   (b) SEQ ID NOs: 191 and 120, respectively;
   (c) SEQ ID NOs: 192 and 122, respectively;
   (d) SEQ ID NOs: 193 and 124, respectively;
   (e) SEQ ID NOs: 194 and 126, respectively;
   (f) SEQ ID NOs: 195 and 128, respectively;
   (g) SEQ ID NOs: 196 and 130, respectively;
   (h) SEQ ID NOs: 197 and 132, respectively;
   (i) SEQ ID NOs: 198 and 134, respectively;
   (j) SEQ ID NOs: 199 and 136, respectively;
   (k) SEQ ID NOs: 200 and 138, respectively;
   (l) SEQ ID NOs: 201 and 140, respectively;
   (m) SEQ ID NOs: 202 and 142, respectively;
   (n) SEQ ID NOs: 203 and 144, respectively;
   (o) SEQ ID NOs: 204 and 146, respectively; and,
   (p) SEQ ID NOs: 205 and 148, respectively.

In some embodiments, an oligonucleotide provided herein (e.g., and RNAi oligonucleotide) for reducing ACC expression comprises:
a sense strand comprising a 2′—F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′—F modified nucleotide at positions 2-5, 7, 10, and 14, a 2′—OMe at positions 1, 6, 8-9, 11-13, and 15-22, a phosphorothioate linkage between positions 1 and 2, positions 2 and 3, positions 20 and 21, and positions 21 and 22, and a 5′terminal nucleotide at position 1 comprising a 4′-phosphate analog, optionally wherein the 5′terminal nucleotide comprises 5′-methoxyphosphonate-4′-oxy-2′—O—methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
   (a) SEQ ID NOs: 160 and 26, respectively;
   (b) SEQ ID NOs: 161 and 28, respectively;
   (c) SEQ ID NOs: 162 and 30, respectively;
   (d) SEQ ID NOs: 163 and 32, respectively;
   (e) SEQ ID NOs: 164 and 34, respectively;
   (f) SEQ ID NOs: 165 and 36, respectively;
   (g) SEQ ID NOs: 166 and 38, respectively;
   (h) SEQ ID NOs: 167 and 40, respectively;
   (i) SEQ ID NOs: 168 and 42, respectively;
   (j) SEQ ID NOs: 169 and 44, respectively;
   (k) SEQ ID NOs: 170 and 46, respectively;
   (l) SEQ ID NOs: 171 and 48, respectively;
   (m) SEQ ID NOs: 172 and 50, respectively;
   (n) SEQ ID NOs: 173 and 52, respectively;
   (o) SEQ ID NOs: 174 and 54, respectively;
   (p) SEQ ID NOs: 175 and 56, respectively;
   (q) SEQ ID NOs: 176 and 58, respectively;
   (r) SEQ ID NOs: 177 and 60, respectively;
   (s) SEQ ID NOs: 178 and 62, respectively;
   (t) SEQ ID NOs: 179 and 64, respectively;
   (u) SEQ ID NOs: 180 and 66, respectively;
   (v) SEQ ID NOs: 181 and 68, respectively;
   (w) SEQ ID NOs: 182 and 70, respectively;
   (x) SEQ ID NOs: 183 and 72, respectively;
   (y) SEQ ID NOs: 184 and 74, respectively;
   (z) SEQ ID NOs: 185 and 76, respectively;
   (aa) SEQ ID NOs: 186 and 78, respectively;
   (bb) SEQ ID NOs: 187 and 80, respectively;
   (cc) SEQ ID NOs: 188 and 82, respectively; and,
   (dd) SEQ ID NOs: 189 and 84, respectively.

In some embodiments, an oligonucleotide provided herein (e.g., and RNAi oligonucleotide) for reducing DGAT2 expression comprises:
a sense strand comprising a 2′—F modified nucleotide at positions 8-11, a 2′-OMe modified nucleotide at positions 1-7, 12-27, and 31-36, a GalNAc-conjugated nucleotide at position 28, 29 and 30; and a phosphorothioate linkage between positions 1 and 2;
an antisense strand comprising a 2′—F modified nucleotide at positions 2-5, 7, 10, and 14, a 2′—OMe at positions 1, 6, 8-9, 11-13, and 15-22, a phosphorothioate linkage between positions 1 and 2, positions 2 and 3, positions 20 and 21, and positions 21 and 22, and a 5′terminal nucleotide at position 1 comprising a 4′-phosphate analog, optionally wherein the 5′terminal nucleotide comprises 5′-methoxyphosphonate-4′-oxy-2′—O—methyluridine [MePhosphonate-4O-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
   (a) SEQ ID NOs: 190 and 118, respectively;
   (b) SEQ ID NOs: 191 and 120, respectively;
   (c) SEQ ID NOs: 192 and 122, respectively;
   (d) SEQ ID NOs: 193 and 124, respectively;
   (e) SEQ ID NOs: 194 and 126, respectively;
   (f) SEQ ID NOs: 195 and 128, respectively;
   (g) SEQ ID NOs: 196 and 130, respectively;
   (h) SEQ ID NOs: 197 and 132, respectively;
   (i) SEQ ID NOs: 198 and 134, respectively;
   (j) SEQ ID NOs: 199 and 136, respectively;
   (k) SEQ ID NOs: 200 and 138, respectively;
   (l) SEQ ID NOs: 201 and 140, respectively;
   (m) SEQ ID NOs: 202 and 142, respectively;
   (n) SEQ ID NOs: 203 and 144, respectively;
   (o) SEQ ID NOs: 204 and 146, respectively; and,
   (p) SEQ ID NOs: 205 and 148, respectively.

In some embodiments, the disclosure provides an oligonucleotide (e.g., an RNAi oligonucleotide) for reducing ACC and/or DGAT2 expression, wherein the oligonucleotide comprises a sense strand and an antisense strand according to:

Sense Strand: 5′-mX-S-mX-fX-mX-mX-mX-mX-fX-fX-fX-mX-fX-fX-mX-mX-mX-fX-mX-mX-mX-mX-mX-mX-mX-mX-mX-mX-[ademA-GalNAc]-[ademA-GalNAc]-[ademA-GalNAc]-mX-mX-mX-mX-mX-mX- 3′; hybridized to:
Antisense Strand: 5′-[MePhosphonate-40-mX]-5-fX-S-mX- S-mX-fX-mX-fX-fX-fX-mX-fX-mX-fX-mX-fX-mX-fX-mX-mX-fX-S-mX-S-mX-3′ ;
wherein mX= 2′-O-methyl modified nucleotide, fX =2′- fluoro modified nucleotide, -S- = phosphorothioate linkage, - = phosphodiester linkage, [MePhosphonate-4O-mX] = 5′-methoxyphosphonate-4-oxy modified nucleotide, and ademA-GalNAc = GalNAc attached to an adenine nucleotide.

In some embodiments, the disclosure provides an oligonucleotide (e.g., an RNAi oligonucleotide) for reducing ACC and/or DGAT2 expression, wherein the oligonucleotide comprises a sense strand and an antisense strand according to:

Sense Strand: 5′-mX-S-mX-mX-mX-mX-mX-mX-fX-fX-fX-fX-mX-mX-mX-mX-mX-mX-mX-mX-mX-mX-mX-mX-mX-mX-mX-mX-[ademA-GalNAc]-[ademA-GalNAc]-[ademA-GalNAc]-mX-mX-mX-mX-mX-mX- 3′; hybridized to:
Antisense Strand: 5′-[MePhosphonate-40-mX]-S-fX-S-fX-mX-fX-mX-fX-mX-mX-fX-mX-mX-mX-fX-mX-mX-mX-mX-mX-mX-S-mX-S-mX-3′ ;
wherein mX= 2′-O-methyl modified nucleotide, fX =2′- fluoro modified nucleotide, -S- = phosphorothioate linkage, - = phosphodiester linkage, [MePhosphonate-4O-mX] = 5′-methoxyphosphonate-4-oxy modified nucleotide, and ademA-GalNAc = GalNAc attached to an adenine nucleotide.

In some embodiments, the disclosure provides an oligonucleotide (e.g., an RNAi oligonucleotide) for reducing ACC and/or DGAT2 expression, wherein the oligonucleotide comprises a sense strand and an antisense strand according to:

Sense Strand: 5′-mX-S-mX-mX-mX-mX-mX-mX-fX-fX-fX-fX-mX-mX-mX-mX-mX-mX-mX-mX-mX-mX-mX-mX-mX-mX-mX-mX-[ademA-GalNAc]-[ademA-GalNAc]-[ademA-GalNAc]-mX-mX-mX-mX-mX-mX - 3′; hybridized to:
Antisense Strand: 5′-[MePhosphonate-40-mX]-S-fX-S-fX-fX-fX-mX-fX-mX-mX-fX-mX-mX-mX-fX-mX-mX-mX-mX-mX-mX-S-mX-S-mX-3′ ;
wherein mX= 2′-O-methyl modified nucleotide, fX =2′- fluoro modified nucleotide, -S- = phosphorothioate linkage, - = phosphodiester linkage, [MePhosphonate-4O-mX] = 5′-methoxyphosphonate-4-oxy modified nucleotide, and ademA-GalNAc = GalNAc attached to an adenine nucleotide.

In some embodiments, the disclosure provides an oligonucleotide (e.g., an RNAi oligonucleotide) for reducing ACC expression, wherein the oligonucleotide comprises a sense strand and an antisense strand comprising nucleotide sequences selected from the group consisting of:
(a) SEQ ID NOs: 206 and 252, respectively;
(b) SEQ ID NOs: 207 and 253, respectively;
(c) SEQ ID NOs: 208 and 254, respectively;
(d) SEQ ID NOs: 209 and 255, respectively;
(e) SEQ ID NOs: 210 and 256, respectively;
(f) SEQ ID NOs: 211 and 257, respectively;
(g) SEQ ID NOs: 212 and 258, respectively;
(h) SEQ ID NOs: 213 and 259, respectively;
(i) SEQ ID NOs: 214 and 260, respectively;
(j) SEQ ID NOs: 215 and 261, respectively;
(k) SEQ ID NOs: 216 and 262, respectively;
(l) SEQ ID NOs: 217 and 263, respectively;
(m) SEQ ID NOs: 218 and 264, respectively;
(n) SEQ ID NOs: 219 and 265, respectively;
(o) SEQ ID NOs: 220 and 266, respectively;
(p) SEQ ID NOs: 221 and 267, respectively;
(q) SEQ ID NOs: 222 and 268, respectively;
(r) SEQ ID NOs: 223 and 269, respectively;
(s) SEQ ID NOs: 224 and 270, respectively;
(t) SEQ ID NOs: 225 and 271, respectively;
(u) SEQ ID NOs: 226 and 272, respectively;
(v) SEQ ID NOs: 227 and 273, respectively;
(w) SEQ ID NOs: 228 and 274, respectively;
(x) SEQ ID NOs: 229 and 275, respectively;
(y) SEQ ID NOs: 230 and 276, respectively;
(z) SEQ ID NOs: 231 and 277, respectively;
(aa) SEQ ID NOs: 232 and 278, respectively;
(bb) SEQ ID NOs: 233 and 279, respectively;
(cc) SEQ ID NOs: 234 and 280, respectively; and,
(dd) SEQ ID NOs: 235 and 281, respectively.

In some embodiments, the disclosure provides an oligonucleotide (e.g., an RNAi oligonucleotide) for reducing ACC expression, wherein the oligonucleotide comprises a sense strand and an antisense strand comprising nucleotide sequences selected from the group consisting of:
(a) SEQ ID NOs: 208 and 254, respectively;
(b) SEQ ID NOs: 209 and 255, respectively; and,
(c) SEQ ID NOs: 215 and 261, respectively.

In some embodiments, the disclosure provides an oligonucleotide (e.g., an RNAi oligonucleotide) for reducing DGAT2 expression, wherein the oligonucleotide comprises a sense strand and an antisense strand comprising nucleotide sequences selected from the group consisting of:
(a) SEQ ID NOs: 236 and 282, respectively;
(b) SEQ ID NOs: 237 and 283, respectively;
(c) SEQ ID NOs: 238 and 284, respectively;
(d) SEQ ID NOs: 239 and 285, respectively;
(e) SEQ ID NOs: 240 and 286, respectively;
(f) SEQ ID NOs: 241 and 287, respectively;
(g) SEQ ID NOs: 242 and 288, respectively;
(h) SEQ ID NOs: 243 and 289, respectively;
(i) SEQ ID NOs: 244 and 290, respectively;
(j) SEQ ID NOs: 245 and 291, respectively;
(k) SEQ ID NOs: 246 and 292, respectively;
(l) SEQ ID NOs: 247 and 293, respectively;
(m) SEQ ID NOs: 248 and 294, respectively;
(n) SEQ ID NOs: 249 and 295, respectively;
(o) SEQ ID NOs: 250 and 296, respectively; and,
(p) SEQ ID NOs: 251 and 297, respectively.

In some embodiments, the disclosure provides an oligonucleotide (e.g., an RNAi oligonucleotide) for reducing DGAT2 expression, wherein the oligonucleotide comprises a sense strand and an antisense strand comprising nucleotide sequences selected from the group consisting of:
(a) SEQ ID NOs: 246 and 292, respectively;
(b) SEQ ID NOs: 249 and 295, respectively;
(c) SEQ ID NOs: 240 and 286, respectively;
(d) SEQ ID NOs: 237 and, 283 respectively;
(e) SEQ ID NOs: 242 and 288, respectively; and
(f) SEQ ID NOs: 236 and 282, respectively.

In some embodiments, a ACC-targeting oligonucleotide for reducing ACC expression provided by the disclosure comprises a sense strand comprising the nucleotide sequence as set forth in SEQ ID NO: 208 and an antisense strand comprising the nucleotide sequence as set forth in SEQ ID NO: 254. In some embodiments, a ACC-targeting oligonucleotide for reducing ACC expression provided by the disclosure comprises a sense strand comprising the nucleotide sequence as set forth in SEQ ID NO: 209 and an antisense strand comprising the nucleotide sequence as set forth in SEQ ID NO: 255. In some embodiments, a ACC-targeting oligonucleotide for reducing ACC expression provided by the disclosure comprises a sense strand comprising the nucleotide sequence as set forth in SEQ ID NO: 215 and an antisense strand comprising the nucleotide sequence as set forth in SEQ ID NO: 261.

In some embodiments, a DGAT2-targeting oligonucleotide for reducing DGAT2 expression provided by the disclosure comprises a sense strand comprising the nucleotide sequence as set forth in SEQ ID NO: 246 and an antisense strand comprising the nucleotide sequence as set forth in SEQ ID NO: 292. In some embodiments, a DGAT2-targeting oligonucleotide for reducing DGAT2 expression provided by the disclosure comprises a sense strand comprising the nucleotide sequence as set forth in SEQ ID NO: 249 and an antisense strand comprising the nucleotide sequence as set forth in SEQ ID NO: 295. In some embodiments, a DGAT2-targeting oligonucleotide for reducing DGAT2 expression provided by the disclosure comprises a sense strand comprising the nucleotide sequence as set forth in SEQ ID NO: 240 and an antisense strand comprising the nucleotide sequence as set forth in SEQ ID NO: 286. In some embodiments, a DGAT2-targeting oligonucleotide for reducing DGAT2 expression provided by the disclosure comprises a sense strand comprising the nucleotide sequence as set forth in SEQ ID NO: 237 and an antisense strand comprising the nucleotide sequence as set forth in SEQ ID NO: 283. In some embodiments, a DGAT2-targeting oligonucleotide for reducing DGAT2 expression provided by the disclosure comprises a sense strand comprising the nucleotide sequence as set forth in SEQ ID NO: 242 and an antisense strand comprising the nucleotide sequence as set forth in SEQ ID NO: 288. In some embodiments, a DGAT2-targeting oligonucleotide for reducing DGAT2 expression provided by the disclosure comprises a sense strand comprising the nucleotide sequence as set forth in SEQ ID NO: 236 and an antisense strand comprising the nucleotide sequence as set forth in SEQ ID NO: 282.

### III. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, an oligonucleotide is formulated in buffer solutions such as phosphate buffered saline solutions, liposomes, micellar structures and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer’s instructions).

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal and rectal administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohol’s such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though several embodiments are directed to liver-targeted delivery of any of the oligonucleotides herein, targeting of other tissues is also contemplated.

### IV. Methods of Use

### I. Reducing ACC And/or DGAT2 Expression in Cells

The disclosure provides methods for contacting or delivering to a cell or population of cells an effective amount any one of oligonucleotides herein for purposes of reducing ACC and/or DGAT2 expression. The methods can include the steps described herein, and these maybe be, but not necessarily, carried out in the sequence as described. Other sequences, however, also are conceivable. Moreover, individual or multiple steps bay be carried out either in parallel and/or overlapping in time and/or individually or in multiply repeated steps. Furthermore, the methods may include additional, unspecified steps.

Methods herein are useful in any appropriate cell type. In some embodiments, a cell is any cell that expresses mRNA (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides herein are delivered using appropriate nucleic acid delivery methods including, but not limited to, injection of a solution containing the oligonucleotides, bombardment by particles covered by the oligonucleotides, exposing the cell or population of cells to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

In some embodiments, reduction of ACC and/or DGAT2 expression can be determined by an appropriate assay or technique to evaluate one or more properties or characteristics of a cell or population of cells associated with ACC and/or DGAT2 expression (e.g., using an ACC and/or DGAT2 expression biomarker) or by an assay or technique that evaluates molecules that are directly indicative of ACC and/or DGAT2 expression (e.g., ACC and/or DGAT2 mRNA or ACC and/or DGAT2 protein). In some embodiments, the extent to which an oligonucleotide herein reduces ACC and/or DGAT2 expression is evaluated by comparing ACC and/or DGAT2 expression in a cell or population of cells contacted with the oligonucleotide to an appropriate control (e.g., an appropriate cell or population of cells not contacted with the oligonucleotide or contacted with a control oligonucleotide). In some embodiments, an appropriate control level of mRNA expression into protein, after delivery of a RNAi molecule may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide herein results in a reduction in ACC and/or DGAT2 expression in a cell or population of cells. In some embodiments, the reduction in ACC and/or DGAT2 or DGAT expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower when compared with an appropriate control level of mRNA. The appropriate control level may be a level of mRNA expression and/or protein translation in a cell or population of cells that has not been contacted with an oligonucleotide herein. In some embodiments, the effect of delivery of an oligonucleotide to a cell according to a method herein is assessed after a finite period. For example, levels of mRNA may be analyzed in a cell at least about 8 hours, about 12 hours, about 18 hours, about 24 hours; or at least about 1, 2, 3, 4, 5, 6, 7 or even up to 14 days after introduction of the oligonucleotide into the cell. For example, in some embodiments, ACC and/or DGAT2 expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotide to the cell or population of cells. In some embodiments, ACC and/or DGAT2 expression is determined in a cell or population of cells at least about 1 month, about 2 months, about 3 months, about 4 months, about 5 months, or about 6 months or more after contacting or delivering the oligonucleotide to the cell or population of cells.

In some embodiments, an oligonucleotide is delivered in the form of a transgene that is engineered to express in a cell the oligonucleotide or strands comprising the oligonucleotide (e.g., its sense and antisense strands). In some embodiments, an oligonucleotide is delivered using a transgene engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

### II. Medical Use

The disclosure also provides oligonucleotides for use, or adaptable for use, to treat a subject (e.g., a human having a disease, disorder or condition associated with ACC and/or DGAT2 expression) that would benefit from reducing ACC and/or DGAT2 expression. In some respects, the disclosure provides oligonucleotides for use, or adapted for use, to treat a subject having a disease, disorder or condition associated with expression of ACC and/or DGAT2. The disclosure also provides oligonucleotides for use, or adaptable for use, in the manufacture of a medicament or pharmaceutical composition for treating a disease, disorder or condition associated with ACC and/or DGAT2 expression. In some embodiments, the oligonucleotides for use, or adaptable for use, target ACC and/or DGAT2 mRNA and reduce ACC and/or DGAT2 expression (e.g., via the RNAi pathway). In some embodiments, the oligonucleotides for use, or adaptable for use, target ACC and/or DGAT2 mRNA and reduce the amount or level of ACC and/or DGAT2 mRNA or DGAT2 mRNA, ACC and/or DGAT2 protein and/or ACC and/or DGAT2 activity.

In addition, the methods below can include selecting a subject having a disease, disorder or condition associated with ACC and/or DGAT2 expression or is predisposed to the same. In some instances, the methods can include selecting an individual having a marker for a disease associated with ACC and/or DGAT2 expression such as elevated blood pressure, insulin resistance, increased abdominal fat or elevated TG or cholesterol or is predisposed to the same.

Likewise, and as detailed below, the methods also may include steps such as measuring or obtaining a baseline value for a marker of ACC and/or DGAT2 expression, and then comparing such obtained value to one or more other baseline values or values obtained after being administered the oligonucleotide to assess the effectiveness of treatment.

### III. Methods of Treatment

The disclosure also provides methods of treating a subject having, suspected of having, or at risk of developing a disease, disorder or condition with an oligonucleotide herein. In some aspects, the disclosure provides methods of treating or attenuating the onset or progression of a disease, disorder or condition associated with ACC and/or DGAT2 expression using the oligonucleotides herein. In other aspects, the disclosure provides methods to achieve one or more therapeutic benefits in a subject having a disease, disorder or condition associated with ACC and/or DGAT2 expression using the oligonucleotides herein. In some embodiments of the methods herein, the subject is treated by administering a therapeutically effective amount of any one or more of the oligonucleotides herein. In some embodiments, treatment comprises reducing ACC and/or DGAT2 expression. In some embodiments, the subject is treated therapeutically. In some embodiments, the subject is treated prophylactically. In some embodiments, the subject has received or is receiving treatment for reducing ACC (e.g., an ACC-targeting oligonucleotide), and is administered treatment for reducing DGAT2 expression (e.g., a DGAT2-targeting oligonucleotide). In some embodiments, the subject has received or is receiving treatment for reducing DGAT2 (e.g., a DGAT2-targeting oligonucleotide), and is administered treatment for reducing ACC expression (e.g., an ACC-targeting oligonucleotide).

In some embodiments of the methods herein, one or more oligonucleotides herein, or a pharmaceutical composition comprising one or more oligonucleotides, is administered to a subject having a disease, disorder or condition associated with ACC and/or DGAT2 expression such that ACC and/or DGAT2 expression is reduced in the subject, thereby treating the subject. In some embodiments, an amount or level of ACC and/or DGAT2 mRNA is reduced in the subject. In some embodiments, an amount or level of ACC and/or DGAT2 and/or protein is reduced in the subject. In some embodiments, an amount or level of ACC and/or DGAT2 activity is reduced in the subject. In some embodiments, an amount or level of triglyceride (TG) (e.g., one or more TG(s) or total TGs) is reduced in the subject. In some embodiments, an amount or level of plasma glucose is reduced in the subject. In some embodiments, an amount or level of blood pressure (e.g. systolic pressure, diastolic pressure or both) is reduced in the subject. In some embodiments, an amount or level of abdominal fat is reduced in the subject. In some embodiments, an amount or level of cholesterol (e.g., total cholesterol, LDL cholesterol, and/or HDL cholesterol) is reduced in the subject. In some embodiments, an amount or level of liver steatosis is reduced in the subject. . In some embodiments, an amount or level of liver fibrosis is reduced in the subject. In some embodiments, the ratio of total cholesterol to HDL cholesterol is altered in the subject. In some embodiments, any combination of the following is reduced or altered in the subject: ACC and/or DGAT2 expression, an amount or level of ACC and/or DGAT2 mRNA, an amount or level of ACC and/or DGAT2 protein, an amount or level of ACC and/or DGAT2 activity, an amount or level of blood glucose, an amount or level of abdominal fat, an amount or level of blood pressure, an amount or level of TG, an amount or level of cholesterol and/or the ratio of total cholesterol to HDL cholesterol, an amount or level of liver steatosis, and amount or level of liver fibrosis.

In some embodiments of the methods herein, an oligonucleotide herein, or a pharmaceutical composition comprising the oligonucleotide, is administered to a subject having a disease, disorder or condition associated with ACC and/or DGAT2 such that ACC and/or DGAT2 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to ACC and/or DGAT2 expression prior to administration of one or more oligonucleotides or pharmaceutical composition. In some embodiments, ACC and/or DGAT2 expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to ACC and/or DGAT2 expression in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with ACC and/or DGAT2 expression such that an amount or level of ACC and/or DGAT2 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of ACC and/or DGAT2 mRNA prior to administration of the oligonucleotide or pharmaceutical composition. In some embodiments, an amount or level of ACC and/or DGAT2 mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of ACC and/or DGAT2 mRNA in a subject (e.g., a reference or control subject) not receiving the oligonucleotide or oligonucleotides or pharmaceutical composition or receiving a control oligonucleotide or oligonucleotides, pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with ACC and/or DGAT2 expression such that an amount or level of ACC and/or DGAT2 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of ACC and/or DGAT2 protein prior to administration of the oligonucleotide or pharmaceutical composition. In some embodiments, an amount or level of ACC and/or DGAT2 protein is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of ACC and/or DGAT2 protein in a subject (e.g., a reference or control subject) not receiving the oligonucleotide or oligonucleotides or pharmaceutical composition or receiving a control oligonucleotide, oligonucleotides or pharmaceutical composition or treatment.

In some embodiments of the methods herein, an oligonucleotide or oligonucleotides herein, or a pharmaceutical composition comprising the oligonucleotide or oligonucleotides, is administered to a subject having a disease, disorder or condition associated with ACC and/or DGAT2 such that an amount or level of ACC and/or DGAT2 activity/expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of ACC and/or DGAT2 activity prior to administration of the oligonucleotide or pharmaceutical composition. In some embodiments, an amount or level of ACC and/or DGAT2 activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of ACC and/or DGAT2 activity in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with ACC and/or DGAT2 expression such that an amount or level of TG (e.g., one or more TGs or total TGs) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of TG prior to administration of the oligonucleotide or pharmaceutical composition. In some embodiments, an amount or level of TG is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of TG in a subject (e.g., a reference or control subject) not receiving the oligonucleotide or pharmaceutical composition or receiving a control oligonucleotide, pharmaceutical composition or treatment.

Generally, a normal or desirable TG range for a human patient is <150 mg/dL of blood, with <100 being considered ideal. In some embodiments, the patient selected for treatment or treated is identified or determined to have an amount or level of TG of ≥150 mg/dL. In some embodiments, the patient selected for treatment or treated is identified or determined to have an amount or level of TG in the range of 150 to 199 mg/dL, which is considered borderline high TG levels. In some embodiments, the patient selected for treatment or treated is identified or determined to have an amount or level of TG in the range of 200 to 499 mg/dL, which is considered high TG levels. In some embodiments, the patient selected for treatment or treated is identified or determined to have an amount or level of TG in the range of 500 mg/dL or higher (i.e., ≥500 mg/dL), which is considered very high TG levels. In some embodiments, the patient selected for treatment or treated is identified or determined to have an amount or level of TG which is ≥150 mg/dL, ≥200 mg/dL or ≥ 500 mg/dL. In some embodiments, the patient selected for treatment or treated is identified or determined to have an amount of level of TG of 200 to 499 mg/dL, or 500 mg/dL or higher. In some embodiments, the patient selected for treatment or treated is identified or determined to have an amount or level of TG which is ≥200 mg/dL.In some embodiments of the methods herein, an oligonucleotide herein, or a pharmaceutical composition comprising the oligonucleotide, is administered to a subject having a disease, disorder or condition associated with ACC and/or DGAT2 expression such that an amount or level of cholesterol (e.g., total cholesterol, LDL cholesterol, and/or HDL cholesterol) is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of cholesterol prior to administration of the oligonucleotide or pharmaceutical composition. In some embodiments, an amount or level of cholesterol is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of cholesterol in a subject (e.g., a reference or control subject) not receiving the oligonucleotide or pharmaceutical composition or receiving a control oligonucleotide, pharmaceutical composition or treatment.

Generally, a normal or desirable cholesterol range (total cholesterol) for an adult human patient is <200 mg/dL of blood. In some embodiments, the patient selected for treatment or treated is identified or determined to have an amount or level of cholesterol of ≥200 mg/dL. In some embodiments, the patient selected for treatment or treated is identified or determined to have an amount or level of cholesterol in the range of 200 to 239 mg/dL, which is considered borderline high cholesterol levels. In some embodiments, the patient selected for treatment or treated is identified or determined to have an amount or level of cholesterol in the range of 240 mg/dL and higher (i.e., ≥240 mg/dL), which is considered high cholesterol levels. In some embodiments, the patient selected from treatment or treated is identified or determined to have an amount or level of cholesterol of 200 to 239 mg/dL, or 240 mg/dL or higher. In some embodiments, the patient selected for treatment or treated is identified or determined to have an amount or level of cholesterol which is ≥200 mg/dL or ≥240 mg/dL or higher.

In some embodiments of the methods herein, an oligonucleotide herein, or a pharmaceutical composition comprising the oligonucleotide, is administered to a subject having a disease, disorder or condition associated with ACC and/or DGAT2 expression such that an amount or level of liver fibrosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of liver fibrosis prior to administration of the oligonucleotide or pharmaceutical composition. In some embodiments, an amount or level of liver fibrosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of liver fibrosis in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with ACC and/or DGAT2 expression such that an amount or level of liver steatosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of liver steatosis prior to administration of the oligonucleotide or pharmaceutical composition. In some embodiments, an amount or level of liver steatosis is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of liver steatosis in a subject (e.g., a reference or control subject) not receiving the oligonucleotide or pharmaceutical composition or receiving a control oligonucleotide, pharmaceutical composition or treatment.

Suitable methods for determining ACC and/or DGAT2 expression, the amount or level of ACC and/or DGAT2 mRNA, ACC and/or DGAT2 protein, ACC and/or DGAT2 activity, TG, plasma glucose or cholesterol amount or activity in the subject, or in a sample from the subject, are known in the art. Further, the Examples set forth herein illustrate methods for determining ACC and/or DGAT2 expression.

In some embodiments, ACC and/or DGAT2 expression, the amount or level of ACC and/or DGAT2 mRNA, ACC and/or DGAT2 protein, ACC and/or DGAT2 activity, TG, plasma glucose, or cholesterol, is reduced in a cell (e.g., a hepatocyte), a population or a group of cells (e.g., an organoid), an organ (e.g., liver), blood or a fraction thereof (e.g., plasma), a tissue (e.g., liver tissue), a sample (e.g., a liver biopsy sample), or any other appropriate biological material obtained or isolated from the subject. In some embodiments, ACC and/or DGAT2 expression, the amount or level of ACC and/or DGAT2 mRNA, ACC and/or DGAT2 protein, ACC and/or DGAT2 activity, TG, plasma glucose or cholesterol or any combination thereof, is reduced in more than one type of cell (e.g., a hepatocyte and one or more other type(s) of cell), more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), or more than one type of sample (e.g., a liver biopsy sample and one or more other type(s) of biopsy sample).

Examples of a disease, disorder or condition associated with ACC and/or DGAT2 expression include, but are not limited to, metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker’s lung, tuberculosis, pulmonary fibrosis, idiopathic pulmonary fibrosis (IPF), and other ACC and/or DGAT2-associated metabolic-related disorders and diseases. Of particular interest herein are metabolic syndrome, hypertriglyceridemia, NASH, obesity or a combination thereof.

Because of their high specificity, the oligonucleotides herein specifically target mRNAs of target genes of diseased cells and tissues. In preventing disease, the target gene may be one which is required for initiation or maintenance of the disease or which has been identified as being associated with a higher risk of contracting the disease. In treating disease, the oligonucleotide can be brought into contact with the cells or tissue exhibiting the disease. For example, an oligonucleotide substantially identical to all or part of a wild-type (i.e., native) or mutated gene associated with a disorder or condition associated with ACC and/or DGAT2 expression may be brought into contact with or introduced into a cell or tissue type of interest such as a hepatocyte or other liver cell.

In some embodiments, the ACC and/or DGAT gene may be an ACC and/or DGAT gene from any mammal, such as a human target. Any ACC and/or DGAT gene may be silenced according to the method described herein.

Methods described herein are typically involve administering to a subject in an effective amount of an oligonucleotide or oligonucleotides, that is, an amount capable of producing a desirable therapeutic result. A therapeutically acceptable amount may be an amount that can therapeutically treat a disease or disorder. The appropriate dosage for any one subject will depend on certain factors, including the subject’s size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intraarterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides herein are administered intravenously or subcutaneously.

As a non-limiting set of examples, the oligonucleotides herein would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments the oligonucleotides herein are administered alone or in combination. In some embodiments the oligonucleotides herein are administered in combination concurrently, sequentially (in any order), or intermittently. For example two oligonucleotides may be co-administered concurrently. Alternatively, one oligonucleotide may be administered and followed any amount of time later (e.g., one hour, one day, one week or one month) by the administration of a second oligonucleotide.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### V. Kits

In some embodiments, the disclosure provides a kit comprising an oligonucleotide herein, and instructions for use. In some embodiments, the kit comprises an oligonucleotide herein, and a package insert containing instructions for use of the kit and/or any component thereof. In some embodiments, the kit comprises, in a suitable container, an oligonucleotide herein, one or more controls, and various buffers, reagents, enzymes and other standard ingredients well known in the art. In some embodiments, the container comprises at least one vial, well, test tube, flask, bottle, syringe or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, a kit comprises an oligonucleotide herein, and a pharmaceutically acceptable carrier, or a pharmaceutical composition comprising the oligonucleotide and instructions for treating or delaying progression of a disease, disorder or condition associated with ACC and/or DGAT2 expression in a subject in need thereof.

### VI. Other Embodiments

The disclosure relates to the following embodiments. Throughout this section, the term embodiment is abbreviated as “E” followed by an ordinal. For example, E1 is equivalent to Embodiment 1.

E1. An oligonucleotide for reducing ACC expression, the oligonucleotide comprising an antisense strand comprising a sequence as set forth in any one of SEQ ID NOs: 2, 30, 32, 44 and 56.

E2. The oligonucleotide of embodiment 1, comprising a sense strand comprising a sequence as set forth in any one of SEQ ID NOs: 1, 29, 31, 43 and 55.

E3. An oligonucleotide for reducing ACC expression, the oligonucleotide comprising an antisense strand of 15 to 30 nucleotides in length and a sense strand of 15 to 40 nucleotides in length, wherein the antisense strand has a region of complementarity to a target sequence of ACC and wherein the region of complementarity is at least 15 contiguous nucleotides in length.

E4. The oligonucleotide of embodiment 3, wherein the region of complementarity is fully complementary to the target sequence of ACC.

E5. The oligonucleotide of any one of embodiments 1 to 4, wherein the antisense strand is 19 to 27 nucleotides in length.

E6. The oligonucleotide of any one of embodiments 1 to 5, wherein the antisense strand is 21 to 27 nucleotides in length, optionally wherein the antisense strand is 22 nucleotides in length.

E7. The oligonucleotide of any one of embodiments 2 to 6, wherein the sense strand forms a duplex region with the antisense strand.

E8. The oligonucleotide of embodiment 7, wherein the sense strand is 19 to 40 nucleotides in length, optionally wherein the sense strand is 36 nucleotides in length.

E9. The oligonucleotide of embodiment 7 or 8, wherein the duplex region is at least 19 nucleotides in length.

E10. The oligonucleotide of any one of embodiments 7 to 9, wherein the duplex region is at least 21 nucleotides in length, optionally wherein the duplex region is 20 nucleotides in length.

E11. The oligonucleotide of any one of embodiments 3 to 10, wherein the region of complementarity to ACC is at least 19 contiguous nucleotides in length.

E12. The oligonucleotide of any one of embodiments 3 to 11, wherein the region of complementarity to ACC is at least 21 contiguous nucleotides in length.

E13. The oligonucleotide of any one of embodiments 3 to 12, wherein the antisense strand comprises a sequence as set forth in any one of SEQ ID NOs: 2, 30, 32, 44 and 56.

E14. The oligonucleotide of any one of embodiments 3 to 13, wherein the sense strand comprises a sequence as set forth in any one of SEQ ID NOs: 1, 29, 31, 43 and 55.

E15. The oligonucleotide of any one of embodiments 3 to 14, wherein the sense strand comprises at its 3′ end a stem-loop set forth as: S1-L-S2, wherein S1 is complementary to S2, and wherein L forms a loop between S1 and S2 of 3 to 5 nucleotides in length.

E16. An oligonucleotide for reducing ACC expression, the oligonucleotide comprising an antisense strand and a sense strand, wherein the antisense strand is 21 to 27 nucleotides in length and has a region of complementarity to ACC, wherein the sense strand comprises at its 3′ end a stem-loop set forth as: S1-L-S2, wherein S1 is complementary to S2, and wherein L forms a loop between S1 and S2 of 3 to 5 nucleotides in length, and wherein the antisense strand and the sense strand form a duplex structure of at least 19 nucleotides in length but are not covalently linked.

E17. The oligonucleotide of embodiment 16, wherein the region of complementarity is fully complementary to at least 19 contiguous nucleotides of ACC mRNA.

E18. The oligonucleotide of any one of embodiments 15 to 17, wherein L is a tetraloop.

E19. The oligonucleotide of any one of embodiments 15 to 18, wherein L is 4 nucleotides in length.

E20. The oligonucleotide of any one of embodiments 15 to 19, wherein L comprises a sequence set forth as GAAA.

E21. The oligonucleotide of any one of embodiments 3 to 20, wherein the antisense strand is 27 nucleotides in length and the sense strand is 25 nucleotides in length, optionally wherein the antisense strand is 22 nucleotides in length and the sense strand is 36 nucleotides in length.

E22. The oligonucleotide of embodiment 21, wherein the antisense strand and sense strand form a duplex region of 25 nucleotides in length, optionally wherein the duplex is 20 nucleotides in length.

E23. The oligonucleotide of any one of embodiments 16 to 20, comprising a 3′-overhang sequence on the antisense strand of 2 nucleotides in length.

E24. The oligonucleotide of any one of embodiments 7 to 16, wherein the oligonucleotide comprises an antisense strand and a sense strand that are each in a range of 21 to 23 nucleotides in length.

E25. The oligonucleotide of embodiment 24, wherein the oligonucleotide comprises a duplex structure in a range of 19 to 21 nucleotides in length.

E26. The oligonucleotide of embodiment 24 or 25, wherein the oligonucleotide comprises a 3′-overhang sequence of one or more nucleotides in length, wherein the 3′-overhang sequence is present on the antisense strand, the sense strand, or the antisense strand and sense strand.

E27. The oligonucleotide of embodiment 24 or 25, wherein the oligonucleotide comprises a 3′-overhang sequence of 2 nucleotides in length, wherein the 3′-overhang sequence is on the antisense strand, and wherein the sense strand is 21 nucleotides in length and the antisense strand is 23 nucleotides in length, such that the sense strand and antisense strand form a duplex of 21 nucleotides in length.

E28. The oligonucleotide of any one of the preceding embodiments, wherein the oligonucleotide comprises at least one modified nucleotide.

E29. The oligonucleotide of embodiment 28, wherein the modified nucleotide comprises a 2′-modification.

E30. The oligonucleotide of embodiment 29, wherein the 2′-modification is a modification selected from 2′-aminoethyl, 2′-fluoro, 2′—O—methyl, 2′—O—methoxyethyl, and 2′-deoxy-2′-fluoro-β-d-arabinonucleic acid.

E31. The oligonucleotide of any one of embodiments 28 to 30, wherein all of the nucleotides of the oligonucleotide are modified.

E32. The oligonucleotide of any one of the preceding embodiments, wherein the oligonucleotide comprises at least one modified internucleotide linkage.

E33. The oligonucleotide of embodiment 32, wherein the at least one modified internucleotide linkage is a phosphorothioate linkage.

E34. The oligonucleotide of any one of the preceding embodiments, wherein the 4′-carbon of the sugar of the 5′-nucleotide of the antisense strand comprises a phosphate analog.

E35. The oligonucleotide of embodiment 34, wherein the phosphate analog is oxymethylphosphonate, vinylphosphonate or malonyl phosphonate.

E36. The oligonucleotide of any one of the preceding embodiments, wherein at least one nucleotide of the oligonucleotide is conjugated to one or more targeting ligands.

E37. The oligonucleotide of embodiment 36, wherein each targeting ligand comprises a carbohydrate, amino sugar, cholesterol, polypeptide or lipid.

E38. The oligonucleotide of embodiment 36, wherein each targeting ligand comprises a saturated fatty acid moiety that in size ranges from C10 to C24 long.

E39. The oligonucleotide of embodiment 38, wherein each targeting ligand comprises a saturated fatty acid moiety that is C16 long.

E40. The oligonucleotide of embodiment 38, wherein each targeting ligand comprises a saturated fatty acid moiety that is C24 long.

E41. The oligonucleotide of embodiment 36, wherein each targeting ligand comprises a N-acetylgalactosamine (GalNAc) moiety.

E42. The oligonucleotide of embodiment 41, wherein the GalNAc moiety is a monovalent GalNAc moiety, a bivalent GalNAc moiety, a trivalent GalNAc moiety or a tetravalent GalNAc moiety.

E43. The oligonucleotide of any one of embodiments 15 to 20, wherein up to 4 nucleotides of L of the stem-loop are each conjugated to a monovalent GalNAc moiety.

E44. The oligonucleotide of any one of the preceding embodiments, wherein the oligonucleotide is an RNAi oligonucleotide.

E45. A pharmaceutical composition comprising the oligonucleotide of any one of the preceding embodiments and a pharmaceutically acceptable carrier, delivery agent or excipient.

E46. An oligonucleotide for reducing DGAT2 expression, the oligonucleotide comprising an antisense strand comprising a sequence as set forth in any one of SEQ ID NOs: 106, 108, 112, 126, 130 and 138.

E47. The oligonucleotide of embodiment 46, comprising a sense strand comprising a sequence as set forth in any one of SEQ ID NOs: 105, 107, 111, 125, 129 and 137.

E48. An oligonucleotide for reducing DGAT2 expression, the oligonucleotide comprising an antisense strand of 15 to 30 nucleotides in length and a sense strand of 15 to 40 nucleotides in length, wherein the antisense strand has a region of complementarity to a target sequence of DGAT2 and wherein the region of complementarity is at least 15 contiguous nucleotides in length.

E49. The oligonucleotide of embodiment 48, wherein the region of complementarity is fully complementary to the target sequence of DGAT2.

E50. The oligonucleotide of any one of embodiments 46 to 49, wherein the antisense strand is 19 to 27 nucleotides in length.

E51. The oligonucleotide of any one of embodiments 46 to 50, wherein the antisense strand is 21 to 27 nucleotides in length, optionally wherein the antisense strand is 22 nucleotides in length.

E52. The oligonucleotide of any one of embodiments 47 to 51, wherein the sense strand forms a duplex region with the antisense strand.

E53. The oligonucleotide of embodiment 52, wherein the sense strand is 19 to 40 nucleotides in length, optionally wherein the sense strand is 36 nucleotides in length.

E54. The oligonucleotide of embodiment 52 or 53, wherein the duplex region is at least 19 nucleotides in length.

E55. The oligonucleotide of any one of embodiments 52 to 54, wherein the duplex region is at least 21 nucleotides in length, optionally wherein the duplex region is 20 nucleotides in length.

E56. The oligonucleotide of any one of embodiments 48 to 55, wherein the region of complementarity to ACC is at least 19 contiguous nucleotides in length.

E57. The oligonucleotide of any one of embodiments 48 to 56, wherein the region of complementarity to DGAT2 is at least 21 contiguous nucleotides in length.

E58. The oligonucleotide of any one of embodiments 48 to 57, wherein the antisense strand comprises a sequence as set forth in any one of SEQ ID NOs: 106, 108, 112, 126, 130 and 138.

E59. The oligonucleotide of any one of embodiments 48 to 58, wherein the sense strand comprises a sequence as set forth in any one of SEQ ID NOs: 105, 107, 111, 125, 129 and 137.

E60. The oligonucleotide of any one of embodiments 48 to 59, wherein the sense strand comprises at its 3′ end a stem-loop set forth as: S1-L-S2, wherein S1 is complementary to S2, and wherein L forms a loop between S1 and S2 of 3 to 5 nucleotides in length.

E61. An oligonucleotide for reducing DGAT2 expression, the oligonucleotide comprising an antisense strand and a sense strand, wherein the antisense strand is 21 to 27 nucleotides in length and has a region of complementarity to DGAT2, wherein the sense strand comprises at its 3′ end a stem-loop set forth as: S1-L-S2, wherein S1 is complementary to S2, and wherein L forms a loop between S1 and S2 of 3 to 5 nucleotides in length, and wherein the antisense strand and the sense strand form a duplex structure of at least 19 nucleotides in length but are not covalently linked.

E62. The oligonucleotide of embodiment 61, wherein the region of complementarity is fully complementary to at least 19 contiguous nucleotides of DGAT2 mRNA.

E63. The oligonucleotide of any one of embodiments 60 to 62, wherein L is a tetraloop.

E64. The oligonucleotide of any one of embodiments 60 to 63, wherein L is 4 nucleotides in length.

E65. The oligonucleotide of any one of embodiments 60 to 64, wherein L comprises a sequence set forth as GAAA.

E66. The oligonucleotide of any one of embodiments 46 to 65, wherein the antisense strand is 27 nucleotides in length and the sense strand is 25 nucleotides in length, optionally wherein the antisense strand is 22 nucleotides in length and the sense strand is 36 nucleotides in length.

E67. The oligonucleotide of embodiment 66, wherein the antisense strand and sense strand form a duplex region of 25 nucleotides in length, optionally wherein the duplex is 20 nucleotides in length.

E68. The oligonucleotide of any one of embodiments 48 to 65, comprising a 3′-overhang sequence on the antisense strand of 2 nucleotides in length.

E69. The oligonucleotide of any one of embodiments 52 to 61, wherein the oligonucleotide comprises an antisense strand and a sense strand that are each in a range of 21 to 23 nucleotides in length.

E70. The oligonucleotide of embodiment 69, wherein the oligonucleotide comprises a duplex structure in a range of 19 to 21 nucleotides in length.

E71. The oligonucleotide of embodiment 69 or 70, wherein the oligonucleotide comprises a 3′-overhang sequence of one or more nucleotides in length, wherein the 3′-overhang sequence is present on the antisense strand, the sense strand, or the antisense strand and sense strand.

E72. The oligonucleotide of embodiment 69 or 70, wherein the oligonucleotide comprises a 3′-overhang sequence of 2 nucleotides in length, wherein the 3′-overhang sequence is on the antisense strand, and wherein the sense strand is 21 nucleotides in length and the antisense strand is 23 nucleotides in length, such that the sense strand and antisense strand form a duplex of 21 nucleotides in length.

E73. The oligonucleotide of any one of the preceding embodiments, wherein the oligonucleotide comprises at least one modified nucleotide.

E74. The oligonucleotide of embodiment 73, wherein the modified nucleotide comprises a 2′-modification.

E75. The oligonucleotide of embodiment 74, wherein the 2′-modification is a modification selected from 2′ aminoethyl, 2′-fluoro, 2′-O-methyl, 2′-O-methoxyethyl, and 2′-deoxy-2′-fluoro-β-d-arabinonucleic acid.

E76. The oligonucleotide of any one of embodiments 73 to 75, wherein all of the nucleotides of the oligonucleotide are modified.

E77. The oligonucleotide of any one of the preceding embodiments, wherein the oligonucleotide comprises at least one modified internucleotide linkage.

E78. The oligonucleotide of embodiment 77, wherein the at least one modified internucleotide linkage is a phosphorothioate linkage.

E79. The oligonucleotide of any one of the preceding embodiments, wherein the 4′-carbon of the sugar of the 5′-nucleotide of the antisense strand comprises a phosphate analog.

E80. The oligonucleotide of embodiment 79, wherein the phosphate analog is oxymethylphosphonate, vinylphosphonate or malonyl phosphonate.

E81. The oligonucleotide of any one of the preceding embodiments, wherein at least one nucleotide of the oligonucleotide is conjugated to one or more targeting ligands.

E82. The oligonucleotide of embodiment 81, wherein each targeting ligand comprises a carbohydrate, amino sugar, cholesterol, polypeptide or lipid

E83. The oligonucleotide of embodiment 82, wherein each targeting ligand comprises a saturated fatty acid moiety that in size ranges from C10 to C24 long.

E84. The oligonucleotide of embodiment 83, wherein each targeting ligand comprises a C16 saturated fatty acid moiety.

E85. The oligonucleotide of embodiment 83, wherein each targeting ligand comprises a C22 saturated fatty acid moiety.

E86. The oligonucleotide of embodiment 82, wherein each targeting ligand comprises a N-acetylgalactosamine (GalNAc) moiety.

E87. The oligonucleotide of embodiment 86, wherein the GalNAc moiety is a monovalent GalNAc moiety, a bivalent GalNAc moiety, a trivalent GalNAc moiety or a tetravalent GalNAc moiety.

E88. The oligonucleotide of any one of embodiments 60 to 65, wherein up to 4 nucleotides of L of the stem-loop are each conjugated to a monovalent GalNAc moiety

E89. The oligonucleotide of any one of the preceding embodiments, wherein the oligonucleotide is an RNAi oligonucleotide.

E90. A pharmaceutical composition comprising the oligonucleotide of any one of the preceding embodiments and a pharmaceutically acceptable carrier, delivery agent or excipient.

E91. A pharmaceutical composition comprising at least a first and second therapeutic agent, wherein the first therapeutic agent is an inhibitor of ACC and wherein the second therapeutic agent is an inhibitor of DGAT2 expression.

E92. A pharmaceutical composition comprising at least a first and a second therapeutic agent, wherein the first therapeutic agent is an oligonucleotide and is an inhibitor of ACC expression and wherein the second therapeutic agent is an inhibitor of DGAT2 expression.

E93. A pharmaceutical composition comprising at least a first and a second therapeutic agent, wherein the first therapeutic agent is an inhibitor of ACC expression and wherein the second therapeutic agent is an oligonucleotide and is an inhibitor of DGAT2 expression.

E94. A pharmaceutical composition comprising at least a first and second therapeutic agent, wherein the first therapeutic agent is an oligonucleotide selected from any one of embodiments 1-45 and is an inhibitor of ACC expression and wherein the second therapeutic agent is an oligonucleotide selected from any one of embodiments 46-89 and is an inhibitor of DGAT expression.

E95. A combination product comprising: (i) an ACC inhibiting oligonucleotide with an antisense strand as set forth in any one of SEQ ID NOs: 2, 30, 32, 44 and 56; and, (ii) a sense strand as set forth in any one of SEQ ID NOs: 1, 29, 31, 43, and 55, and, (iii) a DGAT inhibiting with an antisense strand as set forth in any one of SEQ ID NOs: 106, 108, 112, 126, 130, 138; and, (iv) a sense strand as set forth in any one of SEQ ID NOs: 105, 107, 111, 125, 129 and 137.

E96. The combination product according to embodiment 95 wherein component (i) is an oligonucleotide comprising an antisense strand of 15 to 30 nucleotides in length and wherein component (ii) is a sense strand of 15 to 40 nucleotides in length, and is an inhibitor of ACC expression, and wherein component (iii) is an oligonucleotide comprising an antisense strand of 15 to 30 nucleotides in length and wherein component (iv) is a sense strand of 15 to 40 nucleotides in length and is an inhibitor of DGAT2 expression. The combination product of embodiment 97 wherein the combination product is a composition comprising components (i), (ii), (iii) and (iv) and a pharmaceutically acceptable salt thereof.

E97. The combination product according to embodiment 95 or 96, wherein components (i) and (ii) are formulated in an injectable suspension, a gel, an oil, a pill, a tablet, a suppository, a powder, a capsule, an aerosol, an ointment, a cream, a patch, or means of galenic forms for a prolonged and/or slow release.

E98. The combination product according to any one of embodiments 94 to 96, for the treatment of an inflammatory, metabolic, fibrotic or cholestatic disease.

E99. The combination product for use according to embodiment 98, wherein the disease is selected from the group consisting of, the disease is selected in the group consisting of metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker’s lung, tuberculosis, pulmonary fibrosis, idiopathic pulmonary fibrosis (IPF).

E100. A method of delivering an oligonucleotide to a subject, the method comprising administering pharmaceutical composition selected from any one of embodiments 95-99 to a subject.

E101. A method for reducing ACC expression in a cell, a population of cells or a subject, the method comprising the steps of: contacting the cell or the population of cells with the oligonucleotide of any one of embodiments 1-44, or the pharmaceutical composition of embodiment 45; or administering to the subject the oligonucleotide of any one of embodiments 1 to 44, or the pharmaceutical composition of embodiment 45.

E102. The method of embodiment 101, wherein reducing ACC expression comprises reducing an amount or a level of ACC mRNA, an amount or a level of ACC protein, or both.

E103. The method of a embodiment 101 or 102, wherein the subject has a disease, disorder or condition associated with ACC expression.

E104. A method for treating a subject having a disease, disorder or condition associated with ACC expression, the method comprising administering to the subject a therapeutically effective amount of the oligonucleotide of any one of embodiments 1-44, or the pharmaceutical composition of embodiment 45, thereby treating the subject.

E105. A method for reducing an amount or level of liver fibrosis in a subject, the method comprising administering to the subject the oligonucleotide of any one of embodiments 1-44, or the pharmaceutical composition of embodiment 45.

E106. A method for treating a subject having a disease, disorder or condition associated with ACC expression, the method comprising administering to the subject a therapeutically effective amount of the oligonucleotide of any one of embodiments 1 to 44, or the pharmaceutical composition of embodiment 45, thereby treating the subject, wherein the therapeutically effective amount is 0.03, 0.075, 0.15, 0.3, 0.75, 1.5, 3, 6, 12, 24, 60, 120 or 600 mg/kg.

E107. A method for treating a subject having a disease, disorder or condition associated with ACC expression, the method comprising administering to the subject a therapeutically effective amount of the oligonucleotide of any one of embodiments 1 to 44, or the pharmaceutical composition of embodiment 45, thereby treating the subject, wherein the therapeutically effective amount is 1.5, 3, 6 or 12 mg/kg.

E108. A method for treating a subject having a disease, disorder or condition associated with ACC expression, the method comprising administering to the subject a therapeutically effective amount of an oligonucleotide comprising a (i) sense strand selected from SEQ ID Nos: 1, 29, 31, 43 and 55 and an (ii) antisense strand selected from SEQ ID Nos: 2, 30, 32, 44 and 56, or pharmaceutical composition thereof, thereby treating the subject.

E109. The method of any one of embodiments 103-108, wherein the disease, disorder or condition associated with ACC expression is selected from the group consisting of metabolic liver disease, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial

Intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker’s lung, tuberculosis, pulmonary fibrosis, idiopathic pulmonary fibrosis (IPF).

E110. The method of embodiment 109, wherein the disease, disorder or condition associated with ACC expression is metabolic liver disease, non-alcoholic fatty liver disease (NAFLD) or non-alcoholic steatohepatitis (NASH).

E111. The method of any one of embodiments 103-110, wherein the disease, disorder or condition associated with ACC expression is or non-alcoholic steatohepatitis (NASH).

E112. The method of any one of embodiments 103-110, wherein the oligonucleotide, or pharmaceutical composition, is administered in combination with a second composition or therapeutic agent.

E113. Use of the oligonucleotide of any one of embodiments 1-44, or the pharmaceutical composition of embodiment 45, in the manufacture of a medicament for the treatment of a disease, disorder or condition associated with ACC expression.

E114. The oligonucleotide of any one of embodiments 1-44, or the pharmaceutical composition of embodiment 45, for use, or adaptable for use, in the treatment of a disease, disorder or condition associated with ACC expression.

E115. A kit comprising the oligonucleotide of any one of embodiments 1-44, an optional pharmaceutically acceptable carrier, and a package insert comprising instructions for administration to a subject having a disease, disorder or condition associated with ACC expression.

E116. The use the oligonucleotide(s) of embodiment 114 or the use of the kit of embodiment 115, wherein the disease, disorder or condition associated with ACC expression is selected from the group consisting of metabolic liver disease, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker’s lung, tuberculosis, pulmonary fibrosis, idiopathic pulmonary fibrosis (IPF).

E117. The use the oligonucleotide(s) of embodiment 114 or the use of the kit of embodiment 115, wherein the disease, disorder or condition associated with ACC expression is cardiovascular disease, type II diabetes mellitus, hypertriglyceridemia, NASH, obesity, or a combination thereof.

E118. A method for reducing DGAT2 expression in a cell, a population of cells or a subject, the method comprising the steps of: contacting the cell or the population of cells with the oligonucleotide of any one of embodiments 46 to 88, or the pharmaceutical composition of embodiment 89; or administering to the subject the oligonucleotide of any one of embodiments 46 to 88, or the pharmaceutical composition of embodiment 89.

E119. The method of embodiment 118, wherein reducing DGAT2 expression comprises reducing an amount or a level of DGAT2 mRNA, an amount or a level of DGAT2 protein, or both.

E120. The method of any one of embodiments 118 or 119, wherein the subject has a disease, disorder or condition associated with DGAT2 expression.

E121. A method for treating a subject having a disease, disorder or condition associated with DGAT2 expression, the method comprising administering to the subject a therapeutically effective amount of the oligonucleotide of any one of embodiments 46 to 89, or the pharmaceutical composition of embodiment 90, thereby treating the subject.

E122. A method for reducing an amount or level of liver fibrosis in a subject, the method comprising administering to the subject the oligonucleotide of any one of embodiments 46 to 89, or the pharmaceutical composition of embodiment 90.

E123. A method for treating a subject having a disease, disorder or condition associated with DGAT2 expression, the method comprising administering to the subject a therapeutically effective amount of the oligonucleotide of any one of embodiments 46 to 89, or the pharmaceutical composition of embodiment 90, thereby treating the subject, wherein the therapeutically effective amount is 0.03, 0.075, 0.15, 0.3, 0.75, 1.5, 3, 6, 12, 24, 60, 120 or 600 mg/kg.

E124. A method for treating a subject having a disease, disorder or condition associated with DGAT2 expression, the method comprising administering to the subject a therapeutically effective amount of the oligonucleotide of any one of embodiments 46 to 89, or the pharmaceutical composition of embodiment 90, thereby treating the subject, wherein the therapeutically effective amount is 1.5, 3, 6 or 12 mg/kg.

E125. A method for treating a subject having a disease, disorder or condition associated with DGAT2 expression, the method comprising administering to the subject a therapeutically effective amount of an oligonucleotide comprising a (i) sense strand selected from SEQ ID NOs:105, 107, 111, 125, 129 and 137, and an (ii) antisense strand selected from SEQ ID NOs: 106, 108, 112, 126, 130 and 138 or pharmaceutical composition thereof, thereby treating the subject.

E126. The method of any one of embodiments embodiment 118-125, wherein the disease, disorder or condition associated with DGAT2 expression is selected from the group consisting of metabolic liver disease, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker’s lung, tuberculosis, pulmonary fibrosis, idiopathic pulmonary fibrosis (IPF).

E127. The method of embodiment 126, wherein the disease, disorder or condition associated with DGAT2 expression is metabolic liver disease, non-alcoholic fatty liver disease (NAFLD) or non-alcoholic steatohepatitis (NASH).

E128. The method of any one of embodiments 120-127, wherein the disease, disorder or condition associated with DGAT2 expression is or non-alcoholic steatohepatitis (NASH).

E129. The method of any one of embodiments 120-128 wherein the oligonucleotide, or pharmaceutical composition, is administered in combination with a second composition or therapeutic agent.

E130. Use of the oligonucleotide of any one of embodiments 46-89, or the pharmaceutical composition of embodiment 90, in the manufacture of a medicament for the treatment of a disease, disorder or condition associated with DGAT2 expression.

E131. The oligonucleotide of any one of embodiments 46-89, or the pharmaceutical composition of embodiment 90, for use, or adaptable for use, in the treatment of a disease, disorder or condition associated with DGAT2 expression.

E132. A kit comprising the oligonucleotide of any one of embodiments 46 to 89, an optional pharmaceutically acceptable carrier, and a package insert comprising instructions for administration to a subject having a disease, disorder or condition associated with DGAT2 expression.

E133. The use of the oligonucleotide of embodiment 131, or the kit of embodiment 132, wherein the disease, disorder or condition associated with DGAT2 expression is selected from the group consisting of hypertriglyceridemia, obesity, hyperlipidemia, abnormal lipid and/or cholesterol metabolism, atherosclerosis, type II diabetes mellitus, cardiovascular disease, coronary artery disease, non-alcoholic steatohepatitis (NASH), non-alcoholic fatty liver disease, homozygous and heterozygous familial hypercholesterolemia, and statin-resistant hypercholesterolemia.

E134. The use of the oligonucleotide or pharmaceutical composition for use of embodiment 131, or the kit of embodiment 132, wherein the disease, disorder or condition associated with DGAT2 expression is cardiovascular disease, type II diabetes mellitus, hypertriglyceridemia, NASH, obesity, or a combination thereof.

E135. A method for reducing ACC or DGAT2 expression in a cell, a population of cells or a subject, the method consisting of co-administering to said subject a first and second oligonucleotide, each oligonucleotide comprising a sense sequence of 15-30 nucleotides in length and a complementary antisense sequence of 15-30 nucleotides in length, wherein the first oligonucleotide is an inhibitor of ACC and the second oligonucleotide is an inhibitor DGAT2.

E136. A method for treating a subject having a disease, disorder or condition associated with ACC or DGAT2 expression, the method comprising co-administering to the subject a therapeutically effective amount of two oligonucleotides each comprising a sense strand of 15 to 50 nucleotides in length and an antisense strand of 15 to 30 nucleotides in length, wherein the first sense strand comprises a sequence as set forth in any one of SEQ ID Nos 1, 29, 31, 43, 55, 105, 107, 111, 125, 129 and 137 and wherein the first antisense strand comprises a complementary sequence selected from SEQ ID NOs: 2, 30, 32, 44, 56, 106, 108, 112, 126, 130 and 138 and the second sense strand comprises a sequences as set forth in any one of SEQ ID NOs: 1, 29, 31, 43, 55, 105, 107, 111, 125, 129 and 137 and wherein the second antisense strand comprises a complementary sequence selected from SEQ ID NOs: 2, 30, 32, 44, 56, 106, 108, 112, 126, 130 and 138, provided that the sense strand of the first oligonucleotide and the sense strand of the second oligonucleotide are not the same, thereby treating the subject.

E137. A method for treating a subject having a disease, disorder or condition associated with ACC or DGAT2 expression, the method comprising concurrently, intermittently or sequentially administering, in any order, to the subject a therapeutically effective amount of two oligonucleotides each comprising a sense strand of 15 to 50 nucleotides in length and an antisense strand of 15 to 30 nucleotides in length, wherein the first sense strand comprises a sequence as set forth in any one of SEQ ID Nos: 1, 29, 31, 43, 55, 105, 107, 111, 125, 129 and 137. and wherein the first antisense strand comprises a complementary sequence selected from SEQ ID NOs: 2, 30, 32, 44, 56, 106, 108, 112, 126, 130 and 138 and the second sense strand comprises a sequences as set forth in any one of SEQ ID NOs: : 1, 29, 31, 43, 55, 105, 107, 111, 125, 129 and 137 and wherein the second antisense strand comprises a complementary sequence selected from SEQ ID NOs: : 2, 30, 32, 44, 56, 106, 108, 112, 126, 130 and 138, provided that the sense strand of the first oligonucleotide and the sense strand of the second oligonucleotide are not the same, thereby treating the subject.

E138. The method of any one of embodiments 135-137, wherein reducing ACC or DGAT2 expression comprises reducing an amount or a level of ACC or DGAT2 mRNA, an amount or a level of ACC or DGAT2 protein, or any combination thereof.

E139. A method for reducing an amount or level of Liver Fibrosis in a subject, the method comprising administering to the subject the oligonucleotides of any of embodiments 135-137.

E140. The method of embodiment 135 wherein the subject has a disease, disorder or condition associated with ACC or DGAT2 expression.

E141. A method for treating a subject having a disease, disorder or condition associated with ACC or DGAT2 expression, the method comprising administering to the subject a therapeutically effective amount of the oligonucleotides of any one of embodiments 135-137.

E142. A method for treating a subject having a disease, disorder or condition associated with ACC or DGAT2 expression, the method comprising administering to the subject a therapeutically effective amount of the oligonucleotides of any one of the preceding embodiments, thereby treating the subject, wherein the therapeutically effective amount is 0.03, 0.075, 0.15, 0.3, 0.75, 1.5, 3, 6, 12, 24, 60, 120 or 600 mg/kg of each oligonucleotide.

E143. A method for treating a subject having a disease, disorder or condition associated with ACC or DGAT2 expression, the method comprising administering to the subject a therapeutically effective amount of the oligonucleotides of any one of the preceding embodiments thereby treating the subject, wherein the therapeutically effective amount is 1.5, 3, 6 or 12 mg/kg of each oligonucleotide.

E144. The method of any one of embodiments 140-143, wherein the disease, disorder or condition associated with ACC or DGAT2 expression is selected from the group consisting of metabolic liver disease, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies such as Primary Sclerosing Cholangitis (PSC), Primary Biliary Cholangitis (PBC), biliary atresia, progressive familial intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation- or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, such as COPD, asthma, emphysema, smoker’s lung, tuberculosis, pulmonary fibrosis, idiopathic pulmonary fibrosis (IPF).

E145. The method of embodiment 144, wherein the disease, disorder or condition associated with ACC or DGAT2 expression is cardiovascular disease, type II diabetes mellitus, hypertriglyceridemia, NASH, obesity, or a combination thereof.

E146. Use of the combination product of any one of embodiments 95-99 in the manufacture of a medicament for the treatment of a disease, disorder or condition associated with ACC or DGAT2 expression.

E147. The combination product of any one of embodiments 95-99, for use, or adaptable for use, in the treatment of a disease, disorder or condition associated with ACC or DGAT2 expression.

E148. A kit comprising the combination product of any one of embodiments 95-99, an optional pharmaceutically acceptable carrier, and a package insert comprising instructions for administration to a subject having a disease, disorder or condition associated with ACC or DGAT2 expression.

E149. A method of treating Metabolic Syndrome comprising administering to a patient in need thereof a therapeutically effective amount of a double-stranded RNA (dsRNA) inhibitor of the ACC gene and a dsRNA inhibitor of the DGAT2 gene where such dsRNA molecules suppress or inhibit the expression and/or function of the ACC and DGAT2 genes.

E150. The method of embodiment 149, comprising the simultaneous administration of the dsRNA inhibitor of the ACC gene and the dsRNA inhibitor of the DGAT2 gene.

### EXAMPLES

While the disclosure has been described with reference to the specific embodiments set forth in the following Examples, it should be understood by those skilled in the art that various changes may be made, and equivalents may be substituted without departing from the true spirit and scope of the disclosure. Further, the following Examples are offered by way of illustration and are not intended to limit the scope of the disclosure in any manner. In addition, modifications may be made to adapt to a situation, material, composition of matter, process, process step or steps, to the objective, spirit and scope of the disclosure. All such modifications are intended to be within the scope of the disclosure. Standard techniques well known in the art or the techniques specifically described below were utilized.

### Example 1: Preparation of Double-Stranded RNAi Oligonucleotides

Oligonucleotide Synthesis and PurificationThe double-stranded RNAi (dsRNA) oligonucleotides described in the foregoing Examples are chemically synthesized using methods described herein. Generally, dsRNAi oligonucleotides are synthesized using solid phase oligonucleotide synthesis methods as described for 19-23mer siRNAs (see, e.g., Scaringe et al. (1990) NUCLEIC ACIDS REs. 18:5433-41 and Usman et al. (1987) J. AM. CHEM. SOC. 109:7845-46; see also, U.S. Pat. Nos. 5,804,683; 5,831,071; 5,998,203; 6,008,400; 6,111,086; 6,117,657; 6,353,098; 6,362,323; 6,437,117 and 6,469,158).

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 µm inner diameter and contain ssDNA 100R Gel (Beckman-Coulter). Typically, about 0.6 nmole of oligonucleotide was injected into a capillary, run in an electric field of 444 V/cm and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer’s recommended protocol. Relative molecular masses of all oligomers were obtained, often within 0.2% of expected molecular mass.

### Preparation of Duplexes

Single strand RNA oligomers were resuspended (e.g., at 100 µM concentration) in duplex buffer consisting of 100 mM potassium acetate, 30 mM HEPES, pH 7.5. Complementary sense and antisense strands were mixed in equal molar amounts to yield a final solution of, for example, 50 µM duplex. Samples were heated to 100° C. for 5′ in RNA buffer (IDT) and were allowed to cool to room temperature before use. The dsRNA oligonucleotides were stored at -20° C. Single strand RNA oligomers were stored lyophilized or in nuclease-free water at -80° C.

### Example 2: RNAi Oligonucleotide Inhibition of ACAC Expression In Vivo

### ACACA and ACACB Dual Targeting Tool Compound Sequence Identification

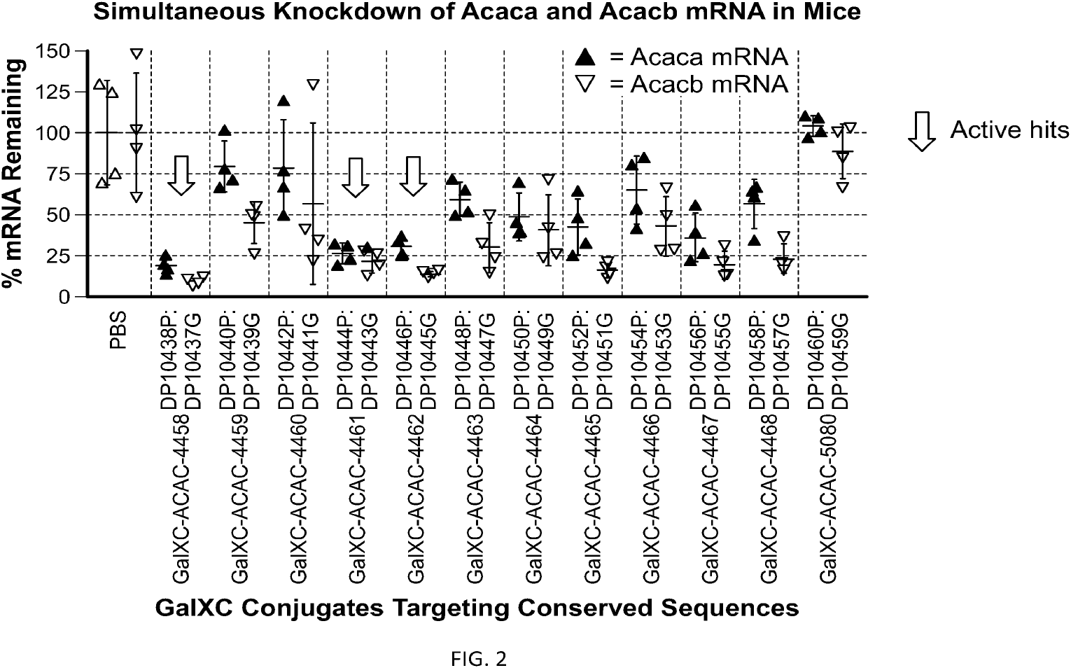

A sequence screen was performed in vivo in mice to identify tool compounds that are active against both ACACA and ACACB. To identify potent sequences, a computer-based algorithm was used to computationally generate ACACA and ACACB target sequences suitable for assaying inhibition of total ACAC expression by the RNAi pathway. The algorithm provides RNAi oligonucleotide guide strand sequences that are complementary to mouse, or all three species (mouse, cynomolgus monkey, human; Table 2). The nucleotide sequences of the 12 selected dsRNAs (Table 1) were used to generate the corresponding double-stranded RNAi oligonucleotides comprising a nicked tetraloop GalNAc-conjugated structure (referred to herein as “GalNAc-conjugated ACAC oligonucleotides”) having a 36-mer passenger strand and a 22-mer guide strand (antisense strand). The 36-mer strands include a sequence that forms a stem loop (SEQ ID NO: 159). Further, the nucleotide sequences comprising the passenger strand and guide strand of the GalNAc-conjugated ACAC oligonucleotides have a distinct pattern of modified nucleotides and phosphorothioate linkages (see FIG. 1A for a schematic of the structure and chemical modification patterns of the GalNAc-conjugated ACAC oligonucleotides used for this screen). The three adenosine nucleotides comprising the tetraloop were each conjugated to a GalNAc moiety (CAS#: 14131-60-3). The screen provided three active hits against ACACA and ACACB mRNA (FIG. 2). GalXC-ACAC-5083 (also referred to as GalXC-ACAC-4458 based on the mouse sequence) is the only sequence that targets ACACA and ACACB in all three species with 100% identity to all six transcripts.

TABLE 1

GalXC Compound Candidates for Identifying Tool Compounds for Studies in Mice.

| Oligonucleotide | DP# | Sequence (sense strand) | SEQ ID NO | Sequence (antisense strand) | SEQ ID NO |
|---|---|---|---|---|---|
| GalXC-ACAC-4458 | DP10438P: DP10437G | AACCACAUCU UCCUCAACUA | 1 | UAGUUGAGGAA GAUGUGGUUGG | 2 |
| GalXC-ACAC-4459 | DP10440P: DP10439G | ACCACAUCUU CCUCAACUUA | 3 | UAAGUUGAGGA AGAUGUGGUGG | 4 |
| GalXC-ACAC-4460 | DP10442P: DP10441G | CCACAUCUUC CUCAACUUUA | 5 | UAAAGUUGAGG AAGAUGUGGGG | 6 |
| GalXC-ACAC-4461 | DP10444P: DP10443G | CACAUCUUCC UCAACUUUGA | 7 | UCAAAGUUGAG GAAGAUGUGGG | 8 |
| GalXC-ACAC-4462 | DP10446P: DP10445G | ACAUCUUCCU CAACUUUGUA | 9 | UACAAAGUUGA GGAAGAUGUGG | 10 |
| GalXC-ACAC-4463 | DP10448P: DP10447G | CAUCUUCCUC AACUUUGUGA | 11 | UCACAAAGUUG AGGAAGAUGGG | 12 |
| GalXC-ACAC4464 | DP10450P: DP10449G | AUCUUCCUCA ACUUUGUGCA | 13 | UGCACAAAGUU GAGGAAGAUGG | 14 |
| GalXC-ACAC-4465 | DP10452P: DP10451G | UCUUCCUCAA CUUUGUGCCA | 15 | UGGCACAAAGU UGAGGAAGAGG | 16 |
| GalXC-ACAC-4466 | DP10454P: DP10453G | CUUCCUCAAC UUUGUGCCCA | 17 | UGGGCACAAAG UUGAGGAAGGG | 18 |
| GalXC-ACAC-4467 | DP10456P: DP10455G | UUCCUCAACU UUGUGCCCAA | 19 | UUGGGCACAAA GUUGAGGAAGG | 20 |
| GalXC-ACAC-4468 | DP10458P: DP10457G | UCCUCAACUU UGUGCCCACA | 21 | UGUGGGCACAA AGUUGAGGAGG | 22 |
| GalXC-ACAC-5080 | DP10460P: DP10459G | UUGUCAUCGG CAAUGACAUA | 23 | UAUGUCAUUGC CGAUGACAAGG | 24 |

### Example 3: RNAi Oligonucleotide Inhibition of ACAC Expression and Studies In Vivo

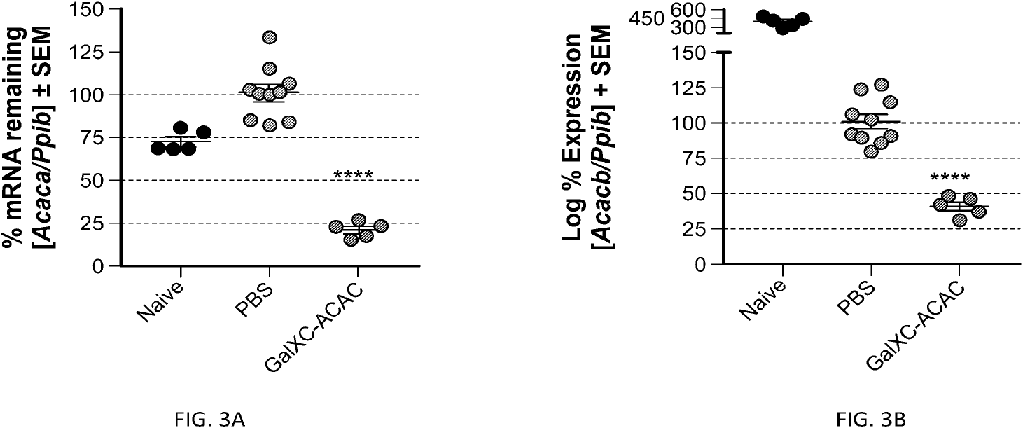

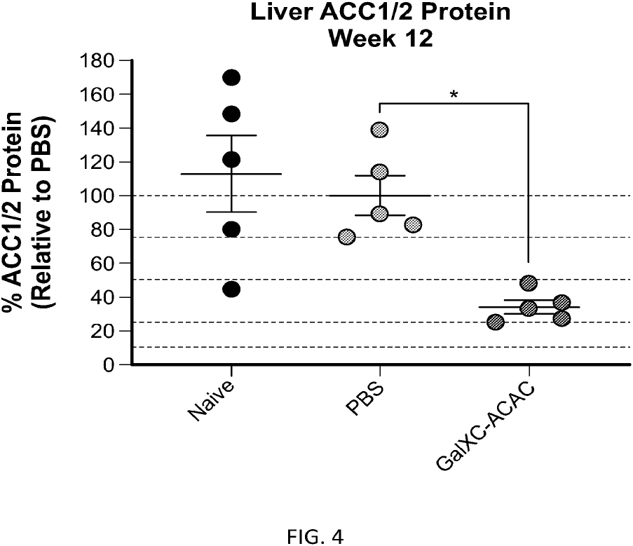

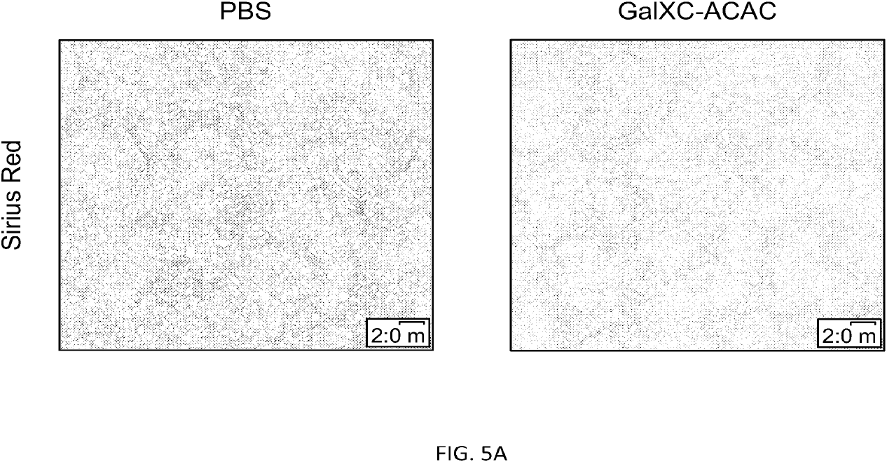

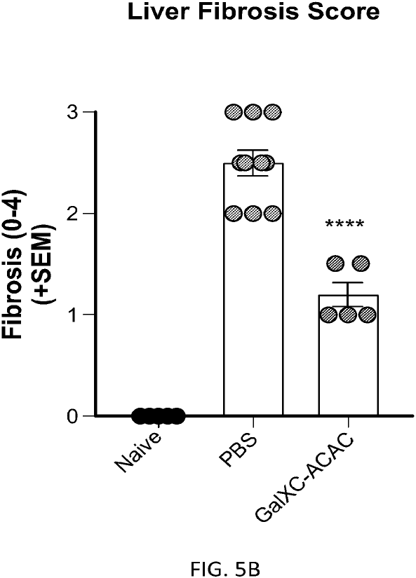

Mouse Studies: GalXC-ACAC-5083 (SEQ ID NOs: 175 and 56 with the modification pattern in FIG. 1A was used as a tool compound for evaluation of NASH phenotype in CDAHFD murine model (choline-deficient, L-amino acid-defined, high-fat diet consisting of 60 kcal% fat and 0.1% methionine by weight). Briefly, 8-week-old male C57BL/6J mice were placed on a CDAHFD for 12 weeks, with a weekly dose of 5 mg/kg GalXC-ACAC-5083 administered subcutaneously starting Week 6. At the end of 12 weeks, liver samples were collected for molecular and histopathology endpoint analyses. Total RNA isolated from mouse livers were used to assess relative ACACA and ACACB mRNA expressions by qRT-PCR. The TaqMan RT-qPCR probes from Life Technologies were used to evaluate ACACA (Mm01304275_g1) and ACACB (Mm01204688_m1). GalXC-ACAC-5083 treatment effectively reduced ACACA and ACACB mRNA of target genes by 75% (FIGS. 3A-3B). Protein expression was determined in mouse livers by Western blot analysis using ACC½ recognizing antibody (Cell Signaling 3662S). The relative protein abundances were quantified by scanning densitometry on Western blots, and is shown in FIG. 4. Immunohistochemistry was performed to visualize protein expression patterns in liver histological sections that were immuno-stained with ACC antibody (Cell signaling, 3662S). ACC½ expression in hepatocytes was significantly decreased by GalXC-ACAC.

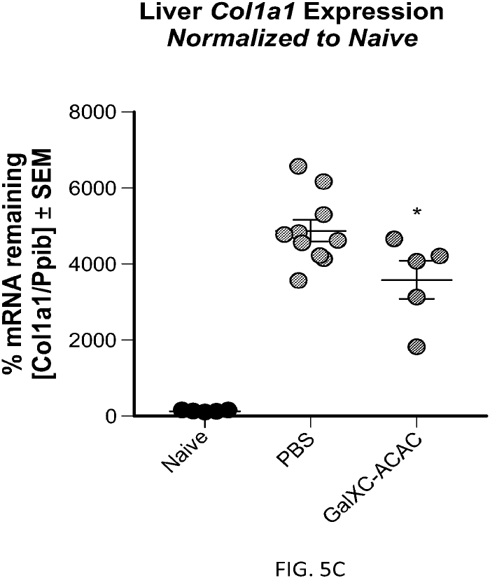

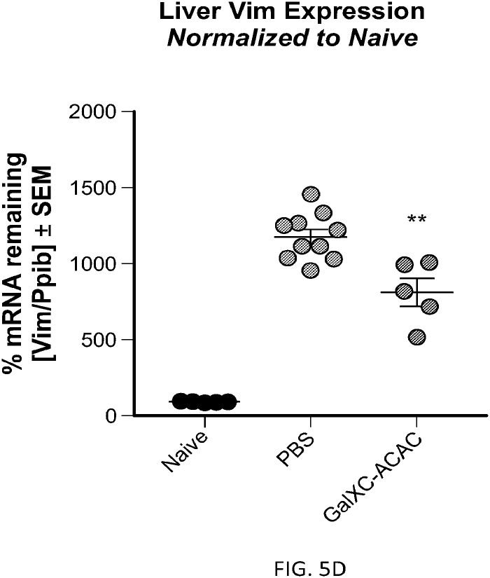

Finally, GalXC-ACAC treated or control liver histological sections were stained by Picro-Sirius Red to visualize hepatic collagen content. As shown in FIG. 5A, collagen expression was reduced indicating reduced liver fibrosis as a result of GalXC-ACAC treatment. In addition, as shown in FIG. 5B, histopathology scores confirmed notable reductions in liver fibrosis in GalXC-ACAC-5083 treated group. In addition, ACAC inhibition resulted in reduced expression of hepatic fibrosis marker genes, Col1a1 and Vim, when compared to PBS treated animals (FIGS. 5C-5D).

### Human/Cynomolgus Monkey ACACA and ACACB Targeting Candidate Sequence Identification

To identify additional RNAi oligonucleotide inhibitors targeting human and cynomolgus monkey ACACA and ACACB, thirty sequences were identified based on complementarity criteria: 100% complementarity to human and monkey ACACA and up to one mismatch in sequence allowed against human ACACB. (Table 2, SEQ ID NO: 150 human ACACA NM_198834, SEQ ID NO: 151 human ACACB NM_001093, SEQ ID NO: 152 Cynomolgus monkey ACACA XM_015438408, SEQ ID NO: 153 Cynomolgus monkey ACACB XM_015430785). Sequence analysis shows that some of the guide strand sequences were also complementary to the corresponding target sequences in mouse (SEQ ID NO: 154 mouse ACACA NM_133360, and SEQ ID NO: 155 mouse ACACB NM_133904; Table 2). A benchmark control which was identified in the screen described herein, GalXC-ACAC-5083, is the only sequence that targets ACACA and ACACB in all three species.

TABLE 2

Target Sequences of Human, Monkey and Mouse ACAC mRNA

| Species | Gene Isoform | Ref | SEQ ID NO |
|---|---|---|---|
| Human (Hs) | ACACA | NM_198834 | 150 |
| Human (Hs) | ACACB | NM_001093 | 151 |
| Cynomolgus monkey (Mf) | ACACA | XM_015438408 | 152 |
| Cynomolgus monkey (Mf) | ACACB | XM_015430785 | 153 |
| Mouse (Mm) | ACACA | NM_133360 | 154 |
| Mouse (Mm) | ACACB | NM_133904 | 155 |

The nucleotide sequences of 30 dsiRNAs hits (Table 3) were selected for evaluation in vivo. Briefly, the nucleotide sequences were used to generate 30 corresponding double-stranded RNAi oligonucleotides comprising a nicked tetraloop GalNAc-conjugated structure (referred to herein as “GalNAc-conjugated ACAC oligonucleotides”) having a 36-mer passenger strand and a 22-mer guide strand. Table 3 shows 20-mer passenger strands lacking the nicked tetraloop sequence, whereas Table 4 shows the 36-mer passenger strands including the nicked tetraloop sequence. Further, the nucleotide sequences comprising the passenger strand and guide strand of the GalNAc-conjugated ACAC oligonucleotides have a distinct pattern of modified nucleotides and phosphorothioate linkages (see FIG. 1B for a schematic of the structure and chemical modification patterns of the GalNAc-conjugated ACAC oligonucleotides). The three adenosine nucleotides comprising the tetraloop were each conjugated to a GalNAc moiety (CAS#: 14131-60-3).

TABLE 3

GalNAc-Conjugated Human/Cynomolgus monkey ACACA and ACACB Targeting candidate Sequences

| Oligonucleotide | DP# | Sequence (20 mer sense strand) | SEQ ID NO | Sequence (antisense strand) | SEQ ID NO |
|---|---|---|---|---|---|
| GalXC-ACAC-1919 | DP13931P: DP13930G | GUUAUGUGAG UGCUGGGACA | 25 | UGUCCCAGCACUCACA UAACGG | 26 |
| GalXC-ACAC-2384 | DP13929P: DP13928G | UUUCAAACAU GGUGGUGGCA | 27 | UGCCACCACCAUGUUU GAAAGG | 28 |
| GalXC-ACAC-2389 | DP13937P: DP13936G | AACAUGGUGG UGGCUUUGAA | 29 | UUCAAAGCCACCACCA UGUUGG | 30 |
| GalXC-ACAC-2392 | DP13943P: DP13942G | AUGGUGGUGG CUUUGAAGGA | 31 | UCCUUCAAAGCCACCA CCAUGG | 32 |
| GalXC-ACAC-2896 | DP13891P: DP13890G | ACCUGUGUGU UUGAGAAGGA | 33 | UCCUUCUCAAACACAC AGGUGG | 34 |
| GalXC-ACAC-2897 | DP13889P: DP13888G | CCUGUGUGUU UGAGAAGGAA | 35 | UUCCUUCUCAAACACA CAGGGG | 36 |
| GalXC-ACAC-3442 | DP13903P: DP13902G | CAGUAUGCUA GCAACAUCAA | 37 | UUGAUGUUGCUAGCAU ACUGGG | 38 |
| GalXC-ACAC-3443 | DP13911P: DP13910G | AGUAUGCUAG CAACAUCACA | 39 | UGUGAUGUUGCUAGCA UACUGG | 40 |
| GalXC-ACAC-4201 | DP13907P: DP13906G | GUGGUGGAAU UCCAGUUCAA | 41 | UUGAACUGGAAUUCCA CCACGG | 42 |
| GalXC-ACAC-4204 | DP13925P: DP13924G | GUGGAAUUCC AGUUCAUGCA | 43 | UGCAUGAACUGGAAUU CCACGG | 44 |
| GalXC-ACAC-4205 | DP13899P: DP13898G | UGGAAUUCCA GUUCAUGCUA | 45 | UAGCAUGAACUGGAAU UCCAGG | 46 |
| GalXC-ACAC-4206 | DP13901P: DP13900G | GGAAUUCCAG UUCAUGCUGA | 47 | UCAGCAUGAACUGGAA UUCCGG | 48 |
| GalXC-ACAC-4207 | DP13913P: DP13912G | GAAUUCCAGU UCAUGCUGCA | 49 | UGCAGCAUGAACUGGA AUUCGG | 50 |
| GalXC-ACAC-4208 | DP13921P: DP13920G | AAUUCCAGUU CAUGCUGCCA | 51 | UGGCAGCAUGAACUGG AAUUGG | 52 |
| GalXC-ACAC-5082 | DP13923P: DP13922G | UAACCACAUC UUCCUCAACA | 53 | UGUUGAGGAAGAUGU GGUUAGG | 54 |
| GalXC-ACAC-5083 | DP13917P: DP13916G | AACCACAUCU UCCUCAACUA | 55 | UAGUUGAGGAAGAUG UGGUUGG | 56 |
| GalXC-ACAC-5084 | DP13935P: DP13934G | ACCACAUCUU CCUCAACUUA | 57 | UAAGUUGAGGAAGAU GUGGUGG | 58 |
| GalXC-ACAC-5305 | DP13905P: DP13904G | UACAAGGAAG UGACUGACUA | 59 | UAGUCAGUCACUUCCU UGUAGG | 60 |
| GalXC-ACAC-5307 | DP13927P: DP13926G | CAAGGAAGUG ACUGACUCCA | 61 | UGGAGUCAGUCACUUC CUUGGG | 62 |
| GalXC-ACAC-5309 | DP13897P: DP13896G | AGGAAGUGAC UGACUCCAGA | 63 | UCUGGAGUCAGUCACU UCCUGG | 64 |
| GalXC-ACAC-6331 | DP13919P: DP13918G | GAUGACUUUG AAGGGGUUUA | 65 | UAAACCCCUUCAAAGU CAUCGG | 66 |
| GalXC-ACAC-6683 | DP13893P: DP13892G | UGGAUUCUGA AGCCAAGAUA | 67 | UAUCUUGGCUUCAGAA UCCAGG | 68 |
| GalXC-ACAC-6684 | DP13895P: DP13894G | GGAUUCUGAA GCCAAGAUAA | 69 | UUAUCUUGGCUUCAGA AUCCGG | 70 |
| GalXC-ACAC-6685 | DP13915P: DP13914G | GAUUCUGAAG CCAAGAUAAA | 71 | UUUAUCUUGGCUUCAG AAUCGG | 72 |
| GalXC-ACAC-6686 | DP13887P: DP13886G | AUUCUGAAGC CAAGAUAAUA | 73 | UAUUAUCUUGGCUUCA GAAUGG | 74 |
| GalXC-ACAC-6755 | DP13941P: DP13940G | AGGCCAUCAA GGACUUCAAA | 75 | UUUGAAGUCCUUGAUG GCCUGG | 76 |
| GalXC-ACAC-6759 | DP13933P: DP13932G | CAUCAAGGAC UUCAACCGGA | 77 | UCCGGUUGAAGUCCUU GAUGGG | 78 |
| GalXC-ACAC-6761 | DP13945P: DP13944G | UCAAGGACUU CAACCGGGAA | 79 | UUCCCGGUUGAAGUCC UUGAGG | 80 |
| GalXC-ACAC-6793 | DP13939P: DP13938G | AUGGUCUUUG CCAACUGGAA | 81 | UUCCAGUUGGCAAAGA CCAUGG | 82 |
| GalXC-ACAC-6845 | DP13909P: DP13908G | ACCAAGUGCU GAAGUUUGGA | 83 | UCCAAACUUCAGCACU UGGUGG | 84 |

TABLE 4

GalNAc-Conjugated Human/Cynomolgus monkey ACACA and ACACB Targeting candidate Sequences

| Oligonucleotide | DP# | Sequence (36 mer sense strand) | SEQ ID NO | Sequence (antisense strand) | SEQ ID NO | Modified 36mer Sense strand SEQ ID NO | Modified 22mer anti-sense strand SEQ ID NO |
|---|---|---|---|---|---|---|---|
| GalXC-ACAC-1919 | DP13931P: DP13930G | GUUAUG UGAGUG CUGGGA CA GCAGCC GAAAGG CUGC | 160 | UGUCCC AGCACU CACAUA ACGG | 26 | 206 | 252 |
| GalXC-ACAC-2384 | DP13929P: DP13928G | UUUCAA ACAUGG UGGUGG CA GCAGCC GAAAGG CUGC | 161 | UGCCAC CACCAU GUUUGA AAGG | 28 | 207 | 253 |
| GalXC-ACAC-2389 | DP13937P: DP13936G | AACAUG GUGGUG GCUUUG AA GCAGCC GAAAGG CUGC | 162 | UUCAAA GCCACC ACCAUG UUGG | 30 | 208 | 254 |
| GalXC-ACAC-2392 | DP13943P: DP13942G | AUGGUG GUGGCU UUGAAG GA GCAGCC GAAAGG CUGC | 163 | UCCUUC AAAGCC ACCACC AUGG | 32 | 209 | 255 |
| GalXC-ACAC-2896 | DP13891P: DP13890G | ACCUGU GUGUUU GAGAAG GA GCAGCC GAAAGG CUGC | 164 | UCCUUC UCAAAC ACACAG GUGG | 34 | 210 | 256 |
| GalXC-ACAC-2897 | DP13889P: DP13888G | CCUGUG UGUUUG AGAAGG AA GCAGCC GAAAGG CUGC | 165 | UUCCUU CUCAAA CACACA GGGG | 36 | 211 | 257 |
| GalXC-ACAC-3442 | DP13903P: DP13902G | CAGUAU GCUAGC AACAUC AA GCAGCC GAAAGG CUGC | 166 | UUGAUG UUGCUA GCAUAC UGGG | 38 | 212 | 258 |
| GalXC-ACAC-3443 | DP13911P: DP13910G | AGUAUG CUAGCA ACAUCA CA GCAGCC GAAAGG CUGC | 167 | UGUGAU GUUGCU AGCAUA CUGG | 40 | 213 | 259 |
| GalXC-ACAC-4201 | DP13907P: DP13906G | GUGGUG GAAUUC CAGUUC AA GCAGCC GAAAGG CUGC | 168 | UUGAAC UGGAAU UCCACC ACGG | 42 | 214 | 260 |
| GalXC-ACAC-4204 | DP13925P: DP13924G | GUGGAA UUCCAG UUCAUG CA GCAGCC GAAAGG CUGC | 169 | UGCAUG AACUGG AAUUCC ACGG | 44 | 215 | 261 |
| GalXC-ACAC-4205 | DP13899P: DP13898G | UGGAAU UCCAGU UCAUGC UA GCAGCC GAAAGG CUGC | 170 | UAGCAU GAACUG GAAUUC CAGG | 46 | 216 | 262 |
| GalXC-ACAC-4206 | DP13901P: DP13900G | GGAAUU CCAGUU CAUGCU GA GCAGCC GAAAGG CUGC | 171 | UCAGCA UGAACU GGAAUU CCGG | 48 | 217 | 263 |
| GalXC-ACAC-4207 | DP13913P: DP13912G | GAAUUC CAGUUC AUGCUG CA GCAGCC GAAAGG CUGC | 172 | UGCAGC AUGAAC UGGAAU UCGG | 50 | 218 | 264 |
| GalXC-ACAC-4208 | DP13921P: DP13920G | AAUUCC AGUUCA UGCUGC CA GCAGCC GAAAGG CUGC | 173 | UGGCAG CAUGAA CUGGAA UUGG | 52 | 219 | 265 |
| GalXC-ACAC-5082 | DP13923P: DP13922G | UAACCA CAUCUU CCUCAA CA GCAGCC GAAAGG CUGC | 174 | UGUUGA GGAAGA UGUGGU UAGG | 54 | 220 | 266 |
| GalXC-ACAC-5083 | DP13917P: DP13916G | AACCAC AUCUUC CUCAAC UA GCAGCC GAAAGG CUGC | 175 | UAGUUG AGGAAG AUGUGG UUGG | 56 | 221 | 267 |
| GalXC-ACAC-5084 | DP13935P: DP13934G | ACCACA UCUUCC UCAACU UA GCAGCC GAAAGG CUGC | 176 | UAAGUU GAGGAA GAUGUG GUGG | 58 | 222 | 268 |
| GalXC-ACAC-5305 | DP13905P: DP13904G | UACAAG GAAGUG ACUGAC UA GCAGCC GAAAGG CUGC | 177 | UAGUCA GUCACU UCCUUG UAGG | 60 | 223 | 269 |
| GalXC-ACAC-5307 | DP13927P: DP13926G | CAAGGA AGUGAC UGACUC CA GCAGCC GAAAGG CUGC | 178 | UGGAGU CAGUCA CUUCCU UGGG | 62 | 224 | 270 |
| GalXC-ACAC-5309 | DP13897P: DP13896G | AGGAAG UGACUG ACUCCA GA GCAGCC GAAAGG CUGC | 179 | UCUGGA GUCAGU CACUUC CUGG | 64 | 225 | 271 |
| GalXC-ACAC-6331 | DP13919P: DP13918G | GAUGAC UUUGAA GGGGUU UA GCAGCC GAAAGG CUGC | 180 | UAAACC CCUUCA AAGUCA UCGG | 66 | 226 | 272 |
| GalXC-ACAC-6683 | DP13893P: DP13892G | UGGAUU CUGAAG CCAAGA UA GCAGCC GAAAGG CUGC | 181 | UAUCUU GGCUUC AGAAUC CAGG | 68 | 227 | 273 |
| GalXC-ACAC-6684 | DP13895P: DP13894G | GGAUUC UGAAGC CAAGAU AA GCAGCC GAAAGG CUGC | 182 | UUAUCU UGGCUU CAGAAU CCGG | 70 | 228 | 274 |
| GalXC-ACAC-6685 | DP13915P: DP13914G | GAUUCU GAAGCC AAGAUA AA GCAGCC GAAAGG CUGC | 183 | UUUAUC UUGGCU UCAGAA UCGG | 72 | 229 | 275 |
| GalXC-ACAC-6686 | DP13887P: DP13886G | AUUCUG AAGCCA AGAUAA UA GCAGCC GAAAGG CUGC | 184 | UAUUAU CUUGGC UUCAGA AUGG | 74 | 230 | 276 |
| GalXC-ACAC-6755 | DP13941P: DP13940G | AGGCCA UCAAGG ACUUCA AA GCAGCC GAAAGG CUGC | 185 | UUUGAA GUCCUU GAUGGC CUGG | 76 | 231 | 277 |
| GalXC-ACAC-6759 | DP13933P: DP13932G | CAUCAA GGACUU CAACCG GA GCAGCC GAAAGG CUGC | 186 | UCCGGU UGAAGU CCUUGA UGGG | 78 | 232 | 278 |
| GalXC-ACAC-6761 | DP13945P: DP13944G | UCAAGG ACUUCA ACCGGG AA GCAGCC GAAAGG CUGC | 187 | UUCCCG GUUGAA GUCCUU GAGG | 80 | 233 | 279 |
| GalXC-ACAC-6793 | DP13939P: DP13938G | AUGGUC UUUGCC AACUGG AA GCAGCC GAAAGG CUGC | 188 | UUCCAG UUGGCA AAGACC AUGG | 82 | 234 | 280 |
| GalXC-ACAC-6845 | DP13909P: DP13908G | ACCAAG UGCUGA AGUUUG GA GCAGCC GAAAGG CUGC | 189 | UCCAAA CUUCAG CACUUG GUGG | 84 | 235 | 281 |

The GalNAc-conjugated ACAC oligonucleotides listed in Table 4 were evaluated in mice engineered to transiently express human ACACA and ACACB mRNA in in vivo mouse hepatocytes. Briefly, 6-8-week-old female CD-1 mice were treated subcutaneously with a GalNAc-conjugated ACAC oligonucleotide at a dose level of 3 mg/kg. Three days later (72 h), the mice were hydrodynamically injected with equal amounts of DNA plasmids encoding the full human ACACA and ACACB gene isoforms under control of a ubiquitous cytomegalovirus (CMV) promoter sequence. Twenty hours after introduction of the plasmid, liver samples were collected. Total RNA derived from these mice was subjected to qRT-PCR analysis for both ACACA and ACACB mRNA, relative to mice treated only with an identical volume of PBS. The TaqMan RT-qPCR probes purchased from Life Technologies to evaluate ACACA (Hs01046048_m1) and ACACB (Hs01565914_m1). The values were normalized for transfection efficiency using the NeoR gene included on the plasmid.

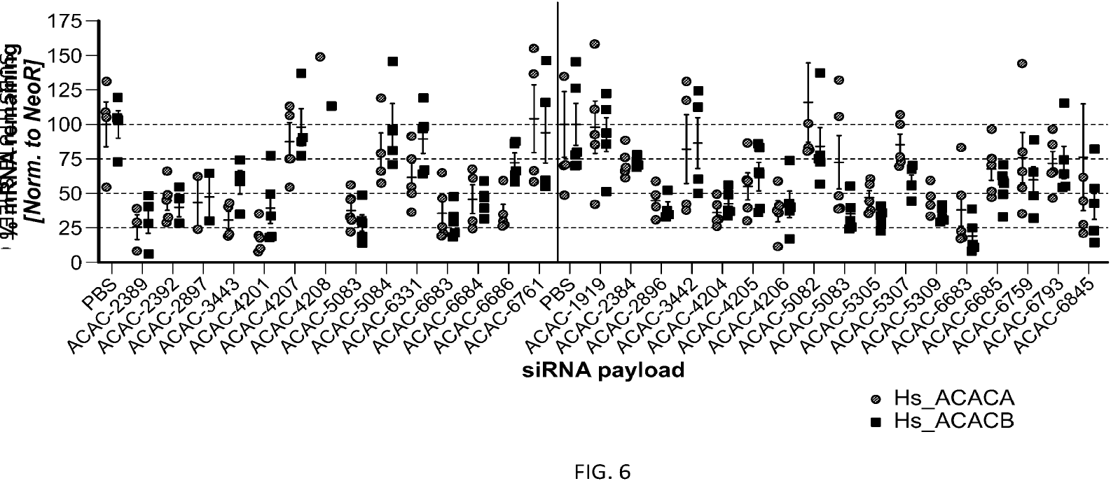

As shown in FIG. 6, twelve of the GalNAc-conjugated ACAC oligonucleotides, including the benchmark oligonucleotide GalXC-ACAC-5083 tested inhibit ACACA and ACACB expression as determined by a reduced amount of ACACA and ACACB mRNA in liver samples from oligonucleotide-treated mice relative to mice treated with PBS. FIG. 6 shows that 11 out of the 30 GalNAc-conjugated ACAC oligonucleotides tested inhibit ACACA and ACACB mRNA expression to a greater extent than the benchmark GalNAc-conjugated ACAC oligonucleotide ACAC-5083.

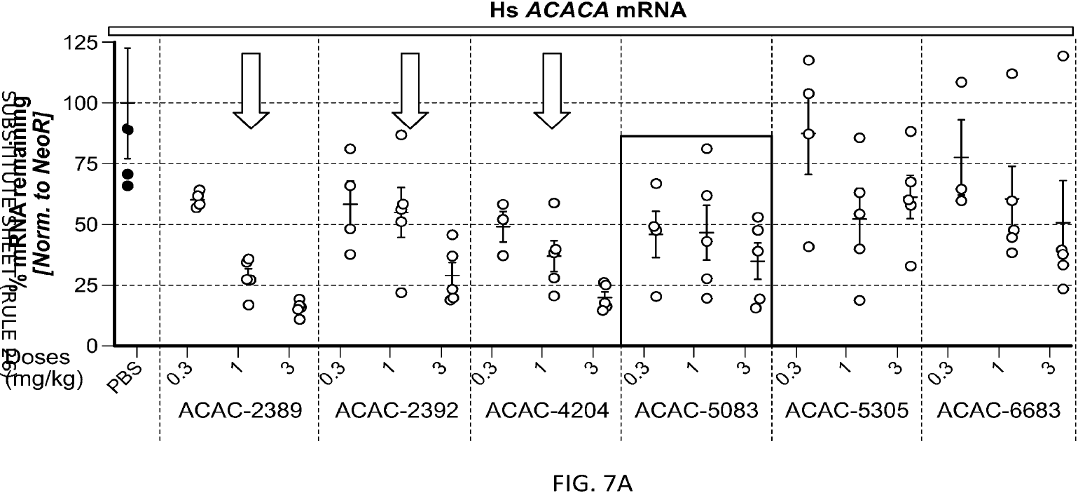

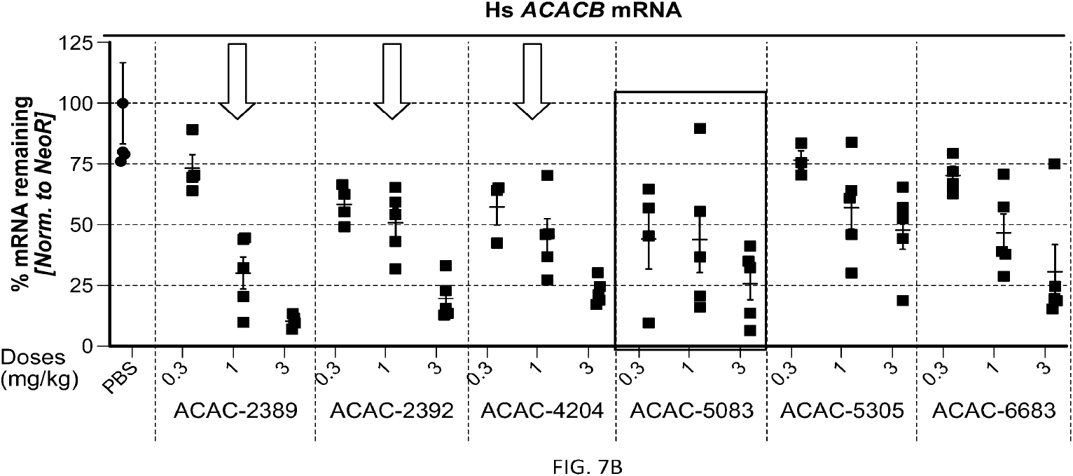

Six compounds including the benchmark that were shown to inhibit ACACA and ACACB expression at 3 mg/kg were tested in a dose response study. Briefly, 6-8-week-old female CD-1 mice were treated subcutaneously with a GalNAc-conjugated ACAC oligonucleotide at doses of 0.3, 1 and 3 mg/kg. Four (4) days later (96 h), the mice were hydrodynamically injected with equal amounts of DNA plasmids encoding the full human ACACA and ACACB gene isoforms under control of a ubiquitous cytomegalovirus (CMV) promoter sequence. Twenty (20) hours after introduction of the plasmid, liver samples were collected. Total RNA derived from these mice were subjected to qRT-PCR analysis for both ACACA and ACACB mRNA, relative to mice treated only with an identical volume of PBS. The values were normalized for transfection efficiency using the NeoR gene included on the plasmid. As shown in FIGS. 7A-7B, GalXC-ACAC-2389, GalXC-ACAC-2392 and GalXC-ACAC-4204 display ED50s of 0.3-1 mg/kg against human ACACA and ACACB.

These three compounds were selected for evaluation of their ability to inhibit ACACA/B expression in non-human primates (NHPs). The GalNAc-conjugated ACAC oligonucleotides listed in Table 5 comprise chemically modified nucleotides having pattern as described in FIG. 1C with the 2′-O-methyl modified nucleotides on the guide strand position 4 changed to 2′Fluoro.

TABLE 5

GalNAc-Conjugated ACAC Oligonucleotides Evaluated in NHPs

| Oligonucleotide | DP# | Sequence (sense strand) | SE Q ID NO | Sequence (antisense strand) | SE Q ID NO | Modified 36mer Sense strand SEQ ID NO | Modified 22mer anti-sense strand SEQ ID NO |
|---|---|---|---|---|---|---|---|
| GalXC-ACAC-2389 | DP13937 P: DP13936 G | AACAUG GUGGUG GCUUUG AA GCAGCC GAAAGG CUGC | 162 | UUCAAA GCCACC ACCAUG UUGG | 30 | 208 | 254 |
| GalXC-ACAC-2392 | DP13943 P: DP13942 G | AUGGUG GUGGCU UUGAAG GA GCAGCC | 163 | UCCUUC AAAGCC ACCACC AUGG | 32 | 209 | 255 |
| | | GAAAGG CUGC | | | | | |
| Gal-XC-ACAC-4204 | DP13925 P: DP13924 G | GUGGAA UUCCAG UUCAUG CA GCAGCC GAAAGG CUGC | 169 | UGCAUG AACUGG AAUUCC ACGG | 44 | 215 | 261 |

### Non-Human Primate (NHP) Studies

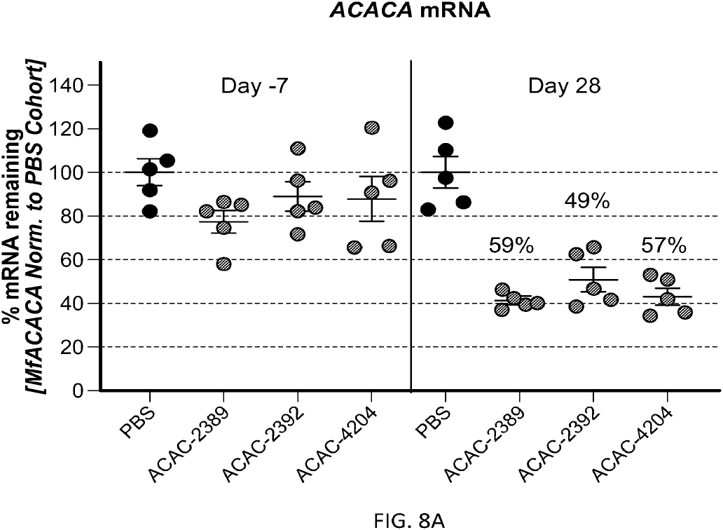

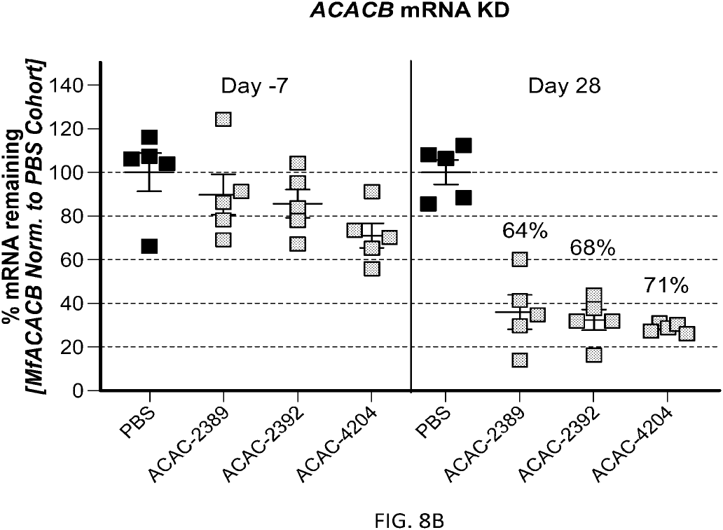

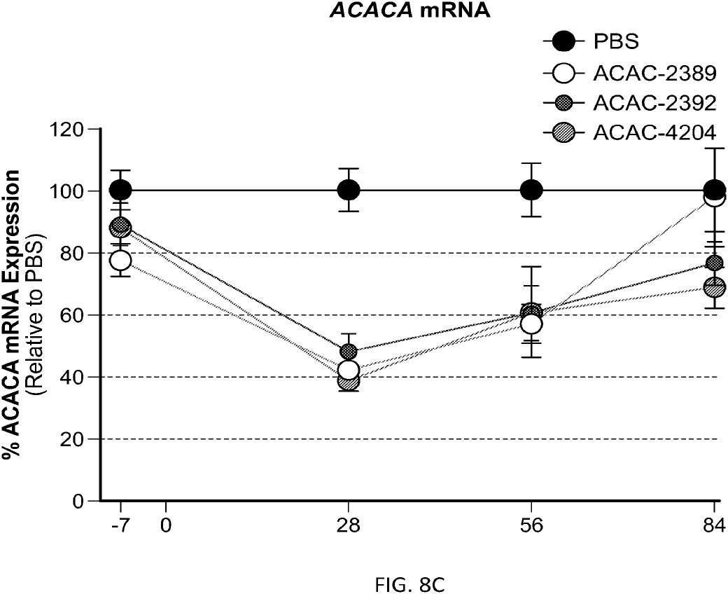

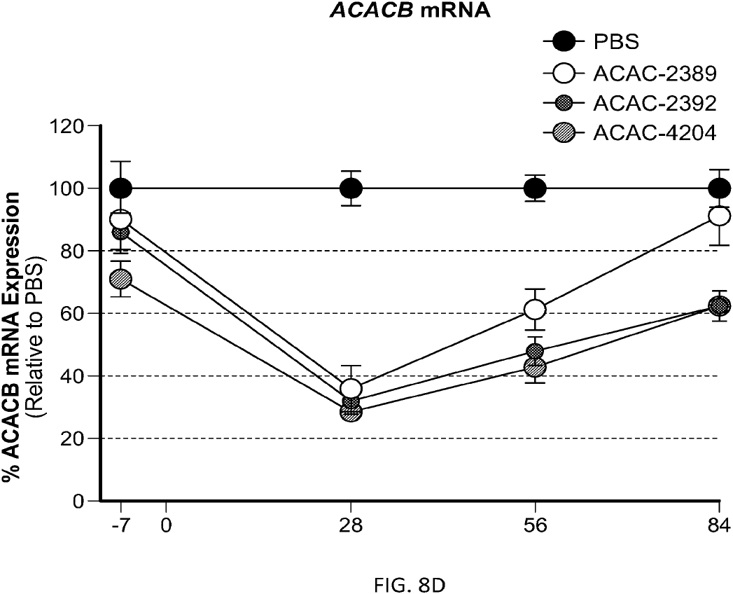

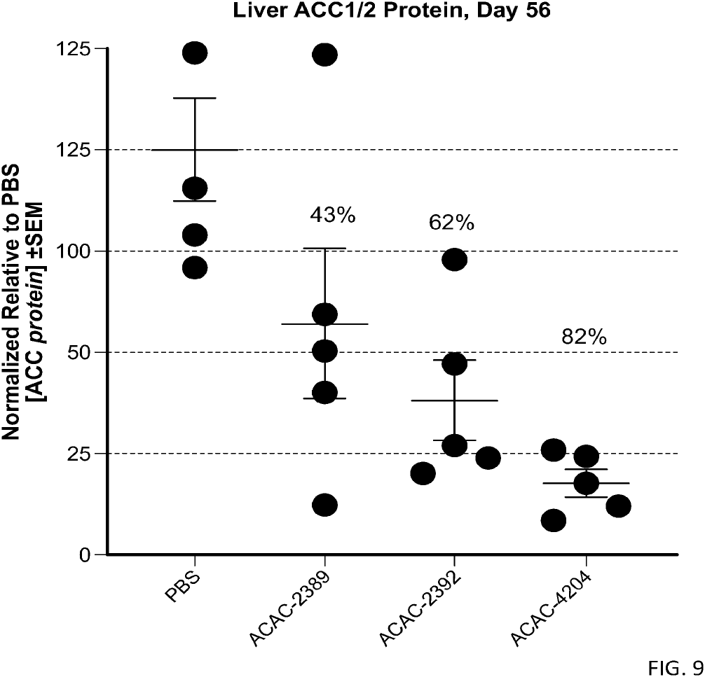

The GalNAc-conjugated ACAC oligonucleotides listed in Table 5 were evaluated in cynomolgus monkeys (Macaca fascicularis). In this study, the monkeys were grouped so that their mean body weights (about 5.4 kg) were comparable between the control and experimental groups. Each cohort contained two male and three female subjects. The GalNAc-conjugated ACAC oligonucleotides were administered subcutaneously at a dose of 6 mg/kg on Study Day 0. Blood samples were collected one week prior to dosing (Day -7), on the dosing date (Day 0) and 28, 54 and 86 after dosing. Ultrasound-guided core needle liver biopsies were collected on Study Days -7, 28, 56 and 84. At each time point, total RNA derived from the liver biopsy samples was subjected to qRT-PCR analysis to measure ACACA and ACACB mRNA in oligonucleotide-treated monkeys relative to those treated with a comparable volume of PBS. To normalize the data, the measurements were made relative to the geometric mean of two reference genes, PPIB and 18S rRNA. The following TaqMan qPCR probes purchased from Life Technologies, Inc, were used to evaluate gene expressions: monkey ACACA Mf01051583_m1, monkey ACACB Mf01565923_m1, PPIB Mf02802985_m1 and r18S Hs99999901_s1. As shown in FIG. 8A and FIG. 8B (Day 28), treating NHPs with the GalNAc-conjugated ACAC oligonucleotides listed in Table 5 inhibited ACACA and ACACB expression in the liver, as determined by a reduced amount of ACACA and ACACB mRNA in liver samples from oligonucleotide-treated NHPs relative to NHPs treated with PBS. The mean percent reduction of ACACA and ACACB mRNA in the liver samples of treated NHPs is indicated above the set of data points for each treatment group and a plot of the mean values over times is shown in FIG. 8C and FIG. 8D. For all time points evaluated, GalXC-ACAC significantly inhibits ACACA and ACACB mRNA expression. In the same samples, ACC½ protein levels were detected by western blot using ACC monoclonal antibody (Cell Signaling, #3676S). As shown in FIG. 9, at the 56-day timepoint, GalXC-ACAC oligonucleotides inhibit ACC½ protein expression, as normalized to the vinculin control. These results demonstrate that treating NHPs with the GalNAc-conjugated ACAC oligonucleotides reduces the amount of ACACA and ACACB mRNA in the liver and concomitantly reduces the amount of ACC ½ protein in the liver.

Taken together, these results show that GalNAc-conjugated ACAC oligonucleotides designed to target human total ACAC mRNA inhibit total ACAC expression in vivo (as determined by the reduction of the amount of ACACA and ACACB mRNA and ACC½ protein).

### Example 4: RNAi Oligonucleotide Inhibition of DGAT2 Expression In Vivo

### DGAT2 Targeting Tool Compound Sequence Identification

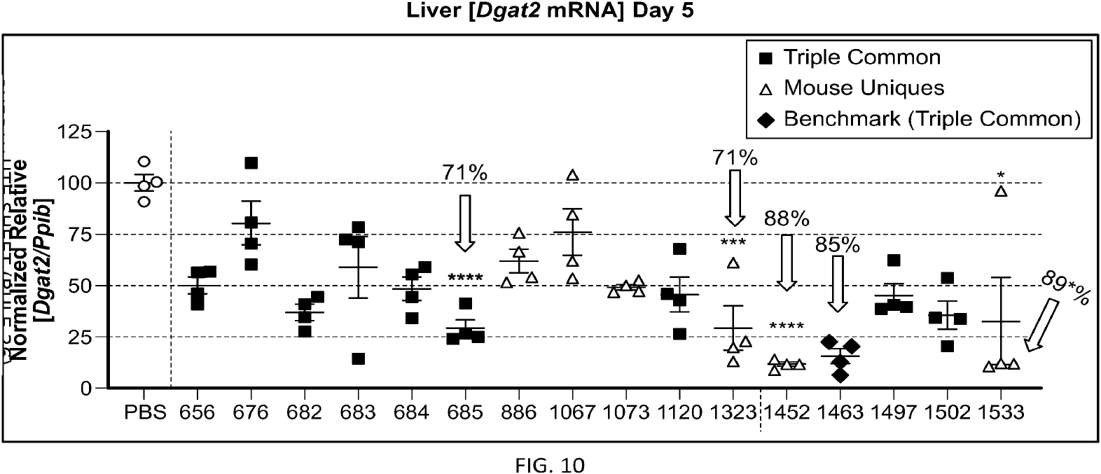

A sequence screen was performed in vivo in mice to identify tool compounds that are active against DGAT2. To identify potent sequences, a computer-based algorithm was used to computationally generate DGAT2 target sequences suitable for assaying inhibition of DGAT2 expression by the RNAi pathway. The algorithm provides RNAi oligonucleotide guide strand sequences that are complementary to mouse, or all three species (mouse, monkey, human). The nucleotide sequences of the 16 selected dsiRNAs were used to generate the corresponding double-stranded RNAi oligonucleotides comprising a nicked tetraloop GalNAc-conjugated structure (referred to herein as “GalNAc-conjugated ACAC oligonucleotides”) having a 36-mer passenger strand and a 22-mer guide strand (Table 6). Further, the nucleotide sequences comprising the passenger strand and guide strand of the GalNAc-conjugated ACAC oligonucleotides have a distinct pattern of modified nucleotides and phosphorothioate linkages (see e.g., FIG. 1C for a schematic of the generic structure and chemical modification patterns of the GalNAc-conjugated DGAT2 oligonucleotides used for this screen). The three adenosine nucleotides comprising the tetraloop were each conjugated to a GalNAc moiety (CAS#: 14131-60-3). The screen provided five active hits against DGAT2 mRNA (FIG. 10).

TABLE 6

GalXC Compound Candidates for Identifying Tool Compounds for Studies in Mice

| Oligonucleotide | DP# | Sequence (sense strand) | SEQ ID NO | Sequence (antisense strand) | SEQ ID NO |
|---|---|---|---|---|---|
| GalXC-DGAT2-1479 | DP18623P:DP1 8622G | UGUGGGUUA UUUAAAAGA AA | 85 | UUUCUUUU AAAUAACC CACAGG | 86 |
| GalXC-DGAT2-1502 | DP18625P:DP1 8624G | GUUAUUUAA AAGAAAUUA UA | 87 | UAUAAUUU CUUUUAAA UAACGG | 88 |
| GalXC-DGAT2-0682 | DP18627P:DP1 8626G | ACCAGGAAC UAUAUCUUU GA | 89 | UCAAAGAU AUAGUUCC UGGUGG | 90 |
| GalXC-DGAT2-0685 | DP18629P:DP1 8628G | AGGAACUAU AUCUUUGGA UA | 91 | UAUCCAAA GAUAUAGU UCCUGG | 92 |
| GalXC-DGAT2-0656 | DP18631P:DP1 8630G | UGGUGAAGA CACACAACCU A | 93 | UAGGUUGU GUGUCUUC ACCAGG | 94 |
| GalXC-DGAT2-0684 | DP18633P:DP1 8632G | CAGGAACUA UAUCUUUGG AA | 95 | UUCCAAAG AUAUAGUU CCUGGG | 96 |
| GalXC-DGAT2-0683 | DP18635P:DP1 8634G | CCAGGAACU AUAUCUUUG GA | 97 | UCCAAAGA UAUAGUUC CUGGGG | 98 |
| GalXC-DGAT2-0676 | DP18637P:DP1 8636G | CUGACCACCA GGAACUAUA A | 99 | UUAUAGUU CCUGGUGG UCAGGG | 100 |
| GalXC-DGAT2-1120 | DP18639P:DP1 8638G | GUCCAGAAG AAGUUCCAG AA | 101 | UUCUGGAA CUUCUUCU GGACGG | 102 |
| GalXC-DGAT2-1067 | DP18641P:DP1 8640G | AGAAUGAGG UAUACAAGC AA | 103 | UUGCUUGU AUACCUCA UUCUGG | 104 |
| GalXC-DGAT2-1452 | DP18643P:DP1 8642G | CUUGAGUGC AUUUUGUUC UA | 105 | UAGAACAA AAUGCACU CAAGGG | 106 |
| GalXC-DGAT2-1533 | DP18645P:DP1 8644G | CAUUGCAAU GUUAGAUGU UA | 107 | UAACAUCU AACAUUGC AAUGGG | 108 |
| GalXC-DGAT2-1073 | DP18647P:DP1 8646G | AGGUAUACA AGCAGGUGA UA | 109 | UAUCACCU GCUUGUAU ACCUGG | 110 |
| GalXC-DGAT2-1323 | DP18649P:DP1 8648G | GGUGAAGCU CUUUGACAA UA | 111 | UAUUGUCA AAGAGCUU CACCGG | 112 |
| GalXC-DGAT2-0886 | DP18651P:DP1 8650G | AUAGACUAC UUGCUCUCCA A | 113 | UUGGAGAG CAAGUAGU CUAUGG | 114 |
| GalXC-DGAT2-1463 | DP18667P:DP1 8666G | CUGUAAAUU UGGAAGCGU CA | 115 | UGACGCUU CCAAAUUU ACAGGG | 116 |

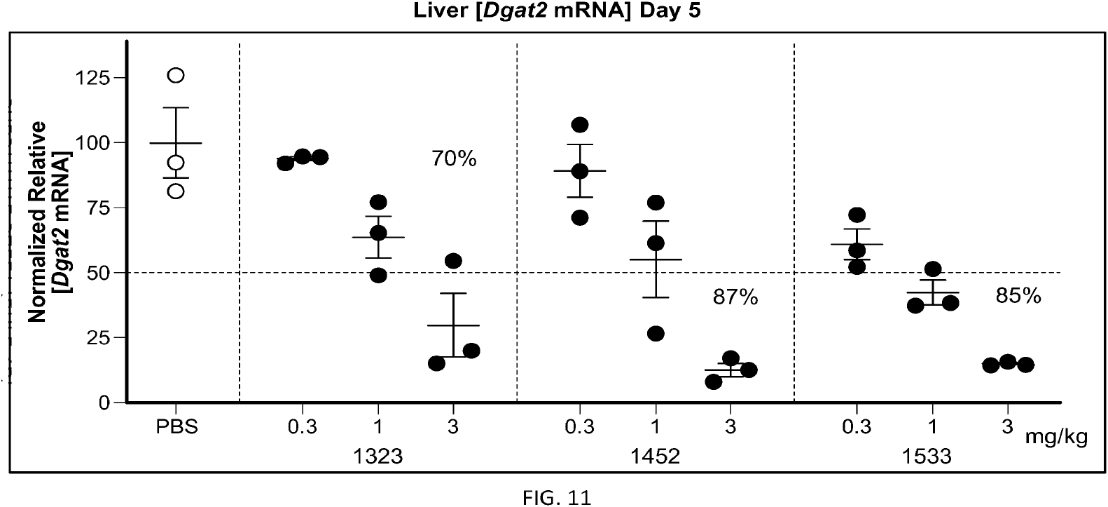

Three compounds that were shown to inhibit DGAT2 expression at 3 mg/kg were tested in a dose response study. Briefly, 6-8-week-old female CD-1 mice were treated subcutaneously with a GalNAc-conjugated DGAT2 oligonucleotide at doses of 0.3, 1 and 3 mg/kg. The mice were euthanized 4 days after dosing. Total RNA derived from these mice were subjected to qRT-PCR analysis for DGAT2 mRNA (Taqman Probe: Mm00499536_m1, Cat. No 4331182), relative to mice treated only with an identical volume of PBS. The values were normalized with Ppib internal control (Taqman Probe: Mm00478295_m1, Cat. No. 4331182). As shown in FIG. 11, GalXC-DGAT2-1452 and GalXC-DGAT2-1533 display ED50s of < 1 mg/kg against mouse DGAT2.

### Example 5: RNAi Oligonucleotide Inhibition of DGAT2 Expression In Vivo

### Human/Cynomolgus Monkey DGAT2 Targeting Candidate Sequence Identification

To identify additional RNAi oligonucleotide inhibitors targeting human and cyno DGAT2, sixteen sequences were identified based on complementarity criteria: 100% complementarity to human and monkey DGAT2. (Table 7, human DGAT2 NM _032564.5 and NM_001253891.1, Cynomolgus DGAT2 XM_005579118.2). Sequence analysis shows that some of the guide strand sequences were also complementary to the corresponding target sequences in mouse (mouse DGAT2 NM_026384.3; Table 7).

TABLE 7

Target Sequences of Human, Monkey and Mouse DGAT2 mRNA

| Species | Gene | GenBank Ref Seq # | SEQ ID NO |
|---|---|---|---|
| Human (Hs) | DGAT2 (variant 1) | NM_032564.5 | 156 |
| Human (Hs) | DGAT2 (variant 2) | NM_001253891.1 | 157 |
| Cynomolgus monkey (Mf) | DGAT2 | XM_005579118.2 | 158 |
| Mouse (Mm) | DGAT2 | NM_026384.3 | 149 |

The nucleotide sequences of sixteen DsiRNAs hits (Table 8) were selected for evaluation in vivo. Briefly, the nucleotide sequences were used to generate 16 corresponding double-stranded RNAi oligonucleotides comprising a nicked tetraloop GalNAc-conjugated structure (referred to herein as “GalNAc-conjugated DGAT2 oligonucleotides”) having a 36-mer passenger strand and a 22-mer guide strand. Table 8 shows 20-mer passenger strands lacking the nicked tetraloop sequence, whereas Table 9 shows the 36-mer passenger strands including the nicked tetraloop sequence. Further, the nucleotide sequences comprising the passenger strand and guide strand of the GalNAc-conjugated DGAT2 oligonucleotides have a distinct pattern of modified nucleotides and phosphorothioate linkages (e.g., see FIG. 1C for a schematic of the generic structure and chemical modification patterns of the GalNAc-conjugated DGAT2 oligonucleotides). The three adenosine nucleotides comprising the tetraloop were each conjugated to a GalNAc moiety (CAS#: 14131-60-3).

TABLE 8

GalNAc-Conjugated Human/Cyno DGAT2 Targeting candidate Sequences

| Oligonucleotide | DP# | Sequence (20 mer sense strand) | SEQ ID NO | Sequence (antisense strand) | SEQ ID NO |
|---|---|---|---|---|---|
| GalXC-DGAT2-1139 | DP19483P :DP19482 G | AAGAAGUUC CAGAAAUAC AA | 117 | UUGUAUUUCU GGAACUUCUU GG | 118 |
| GalXC-DGAT2-0899 | DP19481P :DP19480 G | AUAGACUAU UUGCUUUCA AA | 119 | UUUGAAAGCA AAUAGUCUAU GG | 120 |
| GalXC-DGAT2-1080 | DP19485P :DP19484 G | AGAAUGAAG UGUACAAGC AA | 121 | UUGCUUGUAC ACUUCAUUCUG G | 122 |
| GalXC-DGAT2-1086 | DP19487P :DP19486 G | AAGUGUACA AGCAGGUGA UA | 123 | UAUCACCUGCU UGUACACUUG G | 124 |
| GalXC-DGAT2-0898 | DP19489P :DP19488 G | CAUAGACUA UUUGCUUUC AA | 125 | UUGAAAGCAA AUAGUCUAUG GG | 126 |
| GalXC-DGAT2-0894 | DP19491P :DP19490 G | ACACCAUAG ACUAUUUGC UA | 127 | UAGCAAAUAG UCUAUGGUGU GG | 128 |
| GalXC-DGAT2-1074 | DP19493P :DP19492 G | UUGGAGAGA AUGAAGUGU AA | 129 | UUACACUUCAU UCUCUCCAAGG | 130 |
| GalXC-DGAT2-1083 | DP19495P :DP19494 G | AUGAAGUGU ACAAGCAGG UA | 131 | UACCUGCUUGU ACACUUCAUGG | 132 |
| GalXC-DGAT2-0895 | DP19497P :DP19496 G | CACCAUAGAC UAUUUGCUU A | 133 | UAAGCAAAUA GUCUAUGGUG GG | 134 |
| GalXC-DGAT2-0897 | DP19499P :DP19498 G | CCAUAGACU AUUUGCUUU CA | 135 | UGAAAGCAAA UAGUCUAUGG GG | 136 |
| GalXC-DGAT2-0509 | DP19501P :DP19500 G | CUCAUGUAC AUAUUCUGC AA | 137 | UUGCAGAAUA UGUACAUGAG GG | 138 |
| GalXC-DGAT2-0893 | DP19503P :DP19502 G | GACACCAUA GACUAUUUG CA | 139 | UGCAAAUAGU CUAUGGUGUC GG | 140 |
| GalXC-DGAT2-1138 | DP19505P :DP19504 G | GAAGAAGUU CCAGAAAUA CA | 141 | UGUAUUUCUG GAACUUCUUCG G | 142 |
| GalXC-DGAT2-0896 | DP19507P :DP19506 G | ACCAUAGAC UAUUUGCUU UA | 143 | UAAAGCAAAU AGUCUAUGGU GG | 144 |
| GalXC-DGAT2-0506 | DP19509P :DP19508 G | AUCCUCAUG UACAUAUUC UA | 145 | UAGAAUAUGU ACAUGAGGAU GG | 146 |
| GalXC-DGAT2-1079 | DP19511P :DP19510 G | GAGAAUGAA GUGUACAAG CA | 147 | UGCUUGUACAC UUCAUUCUCGG | 148 |

TABLE 9

GalNAc-Conjugated Human/Cyno DGAT2 Targeting candidate Sequences

| Oligonucleotide | DP# | Sequence (36 mer sense strand) | SE Q ID NO | Sequence (antisens e strand) | SE Q ID NO | Modified 36mer Sense strand SEQ ID NO | Modified 22mer anti-sense strand SEQ ID NO |
|---|---|---|---|---|---|---|---|
| GalXC-DGAT2-1139 | DP19483 P:DP1948 2G | AAGAA GUUCC AGAAA UACAA GCAGC CGAAA GGCUG C | 190 | UUGUA UUUCU GGAAC UUCUU GG | 118 | 236 | 282 |
| GalXC-DGAT2-0899 | DP19481 P:DP1948 0G | AUAGA CUAUU UGCUU UCAAA GCAGC CGAAA GGCUG C | 191 | UUUGA AAGCA AAUAG UCUAU GG | 120 | 237 | 283 |
| GalXC-DGAT2-1080 | DP19485 P:DP1948 4G | AGAAU GAAGU GUACA AGCAA GCAGC CGAAA GGCUG C | 192 | UUGCU UGUAC ACUUC AUUCU GG | 122 | 238 | 284 |
| GalXC-DGAT2-1086 | DP19487 P:DP1948 6G | AAGUG UACAA GCAGG UGAUA GCAGC CGAAA GGCUG C | 193 | UAUCA CCUGC UUGUA CACUU GG | 124 | 239 | 285 |
| GalXC-DGAT2-0898 | DP19489 P:DP1948 8G | CAUAG ACUAU UUGCU UUCAA GCAGC CGAAA GGCUG C | 194 | UUGAA AGCAA AUAGU CUAUG GG | 126 | 240 | 286 |
| GalXC-DGAT2-0894 | DP19491 P:DP1949 0G | ACACC AUAGA CUAUU UGCUA GCAGC CGAAA GGCUG C | 195 | UAGCA AAUAG UCUAU GGUGU GG | 128 | 241 | 287 |
| GalXC-DGAT2-1074 | DP19493 P:DP1949 2G | UUGGA GAGAA UGAAG UGUAA GCAGC CGAAA GGCUG C | 196 | UUACA CUUCA UUCUC UCCAA GG | 130 | 242 | 288 |
| GalXC-DGAT2-1083 | DP19495 P:DP1949 4G | AUGAA GUGUA CAAGC AGGUA GCAGC CGAAA GGCUG C | 197 | UACCU GCUUG UACAC UUCAU GG | 132 | 243 | 289 |
| GalXC-DGAT2-0895 | DP19497 P:DP1949 6G | CACCA UAGAC UAUUU GCUUA GCAGC CGAAA GGCUG C | 198 | UAAGC AAAUA GUCUA UGGUG GG | 134 | 244 | 290 |
| GalXC-DGAT2-0897 | DP19499 P:DP1949 8G | CCAUA GACUA UUUGC UUUCA GCAGC CGAAA GGCUG C | 199 | UGAAA GCAAA UAGUC UAUGG GG | 136 | 245 | 291 |
| GalXC-DGAT2-0509 | DP19501 P:DP1950 0G | CUCAU GUACA UAUUC UGCAA GCAGC CGAAA GGCUG C | 200 | UUGCA GAAUA UGUAC AUGAG GG | 138 | 246 | 292 |
| GalXC-DGAT2-0893 | DP19503 P:DP1950 2G | GACAC CAUAG ACUAU UUGCA GCAGC CGAAA GGCUG C | 201 | UGCAA AUAGU CUAUG GUGUC GG | 140 | 247 | 293 |
| GalXC-DGAT2-1138 | DP19505 P:DP1950 4G | GAAGA AGUUC CAGAA AUACA GCAGC CGAAA GGCUG C | 202 | UGUAU UUCUG GAACU UCUUC GG | 142 | 248 | 294 |
| GalXC-DGAT2-0896 | DP19507 P:DP1950 6G | ACCAU AGACU AUUUG CUUUA GCAGC CGAAA GGCUG C | 203 | UAAAG CAAAU AGUCU AUGGU GG | 144 | 249 | 295 |
| GalXC-DGAT2-0506 | DP19509 P:DP1950 8G | AUCCU CAUGU ACAUA UUCUA GCAGC CGAAA GGCUG C | 204 | UAGAA UAUGU ACAUG AGGAU GG | 146 | 250 | 296 |
| GalXC-DGAT2-1079 | DP19511 P:DP1951 0G | GAGAA UGAAG UGUAC AAGCA GCAGC CGAAA GGCUG C | 205 | UGCUU GUACA CUUCA UUCUC GG | 148 | 251 | 297 |

The GalNAc-conjugated DGAT2 oligonucleotides listed in Table9 were evaluated in mice engineered to transiently express human DGAT2 mRNA in vivo in mouse hepatocytes. Briefly, 6-8-week-old female CD-1 mice were treated subcutaneously with a GalNAc-conjugated DGAT2 oligonucleotide at a dose level of 3 mg/kg. Four days later (96 h), the mice were hydrodynamically injected with equal amounts of DNA plasmids encoding the full human DGAT2 gene under control of a ubiquitous cytomegalovirus (CMV) promoter sequence. Twenty hours after introduction of the plasmid, liver samples were collected. Total RNA derived from these mice was subjected to qRT-PCR analysis for DGAT2 mRNA, relative to mice treated only with an identical volume of PBS. The TaqMan RT-qPCR probes Hs01045913 (Cat No 4331182) purchased from Life Technologies to evaluate DGAT2. The values were normalized for transfection efficiency using the NeoR gene included on the plasmid.

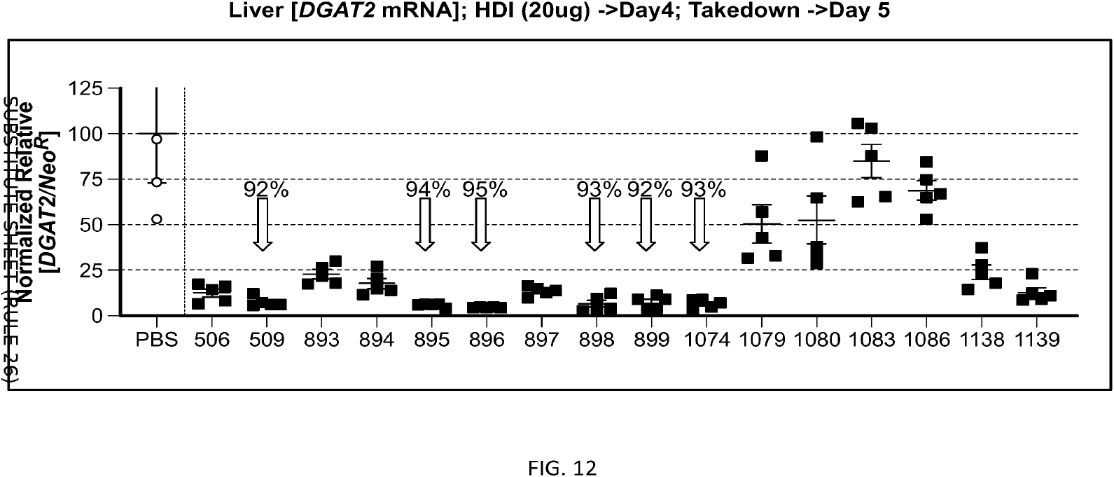

As shown in FIG. 12, twelve of the GalNAc-conjugated DGAT2 oligonucleotides tested inhibit DGAT2 expression as determined by a reduced amount of DGAT2 mRNA in liver samples from oligonucleotide-treated mice relative to mice treated with PBS.

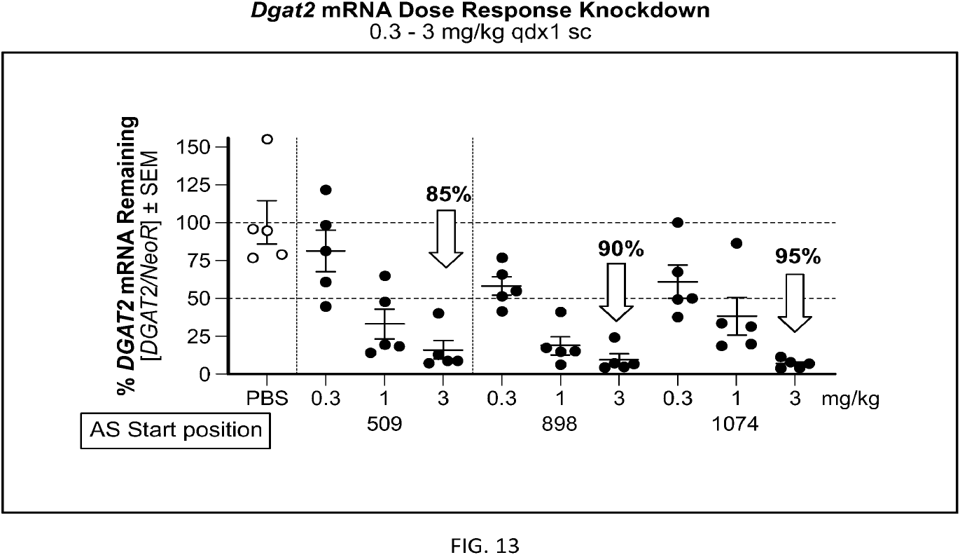

Three compounds that were shown to inhibit DGAT2 expression at 3 mg/kg were tested in a dose response study. Briefly, 6-8-week-old female CD-1 mice were treated subcutaneously with a GalNAc-conjugated DGAT2 oligonucleotide at doses of 0.3, 1 and 3 mg/kg. Four days later (96 h), the mice were hydrodynamically injected with equal amounts of DNA plasmids encoding the full human DGAT2 gene isoforms under control of a ubiquitous cytomegalovirus (CMV) promoter sequence. 20 hours after introduction of the plasmid, liver samples were collected. Total RNA derived from these mice were subjected to qRT-PCR analysis for DGAT2 mRNA, relative to mice treated only with an identical volume of PBS. The values were normalized for transfection efficiency using the NeoR gene included on the plasmid. As shown in FIG. 13, GalXC-DGAT2-0509, GalXC-DGAT2-0898 and GalXC-DGAT2-1074 display ED50s of < 1 mg/kg against human DGAT2.

### Non-Human Primate (NHP) Studies

Of the compounds tested in FIG. 12, six were selected for evaluation of their ability to inhibit DGAT2 expression in non-human primates (NHPs). The GalNAc-conjugated DGAT2 oligonucleotides listed in Table 10 comprise chemically modified nucleotides having pattern as described in FIG. 1C with the 2′-O-methyl modified nucleotides on the guide strand position 4 changed to 2′Floro.

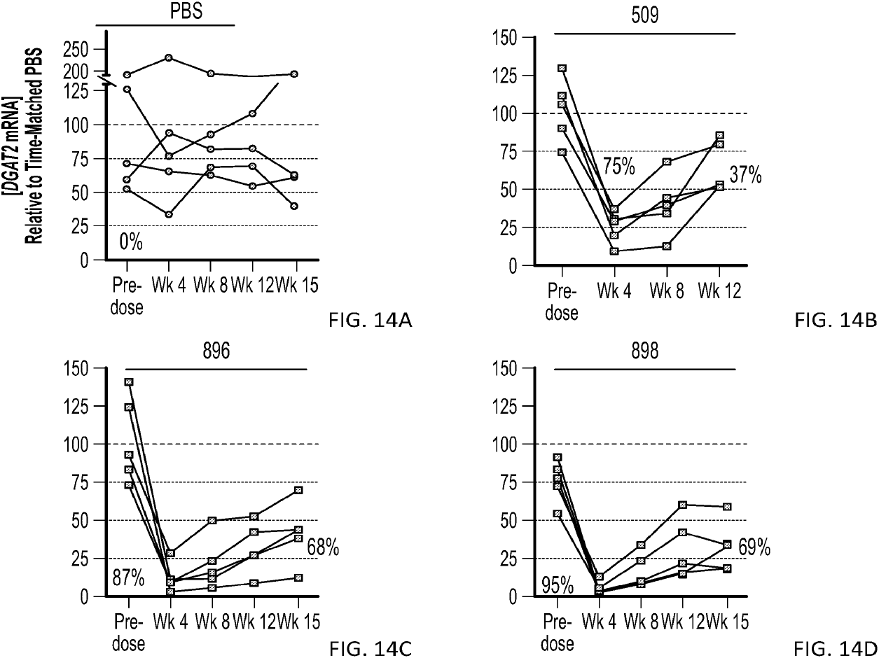

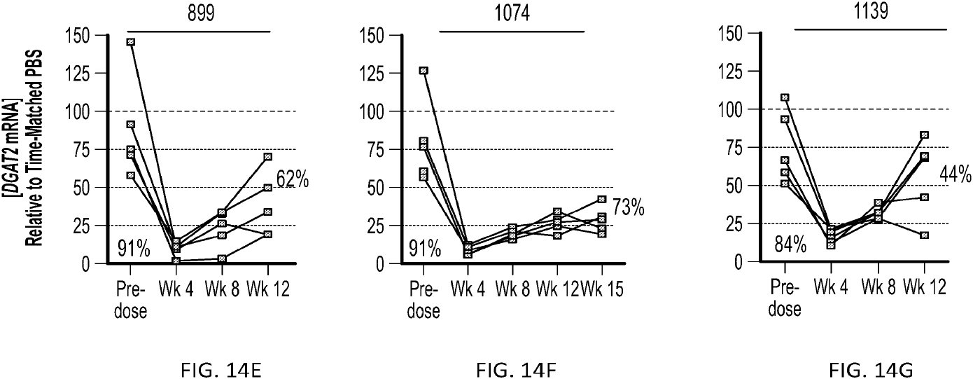

The GalNAc-conjugated DGAT2 oligonucleotides listed in Table 10 were evaluated in cynomolgus monkeys (Macaca fascicularis). In this study, the monkeys were grouped so that their mean body weights (about 6 kg) were comparable between the control and experimental groups. Each cohort contained male and female subjects. The GalNAc-conjugated DGAT2 oligonucleotides were administered subcutaneously at a dose of 6 mg/kg on Study Day 0. Blood samples were collected one week prior to dosing (Day -7), on the dosing date (Day 0) and 28, 54 and 86 after dosing. Ultrasound-guided core needle liver biopsies were collected on Study Days -7, 28, 56 and 84. At each time point, total RNA derived from the liver biopsy samples was subjected to qRT-PCR analysis to measure DGAT2 mRNA in oligonucleotide-treated monkeys relative to those treated with a comparable volume of PBS. To normalize the data, the measurements were made relative to the geometric mean of two reference genes, PPIB and 18S rRNA. The following TaqMan qPCR probes purchased from Life Technologies, Inc, were used to evaluate gene expressions as described in Example 3. As shown in FIGS. 14A-14G, treating NHPs with the GalNAc-conjugated DGAT2 oligonucleotides listed in Table 10 inhibited DGAT2 expression in the liver, as determined by a reduced amount of DGAT2 mRNA in liver samples from oligonucleotide-treated NHPs relative to NHPs treated with PBS. The mean percent reduction of DGAT2 mRNA in the liver samples of treated NHPs is indicated for week 4 (day 28) and week 12 (day 84) along with the plot of the mean values over times in FIGS. 14B-14G. All GalNAc-conjugated oligonucleotides evaluated reduced DGAT2 expression by at least 75 percent (%) at week 4. These results demonstrate that treating NHPs with the GalNAc-conjugated DGAT2 oligonucleotides reduces the amount of DGAT2 mRNA in the liver.

Taken together, these results show that GalNAc-conjugated DGAT2 oligonucleotides designed to target human DGAT2 mRNA inhibit expression in vivo.

TABLE 10

GalNAc-Conjugated DGAT2 Oligonucleotides Evaluated in NHPs

| Oligonucleotide | DP# | Sequence (36 mer sense strand) | SE Q ID NO | Sequence (antisens e strand) | SE Q ID NO | Modified 36mer Sense strand SEQ ID NO | Modified 22mer anti-sense strand SEQ ID NO |
|---|---|---|---|---|---|---|---|
| GalXC-DGAT2-0509 | DP19501P :DP19500 G | CUCAUG UACAUA UUCUGC AA GCAGCC GAAAGG CUGC | 200 | UUGCA GAAUA UGUAC AUGAG GG | 138 | 246 | 292 |
| GalXC-DGAT2-0896 | DP19507P :DP19506 G | ACCAUA GACUAU UUGCUU UA GCAGCC GAAAGG CUGC | 203 | UAAAG CAAAU AGUCU AUGGU GG | 144 | 249 | 295 |
| GalXC-DGAT2-0898 | DP19489P :DP19488 G | CAUAGA CUAUUU GCUUUC AA GCAGCC GAAAGG CUGC | 194 | UUGAA AGCAA AUAGU CUAUG GG | 126 | 240 | 286 |
| GalXC-DGAT2-0899 | DP19481P :DP19480 G | AUAGAC UAUUUG CUUUCA AA GCAGCC GAAAGG CUGC | 191 | UUUGA AAGCA AAUAG UCUAU GG | 120 | 237 | 283 |
| GalXC-DGAT2-1074 | DP19493P :DP19492 G | UUGGAG AGAAUG AAGUGU AA GCAGCC GAAAGG CUGC | 196 | UUACA CUUCA UUCUC UCCAA GG | 130 | 242 | 288 |
| GalXC-DGAT2-1139 | DP19483P :DP19482 G | AAGAAG UUCCAG AAAUAC AA GCAGCC GAAAGG CUGC | 190 | UUGUA UUUCU GGAAC UUCUU GG | 118 | 236 | 282 |

### Example 6: Combination RNAi Oligonucleotide Inhibition of ACAC and DGAT2 Expression In Vivo

### Mouse Studies

GalXC-ACAC-4458 (chemical modification pattern as shown in FIG. 1A) and GalXC-DGAT2-1463 (chemical modification pattern as shown in FIG. 1C) oligonucleotides were evaluated in combination in GAN-NASH fed mice. Briefly, GAN-NASH fed mice, fed a diet consisting of 40 kcal% from fat, 20 kcal% from fructose and 2% cholesterol, were obtained from Taconic Biosciences Inc. after 20 weeks on diet. Weekly concurrent, subcutaneous dosing was initiated on week 27 according to the study design shown in Table 11.

TABLE 11

Study Design for Combination Oligonucleotide Inhibition of ACAC and DGAT2

| Treatment Group | Diet | Number of mice | Dose mg/kg |
|---|---|---|---|
| PBS | Control | 15 | N/A |
| PBS | GAN-NASH | 10 | N/A |
| GalXC-ACAC-4458 | GAN-NASH | 10 | 5 |
| GalXC-DGAT2-1463 | GAN-NASH | 10 | 5 |
| GalXC-ACAC-4458 + GalXC-DGAT2-1463 | GAN-NASH | 10 | 5+5 |

After a sixth weekly dose, mice harvested and prepped for study on week 33, one week after the last dose, and subjected to a full necropsy. In addition to target mRNA levels, the following categories of endpoints were investigated: systemic (e.g., cholesterol), liver Steatosis, liver inflammation and liver fibrosis. A detailed summary of results is found in Table 12, with combination data highlighted if p value >0.05 vs. GalXC-ACAC treatment alone.

TABLE 12

Summary of endpoints investigated ACAC and DGAT2 Combination Study

| Category | Endpoint | Fold induction vs. normal diet | | | |
|---|---|---|---|---|---|
| | | PBS | GalXC-ACAC* | GalXC-DGAT2* | Combination** |
| Systemic | ALT | 5.2 | 3.3 | 4.2 | 2.4 |
| | AST | 7.8 | 3.3 | 7.5 | 3.8 |
| | Total Chol | 2.8 | 2.1 | 2.5 | 1.7 |
| | LDL-C | 6.9 | 5.6 | 8.2 | 5.6 |
| | HDL-C | 1.6 | 1.2 | 1.5 | 1.1 |
| | ALP¹ | 2.8 | 2.6 | 6.3 | 2.8 |
| Liver Steatosis | % Area | 36.0 | 25.6 | 25.7 | 21.3 |
| | Liver TG | 3.5 | 3.1 | 1.4 | 1.5 |
| Liver Inflammation | IL1b mRNA | 3.0 | 2.3 | 2.9 | 2.7 |
| | IL6 mRNA | 1.4 | 1.4 | 0.9 | 0.7 |
| | Tnfa mRNA | 5.6 | 3.6 | 6.0 | 4.2 |
| | IL12b mRNA | 8.9 | 5.7 | 6.8 | 3.6 |
| | Crown-Like Structures | 606.4 | 273.3 | 588.0 | 478.8 |
| | Path Score | 0.1 | 1.2 | 1.3 | 1.1 |
| Liver Fibrosis | Col1a1 mRNA | 12.0 | 6.0 | 6.6 | 4.0 |
| | Timp1 mRNA | 32.9 | 15.8 | 16.7 | 7.7 |
| | Acta2 mRNA | 1.9 | 1.5 | 1.1 | 1.2 |
| | Tgfb1 mRNA | 2.6 | 1.8 | 2.4 | 2.0 |
| | SMA IHC | 2.1 | 1.8 | 1.6 | 1.6 |
| | SMA Score | 2.8 | 2.1 | 1.8 | 1.7 |
| | Sirius Red² | 1.6 | 2.6 | 1.5 | 2.3 |
| | Path score | 3.5 | 5.0 | 2.5 | 4.5 |

1- ALP elevated in DGAT2 cohort (relative to diet alone) but normalized in combination cohort
2 Sirius Red staining robust 48-week post GAN-NASH challenge.

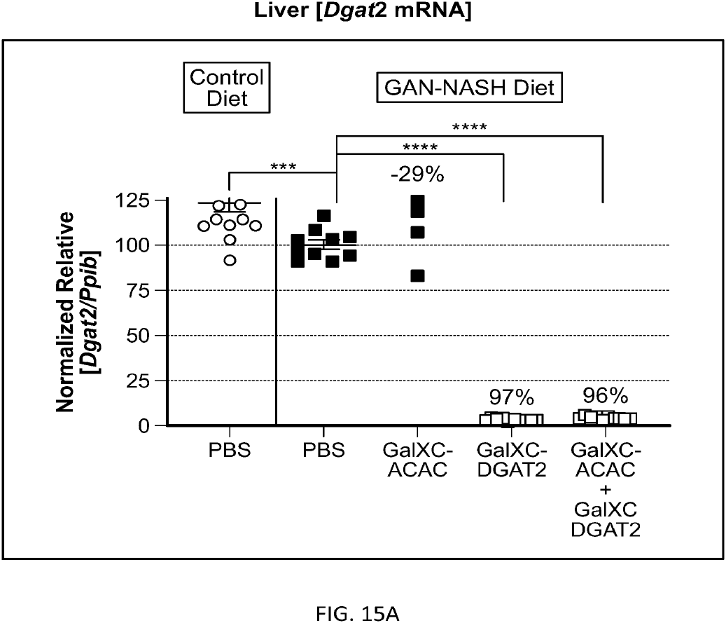

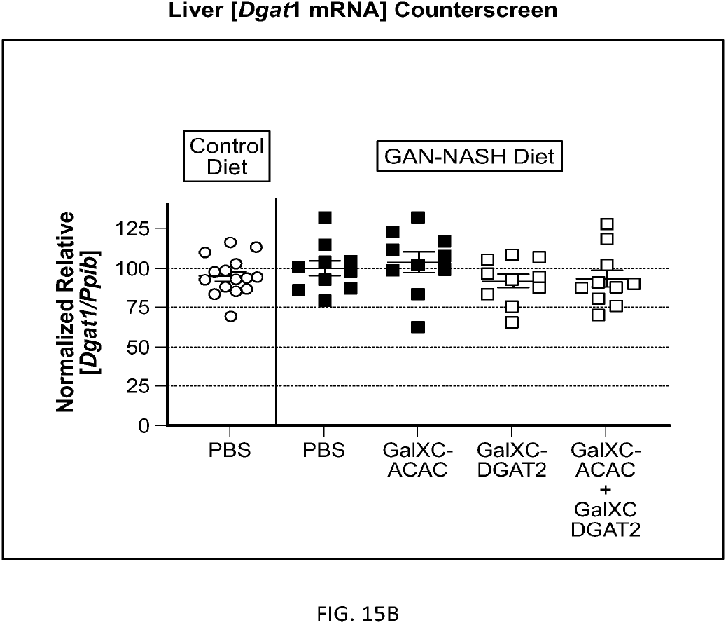

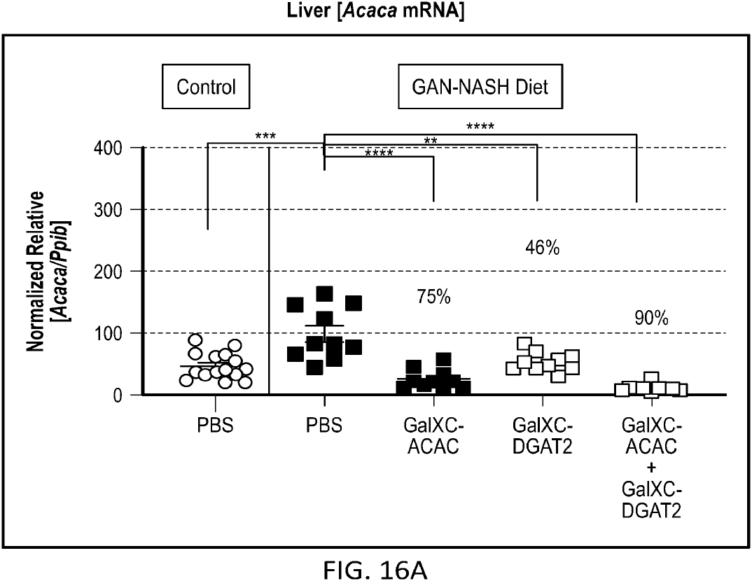

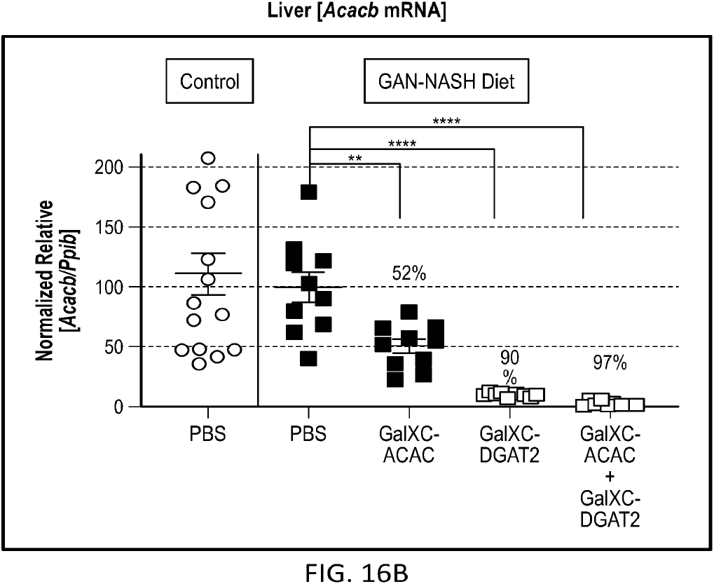

Measurement of target mRNA levels in response to GalXC-ACAC and GalXC-DGAT2 oligonucleotide combination were measured in liver. DGAT2 mRNA knockdown of > 95% is shown in both GalXC-DGAT2 treatment alone and in combination treatment (FIG. 15A) while no knockdown of DGAT1 mRNA is observed (FIG. 15B). Knockdown of 75% and 52% in ACACA and ACACB mRNA respectively was observed in the GalXC-ACAC alone treatment group (FIGS. 16A and 16B). Of note, while no combination effect was observed on DGAT2 mRNA levels, the combination of GalXC-ACAC with GalXC-DGAT2 resulted in greater knockdown of ACACA and ACACB mRNA than in the GalXC-ACAC alone treatment group. Inhibition of ACACA increased from 75% to 90% in response to the combination while inhibition of ACACB mRNA increased from 52% to 97% in response to the combination. This data represents an unexpected synergistic effect of GalXC-ACAC and GalXC-DGAT2 combination treatment on the ACACA and ACACB target mRNA levels.

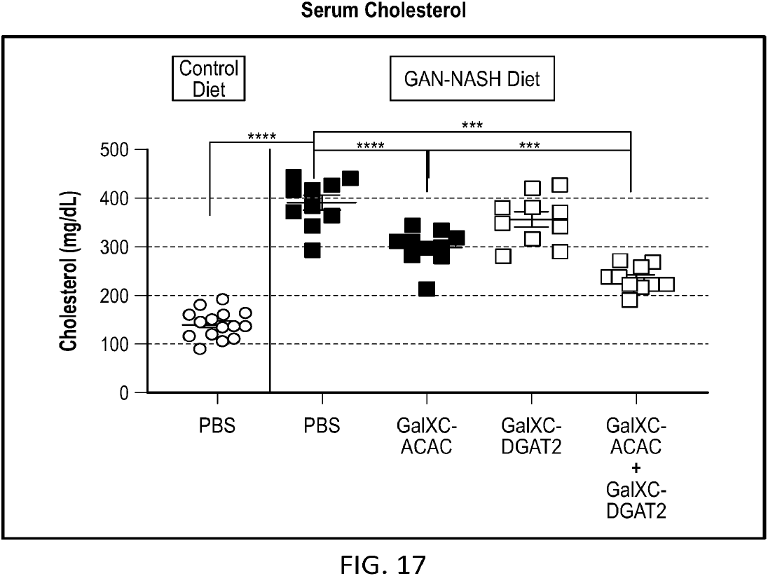

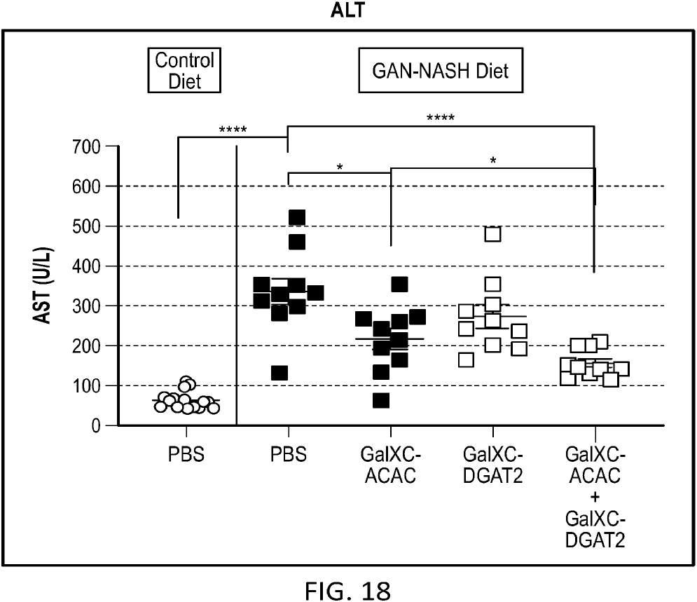

To assess systemic response to the GalXC-ACAC and GalXC-DGAT2 oligonucleotide combination in vivo, total serum cholesterol, as well as the liver enzyme alanine transaminase (ALT) were measured at the conclusion of the study. Serum cholesterol (FIG. 17) and ALT levels (FIG. 18) were decreased in the combination treatment group when compared to PBS or either single treatment group alone. These data show that GalXC-ACAC/GalXC-DGAT2 combination treatment may confer an additive or synergistic effect in the treatment of the systemic effects of liver disease as compared to either single therapy alone.

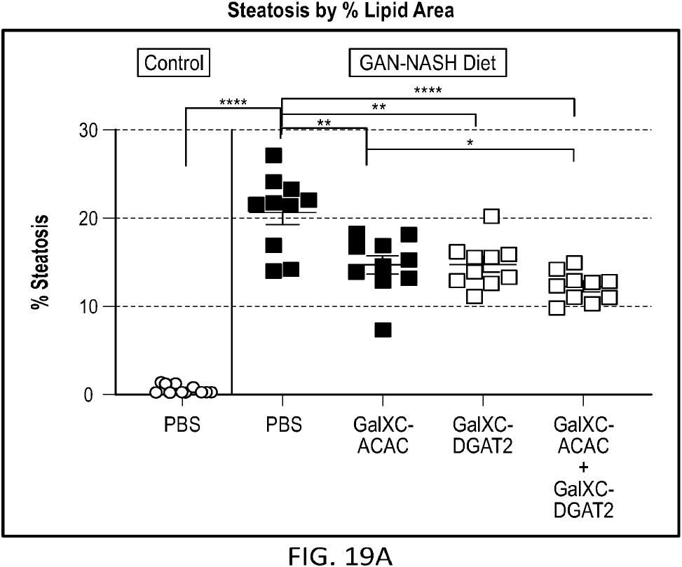

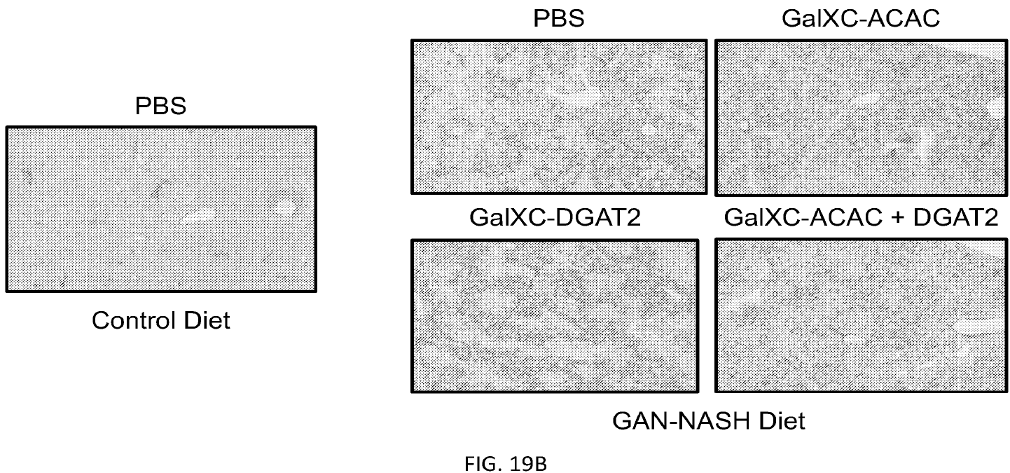

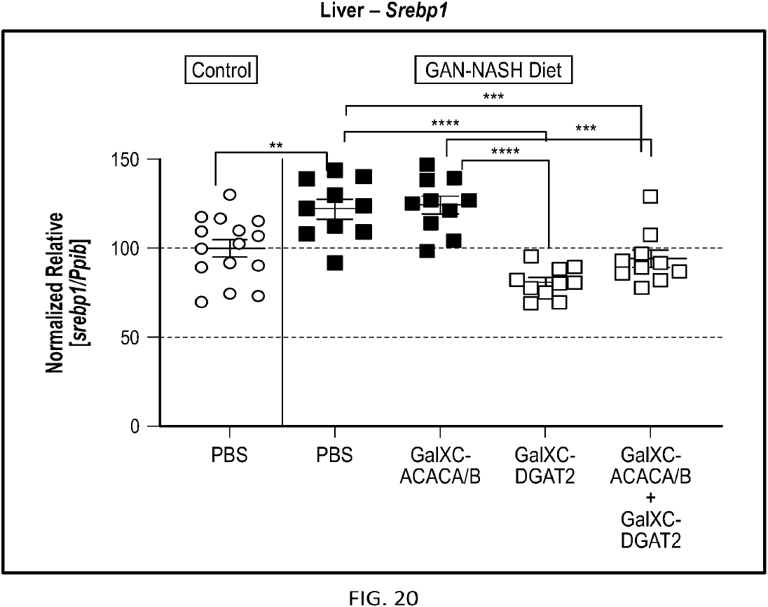

To investigate the effect of the GalXC-ACAC and GalXC-DGAT2 oligonucleotide combination on liver steatosis, steatosis as assessed by % area of liver lipids was quantified (FIGS. 19A and 19B). In addition, liver TGs (Table 12) as well as Srebp1, an mRNA marker of liver steatosis, were measured as shown in FIG. 20. Liver steatosis as assessed by % area of liver lipids was shown to be decreased in the combination treatment group when compared to either single treatment group alone, while liver TGs and Srebp1 mRNA levels return to at or below baseline levels with combination treatment.

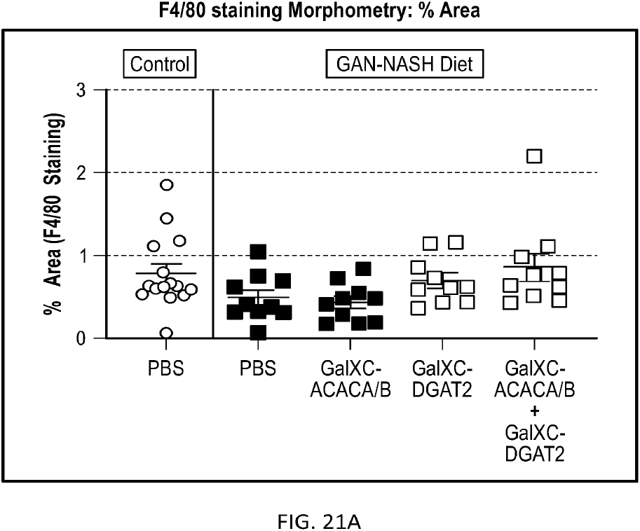

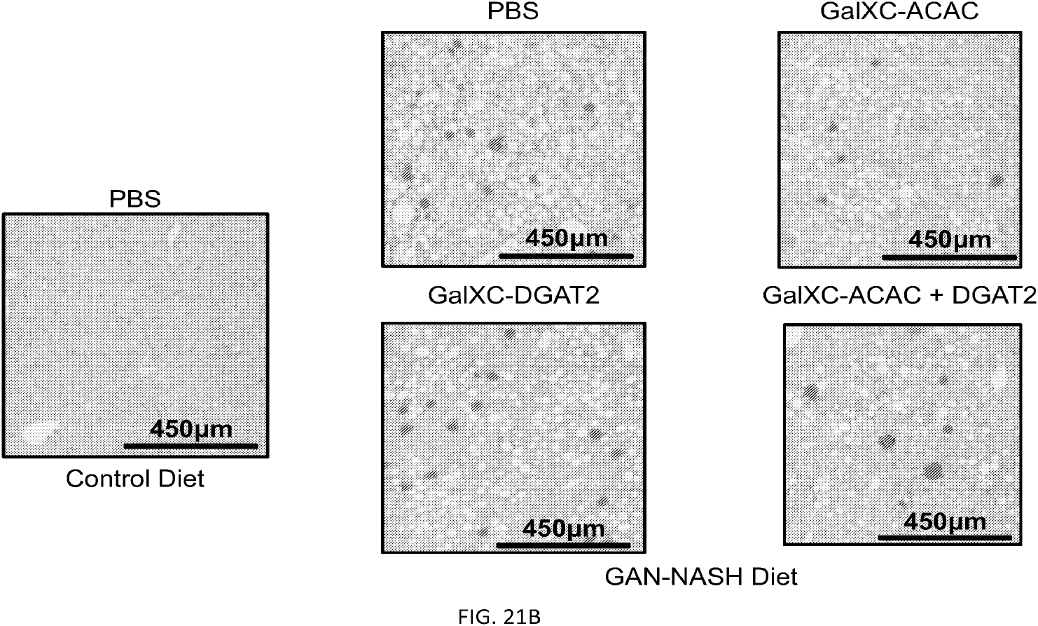

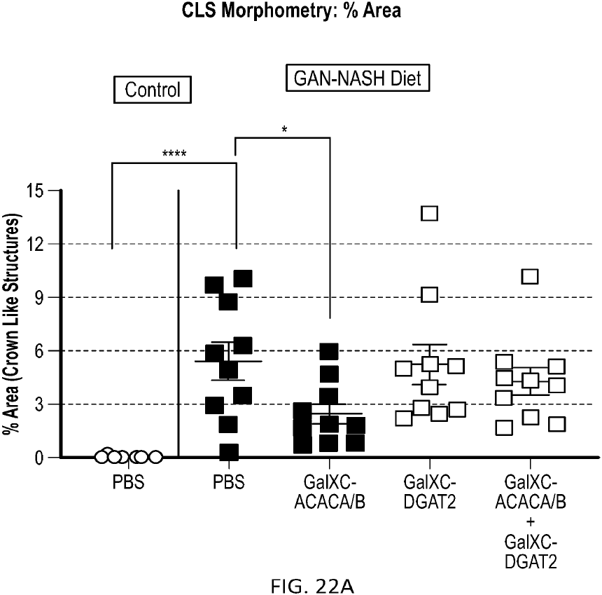

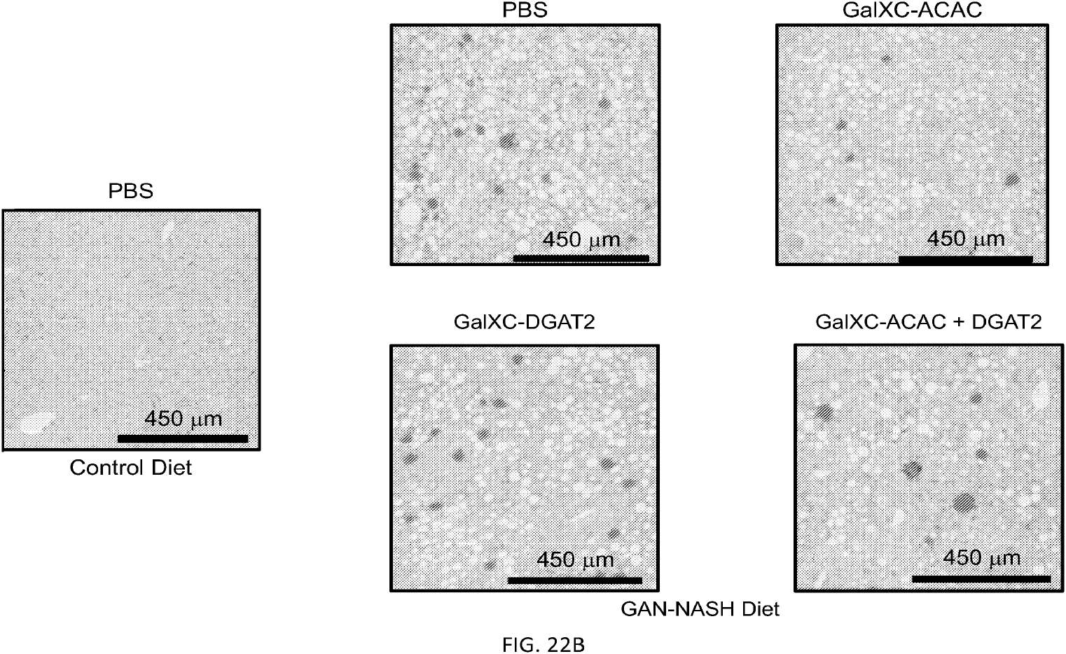

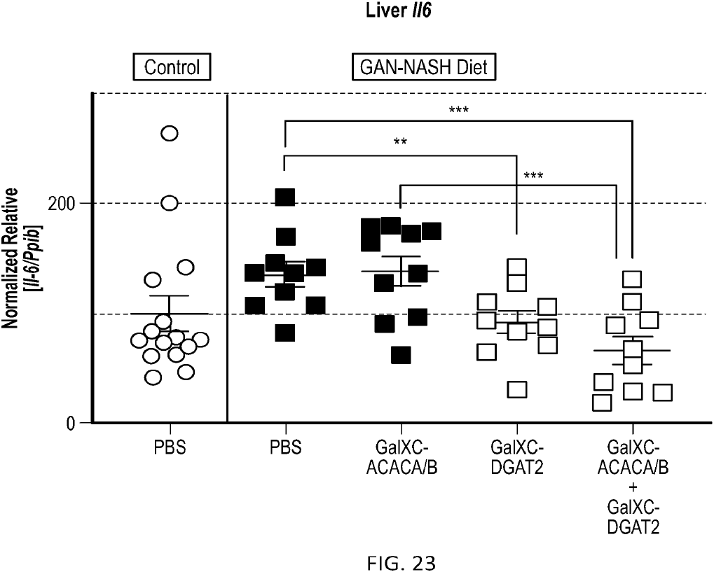

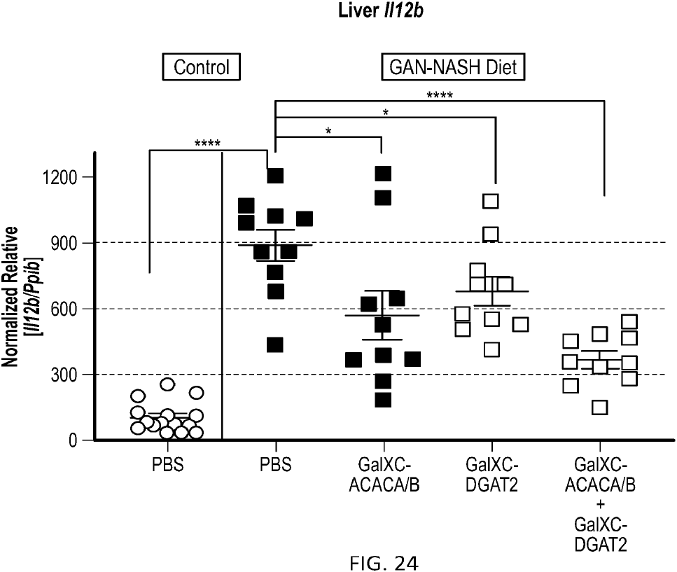

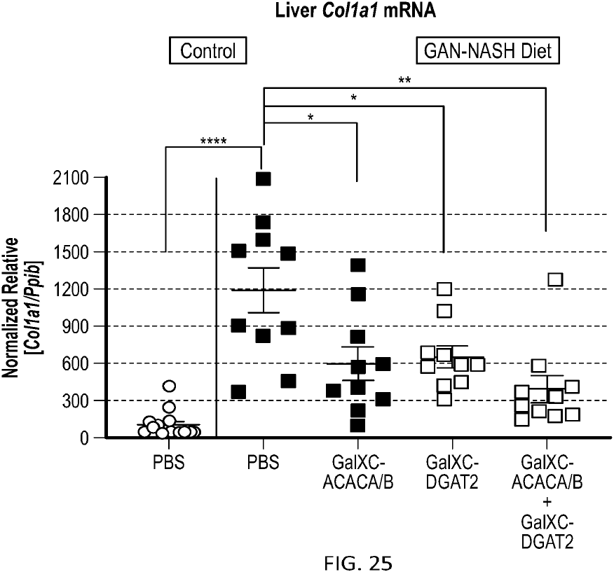

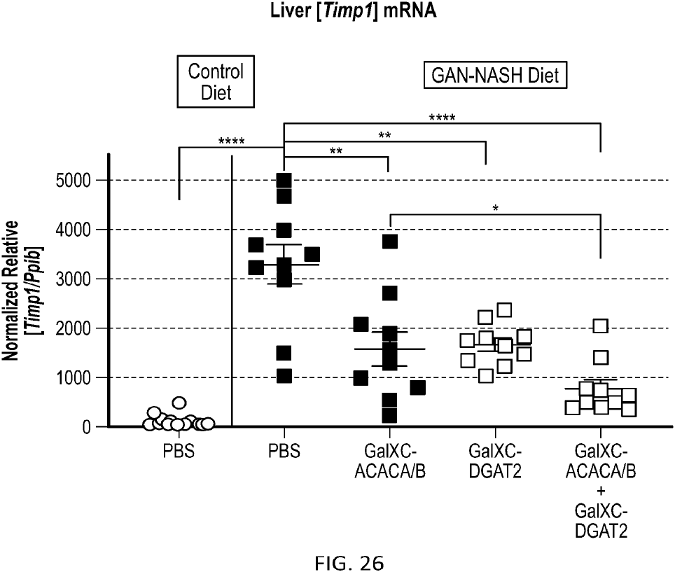

To investigate the effect of the GalXC-ACAC and GalXC-DGAT2 oligonucleotide combination on liver inflammation, macrophage infiltration and pathogenesis was assessed by visualization of the F4/80 macrophage marker (FIGS. 21A and 21B) as well as assaying for Crown Like Structures (CLS) (FIGS. 22A and 22B). No significant change was observed in F4/80 staining or the CLS assay as compared to PBS or either the GalXC-ACAC or GalXC-DGAT2 single treatments groups or the combination treatment group. Liver levels of IL6 and IL12b, cytokine markers of liver inflammation, were also measured. Liver levels of IL6 (FIG. 23) and IL12b (FIG. 24) were decreased in the combination treatment group when compared to PBS or either single treatment group alone. These data taken together suggest that combination treatment does not confer liver inflammation. Finally, the effect of the GalXC-ACAC and GalXC-DGAT2 oligonucleotide combination on liver inflammation was explored through the measurement of expression of hepatic fibrosis marker genes, Col1a1 and Timp1. Both Col1a1 (FIG. 25) and Timp1 (FIG. 26) mRNA levels were decreased in the combination treatment group as compared to PBS or either the GalXC-ACAC or GalXC-DGAT2 single treatments groups. These data show that GalXC-ACAC/GalXC-DGAT2 combination treatment may confer an additive or synergistic effects in the treatment of liver fibrosis as compared to either single therapy alone or the sum of the individual treatments alone. At a dosage of 5 mg/kg for each of the molecules, administered simultaneously, the liver data demonstrate an enhanced suppression of the target genes not seen in the use of either siRNA alone. These data demonstrate the synergistic effect of the combination of ACC siRNA and DGAT2 siRNA inhibitors of the current disclosure in protein knockdown. These effects in turn may lead to increased relief from the pathologies associated with Metabolic Syndrome as provided herein. In conclusion, when taken as a whole, the data from this investigation of the GalXC-ACAC/GalXC-DGAT2 oligonucleotide combination supports a hypothesis that combination treatment may have an improved effect on measurable endpoints of systemic liver disease, liver steatosis and liver fibrosis as compared to single agent treatments. In addition, the combination treatment did not confer any increase in liver inflammation as measured by macrophage infiltration and pathogenicity assays or by liver cytokine levels. Also noteworthy, the GalXC-ACAC/GalXC-DGAT2 oligonucleotide combination shows synergistic activity in the degree of ACAC mRNA knockdown as compared to GalXC-ACAC treatment alone, suggestion an as of yet undescribed combination mechanism of action.

SEQUENCE LISTING

| SEQ ID NO | Description | Sequence |
|---|---|---|
| 1 | GalXC-ACAC-4458 (sense strand) | AACCACAUCUUCCUCAACUA |
| 2 | GalXC-ACAC-4458 (antisense strand) | UAGUUGAGGAAGAUGUGGUUGG |
| 3 | GalXC-ACAC-4459 (sense strand) | ACCACAUCUUCCUCAACUUA |
| 4 | GalXC-ACAC-4459 (antisense strand) | UAAGUUGAGGAAGAUGUGGUGG |
| 5 | GalXC-ACAC-4460 (sense strand) | CCACAUCUUCCUCAACUUUA |
| 6 | GalXC-ACAC-4460 (antisense strand) | UAAAGUUGAGGAAGAUGUGGGG |
| 7 | GalXC-ACAC-4461 (sense strand) | CACAUCUUCCUCAACUUUGA |
| 8 | GalXC-ACAC-4461 (antisense strand) | UCAAAGUUGAGGAAGAUGUGGG |
| 9 | GalXC-ACAC-4462 (sense strand) | ACAUCUUCCUCAACUUUGUA |
| 10 | GalXC-ACAC-4462 (antisense strand) | UACAAAGUUGAGGAAGAUGUGG |
| 11 | GalXC-ACAC-4463 (sense strand) | CAUCUUCCUCAACUUUGUGA |
| 12 | GalXC-ACAC-4463 (antisense strand) | UCACAAAGUUGAGGAAGAUGGG |
| 13 | GalXC-ACAC-4464 (sense strand) | AUCUUCCUCAACUUUGUGCA |
| 14 | GalXC-ACAC-4464 (antisense strand) | UGCACAAAGUUGAGGAAGAUGG |
| 15 | GalXC-ACAC-4465 (sense strand) | UCUUCCUCAACUUUGUGCCA |
| 16 | GalXC-ACAC-4465 (antisense strand) | UGGCACAAAGUUGAGGAAGAGG |
| 17 | GalXC-ACAC-4466 (sense strand) | CUUCCUCAACUUUGUGCCCA |
| 18 | GalXC-ACAC-4466 (antisense strand) | UGGGCACAAAGUUGAGGAAGGG |
| 19 | GalXC-ACAC-4467 (sense strand) | UUCCUCAACUUUGUGCCCAA |
| 20 | GalXC-ACAC-4467 (antisense strand) | UUGGGCACAAAGUUGAGGAAGG |
| 21 | GalXC-ACAC-4468 (sense strand) | UCCUCAACUUUGUGCCCACA |
| 22 | GalXC-ACAC-4468 (antisense strand) | UGUGGGCACAAAGUUGAGGAGG |
| 23 | GalXC-ACAC-5080 (sense strand) | UUGUCAUCGGCAAUGACAUA |
| 24 | GalXC-ACAC-5080 (antisense strand) | UAUGUCAUUGCCGAUGACAAGG |
| 25 | GalXC-ACAC-1919 (sense strand) | GUUAUGUGAGUGCUGGGACA |
| 26 | GalXC-ACAC-1919 (antisense strand) | UGUCCCAGCACUCACAUAACGG |
| 27 | GalXC-ACAC-2384 (sense strand) | UUUCAAACAUGGUGGUGGCA |
| 28 | GalXC-ACAC-2384 (antisense strand) | UGCCACCACCAUGUUUGAAAGG |
| 29 | GalXC-ACAC-2389 (sense strand) | AACAUGGUGGUGGCUUUGAA |
| 30 | GalXC-ACAC-2389 (antisense strand) | UUCAAAGCCACCACCAUGUUGG |
| 31 | GalXC-ACAC-2392 (sense strand) | AUGGUGGUGGCUUUGAAGGA |
| 32 | GalXC-ACAC-2392 (antisense strand) | UCCUUCAAAGCCACCACCAUGG |
| 33 | GalXC-ACAC-2896 (sense strand) | ACCUGUGUGUUUGAGAAGGA |
| 34 | GalXC-ACAC-2896 (antisense strand) | UCCUUCUCAAACACACAGGUGG |
| 35 | GalXC-ACAC-2897 (sense strand) | CCUGUGUGUUUGAGAAGGAA |
| 36 | GalXC-ACAC-2897 (antisense strand) | UUCCUUCUCAAACACACAGGGG |
| 37 | GalXC-ACAC-3442 (sense strand) | CAGUAUGCUAGCAACAUCAA |
| 38 | GalXC-ACAC-3442 (antisense strand) | UUGAUGUUGCUAGCAUACUGGG |
| 39 | GalXC-ACAC-3443 (sense strand) | AGUAUGCUAGCAACAUCACA |
| 40 | GalXC-ACAC-3443 (antisense strand) | UGUGAUGUUGCUAGCAUACUGG |
| 41 | GalXC-ACAC-4201 (sense strand) | GUGGUGGAAUUCCAGUUCAA |
| 42 | GalXC-ACAC-4201 (antisense strand) | UUGAACUGGAAUUCCACCACGG |
| 43 | GalXC-ACAC-4204 (sense strand) | GUGGAAUUCCAGUUCAUGCA |
| 44 | GalXC-ACAC-4204 (antisense strand) | UGCAUGAACUGGAAUUCCACGG |
| 45 | GalXC-ACAC-4205 (sense strand) | UGGAAUUCCAGUUCAUGCUA |
| 46 | GalXC-ACAC-4205 (antisense strand) | UAGCAUGAACUGGAAUUCCAGG |
| 47 | GalXC-ACAC-4206 (sense strand) | GGAAUUCCAGUUCAUGCUGA |
| 48 | GalXC-ACAC-4206 (antisense strand) | UCAGCAUGAACUGGAAUUCCGG |
| 49 | GalXC-ACAC-4207 (sense strand) | GAAUUCCAGUUCAUGCUGCA |
| 50 | GalXC-ACAC-4207 (antisense strand) | UGCAGCAUGAACUGGAAUUCGG |
| 51 | GalXC-ACAC-4208 (sense strand) | AAUUCCAGUUCAUGCUGCCA |
| 52 | GalXC-ACAC-4208 (antisense strand) | UGGCAGCAUGAACUGGAAUUGG |
| 53 | GalXC-ACAC-5082 (sense strand) | UAACCACAUCUUCCUCAACA |
| 54 | GalXC-ACAC-5082 (sense strand) | UGUUGAGGAAGAUGUGGUUAGG |
| 55 | GalXC-ACAC-5083 (sense strand) | AACCACAUCUUCCUCAACUA |
| 56 | GalXC-ACAC-5083 (sense strand) | UAGUUGAGGAAGAUGUGGUUGG |
| 57 | GalXC-ACAC-5084 (sense strand) | ACCACAUCUUCCUCAACUUA |
| 58 | GalXC-ACAC-5084 (sense strand) | UAAGUUGAGGAAGAUGUGGUGG |
| 59 | GalXC-ACAC-5305 (sense strand) | UACAAGGAAGUGACUGACUA |
| 60 | GalXC-ACAC-5305 (antisense strand) | UAGUCAGUCACUUCCUUGUAGG |
| 61 | GalXC-ACAC-5307 (sense strand) | CAAGGAAGUGACUGACUCCA |
| 62 | GalXC-ACAC-5307 (antisense strand) | UGGAGUCAGUCACUUCCUUGGG |
| 63 | GalXC-ACAC-5309 (sense strand) | AGGAAGUGACUGACUCCAGA |
| 64 | GalXC-ACAC-5309 (antisense strand) | UCUGGAGUCAGUCACUUCCUGG |
| 65 | GalXC-ACAC-6331 (sense strand) | GAUGACUUUGAAGGGGUUUA |
| 66 | GalXC-ACAC-6331 (antisense strand) | UAAACCCCUUCAAAGUCAUCGG |
| 67 | GalXC-ACAC-6683 (sense strand) | UGGAUUCUGAAGCCAAGAUA |
| 68 | GalXC-ACAC-6683 (antisense strand) | UAUCUUGGCUUCAGAAUCCAGG |
| 69 | GalXC-ACAC-6684 (sense strand) | GGAUUCUGAAGCCAAGAUAA |
| 70 | GalXC-ACAC-6684 (antisense strand) | UUAUCUUGGCUUCAGAAUCCGG |
| 71 | GalXC-ACAC-6685 (sense strand) | GAUUCUGAAGCCAAGAUAAA |
| 72 | GalXC-ACAC-6685 (antisense strand) | UUUAUCUUGGCUUCAGAAUCGG |
| 73 | GalXC-ACAC-6686 (sense strand) | AUUCUGAAGCCAAGAUAAUA |
| 74 | GalXC-ACAC-6686 (antisense strand) | UAUUAUCUUGGCUUCAGAAUGG |
| 75 | GalXC-ACAC-6755 (sense strand) | AGGCCAUCAAGGACUUCAAA |
| 76 | GalXC-ACAC-6755 (antisense strand) | UUUGAAGUCCUUGAUGGCCUGG |
| 77 | GalXC-ACAC-6759 (sense strand) | CAUCAAGGACUUCAACCGGA |
| 78 | GalXC-ACAC-6759 (antisense strand) | UCCGGUUGAAGUCCUUGAUGGG |
| 79 | GalXC-ACAC-6761 (sense strand) | UCAAGGACUUCAACCGGGAA |
| 80 | GalXC-ACAC-6761 (antisense strand) | UUCCCGGUUGAAGUCCUUGAGG |
| 81 | GalXC-ACAC-6793 (sense strand) | AUGGUCUUUGCCAACUGGAA |
| 82 | GalXC-ACAC-6793 (antisense strand) | UUCCAGUUGGCAAAGACCAUGG |
| 83 | GalXC-ACAC-6845 (sense strand) | ACCAAGUGCUGAAGUUUGGA |
| 84 | GalXC-ACAC-6845 (antisense strand) | UCCAAACUUCAGCACUUGGUGG |
| 85 | GalXC-DGAT2-1479 (sense strand) | UGUGGGUUAUUUAAAAGAAA |
| 86 | GalXC-DGAT2-1479 (antisense strand) | UUUCUUUUAAAUAACCCACAGG |
| 87 | GalXC-DGAT2-1502 (sense strand) | GUUAUUUAAAAGAAAUUAUA |
| 88 | GalXC-DGAT2-1502 (antisense strand) | UAUAAUUUCUUUUAAAUAACGG |
| 89 | GalXC-DGAT2-0682 (sense strand) | ACCAGGAACUAUAUCUUUGA |
| 90 | GalXC-DGAT2-0682 (antisense strand) | UCAAAGAUAUAGUUCCUGGUGG |
| 91 | GalXC-DGAT2-0685 (sense strand) | AGGAACUAUAUCUUUGGAUA |
| 92 | GalXC-DGAT2-0685 (antisense strand) | UAUCCAAAGAUAUAGUUCCUGG |
| 93 | GalXC-DGAT2-0656 (sense strand) | UGGUGAAGACACACAACCUA |
| 94 | GalXC-DGAT2-0656 | UAGGUUGUGUGUCUUCACCAGG |
| | (antisense strand) | |
| 95 | GalXC-DGAT2-0684 (sense strand) | CAGGAACUAUAUCUUUGGAA |
| 96 | GalXC-DGAT2-0684 (antisense strand) | UUCCAAAGAUAUAGUUCCUGGG |
| 97 | GalXC-DGAT2-0683 (sense strand) | CCAGGAACUAUAUCUUUGGA |
| 98 | GalXC-DGAT2-0683 (antisense strand) | UCCAAAGAUAUAGUUCCUGGGG |
| 99 | GalXC-DGAT2-0676 (sense strand) | CUGACCACCAGGAACUAUAA |
| 100 | GalXC-DGAT2-0676 (antisense strand) | UUAUAGUUCCUGGUGGUCAGGG |
| 101 | GalXC-DGAT2-1120 (sense strand) | GUCCAGAAGAAGUUCCAGAA |
| 102 | GalXC-DGAT2-1120 (antisense strand) | UUCUGGAACUUCUUCUGGACGG |
| 103 | GalXC-DGAT2-1067 (sense strand) | AGAAUGAGGUAUACAAGCAA |
| 104 | GalXC-DGAT2-1067 (antisense strand) | UUGCUUGUAUACCUCAUUCUGG |
| 105 | GalXC-DGAT2-1452 (sense strand) | CUUGAGUGCAUUUUGUUCUA |
| 106 | GalXC-DGAT2-1452 (antisense strand) | UAGAACAAAAUGCACUCAAGGG |
| 107 | GalXC-DGAT2-1533 (sense strand) | CAUUGCAAUGUUAGAUGUUA |
| 108 | GalXC-DGAT2-1533 | UAACAUCUAACAUUGCAAUGGG |
| | (antisense strand) | |
| 109 | GalXC-DGAT2-1073 (sense strand) | AGGUAUACAAGCAGGUGAUA |
| 110 | GalXC-DGAT2-1073 (antisense strand) | UAUCACCUGCUUGUAUACCUGG |
| 111 | GalXC-DGAT2-1323 (sense strand) | GGUGAAGCUCUUUGACAAUA |
| 112 | GalXC-DGAT2-1323 (antisense strand) | UAUUGUCAAAGAGCUUCACCGG |
| 113 | GalXC-DGAT2-0886 (sense strand) | AUAGACUACUUGCUCUCCAA |
| 114 | GalXC-DGAT2-0886 (antisense strand) | UUGGAGAGCAAGUAGUCUAUGG |
| 115 | GalXC-DGAT2-1463 (sense strand) | CUGUAAAUUUGGAAGCGUCA |
| 116 | GalXC-DGAT2-1463 (antisense strand) | UGACGCUUCCAAAUUUACAGGG |
| 117 | GalXC-DGAT2-1139 (sense strand) | AAGAAGUUCCAGAAAUACAA |
| 118 | GalXC-DGAT2-1139 (antisense strand) | UUGUAUUUCUGGAACUUCUUGG |
| 119 | GalXC-DGAT2-0899 (sense strand) | AUAGACUAUUUGCUUUCAAA |
| 120 | GalXC-DGAT2-0899 (antisense strand) | UUUGAAAGCAAAUAGUCUAUGG |
| 121 | GalXC-DGAT2-1080 (sense strand) | AGAAUGAAGUGUACAAGCAA |
| 122 | GalXC-DGAT2-1080 | UUGCUUGUACACUUCAUUCUGG |
| | (antisense strand) | |
| 123 | GalXC-DGAT2-1086 (sense strand) | AAGUGUACAAGCAGGUGAUA |
| 124 | GalXC-DGAT2-1086 (antisense strand) | UAUCACCUGCUUGUACACUUGG |
| 125 | GalXC-DGAT2-0898 (sense strand) | CAUAGACUAUUUGCUUUCAA |
| 126 | GalXC-DGAT2-0898 (antisense strand) | UUGAAAGCAAAUAGUCUAUGGG |
| 127 | GalXC-DGAT2-0894 (sense strand) | ACACCAUAGACUAUUUGCUA |
| 128 | GalXC-DGAT2-0894 (antisense strand) | UAGCAAAUAGUCUAUGGUGUGG |
| 129 | GalXC-DGAT2-1074 (sense strand) | UUGGAGAGAAUGAAGUGUAA |
| 130 | GalXC-DGAT2-1074 (antisense strand) | UUACACUUCAUUCUCUCCAAGG |
| 131 | GalXC-DGAT2-1083 (sense strand) | AUGAAGUGUACAAGCAGGUA |
| 132 | GalXC-DGAT2-1083 (antisense strand) | UACCUGCUUGUACACUUCAUGG |
| 133 | GalXC-DGAT2-0895 (sense strand) | CACCAUAGACUAUUUGCUUA |
| 134 | GalXC-DGAT2-0895 (antisense strand) | UAAGCAAAUAGUCUAUGGUGGG |
| 135 | GalXC-DGAT2-0897 (sense strand) | CCAUAGACUAUUUGCUUUCA |
| 136 | GalXC-DGAT2-0897 | UGAAAGCAAAUAGUCUAUGGGG |
| | (antisense strand) | |
| 137 | GalXC-DGAT2-0509 (sense strand) | CUCAUGUACAUAUUCUGCAA |
| 138 | GalXC-DGAT2-0509 (antisense strand) | UUGCAGAAUAUGUACAUGAGGG |
| 139 | GalXC-DGAT2-0893 (sense strand) | GACACCAUAGACUAUUUGCA |
| 140 | GalXC-DGAT2-0893 (antisense strand) | UGCAAAUAGUCUAUGGUGUCGG |
| 141 | GalXC-DGAT2-1138 (sense strand) | GAAGAAGUUCCAGAAAUACA |
| 142 | GalXC-DGAT2-1138 (antisense strand) | UGUAUUUCUGGAACUUCUUCGG |
| 143 | GalXC-DGAT2-0896 (sense strand) | ACCAUAGACUAUUUGCUUUA |
| 144 | GalXC-DGAT2-0896 (antisense strand) | UAAAGCAAAUAGUCUAUGGUGG |
| 145 | GalXC-DGAT2-0506 (sense strand) | AUCCUCAUGUACAUAUUCUA |
| 146 | GalXC-DGAT2-0506 (antisense strand) | UAGAAUAUGUACAUGAGGAUGG |
| 147 | GalXC-DGAT2-1079 (sense strand) | GAGAAUGAAGUGUACAAGCA |
| 148 | GalXC-DGAT2-1079 (antisense strand) | UGCUUGUACACUUCAUUCUCGG |
| 149 | DGAT2 Mouse (Mm) NM 026384.3 (GenBank RefSeq #) | CCGCGCTTCGCTGGCTTTCTGCTCATCTAGGGTGGCAGC GGCTACCTACCTCAGCTCTCGCCCTGCTGCCGCCACGGC CTGGGCGCTGTCCCTCAGCTCCCGGAGCTCAGCGCGAAG CCCTGGCCCCGGCGGCCGGGGCATGGGTCAGGGGCGCG GCGTGAGGCGGCTTTCTGCACGGCCGTGACGTGCATTGG CTTCAGCATGAAGACCCTCATCGCCGCCTACTCCGGGGT |
| | | CCTGCGGGGTGAGCGTCGGGCGGAAGCTGCCCGCAGCG AAAACAAGAATAAAGGATCTGCCCTGTCACGCGAGGGG TCTGGGCGATGGGGCACTGGCTCCAGCATCCTCTCAGCC CTCCAAGACATCTTCTCTGTCACCTGGCTCAACAGATCT AAGGTGGAAAAACAGCTGCAGGTCATCTCAGTACTACA ATGGGTCCTATCCTTCCTGGTGCTAGGAGTGGCCTGCAG TGTCATCCTCATGTACACCTTCTGCACAGACTGCTGGCTG ATAGCTGTGCTCTACTTCACCTGGCTGGCATTTGACTGG AACACGCCCAAGAAAGGTGGCAGGAGATCGCAGTGGGT GCGAAACTGGGCCGTGTGGCGCTACTTCCGAGACTACTT TCCCATCCAGCTGGTGAAGACACACAACCTGCTGACCAC CAGGAACTATATCTTTGGATACCACCCCCATGGCATCAT GGGCCTGGGTGCCTTCTGTAACTTCAGCACAGAGGCTAC TGAAGTCAGCAAGAAGTTTCCTGGCATAAGGCCCTATTT GGCTACGTTGGCTGGTAACTTCCGGATGCCTGTGCTTCG CGAGTACCTGATGTCTGGAGGCATCTGCCCTGTCAACCG AGACACCATAGACTACTTGCTCTCCAAGAATGGGAGTGG CAATGCTATCATCATCGTGGTGGGAGGTGCAGCTGAGTC CCTGAGCTCCATGCCTGGCAAGAACGCAGTCACCCTGAA GAACCGCAAAGGCTTTGTGAAGCTGGCCCTGCGCCATGG AGCTGATCTGGTTCCCACTTATTCCTTTGGAGAGAATGA GGTATACAAGCAGGTGATCTTTGAGGAGGGTTCCTGGGG CCGATGGGTCCAGAAGAAGTTCCAGAAGTATATTGGTTT CGCCCCCTGCATCTTCCATGGCCGAGGCCTCTTCTCCTCT GACACCTGGGGGCTGGTGCCCTACTCCAAGCCCATCACC ACCGTCGTGGGGGAGCCCATCACTGTCCCCAAGCTGGAG CACCCGACCCAGAAAGACATCGACCTGTACCATGCCATG TACATGGAGGCCCTGGTGAAGCTCTTTGACAATCACAAG ACCAAATTTGGCCTTCCAGAGACTGAGGTGCTGGAGGTG AACTGACCCAGCCCTCGCGTGCCAGCTCCTGGGAGGGAC GACTGCAGATCCTTTTCTACCGAGTTCTTGAGTGCATTTT GTTCTGTAAATTTGGAAGCGTCATGGGTGTCTGTGGGTT ATTTAAAAGAAATTATAATTTGTTAAACCATTGCAATGT TAGATGTTTTTTAAGAAGGGAAGAGTCAGTATTTTAAGC TCACTTCTAGTGTGTCCTGCTCAAGGTGGAGGCTGATAT TTATGGGCCTTGGTGGTTTCTTACCCACCCCTTCTAGCGT TCCCCAGACGACAGACACTTGGCCCTGGCTAGCTGGGCA AGGGCAGTCCTTAGTGACTCCAGGGATTCTTGAGAGGCA GAGGCCATGTCCCACCCGTGGCTGCAGGTCGGGTTCCTC GTACCAAGGGGAGGCTGAGGGCACAGCTGGCCCCACTT GGGGAGGGTAGATAACATCTGGACTGCCCGGCTTGGGTC TCTGCTCCTCACCCTAGCCCTCTTCTCCAATCTGAGCCTA CCCTGGCCTCCTGTCTCCTGGCTAGGGACACGGCTGTCC CACAGGTGCCGTCTTGGGTTATCTCGCTGCTGTTGGCTG GTTTCACTCTGGAGGTTGGCACCATGGACACAGCTCAGC GTTGCTCTGGCGCATATCCTCCTGAGCCACACCCCAAGT CTGGTGTGAGGAAGGGCTTCTCTTCTCTTCACAGAGGTG CCTGGCTTCCTGTGCAGCACACTGGGTCCAGGACAGGAG GCCCCCCCCCCAAACCAAGCCTCACGTGTGTGCCTTTAT GAGGCGTTGGGAGAAAGCTACCCTCCTGTGTATTCTGTT |
| | | TTCTCCATGAGATTGTTGTGCCATGTCACACTTTTGTATA TTCCTAGACTAATAAATGGAAACAAGAA |
| 150 | ACACA Human (Hs) NM_198834 (GenBank RefSeq #) | GCCGCCTCCGCCCCTCGGCCGTGGAGGCCCCCGCCGGGT GCTGAGCGCCCGCCACCGCCCTCTTGGCCTTTTCCCGGTC CCCGCCGCGCGCCTGCTGCTGTCCCCGTGCCCGAGGACT GGGAGGATGGGGCTGAGCCGCTTGCCTGACTTTTGATCC GACCAGTAATCACTTTGCCCGTGTGGCGCGTGGCCCGTT GCCTGAGGCTTCCTGGCGCGTGGGGCTGTTTGGTCCCCT CCGGAGTGCGGTGAGGCGGGCCGAGCCGGGACTGCCTG GGTTTGGGGATAACGTTCCCATCTCCACCCCTGTTGCAG CAAGGGAAATTGAGGCTGAGGGAACTGGGCCCAGGGAC GGCGAGCCGTGGCGCCTCTCCAGTCCGGGCCCCGAAGG GCTGCTCGTGGATGAACCAGACTGATTTTAAGGGGTGAA GAGGGTGCGTTTCAATCAGATGCTCCTGGAACGTCGAAA TTGTCTTCTTTGGAAAGAACCATCCCCTCTTTGGGCTTCA GAGGCCCAAATTGAGGCGCAATGATGAGAGGATGTGGT GGTCTACTCTGATGTCAATCTTGAGGGCTAGGTCTTTTTG GAAGTGGATATCTACTCAGACAGTAAGAATTATAAGAG CTGTAAGAGCTCATTTTGGAGGAATAATGGATGAACCAT CTCCCTTGGCCCAACCTCTGGAGCTGAACCAGCACTCTC GATTCATAATAGGTTCTGTGTCTGAAGATAACTCAGAGG ATGAGATCAGCAACCTGGTGAAGTTGGACCTACTGGAG GAGAAGGAGGGCTCCTTGTCACCTGCTTCTGTTGGCTCA GATACACTCTCTGATTTGGGGATCTCTAGCCTACAGGAT GGCTTGGCCTTGCACATAAGGTCCAGCATGTCTGGCTTG CACCTAGTAAAGCAGGGCCGAGACAGAAAGAAAATAGA TTCTCAACGAGATTTCACTGTGGCTTCTCCAGCAGAATTT GTTACTCGCTTTGGGGGAAATAAAGTGATTGAGAAGGTT CTTATTGCTAACAATGGCATTGCAGCAGTGAAATGCATG CGGTCTATCCGTAGGTGGTCTTATGAAATGTTTCGAAAT GAACGTGCAATTAGATTCGTTGTCATGGTCACACCTGAA GACCTTAAAGCCAATGCAGAATACATTAAGATGGCAGA TCACTATGTGCCAGTGCCTGGAGGACCAAACAACAACA ACTATGCAAATGTGGAATTAATTCTTGATATTGCTAAAA GGATCCCAGTACAAGCAGTGTGGGCTGGCTGGGGTCATG CTTCTGAGAATCCCAAACTACCGGAACTTCTCTTGAAAA ATGGCATTGCCTTCATGGGTCCTCCAAGCCAGGCCATGT GGGCTTTAGGGGATAAGATTGCATCTTCCATAGTGGCTC AAACTGCAGGTATCCCAACTCTTCCCTGGAGCGGCAGTG GTCTTCGTGTGGACTGGCAGGAAAATGATTTTTCAAAAC GTATCTTAAATGTTCCCCAGGAGCTATATGAAAAAGGTT ATGTGAAAGATGTGGATGATGGGCTACAGGCAGCTGAG GAAGTTGGATATCCAGTAATGATCAAGGCCTCAGAGGG AGGAGGAGGGAAGGGAATTAGAAAAGTCAACAATGCAG ATGACTTCCCTAATCTCTTCAGACAGGTTCAAGCTGAAG TTCCTGGATCTCCCATATTTGTGATGAGACTAGCCAAAC AATCTCGTCATCTGGAGGTGCAGATCTTAGCGGACCAAT ATGGCAATGCTATCTCTTTGTTTGGTCGTGATTGCTCTGT ACAACGCAGGCATCAGAAGATTATTGAAGAAGCACCTG CTACTATTGCTACTCCAGCAGTATTTGAACACATGGAAC |
| | | AGTGTGCGGTGAAACTTGCCAAAATGGTGGGTTATGTGA GTGCTGGGACTGTGGAATACCTGTACAGCCAGGATGGCA GCTTCTACTTTCTGGAATTGAATCCTCGGCTGCAGGTAG AGCACCCTTGTACAGAGATGGTGGCTGATGTCAATCTCC CTGCAGCACAGCTCCAGATTGCCATGGGGATTCCTCTAT ATAGAATCAAGGATATCCGTATGATGTATGGGGTATCTC CCTGGGGTGATTCTCCCATTGATTTTGAAGATTCTGCACA CGTTCCTTGTCCAAGGGGCCATGTTATTGCTGCTCGGATC ACTAGTGAAAATCCAGATGAGGGTTTTAAGCCCAGCTCA GGAACAGTTCAGGAGCTAAATTTCCGCAGCAATAAGAA TGTTTGGGGATATTTCAGTGTTGCTGCTGCAGGGGGACT TCATGAATTTGCTGATTCTCAGTTTGGTCACTGCTTTTCT TGGGGAGAAAACAGAGAAGAGGCAATTTCAAACATGGT GGTGGCTTTGAAGGAGCTGTCTATTCGGGGTGACTTTCG AACTACAGTTGAATACCTGATCAAATTGTTAGAGACTGA AAGCTTTCAGATGAACAGAATTGATACTGGCTGGCTGGA CAGACTGATAGCAGAAAAAGTACAGGCTGAGCGACCTG ACACCATGTTGGGGGTTGTGTGTGGTGCCCTCCACGTGG CAGATGTGAGCCTGCGGAATAGCGTCTCTAACTTCCTTC ACTCCTTAGAAAGGGGTCAAGTCCTTCCTGCTCATACAC TTCTGAATACAGTAGATGTTGAACTTATCTATGAGGGAG TCAAGTATGTACTTAAGGTGACTCGACAGTCCCCCAACT CCTATGTGGTGATCATGAATGGCTCATGTGTAGAAGTAG ATGTACATCGGCTGAGTGACGGTGGACTGCTCTTGTCCT ATGATGGCAGCAGTTATACTACGTATATGAAAGAGGAA GTGGATAGATATCGCATCACAATTGGCAATAAAACCTGT GTGTTTGAGAAGGAAAATGACCCATCGGTGATGCGCTCA CCTTCTGCTGGGAAGTTAATCCAGTACATTGTAGAAGAT GGAGGTCATGTGTTTGCCGGCCAGTGCTATGCTGAGATT GAGGTAATGAAGATGGTAATGACCTTAACAGCTGTGGA GTCTGGCTGTATCCATTACGTCAAGCGACCTGGAGCAGC TCTTGACCCTGGCTGTGTACTAGCCAAAATGCAACTGGA CAACCCCAGCAAGGTTCAGCAGGCTGAACTTCACACAG GTAGTCTGCCACGGATCCAGAGCACGGCACTCAGAGGC GAGAAACTCCATCGAGTGTTCCATTATGTCCTGGATAAT CTGGTCAATGTAATGAATGGATACTGCCTTCCAGATCCT TTCTTTAGCAGCAAGGTAAAAGACTGGGTAGAGCGATTG ATGAAAACCCTCAGAGATCCCTCCCTGCCTCTCCTAGAA TTGCAAGATATTATGACCAGTGTGTCTGGCCGCATTCCC CCCAATGTGGAGAAGTCTATCAAGAAGGAAATGGCTCA GTATGCTAGCAACATCACATCAGTCCTCTGTCAGTTTCCC AGCCAGCAGATTGCAAACATCCTAGATAGCCATGCAGCT ACATTGAACCGGAAATCTGAACGGGAAGTCTTCTTTATG AATACTCAGAGCATTGTTCAGCTGGTACAGAGGTACCGA AGTGGCATCCGAGGCCACATGAAGGCTGTGGTGATGGA TCTGCTCCGGCAGTACCTGCGAGTAGAGACACAATTCCA GAATGGTCACTATGACAAATGTGTATTCGCCCTCCGAGA AGAGAATAAAAGTGACATGAACACTGTACTGAACTACA TCTTCTCTCACGCTCAAGTCACCAAGAAGAATCTTCTGG TCACAATGCTTATTGATCAGTTGTGTGGCCGGGACCCTA |
| | | CTCTCACTGATGAGCTGCTGAATATTCTCACAGAGCTAA CTCAACTCAGTAAGACCACCAATGCCAAAGTAGCACTTC GAGCACGCCAGGTTCTTATTGCCTCCCATTTGCCATCATA TGAGCTTCGCCATAACCAAGTAGAGTCTATCTTCCTATC AGCTATTGACATGTATGGACATCAATTTTGCATTGAGAA CCTGCAGAAACTCATCCTATCAGAAACATCTATTTTTGA TGTCCTACCAAACTTCTTCTATCACAGCAACCAAGTAGT GAGGATGGCAGCTCTGGAGGTGTATGTTCGAAGGGCTTA TATTGCCTATGAACTTAACAGCGTACAACACCGCCAGCT TAAGGACAACACCTGTGTGGTGGAATTCCAGTTCATGCT GCCCACATCTCATCCAAACAGAGGGAACATCCCTACGCT AAACAGAATGTCCTTCTCCTCCAACCTCAACCACTATGG CATGACCCATGTAGCTAGTGTCAGCGATGTACTGTTGGA CAACTCATTCACTCCACCTTGTCAGCGGATGGGCGGAAT GGTCTCTTTTCGGACTTTTGAAGATTTTGTCAGGATCTTT GATGAAGTGATGGGCTGCTTCTCTGACTCCCCACCCCAG AGTCCCACATTCCCTGAGGCAGGTCACACGTCTCTTTAT GATGAGGATAAGGTTCCCAGGGATGAACCAATTCACATT CTCAATGTGGCTATCAAGACTGACTGTGATATTGAGGAT GACAGGCTGGCAGCTATGTTCAGAGAATTTACCCAGCAA AATAAAGCTACCCTGGTTGACCATGGGATCCGGCGCCTT ACTTTCCTGGTTGCACAAAAGGATTTCAGAAAGCAGGTC AACTATGAGGTGGATCGGAGATTTCATAGAGAATTCCCT AAATTTTTTACATTCCGAGCAAGGGATAAGTTTGAGGAG GATCGTATCTATCGTCATCTGGAGCCTGCTCTGGCTTTCC AGTTAGAGCTGAACCGGATGAGAAATTTTGACCTCACTG CCATTCCATGTGCTAATCACAAGATGCACCTGTATCTCG GGGCAGCCAAGGTGGAAGTGGGCACAGAAGTGACAGAC TACAGGTTCTTTGTTCGTGCAATCATCAGGCATTCTGATC TGGTCACCAAGGAAGCTTCTTTTGAATATCTGCAAAATG AAGGGGAGCGGCTACTCCTGGAAGCCATGGATGAGTTG GAAGTTGCTTTTAACAATACAAATGTCCGCACTGACTGT AACCACATCTTCCTCAACTTTGTGCCCACGGTTATCATGG ACCCATCAAAGATTGAGGAATCCGTGCGGAGCATGGTA ATGCGGTATGGAAGTCGCCTGTGGAAATTGCGCGTCCTC CAGGCAGAACTGAAAATCAACATTCGCCTGACGCCAACT GGAAAAGCAATTCCCATCCGCCTCTTCCTGACAAACGAG TCTGGCTATTACTTGGATATCAGCCTATACAAGGAAGTG ACTGACTCCAGGACAGCACAGATCATGTTTCAGGCATAT GGAGACAAACAGGGACCACTGCATGGAATGTTAATCAA TACTCCATATGTGACCAAAGACCTGCTGCAATCAAAGAG GTTCCAGGCACAATCCTTAGGGACAACATACATATATGA TATCCCAGAGATGTTTCGGCAGTCCCTGATCAAACTCTG GGAGTCTATGTCCACTCAAGCATTTCTTCCATCTCCCCCT CTGCCTTCTGACATGCTGACTTACACTGAACTGGTACTG GATGATCAAGGTCAGCTGGTCCACATGAACAGGCTTCCA GGAGGAAATGAGATTGGCATGGTAGCTTGGAAAATGAC CTTTAAAAGTCCTGAATATCCAGAAGGCCGAGATATCAT TGTTATTGGCAATGACATCACATACCGAATTGGGTCCTT TGGGCCTCAAGAGGATTTGTTATTTCTCAGAGCTTCCGA |
| | | ACTTGCTAGGGCAGAAGGTATTCCACGCATCTATGTATC AGCCAACAGTGGAGCAAGAATCGGACTGGCAGAAGAAA TTCGCCATATGTTTCATGTGGCCTGGGTAGATCCTGAGG ATCCTTACAAGGGATACAGGTATTTATATCTGACTCCTC AAGATTATAAGAGAGTCAGTGCTCTCAACTCTGTCCATT GTGAACACGTGGAAGATGAAGGAGAATCCAGGTACAAG ATAACAGATATTATTGGGAAAGAAGAGGGAATTGGACC CGAGAACCTTCGAGGTTCTGGAATGATTGCTGGAGAATC CTCATTGGCCTATAATGAGATCATTACCATCAGCCTGGT GACGTGCCGGGCCATTGGGATTGGGGCTTACCTTGTCCG GCTGGGACAGAGAACCATCCAGGTTGAGAATTCTCACTT AATTCTAACAGGAGCTGGAGCCCTCAACAAAGTCCTCGG GCGGGAAGTGTACACCTCCAATAACCAGCTGGGGGGCA TCCAGATTATGCACAACAATGGGGTGACCCACTGCACTG TGTGTGATGACTTTGAAGGGGTTTTCACTGTCCTGCACTG GCTGTCTTACATGCCCAAGAGCGTGCACAGTTCAGTTCC TCTTCTGAACTCAAAGGATCCTATAGACAGAATCATCGA GTTTGTTCCCACAAAGACCCCATACGATCCTCGATGGAT GCTAGCAGGCCGTCCTCACCCAACCCAAAAAGGTCAGTG GTTGAGTGGCTTTTTTGACTATGGATCTTTCTCAGAGATT ATGCAGCCCTGGGCACAGACTGTGGTGGTTGGTAGAGCC AGGCTAGGAGGAATACCTGTGGGAGTTGTTGCTGTAGAA ACCCGAACAGTAGAACTAAGTATCCCAGCTGATCCAGCA AACCTGGATTCTGAAGCCAAGATAATCCAGCAGGCTGGC CAGGTTTGGTTCCCAGATTCTGCGTTTAAGACGTATCAG GCCATCAAGGACTTCAACCGGGAAGGGCTGCCTCTGATG GTCTTTGCCAACTGGAGAGGCTTCTCTGGTGGAATGAAA GATATGTACGACCAAGTGCTGAAGTTTGGTGCTTACATT GTGGATGGCTTGAGGGAGTGCTGCCAGCCTGTGCTGGTT TACATTCCTCCCCAGGCTGAGCTGCGGGGTGGCTCCTGG GTGGTGATTGACTCCTCCATCAACCCCCGGCACATGGAG ATGTATGCTGACCGAGAAAGCAGGGGATCTGTTCTGGAG CCAGAAGGGACAGTAGAAATCAAATTCCGCAGAAAGGA TCTGGTGAAAACCATGCGTCGGGTGGACCCAGTCTACAT CCACTTGGCTGAGCGATTGGGGACCCCAGAGCTAAGCAC AGCTGAGCGGAAGGAGTTGGAGAACAAGTTGAAGGAGC GGGAGGAATTCCTAATTCCCATTTACCATCAGGTAGCCG TGCAGTTTGCTGACTTGCACGACACACCAGGCCGGATGC AGGAGAAGGGTGTTATTAGCGATATCCTGGATTGGAAA ACATCCCGTACCTTCTTCTACTGGCGGCTGAGGCGTCTTC TGCTGGAGGACCTGGTCAAGAAGAAAATCCACAATGCC AACCCTGAGCTGACTGATGGCCAGATTCAAGCCATGTTA AGGCGCTGGTTTGTGGAAGTGGAAGGAACAGTGAAGGC TTATGTTTGGGACAATAATAAGGATCTGGCGGAGTGGCT AGAGAAACAGCTGACAGAGGAGGATGGTGTTCACTCGG TAATAGAGGAAAACATCAAATGCATCAGCAGAGACTAC GTCCTCAAGCAAATCCGCAGCTTGGTCCAGGCCAATCCA GAGGTTGCCATGGATTCCATCATCCATATGACGCAGCAC ATATCACCCACTCAGCGAGCAGAAGTCATACGGATCCTC TCCACAATGGATTCCCCTTCCACGTAGGAAGAGCTTCCT |
| | | GCCTGTCCCTGCCCTGTCTCTGGAGAAAAGGGCTAGAGC TGCCTTTTACAACTGTAACCACTGTAATGAGAAGGCACA GGAGACCCAGCACTGGAGTCAAATGGCATTTTACTTCCT CTCGTTTCAGGTTATGCATGACATCCTGGGATGTAAGAT CACAGAATCCCCCTCCAGCCCACCAGTCACACCTACCCC ATTCAGTATTTATTACCCTGGCCAGGCCTAGTCCTCCACT CCCTGCACAGGACTGAGAAGGCAATGAAAGGTACAAAC ATGTACCATGAGGTCTTACTAACCAAAGTAGGGCTGCCC CTCCTGTCCTGACAGCCCCTTGGCCTCCCAGCATGGGGA AGCGTGAGGAGTTGCCCAGCAGTGAGCAGCCCCCCTCAC TCCTGGCCCCATGAGCCGCAGCCACAGGCAGCAGAGGA GGGCTAAGGAGAGGAGGAAGCCTCAAGTCCATTGTTTAT TACCCCGACTCTTAGCCCAGCACACAGTAGGCACTGGAG AGGAATGATTCCCAGTTTAACCACACTACGGTACCTTTT ATGAAGAAAAATTAGAGCATAAAATCTACTACAAGCTC CATAGGAACTCAAAGATGAGGGCAAAACTGTGAGCCAA GAAGCAGAGAAAGAAAATAGAACCAGTTATTCTTGATTT AGGGGACCTCAACCTTGGGTTCAGTCTCTGAGGACAAAG GGAAAGGTAGTTGTTGGCCTGCCTCTCGCCTGCACGTCA CTGCTGGACTAGCTGTCGCATGTGGCTGGGAGCTGCAAG GCCAGTGCTTGAGGGGCCCCAGCAGTTCCACAGGTGGTG AAGCCTGAGTTGGCAGAGGAGGAGCCAGAAGAGAACTG CCCTTTCTGCACTGGTGGAAACTAGTTATTTATGCCATGT GGAGAGCCAGTGAGATAGATAGATAGTCTGTTTGTTTTG AGGACTTGGAAAGTTGTTCCTATGAAGCCTGGAGCTTGG ATGGTTTTGAGAGGTTAATGGTGCCTCCACACTCACTCTT CCCTAGTTCCAGGATTACTGTCCTAGCAGCTAACGGTTC TACTCTCTTCCCCAGAGTGTAGACAGGCAGCAGGTCTCC CCACAGCTCTGAAAGGACCCTGGTGACAGCTACACCCTC AGCACCAGGAGCTGGCCTTCCTGATGAGGGAGGCTTCCA GGAAACACAGAATCCACATGACCTTAAGATTATTTACAA CTCAGTCATGGTGCTGCTGTCCTCCAGGCTTACTGGCCCC TCCTGACTGGCATCAGGGGCTTCCTCAGGTGGTGGAGAG AGTTTACTTTCAACAACTAGTTTATTCAAGAAAAGAACT TACTGATTCCTCTGTTCCTAAAGCAAGAGTGGCAGGTGA TCAGGGCTGGTGTAGCATCCGGTTCCTTTAGTGCAGCTA ACTGCATTTGTCACTGATGACCAAGGAGGAAATCACTAA GACATTTGAGAAGCAGTGGTATGAACGTTCTTGGACAAG CCACAGTTCTGAGCCTTAACCCTGTAGTTTGCACACAAG AACGAGCTCCACCTCCCCTTCTTCAGGAGGAATCTGTGC GGATAGATTGGCTGGACTTTTCAATGGTTCTGGGTTGCA GGTGGGCACTGTATGGCTGGGTATGGAGCGGACAGCCC CCAGGAGTCAGAGCCTCAGCCCGGCTGCCCTGGTGGAA GGTACAGGTGTTCAGCACCTTCAGAAAAGGGCATAAAG TGGTGGGGGACAATTCTCAGTCCAGGAAAATGCATTGAC CATTGCTGGCTATTTGCTTACCTAGTAAGAATTGGATTCA TTTTTGACCAGATTATTCTTCTATGCTTTTTTGCAATAAA TCAAATCCCACATATCTACAAGTGGTATGAAGTCCTGCA CCCCCCAGGAGGCCTGTCCAGGCATGTCTTCAGAGGCAG GGTGGGTTACACTCATTTACCTCCCCTCTCCCCACCAAAT |
| | | TATGACACAAACGAGTATGTTTCCTCTCTAGAACCCTGT AATGCCTCCTCCCCCATCCCCAGAGCTCCTTACTGTAGGT CTTACCCTGGACAAGGATTTTTTCAAGTTGGAGGCACAG AACATGAGCAATCTGACATTCCCACAGCCCCTCAAACAT GCAAGGCTACTAAGGCAGGAGGAGTATAAATGATGGAT ATTGACCAAGACCTGCTTGGACGGAGACCGCCATATTAT CTGTTCTCTTCGTTCACAAAACAGCCTTCACTTGTCTCAG AATTTGATGGACACATACTGTGATGAGCAGGAGCTTCAG ATGCACTCTTTACACATTTTGTTGAAATAAACCTCTACAT TTGTAGAAGA |
| 151 | ACACB Human (Hs) NM_001093 (GenBank RefSeq #) | AGAGTAAGCAGCTAGCAGGCTTAGATTCAGGCCCTCAGC AAACAAGGAACCTGGAAAATGTAACCCTGAATGCACGGT GGGGAGGACATGGCAAGAGAAAAGCGGCAGGAATAAAG TGATTTTCTGAATGGTCTTGCTTCTTTGTCTATCTTGTCTG ATTTTCTCCTGTCTGACCTTTTCCTGGTTAAAAATCTGGGG GAAAATGACGGACTCCAAGCCGATCACCAAGAGTAAATC AGAAGCAAACCTCATCCCGAGCCAGGAGCCCTTTCCAGC CTCTGATAACTCAGGGGAGACACCGCAGAGAAATGGGGA GGGCCACACTCTGCCCAAGACACCCAGCCAGGCCGAGCC AGCCTCCCACAAAGGCCCCAAAGATGCCGGTCGGCGGAG AAACTCCCTACCACCCTCCCACCAGAAGCCCCCAAGAAA CCCCCTTTCTTCCAGTGACGCAGCACCCTCCCCAGAGCTT CAAGCCAACGGGACTGGGACACAAGGTCTGGAGGCCACA GATACCAATGGCCTGTCCTCCTCAGCCAGGCCCCAGGGCC AGCAAGCTGGCTCCCCCTCCAAAGAAGACAAGAAGCAGG CAAACATCAAGAGGCAGCTGATGACCAACTTCATCCTGG GCTCTTTTGATGACTACTCCTCCGACGAGGACTCTGTTGC TGGCTCATCTCGTGAGTCTACCCGGAAGGGCAGCCGGGC CAGCTTGGGGGCCCTGTCCCTGGAGGCTTATCTGACCACA GGTGAAGCTGAGACCCGCGTCCCCACTATGAGGCCGAGC ATGTCGGGACTCCACCTGGTGAAGAGGGGACGGGAACAC AAGAAGCTGGACCTGCACAGAGACTTTACCGTGGCTTCTC CCGCTGAGTTTGTCACACGCTTTGGGGGGGATCGGGTCAT CGAGAAGGTGCTTATTGCCAACAACGGGATTGCCGCCGT GAAGTGCATGCGCTCCATCCGCAGGTGGGCCTATGAGAT GTTCCGCAACGAGCGGGCCATCCGGTTTGTTGTGATGGTG ACCCCCGAGGACCTTAAGGCCAACGCAGAGTACATCAAG ATGGCGGATCATTACGTCCCCGTCCCAGGAGGGCCCAAT AACAACAACTATGCCAACGTGGAGCTGATTGTGGACATT GCCAAGAGAATCCCCGTGCAGGCGGTGTGGGCTGGCTGG GGCCATGCTTCAGAAAACCCTAAACTTCCGGAGCTGCTGT GCAAGAATGGAGTTGCTTTCTTAGGCCCTCCCAGTGAGGC CATGTGGGCCTTAGGAGATAAGATCGCCTCCACCGTTGTC GCCCAGACGCTACAGGTCCCAACCCTGCCCTGGAGTGGA AGCGGCCTGACAGTGGAGTGGACAGAAGATGATCTGCAG CAGGGAAAAAGAATCAGTGTCCCAGAAGATGTTTATGAC AAGGGTTGCGTGAAAGACGTAGATGAGGGCTTGGAGGCA GCAGAAAGAATTGGTTTTCCATTGATGATCAAAGCTTCTG AAGGTGGCGGAGGGAAGGGAATCCGGAAGGCTGAGAGT GCGGAGGACTTCCCGATCCTTTTCAGACAAGTACAGAGT |
| | | GAGATCCCAGGCTCGCCCATCTTTCTCATGAAGCTGGCCC AGCACGCCCGTCACCTGGAAGTTCAGATCCTCGCTGACCA GTATGGGAATGCTGTGTCTCTGTTTGGTCGCGACTGCTCC ATCCAGCGGCGGCATCAGAAGATCGTTGAGGAAGCACCG GCCACCATCGCCCCGCTGGCCATATTCGAGTTCATGGAGC AGTGTGCCATCCGCCTGGCCAAGACCGTGGGCTATGTGA GTGCAGGGACAGTGGAATACCTCTATAGTCAGGATGGCA GCTTCCACTTCTTGGAGCTGAATCCTCGCTTGCAGGTGGA ACATCCCTGCACAGAAATGATTGCTGATGTTAATCTGCCG GCCGCCCAGCTACAGATCGCCATGGGCGTGCCACTGCAC CGGCTGAAGGATATCCGGCTTCTGTATGGAGAGTCACCAT GGGGAGTGACTCCCATTTCTTTTGAAACCCCCTCAAACCC TCCCCTCGCCCGAGGCCACGTCATTGCCGCCAGAATCACC AGCGAAAACCCAGACGAGGGTTTTAAGCCGAGCTCCGGG ACTGTCCAGGAACTGAATTTCCGGAGCAGCAAGAACGTG TGGGGTTACTTCAGCGTGGCCGCTACTGGAGGCCTGCACG AGTTTGCGGATTCCCAATTTGGGCACTGCTTCTCCTGGGG AGAGAACCGGGAAGAGGCCATTTCGAACATGGTGGTGGC TTTGAAGGAACTGTCCATCCGAGGCGACTTTAGGACTACC GTGGAATACCTCATTAACCTCCTGGAGACCGAGAGCTTCC AGAACAACGACATCGACACCGGGTGGTTGGACTACCTCA TTGCTGAGAAAGTGCAGGCGGAGAAACCGGATATCATGC TTGGGGTGGTATGCGGGGCCTTGAACGTGGCCGATGCGA TGTTCAGAACGTGCATGACAGATTTCTTACACTCCCTGGA AAGGGGCCAGGTCCTCCCAGCGGATTCACTACTGAACCT CGTAGATGTGGAATTAATTTACGGAGGTGTTAAGTACATT CTCAAGGTGGCCCGGCAGTCTCTGACCATGTTCGTTCTCA TCATGAATGGCTGCCACATCGAGATTGATGCCCACCGGCT GAATGATGGGGGGCTCCTGCTCTCCTACAATGGGAACAG CTACACCACCTACATGAAGGAAGAGGTTGACAGTTACCG AATTACCATCGGCAATAAGACGTGTGTGTTTGAGAAGGA GAACGATCCTACAGTCCTGAGATCCCCCTCGGCTGGGAA GCTGACACAGTACACAGTGGAGGATGGGGGCCACGTTGA GGCTGGGAGCAGCTACGCTGAGATGGAGGTGATGAAGAT GATCATGACCCTGAACGTTCAGGAAAGAGGCCGGGTGAA GTACATCAAGCGTCCAGGTGCCGTGCTGGAAGCAGGCTG CGTGGTGGCCAGGCTGGAGCTCGATGACCCTTCTAAAGTC CACCCGGCTGAACCGTTCACAGGAGAACTCCCTGCCCAG CAGACACTGCCCATCCTCGGAGAGAAACTGCACCAGGTC TTCCACAGCGTCCTGGAAAACCTCACCAACGTCATGAGTG GCTTTTGTCTGCCAGAGCCCGTTTTTAGCATAAAGCTGAA GGAGTGGGTGCAGAAGCTCATGATGACCCTCCGGCACCC GTCACTGCCGCTGCTGGAGCTGCAGGAGATCATGACCAG CGTGGCAGGCCGCATCCCCGCCCCTGTGGAGAAGTCTGTC CGCAGGGTGATGGCCCAGTATGCCAGCAACATCACCTCG GTGCTGTGCCAGTTCCCCAGCCAGCAGATAGCCACCATCC TGGACTGCCATGCAGCCACCCTGCAGCGGAAGGCTGATC GAGAGGTCTTCTTCATCAACACCCAGAGCATCGTGCAGTT GGTCCAGAGATACCGCAGCGGGATCCGCGGCTATATGAA AACAGTGGTGTTGGATCTCCTGAGAAGATACTTGCGTGTT |
| | | GAGCACCATTTTCAGCAAGCCCACTACGACAAGTGTGTG ATAAACCTCAGGGAGCAGTTCAAGCCAGACATGTCCCAG GTGCTGGACTGCATCTTCTCCCACGCACAGGTGGCCAAGA AGAACCAGCTGGTGATCATGTTGATCGATGAGCTGTGTG GCCCAGACCCTTCCCTGTCGGACGAGCTGATCTCCATCCT CAACGAGCTCACTCAGCTGAGCAAAAGCGAGCACTGCAA AGTGGCCCTCAGAGCCCGGCAGATCCTGATTGCCTCCCAC CTCCCCTCCTACGAGCTGCGGCATAACCAGGTGGAGTCCA TTTTCCTGTCTGCCATTGACATGTACGGCCACCAGTTCTG CCCCGAGAACCTCAAGAAATTAATACTTTCGGAAACAAC CATCTTCGACGTCCTGCCTACTTTCTTCTATCACGCAAAC AAAGTCGTGTGCATGGCGTCCTTGGAGGTTTACGTGCGGA GGGGCTACATCGCCTATGAGTTAAACAGCCTGCAGCACC GGCAGCTCCCGGACGGCACCTGCGTGGTAGAATTCCAGT TCATGCTGCCGTCCTCCCACCCAAACCGGATGACCGTGCC CATCAGCATCACCAACCCTGACCTGCTGAGGCACAGCAC AGAGCTCTTCATGGACAGCGGCTTCTCCCCACTGTGCCAG CGCATGGGAGCCATGGTAGCCTTCAGGAGATTCGAGGAC TTCACCAGAAATTTTGATGAAGTCATCTCTTGCTTCGCCA ACGTGCCCAAAGACACCCCCCTCTTCAGCGAGGCCCGCA CCTCCCTATACTCCGAGGATGACTGCAAGAGCCTCAGAG AAGAGCCCATCCACATTCTGAATGTGTCCATCCAGTGTGC AGACCACCTGGAGGATGAGGCACTGGTGCCGATTTTACG GACATTCGTACAGTCCAAGAAAAATATCCTTGTGGATTAT GGACTCCGACGAATCACATTCTTGATTGCCCAAGAGAAA GAATTTCCCAAGTTTTTCACATTCAGAGCAAGAGATGAGT TTGCAGAAGATCGCATTTACCGTCACTTGGAACCTGCCCT GGCCTTCCAGCTGGAACTTAACCGGATGCGTAACTTCGAT CTGACCGCCGTGCCCTGTGCCAACCACAAGATGCACCTTT ACCTGGGTGCTGCCAAGGTGAAGGAAGGTGTGGAAGTGA CGGACCATAGGTTCTTCATCCGCGCCATCATCAGGCACTC TGACCTGATCACAAAGGAAGCCTCCTTCGAATACCTGCA GAACGAGGGTGAGCGGCTGCTCCTGGAGGCCATGGACGA GCTGGAGGTGGCGTTCAATAACACCAGCGTGCGCACCGA CTGCAACCACATCTTCCTCAACTTCGTGCCCACTGTCATC ATGGACCCCTTCAAGATCGAGGAGTCCGTGCGCTACATG GTTATGCGCTACGGCAGCCGGCTGTGGAAACTCCGTGTGC TACAGGCTGAGGTCAAGATCAACATCCGCCAGACCACCA CCGGCAGTGCCGTTCCCATCCGCCTGTTCATCACCAATGA GTCGGGCTACTACCTGGACATCAGCCTCTACAAAGAAGT GACTGACTCCAGATCTGGAAATATCATGTTTCACTCCTTC GGCAACAAGCAAGGGCCCCAGCACGGGATGCTGATCAAT ACTCCCTACGTCACCAAGGATCTGCTCCAGGCCAAGCGAT TCCAGGCCCAGACCCTGGGAACCACCTACATCTATGACTT CCCGGAAATGTTCAGGCAGGCTCTCTTTAAACTGTGGGGC TCCCCAGACAAGTATCCCAAAGACATCCTGACATACACT GAATTAGTGTTGGACTCTCAGGGCCAGCTGGTGGAGATG AACCGACTTCCTGGTGGAAATGAGGTGGGCATGGTGGCC TTCAAAATGAGGTTTAAGACCCAGGAGTACCCGGAAGGA CGGGATGTGATCGTCATCGGCAATGACATCACCTTTCGCA |
| | | TTGGATCCTTTGGCCCTGGAGAGGACCTTCTGTACCTGCG GGCATCCGAGATGGCCCGGGCAGAGGGCATTCCCAAAAT TTACGTGGCAGCCAACAGTGGCGCCCGTATTGGCATGGC AGAGGAGATCAAACACATGTTCCACGTGGCTTGGGTGGA CCCAGAAGACCCCCACAAAGGATTTAAATACCTGTACCT GACTCCCCAAGACTACACCAGAATCAGCTCCCTGAACTCC GTCCACTGTAAACACATCGAGGAAGGAGGAGAGTCCAGA TACATGATCACGGATATCATCGGGAAGGATGATGGCTTG GGCGTGGAGAATCTGAGGGGCTCAGGCATGATTGCTGGG GAGTCCTCTCTGGCTTACGAAGAGATCGTCACCATTAGCT TGGTGACCTGCCGAGCCATTGGGATTGGGGCCTACTTGGT GAGGCTGGGCCAGCGAGTGATCCAGGTGGAGAATTCCCA CATCATCCTCACAGGAGCAAGTGCTCTCAACAAGGTCCTG GGAAGAGAGGTCTACACATCCAACAACCAGCTGGGTGGC GTTCAGATCATGCATTACAATGGTGTCTCCCACATCACCG TGCCAGATGACTTTGAGGGGGTTTATACCATCCTGGAGTG GCTGTCCTATATGCCAAAGGATAATCACAGCCCTGTCCCT ATCATCACACCCACTGACCCCATTGACAGAGAAATTGAA TTCCTCCCATCCAGAGCTCCCTACGACCCCCGGTGGATGC TTGCAGGAAGGCCTCACCCAACTCTGAAGGGAACGTGGC AGAGCGGATTCTTTGACCACGGCAGTTTCAAGGAAATCA TGGCACCCTGGGCGCAGACCGTGGTGACAGGACGAGCAA GGCTTGGGGGGATTCCCGTGGGAGTGATTGCTGTGGAGA CACGGACTGTGGAGGTGGCAGTCCCTGCAGACCCTGCCA ACCTGGATTCTGAGGCCAAGATAATTCAGCAGGCAGGAC AGGTGTGGTTCCCAGACTCAGCCTACAAAACCGCCCAGG CCGTCAAGGACTTCAACCGGGAGAAGTTGCCCCTGATGA TCTTTGCCAACTGGAGGGGGTTCTCCGGTGGCATGAAAG ACATGTATGACCAGGTGCTGAAGTTTGGAGCCTACATCGT GGACGGCCTTAGACAATACAAACAGCCCATCCTGATCTA TATCCCGCCCTATGCGGAGCTCCGGGGAGGCTCCTGGGTG GTCATAGATGCCACCATCAACCCGCTGTGCATAGAAATGT ATGCAGACAAAGAGAGCAGGGGTGGTGTTCTGGAACCAG AGGGGACAGTGGAGATTAAGTTCCGAAAGAAAGATCTGA TAAAGTCCATGAGAAGGATCGATCCAGCTTACAAGAAGC TCATGGAACAGCTAGGGGAACCTGATCTCTCCGACAAGG ACCGAAAGGACCTGGAGGGCCGGCTAAAGGCTCGCGAGG ACCTGCTGCTCCCCATCTACCACCAGGTGGCGGTGCAGTT CGCCGACTTCCATGACACACCCGGCCGGATGCTGGAGAA GGGCGTCATATCTGACATCCTGGAGTGGAAGACCGCACG CACCTTCCTGTATTGGCGTCTGCGCCGCCTCCTCCTGGAG GACCAGGTCAAGCAGGAGATCCTGCAGGCCAGCGGGGAG CTGAGTCACGTGCATATCCAGTCCATGCTGCGTCGCTGGT TCGTGGAGACGGAGGGGGCTGTCAAGGCCTACTTGTGGG ACAACAACCAGGTGGTTGTGCAGTGGCTGGAACAGCACT GGCAGGCAGGGGATGGCCCGCGCTCCACCATCCGTGAGA ACATCACGTACCTGAAGCACGACTCTGTCCTCAAGACCAT CCGAGGCCTGGTTGAAGAAAACCCCGAGGTGGCCGTGGA CTGTGTGATATACCTGAGCCAGCACATCAGCCCAGCTGA GCGGGCGCAGGTCGTTCACCTGCTGTCTACCATGGACAGC |
| | | CCGGCCTCCACCTGACCGTGGCCCGCCCAGCCACTCCCGG GACCACGGCAAAAGGAACCACCCAGACCCACCACCCGTA CACCCTCAGCAGACCCTGAAGACTTGCTTTTAAACAAAG AAAATCCTGGGCACTTCTGCAGGGCTGCTGGTTCCGAGCT GACACCCGTCTTAACAAAAGGCCCAGGAGTGCCTCTTCC AAACAAAAACAGCCTCCTCTCCATAGCTGGGAAGTTTATT TTGTTTTGTCTCTGAAGACAGCAGTTTTATTGCATCACTA AATCTAATCAAGCTAAAACATCCCTGTTTCCTTTTGCAAA ACAGTGCCTGGCATGTGGGATCCAGGCGTTCTTTAGGATC CTTGGATACCACATCGTGAAATCTTTTATTTTTTTACTCTG AGACCAGCACCAGATGTAAGTAAGCATCTCATATATTTCA GCCAAATAAATGGGCCAAGGGAAAAAAATATATATATAT AGACAGGACTAGAGAAAACCTATTTTTGTAATGATGTTTC TTTGGATACTGTCTAGTCACCCAGAAAAATGTATGGATGA ATTTTTTTTTTTTTTTTTGAGACAAAGTCTCACTGTGTCAT GCAGGCTGGAGTGCAGTGGCATGATCTCACTGCAACCTC CATCTCCTGTCTCAGCCTCCTAGATAACTGGGATTACAGG TGCCCACCACCATGCCCGGCTAATTTTTGTATTTTTGGTA GAGACAGAGTTTCACCAGGTTGGTCAGGCTGGTCTCAAA CTCTTGACTTCAGGTAATCCACCCACCTTGGCCTCCCAAA GTGCTGGGATTACAGGCATGAGCCACCATCTTCAGCCAG ATGATTTTTTTATTGAGAGAGTGAAATGCTATTTTGTTCCC CAAATGGCGCTAGTGAATCACTAGGAGGGTCCCACTGAT AGGCCATGTTTAGCACTGGTTGCCAGGGATTCTCTTTTTG AGAGAGGGAAAGCAAAATGAATGGAAGTACCCAGCTGG AGGTTTCAGGGCTTCTGGAGGATGCTCTCGCATAGCTCGA GGTCCTCTGCCCACCTCTTCTCTCCAAGGAAAATGAGGAC TGCCCCTTCCCCCTGCAGGATTGGCCCCCAGCCTGCGCAT GCACCCTCCTCTTGCCCAAGTGGGGAGCACAGAGGCGGA GAGGAATCCCTTACCACACCCACGGCCCAGCTTGCTCACG AGTGTCACCTCTGTGACGGTCACCACTGCTCCCTTGGAGG GCCACTTGAGTTACTGTTGCTTCCTCGCCTGCTGGCTTGAT GAGCACCGATGGTGGGATCTGACCCCGAGGGGCAGAGCT GTCGGTGACTGAGGACTGGACTGTGGTGACCATGCCGAT TTGCTCAGGGAGAACGTTGCAATGCACCCAGCAGCTCCT GGCTCTGCAGGCGGCACAGCCTGGGGCCCTGTGATCCTCT GGTTTCTTCCATTGGGGCGGAGTCGGGGATGGAGGGAGC TGGCCACAACCCACTGCTCTGATGGGTGGTTTGTCCAAGG ATGCTGAATGTAATGCCTGGTCAATGTGGAAGCCCATGA GGTTGCCCAGGGAAGCCTCCAAAAGCTGGGATGCTTGAG GGTATCCAAGTTGAAAAAGACAAAATCTGACCATCAGCC AGTGACAGTCCTGGCAAATGAAGGTGGGGCGGGGCAGTG AGGGGTGGGAGAAGGTGAATGATTCATTATTCCACCCCG AGGTTTGCTGGGGTGAGGGGAAGAATCGATGCTGCTTTG GGAACTGAAGGTTTTTCTGTTGGGAAGGCCCTCTTGGTTT TGGAGAGAAAGACAAGTTATGAGTAGCTGCTACCCTGGA ACGGTGGGCAGAGAGCCTACTAGGAAATGTGCAGAATAA ACTATTTTTTGAAGGAAA |
| 152 | ACACA | GCCCCTCGGCTGTGGAGGCCCCCAGCGGGTGCTGAGCGC CAGCCGCTGCCCCCTTGGCCTTTTCCCGGCCACCGCCCG |
| | Cynomolgus monkey (Mf) XM_01543840 8 (GenBank RefSeq #) | GCGCCCGTCGCTGTCCCTGTGCCTGAGGACTGGGAGGAT GGGGCTAAGCCGCTTGCCTGACTTTTGATCCGCCAAGTA ATCACTTTGTCCGTGTGGCGCGTGGCCCGTTGCCGGAGG CTTCCCAGCGCGTGGGGCCGTTTGGTCCCCTCCGGAGTG CGGTGAGGCGGGCCAAGCCGGGACTGCCTGGGTTTGGG GATAACGTTCCCACCCCCACCCCTGTTGCAGCAAGGGAA ATTGAGGCTGAGGGAACTGGGCCCAGGGACGGCGAGCC GTGGCTGCCTCTCCAGTCCGGGCCCTGAAGGGCTGCTCG TGGATGAACCAGACTGATTTTTAAGGGGTGAAGAGGGT GCGTTTCAATCAGATGCTTCTGGAACGTCGAAATTGTCT TCTTTGGAAAGAACCATCCCCTCTTTGGGCTTCAGAGGC CCAAATTGAGGCGCGATGATGAGAGGATGTGGTGGTCT ACTCTGATGTCAATCTTGAGGGCTAGCTCTAAGAGCTCA TTTTGGAGGAATAATGGATGAACCATCTCCCTTGGCCAA ACCTCTGGAGCTGAACCAGCACTCTCGATTCATAATTGG TTCTGTGTCTGAAGATAACTCAGAGGATGAGATCAGCAA CCTGGTGAAGCTGGACCTACTGGAGGAGAAGGAGGGTT CTTTGTCACCTGCTTCTGTTGGCTCAGATACACTCTCTGA TTTGGGGATCTCTAGCCTACAGGATGGCCTGGCCTTGCA CATAAGGTCCAGCATGTCTGGCTTGCACCTAGTAAAGCA GGGCCGAGACAGAAAGAAAATAGATTCTCAACGAGATT TCACTGTGGCTTCTCCAGCAGAATTTGTTACTCGCTTTGG GGGAAATAAAGTGATTGAGAAGGTTCTTATTGCTAACAA TGGCATTGCAGCAGTGAAATGCATGCGGTCTATCCGTAG GTGGTCTTATGAAATGTTTCGAAATGAACGTGCAATTCG ATTCGTTGTCATGGTCACACCTGAAGACCTTAAAGCCAA TGCAGAATACATTAAGATGGCAGATCACTACGTGCCGGT GCCTGGAGGACCAAACAACAACAACTATGCAAATGTGG AATTAATTCTTGATATTGCTAAAAGGATCCCAGTACAAG CAGTGTGGGCTGGCTGGGGTCATGCTTCTGAGAATCCCA AACTACCGGAACTTCTCTTGAAAAATGGCATTGCCTTCA TGGGTCCTCCAAGCCAGGCCATGTGGGCTTTAGGGGATA AGATTGCATCTTCCATAGTGGCTCAAACTGCAGGTATCC CAACTCTTCCCTGGAGTGGCAGTGGTCTTCGTATGGACT GGCAGGAAAATGATTTTTCAAAACGTATCTTAAATGTTC CCCAGGAGCTATATGAAAAAGGTTATGTGAAAGATGTG GATGACGGGCTACAGGCAGCTGAGGAAGTTGGATATCC AGTAATGATCAAGGCCTCAGAAGGAGGAGGAGGGAAGG GAATTAGAAAAGTCAACAATGCAGATGACTTCCCTAACC TCTTCAGACAGGTTCAAGCTGAAGTCCCTGGATCTCCCA TATTTGTGATGAGACTAGCCAAACAATCTCGTCATCTGG AGGTGCAGATCTTAGCGGACCAATATGGCAATGCCATCT CTTTGTTTGGTCGTGATTGCTCTGTACAACGCAGGCATCA GAAGATTATTGAAGAGGCACCAGCTACTATTGCTACTCC AGCAGTATTTGAACACATGGAACAGTGTGCGGTGAAACT TGCCAAAATGGTGGGTTATGTGAGTGCTGGGACTGTGGA ATACCTGTACAGCCAGGACGGCAGCTTCTACTTTCTGGA ATTGAATCCTCGGCTACAGGTAGAGCACCCTTGTACAGA GATGGTGGCTGATGTCAATCTCCCTGCAGCTCAGCTCCA GATTGCCATGGGGATTCCTCTATATAGAATCAAGGATAT |
| | | CCGTATGATGTACGGGGTATCTCCCTGGGGTGATTCTCC CATTGATTTTGAAGATTCTGCACATGTTCCTTGCCCAAGG GGCCATGTTATTGCTGCTCGGATCACTAGTGAAAATCCA GATGAGGGTTTTAAGCCCAGTTCAGGAACAGTTCAGGAA CTAAATTTCCGCAGCAATAAGAATGTTTGGGGATACTTC AGTGTTGCTGCTGCAGGGGGACTTCATGAATTTGCTGAT TCTCAGTTTGGTCACTGCTTTTCTTGGGGAGAAAACAGA GAGGAGGCAATTTCAAACATGGTGGTGGCTTTGAAGGA GCTGTCTATTCGGGGTGACTTTCGAACTACAGTTGAATA CCTGATCAAATTGTTAGAGACTGAAAGCTTTCAGATGAA CAGAATTGATACTGGCTGGCTGGACAGACTGATAGCAG AAAAAGTACAGGCTGAGCGACCTGACACCATGTTGGGG GTTGTATGTGGGGCCCTCCACGTGGCAGACGTGAGCCTG CGGAATAGCGTCTCTAACTTCCTTCACTCCTTAGAAAGG GGTCAAGTCCTTCCTGCTCATACACTGTTGAATACAGTA GATGTTGAACTTATCTATGAGGGAGTCAAGTATGTACTT AAGGTGACTCGACAGTCCCCCAACTCCTATGTGGTGATC ATGAATGGCTCATGTGTAGAAGTAGATGTACATCGGCTG AGTGACGGTGGACTGCTCTTGTCCTATGATGGCAGCAGT TATACTACGTACATGAAGGAGGAAGTGGATAGATATCG CATCACAATTGGCAATAAAACCTGTGTGTTTGAGAAGGA AAATGACCCATCAGTGATGCGCTCACCTTCTGCTGGGAA GTTAATCCAGTACATTGTAGAAGATGGAGGTCATGTGTT TGCTGGCCAGTGCTATGCTGAGATTGAGGTGATGAAGAT GGTAATGACCTTAACAGCTGTGGAGTCTGGCTGTATCCA TTACGTCAAGCGACCTGGAGCAGCTCTTGACCCTGGCTG TGTACTAGCCAGAATGCAACTGGACAACCCCAGCAAGG TTCAGCAGGCTGAGCTTCACACAGGTAGTCTGCCACGGA TCCAGAGCACGGCACTCAGAGGCGAGAAACTGCATCGA GTGTTCCATTATGTCCTGGATAATCTGGTCAACGTAATG AATGGATACTGCCTTCCAGATCCTTTCTTTAGCAGCAAG GTAAAAGACTGGGTAGAGCGATTGATGAAAACCCTCAG AGATCCCTCCCTGCCTCTCCTAGAATTGCAAGATATCAT GACTAGCGTGTCTGGCCGCATTCCCCCCAATGTGGAGAA GTCTATCAAGAAGGAAATGGCTCAGTATGCTAGCAACAT CACATCAGTCCTCTGTCAGTTTCCCAGTCAGCAGATTGC CAACATCCTAGATAGCCATGCAGCTACATTGAACCGGAA ATCTGAACGGGAGGTCTTCTTTATGAATACTCAGAGCAT TGTTCAGCTGGTACAGAGGTACCGAAGTGGCATCCGAGG CCACATGAAGGCTGTGGTGATGGATCTGCTCCGGCAGTA CCTGCGAGTAGAGACACAATTCCAGAATGGTCACTATGA CAAATGTGTATTCGCCCTCCGGGAAGAGAATAAAAGTG ACATGAACACTGTACTGAACTACATCTTCTCTCACGCTC AAGTCACCAAGAAGAATCTTCTGGTCACAATGCTTATCG ATCAGTTGTGTGGCCGGGACCCTACTCTCACTGATGAGC TGCTGAATATTCTCACAGAGCTAACTCAACTCAGTAAGA CTACCAATGCTAAAGTAGCACTTCGAGCACGCCAGGTTC TTATTGCCTCCCATTTGCCATCATACGAGCTTCGCCATAA CCAAGTAGAGTCTATCTTCCTATCAGCTATTGACATGTAT GGACATCAATTTTGCATTGAGAACCTGCAGAAACTCATC |
| | | CTATCAGAAACATCTATTTTTGATGTCCTACCAAACTTCT TCTATCACAGCAACCAAGTAGTGAGGATGGCAGCTCTGG AGGTGTATGTTCGAAGGGCTTATATTGCCTACGAACTTA ACAGCGTACAACACCGCCAGCTTAAGGACAACACCTGT GTGGTGGAATTCCAGTTCATGCTGCCCACATCTCATCCA AACAGAGGGAACATCCCTACGCTAAACAGAATGTCCTTC TCCTCCAACCTCAACCACTATGGCATGACCCATGTAGCT AGTGTCAGCGATGTGCTGTTGGACAACTCATTCACTCCA CCTTGTCAGCGGATGGGCGGAATGGTCTCTTTTCGGACT TTCGAAGATTTTGTCAGGATCTTTGATGAAGTGATGAGC TGCTTCTCTGACTCCCCACCCCAGAGTCCCACATTCCCTG AGGCAGGTCACACGTCTCTTTATGATGAAGATAAGGTTC CTAGGGATGAACCAATTCACATTCTCAATGTGGCTATCA AGACCGACTGTGATATTGAGGATGACAGGCTGGCAGCT ATGTTCAGAGAATTTACCCAGCAAAATAAAGCTACCCTG GTTGACCATGGGATCCGGCGCCTTACTTTCCTGGTTGCA CAAAAGGATTTCAGAAAACAGGTCAACTATGAGGTGGA TCGGAGATTTCATAGAGAATTCCCTAAATTTTTTACATTC CGAGCAAGGGATAAGTTTGAGGAGGATCGTATCTATCGT CATCTGGAGCCTGCTCTGGCTTTCCAGTTAGAGCTGAAC CGGATGAGAAATTTTGACCTCACTGCCATTCCATGTGCT AATCACAAGATGCACCTGTATCTCGGGGCAGCCAAGGTG GAAGTGGGCACAGAAGTGACAGACTACAGGTTCTTTGTT CGTGCAATCATCAGGCATTCTGATCTGGTCACCAAGGAA GCTTCTTTTGAATATCTGCAAAATGAAGGGGAGCGGCTA CTCCTGGAAGCCATGGATGAGTTGGAAGTTGCTTTTAAC AATACAAATGTCCGCACTGACTGTAACCACATCTTCCTC AACTTTGTGCCCACGGTTATCATGGACCCATCAAAGATT GAGGAATCTGTGCGGAGCATGGTAATGCGGTATGGAAG TCGCCTGTGGAAATTGCGCGTCCTCCAGGCAGAACTGAA AATCAACATTCGCCTGACGCCAACTGGAAAAGCAATTCC CATCCGCCTCTTCCTGACAAACGAGTCTGGCTATTACTTG GATATCAGCCTATACAAGGAAGTGACTGACTCCAGGAC AGCACAGATCATGTTTCAGGCATATGGAGACAAACAGG GACCACTTCATGGAATGTTAATCAATACTCCTTATGTGA CCAAAGACCTGCTGCAATCAAAGAGGTTCCAGGCACAA TCCTTAGGGACAACATACATATATGATATCCCAGAGATG TTTCGGCAGTCCCTGATCAAACTCTGGGAGTCTATGTCC ACTCAAGCATTTCTTCCATCTCCCCCTCTGCCTTCTGACA TGCTGACTTACACCGAACTGGTATTGGATGATCAAGGTC AGCTGGTCCACATGAACAGGCTTCCAGGAGGAAATGAG ATTGGCATGGTAGCTTGGAAAATGACCTTTAAAAGTCCT GAATATCCAGAAGGCCGAGATATCATTGTTATTGGCAAT GACATCACATACCGAATTGGGTCCTTTGGGCCTCAAGAG GATTGGTTATTTCTCAGAGCTTCTGAACTTGCCAGGGCA GAAGGTATTCCACGCATCTATGTATCAGCCAACAGTGGA GCAAGAATCGGACTGGCAGAAGAAATTCGTCATATGTTT CATGTGGCCTGGGTAGATCCTGAGGATCCTTACAAGGGA TACAGGTATTTATATCTGACCCCTCAAGATTATAAGAGA GTCAGTGCTCTCAACTCTGTCCATTGTGAACACGTGGAG |
| | | GATGAAGGAGAATCCAGGTACAAGATAACAGATATTAT TGGGAAAGAAGAGGGAATTGGACCTGAGAACCTTCGAG GTTCTGGAATGATTGCTGGAGAATCCTCATTGGCCTATA ATGAGATCATTACCATCAGCCTGGTGACGTGCCGGGCCA TTGGGATTGGGGCTTACCTTGTCCGGCTGGGACAGAGAA CCATCCAGGTTGAGAATTCTCACTTAATTCTAACAGGAG CTGGAGCCCTCAACAAAGTCCTCGGGCGGGAAGTGTAC ACCTCCAATAACCAGCTCGGGGGCATCCAGATCATGCAC AACAATGGGGTGACCCACTGCACTGTGTGTGATGACTTT GAAGGGGTTTTCACTGTCCTGCACTGGCTGTCTTACATG CCCAAGAGCGTGCACAGTTCAGTTCCTCTTCTGAACTCA AAGGATCCTATAGACAGAATCATCGAGTTTGTTCCCACA AAGGCCCCATATGATCCTCGATGGATGCTAGCAGGCCGT CCTCATCCAACCCAAAAAGGTCAATGGTTGAGTGGCTTT TTTGACTATGGATCTTTCTCAGAGATTATGCAGCCCTGG GCACAGACTGTGGTGGTTGGTAGAGCCAGGCTAGGAGG AATACCTGTGGGAGTTGTTGCTGTAGAAACCCGAACAGT AGAGCTAAGTATCCCAGCTGATCCAGCAAACTTGGATTC TGAAGCCAAGATAATCCAGCAGGCTGGCCAGGTTTGGTT CCCAGATTCTGCGTTTAAGACGTATCAGGCCATCAAGGA CTTCAACCGGGAAGGGCTGCCTCTGATGGTCTTTGCCAA CTGGAGAGGCTTCTCTGGTGGAATGAAAGATATGTACGA CCAAGTGCTGAAGTTTGGTGCTTACATTGTGGACGGCTT GAGGGAGTGCTCCCAGCCTGTGCTGGTTTACATTCCTCC CCAGGCTGAGCTGCGGGGCGGCTCCTGGGTAGTGATTGA CTCCTCCATCAATCCCCGGCACATGGAGATGTATGCTGA CCGAGAAAGCAGGGGATCTGTTCTGGAGCCAGAAGGGA CAGTAGAAATCAAATTCCGCAGAAAGGATCTGGTGAAA ACCATGCGTCGGGTGGACCCAGTCTACATCCACTTGGCT GAGCGATTGGGGACCCCGGAGCTAAGCACAGCTGAACG GAAGGAGTTGGAGAACAAGTTGAAGGAGCGGGAGGAAT TCCTAATTCCCATTTACCATCAGGTAGCCGTGCAGTTTGC TGACTTGCACGACACACCAGGCCGGATGCAGGAGAAGG GTGTTATTAGCGATATCCTGGATTGGAAGACATCCCGTA CCTTCTTCTACTGGCGGCTGAGGCGTCTCCTGCTGGAGG ACCTGGTCAAGAAGAAAATCCACAATGCCAACCCTGAG CTGACTGACGGCCAGATTCAAGCCATGTTAAGGCGCTGG TTTGTGGAAGTGGAAGGTACAGTGAAGCTTGGTCCAGGC CAATCCAGAGGTCGCCATGGATTCCATCATCCATATGAC CCAGCACATATCACCTACTCAGCGAGCAGAAGTTGTACG GATCCTCTCCACAATGGACTCCCCTTCCACGTAGGAAAA GCTTCCTGCCTGTCCCTGCCCTGTCTCTGGAGAAAAGGG CTAGAGCTGCCTTTTACAACTGCAGCCACTGTAATGAGA AGGCACAGGAGACCCAGCACTGGAGTCAAAGTGGCATT TTACTTCCTCCCAAGTGTTTCAGGTTACGCATGACATCCT GGGATGTAAGATCACAGAACCCCTCCAGCCCACCAGTCA CACCCACCCCATTCAGTATTTATTACCCCAGCCAGGCCT AGTCCTCCACTCCCTGCACAGGACTGAGAAGGCAATGAA AGGTACAAACATGTACCATGAGGTCTTACTACCCAAAGC AGGGCTGCCCCTCCTGTCCTGACAGCCCCTTGGCCCCCA |
| | | GCATGGGGAAGCGTGAGGAGTTGGCGTTGCCAGGCAGC AGCCCCTCTCACCCCTGGCCCCATGAGCCGCAGCCACAG GCAGCAGAGGAGGGCTAAGGAGAGGAGGAAGCCTCAA GTCCATTGTTTATTACCCTGACTCTTAGCCCAGCACACAG TAGGCACTGGAGAGGAATGATGCCCAGTTTAACCACACC ATGGTACCTTTTATGACGAAAAATTAGAGCATAAAATCT ATCACAAGCTCCATAGGAACGCAAAGATGAGGGCAAAA CTGTGAGCCAAGAAGCAGAGAAAGAAAATAGACCCAGT TATTCTTGATTTAGGGGACTTCAACCTTGGGTTCAGTCTC TGAGGACAGAGGGAAAGGTAGTTGGCCTACCTCTCACCT GCATGTTACTGCTGGACTAGCTGTTGCATGTGGCTGGGA GCTGCAAGGCCCCAGTGCGTGAGGGGCCCCAGCAGTTCC ATAGGCGGTGAAGCCTGAGTTGGCAGAGGAGGAGCCAG AGAGAACTGCTCTTTCTGCACTGGTGGAAACTAGTTACT TATGCCATGTGGAGGGCCAGCCACATAGTCTGTTTTGAG GACTTAGAAAGTTGTTCCTATGAAGCCTGGAGCTTGGAT GGTTTTGAGGGGTTAATGGTGCCTCCACAGTCACTCTTC CCTAGTTCCAGGATTACTGTCCTAGCAGCTAACGTTTCTA CTGTCTTCCCCAGAATGTAGACAGGCAGCAGATGTAGCA GCTCTCCCCACAGTTCTGAAAGGACCCTGGTGACAGCCA CGCCCTCAGCACCAGGAGCTGGCCTTCCCGATGAGGGAG GCTTCCAGGAAACACAGAATCCACACGACCTTAAGATTC TTTACAACTCAGTCATGGTGCTGCTGTCCTCCAGGCTTAC TGGCCCCTCCTGACCGGCATCGGGGGCTTCCTCAGGTGG TAGAGAGAGTTTACTTTCAACAACTACTTTATTCAAGAA AAGAACTTACTGATTCCCCTGTTCCTAAAGCAAGAGTGG CAGGTGATCAGGGCTGGTGTAGCATCCAGTTCCTTTAGT GCAGCTAATTGCATTTGTCACTGACGACCAAGGAGGAAA TCACTAAGGCGTTTGAGAAGCAGTGGTATGAACGCTCTT GGACGAGCTACAGTTCTGAGCCTTAACCCTGTAGTTCGC ACACAAGAGCGGAGCTCCACCTCCCCTTCTTCGGGAGGA ATCTGTGCAGATGGATTGGCTGAACTTAATGGTTCTGGG TTGCAGGTGGGTACTGTATGGCTGGGTATGGAGCGGACT GCCCCCAGGAGTCAGAGCCTCAGCCTGGCTGCCCTGGTG GAAGGTGTGGGTGTTCAGCACCTTCAGAGAAGGGCATA AAGTGGTGGGGGACAATTCTCAGTCCAGGAAAATGCATT GACCATTGCTGGCTATTTGCTTGCCTAGTAAGAATTGGA TTCATTTTTGACCAGATTATTCTTCTATGCTTTTTTGCAAT AAATCAAATCCCACATATCTACAAGTGGTATGAAGTCCT GCACCCCCCAGGAGGCCTGTCCAGGCATCTCTTCAGAGG CAGGGTGGGTTACACTCATTTACCTCCCCTCTCCCCACCA AATTATGACACAAATGAGTATGTTTCCTCTCTAGAACCC TGTAATGCCTCCTCCCCGGTCCCCAGAGCTCCTCACTGTC GGTCTCACCCTGGACAAGGATTTTTTAGTCAGAGCCACA GAACATGGGCACTCTGACATTCCCACAGCCCCTCGCACA TGCGAGGCTACCAAGGCAGGAGAGTATAAATGATGGCT ATTGACCAAGACCTGCCTGGACGGGGACTGCCATATTAT CTGTTCTCTTCGTTCACAAAACAGCCTTCATTTGTCTCAG AATTCAATGGACGCATACCGTGATGAGCAGGAGCTTCAG |
| | | ATGCACTCTTTACACATTTTGTTGAAATAAACCTCTACAT TTGTAGAAGA |
| 153 | ACACB Cynomolgus monkey (Mf) XM_01543078 5 (GenBank RefSeq #) | TTTAAACCGTTTTGCACAGATCCCGTGCTCAGCGCTCTCG CCAGTTTCTCCACTTGCTGTTTAGTAAGGCCATCCTTGTT GGCAGGAAGTGGGGCCACTGAACTGCTGTTCCCCCTTCC CTGTTGCCTGAGCACAATGTGCTCCTATGTGGTCTCCGCT TTCCTGCTGCCTAGTTGGCTGGGCTGCTCCAGCTGGGCCT TAGAGGCGACCTTTGTCCCACATGCCAGCCCTCCCCACG TTGCAGAACACTGTGTAACTGCAGTGTGTCCTGAATTTC CTCTTCTCTGCTCAACCCTAGCAAGAGCCAGTCGACCCT GAATGAGAATGTGGGAATGGAGCTGTTTTTTTCTTTTATC CCCACGTCACAAGAAATTGGAAACATTCCATTAAGTGGC GTGATTCGAGAGAGTTTCGCCAGATCTTTTCAGGGGAGA GAAGCCAGGTGACCTGATAGCCATCATCACCTTCCCATT CACGGTTTTCACCGGGGGCTTCTCAAATAACCAACACCT GTAGGCAGTTAATTTTCAGCATGTCACAGGAGGGCCCAA ATGACTTTGTGCCTAATGCTTACGACAACATGGACTTTA GATTCCTCGTGGTTTCAATAGCGAAGAATTATGAAATAA AGCGAGTTATGGGATTAAAAAGCAGATAGCAGAACTCC CCCAGGAGTAGCCAGGGCTGCGCAAGTGGTGTGGCGTG GGATCTTTCTTGTCTTTGCCCTGGGTTGTTGCTGCTTTGA AGGTTAAGTGCTCCAGGGATATTTTTAAACAGCAGGAGC TGGTTAATCTAAAAGTGACATTTGCAGTTCCCACAGTAA GCAGCTAGCAGGCTTAGATTCAGGCCCTCAGCAAACACG GAACCTGGAAAATGTAACCCTGAATGCACGGTTGGGAG GACATGGCAAGAGAAAAGCAGCAGGAAGAAAGTGATTT TCTGAATGGTCTTTCTTCTTTGTCTATCTTGTCTGATTTTC TCCTGTCTGACCTTTTCCTGGTTAAAAATCTGGGGGAAA ATGACGGACTCCCAGCCAATCACCAAGAGTACATCAGA AGCAAACTTCATCCTGAGCCAGGAGCCCTTTCCAGCCTC TGACAACTCAGGGGAGACACCGCAGAGAAATGGGGAGG ACCACTCTCTGCCCAAGACTCCCAGCCAGGCCGAGCCAG CCTCCCACAAAGGCCCCAAAGATGCTGGTCGGCGGAGA AACTCCCTGCCACCCTCCCACCAGAAGCCCCCAAAAAAC CCCCTTTCTTCCAGTGACGCAGCACCCTCCCCAGAGCTC CAAGCCAACGGGACCGGGACACAAGGTCTGGAGGCCCC AGATACCAATGGCCTGTCTTCCTCAGCCAGGCCCCAGGG CCAGCAAGCTGGCTCCCCCTCCAAAGAAGACAGCAAGC AGGCAAACATCAAGAGGCAGCTGATGACCAACTTCATC CTGGGCTCTTTTGATGACTACTCCTCCGACGAGGACTCT GTTGCCGGCTCATCTCGTGAGTCTACCAGGAAGGGCAGC CGGGCCAGCTTGGGGGCCCTGTCCCTGGAGGCTGCTCTG ACCGCAGGTGAAGCTGAGACCCGTGTCCCCACTATGAGG CCGAGCATGTCAGGACTCCACCTGGTGAAGAGGGGACG GGAACACAAGAAGCTGGACCTGCACAGAGACTTTACAG TGGCTTCTCCCGCTGAGTTTGTCACACGCTTTGGCGGGG ATCGGGTCATCGAGAAGGTGCTTATTGCCAACAACGGGA TCGCCGCCGTGAAGTGCATGCGCTCCATCCGCAGGTGGG CCTATGAGATGTTCCGCAACGAGCGGGCCATCCGGTTTG TTGTGATGGTGACCCCTGAGGACCTTAAGGCCAACGCAG |
| | | AGTACATCAAGATGGCGGATCAGTACGTCCCCGTCCCAG GAGGGCCTAATAACAACAACTATGCCAACGTGGAGCTG ATTGTGGACATTGCCAAGAGAATCCCTGTGCAGGCGGTG TGGGCTGGCTGGGGCCATGCTTCCGAAAACCCTAAACTT CCGGAGCTGCTGTGCAAGAATGGAGTTGCTTTCTTAGGC CCTCCCAGTGAGGCCATGTGGGCCTTAGGAGATAAGATC GCCTCCACCATTGTCGCCCAGACGCTGCAGGTCCCAACC CTGCCCTGGAGTGGAAGCGGCCTGACAGTGGAGTGGAC AGAAGATGATCTGCAGCAGGGAAAAATAATCAGTGTCC CAGAAGATGTTTATGACCAGGGTTGTGTGAAAGACGTAG ATGAGGGCTTGGAGGCAGCAGAAAAAATTGGTTTTCCAT TGATGATCAAAGCTTCTGAAGGTGGCGGAGGGAAGGGA ATCCGGAAGGCTGAGAGTGCGGAGGACTTCCCGATCCTT TTCAGACAAGTACAGAGTGAGATCCCAGGCTCGCCCATC TTTCTCATGAAGCTGGCCCAGCACGCCCGTCACCTGGAA GTTCAGATCCTCGCCGACCAGTATGGGAATGCTGTGTCT CTGTTTGGTCGCGACTGCTCCATCCAGCGGCGGCATCAG AAGATTGTTGAGGAAGCACCGGCCACCATTGCCCCGCTG GCCATATTCGAGTTCATGGAGCAGTGTGCCGTCCGCCTG GCCAAGACCGTGGGCTATGTGAGTGCAGGGACAGTGGA ATACCTCTACAGCCAGGACGGCAGCTTCCACTTCTTGGA GCTGAATCCTCGTTTGCAGGTGGAACATCCCTGCACGGA AATGATCGCTGACGTCAATCTGCCGGCTGCCCAGCTACA GATCGCCATGGGCGTGCCACTGCACCGGCTGAAGGATAT CCGGCTTCTGTACGGAGAGTCGCCATGGGGAGTGACTCC CATTTCTTTCGAAACCCCGTCAAACCCTCCCCTCGCCCGA GGTCACGTCATTGCCGCCAGAATCACCAGTGAAAACCCA GATGAGGGGTTTAAGCCGAGCTCCGGGACTGTCCAGGA ACTGAATTTCCGCAGCAGCAAGAACGTGTGGGGTTACTT CAGTGTGGCTGCTACTGGAGGCCTGCATGAGTTTGCGGA TTCCCAGTTTGGGCACTGCTTCTCCTGGGGAGAGAACCG GGAAGAGGCCATTTCGTCAGTATCTCTCTCCCTTCCTTCT TTTCTTTCCTTCCTTCCTTCCTTCCTTCCTTCCTTCCTTCCT TCCACCCAGGCGGTGACAAGGGGCCTGTCCCCACAGGTG GCCCGGCAGTCTCTGACCATGTTCGTTCTCATCATGAAT GGCTGCCACATCGAGATTGATGCCCACCGGCTGAACGAT GGGGGGCTCCTGCTCTCCTACAATGGGAACAGCTACACC ACCTACATGAAGGAAGAGGTTGACAGTTACCGAATTACC ATCGGCAATAAGACATGTGTGTTTGAGAAGGAGAATGA TCCTACGGTCCTGAGATCCCCCTCGGCTGGGAAGCTGAC ACAGTACACAGTGGAGGACGGGGGCCACGTTGAGGCTG GGAGCAGCTACGCTGAGATGGAGGTGATGAAGATGATC ATGACTCTGAACGTGCAGGAAAGTGGCCGGGTGAAGTA CATCAAGCGTCCAGGGGCCGTGCTGGAAGCAGGCTGCG TGGTGGCCAGGCTGGAGCTTGATGACCCTTCTAAGGTCC ACCCGGCTGAACCGTTCACAGGAGAACTCCCTGCCCAGC AGACACTGCCCATCCTTGGAGAGAAACTGCACCAGGTCT TCCACAGCGTCCTGGAAAACCTCACCAACGTCATGAGTG GCTTTTGTCTGCCGGAGCCCATTTTTAGCATAAAGTTGA AGGAGTGGGTGCAGAAGCTCATGATGACCCTCCGGCATC |
| | | CGTCACTGCCGCTGCTGGAGCTGCAGGAGATCATGACCA GCGTGGCAGGCCGCATCCCGGCCCCCGTGGAGAAGTCA GTCCGCAGGGTGATGGCCCAGTATGCCAGCAACATCACC TCGGTGCTGTGCCAGTTCCCCAGCCAGCAGATAGCCACC ATCCTGGACTGCCACGCGGCCACCCTGCAGCGGAAGGCT GACCGAGAGGTCTTCTTCCTCAACACCCAGAGCATCGTG CAGTTGGTCCAGAGATACCGCAGCGGGATCCGCGGCTAT ATGAAAACAGTGGTGTTGGATCTCCTGAGAAGATACTTG CATGTCGAGCACCATTTTCAGCAAGCCCATTACGACAAG TGTGTGATAAACCTCAGGGAGCAGTTCAAGCCAGACATG TCCCAGGTGCTGGACTGCATCTTCTCCCACGCACAGGTG GCCAAGAAGAACCAGCTGGTGATCATGCTGATCGATGA GCTGTGTGGCCCAGACCCTTCCCTATCGGACGAGCTGAC CTCCATCCTTAATGAGCTCACTCAGCTGAGCAAAAGCGA GCACTGCAAAGTGGCCCTCAGAGCCCGGCAGATCCTGAT TGCCTCCCACCTCCCCTCCTATGAGCTGCGGCACAACCA GGTGGAGTCCATTTTCCTGTCTGCCATTGACATGTACGG CCATCAGTTCTGCCCCGAGAACCTCAAGAAGTTAATAAT TTCAGAAACAACCATCTTCGACGTCCTGCCTGCTTTCTTC TATCACGCTAACAAAGTCGTGTGCATGGCATCCTTGGAG GTTTACGTGCGGAGGGGCTACATCGCCTATGAGTTAAAC AGCCTGCAGCACCGGCAGCTCCCGGACGGCACCTGCGTG GTAGAATTCCAGTTCATGCTGCCCTCCTCCCACCCAAAC CGGATGACCGTGCCCATCAGCATCACCAACCCTGACCTG CTGAGGCACAGCACAGAGCTCTTCATGGACAGCGGCTTC TCCCCACTGTGCCAGCGCATGGGGGCCATGGTAGCCTTC AGGAGATTCGAGGACTTCACCAGAAATTTTGATGAAGTC ATCTCTTGCTTTGCCAACGTGCCCAAAGACACCCCCCTCT TCAGCGAGGCCCGCACCTCCCTGTACTCCGAGGATGACT GCAAGAGCCTCAGAGAAGACCCCATCCACATTCTGAATG TGTCCATCCAGTGTGCAGACCACCTGGAGGATGAGGCAC TGGTGCCGATTTTACGGACGTTCGTACAGTCCAAGAAAA ATATCCTTGTGGATTACGGACTCCGACGAATCACATTCC TGATTGCCCAAGAGAAAGAATTTCCCAAGTTTTTCACAT TCAGAGCAAGAGATGAGTTTGCAGAAGATCGCATTTACC GTCACTTGGAGCCTGCCCTGGCCTTCCAACTGGAGCTCA GCCGGATGCGTAACTTCGATCTGACCGCCGTGCCCTGTG CCAACCACAAGATGCACCTTTACCTGGGGGCTGCCAAGG TGAAGGAAGGGGCGGAAGTGACGGACCATAGGTTCTTC ATCCGCGCCATCATCAGGCACTCAGACCTGATTACAAAG GAAGCCTCCTTCGAGTACCTGCAGAACGAGGGTGAGCG GCTGCTCCTGGAGGCCATGGACGAGCTGGAGGTGGCGTT CAACAACACCAGCGTGCGCACCGACTGCAACCACATCTT CCTCAACTTCGTGCCCACTGTCATCATGGACCCCTTGAA GATCGAGGAGTCCGTGCGCTCCATGGTTATGCGCTACGG CAGCCGGTTGTGGAAACTCCGCGTGCTGCAGGCTGAAGT CAAGATCAACATACGCCAGACCACCACCGGCAGCGCTG TTCCCATCCGCCTGTTCATCACCAATGAGTCGGGCTACT ACCTGGACATCAGCCTCTACAAAGAAGTGACTGACTCCA GATCCGGAAATATCATGTTTCACTCCTTTGGCAACAAGC |
| | | AAGGGCCCCAGCACGGGATGCTGATCAATACTCCCTACG TCACCAAGGATCTGCTCCAGGCCAAGCGATTCCAGGCCC AGTCCCTGGGAACTACCTACATCTATGACTTCCCAGAAA TATTCAGACAGGCTCTCTTTAAACTATGGGGCTCCCCAG ACAAGTATCCCAAAGACATCCTGACATACACTGAATTAG TGTTGGACTCTCAGGGCCAGCTGGTGGAGATGAACCGAC TTCCTGGTGGAAATGAGGTGGGCATGGTGGCCTTCAAAA TGAGGTTTAAGACCCAGGAGTACCCTGAAGGACGGGAC ATGATCGTCATCGGCAACGATATCACCTTTCGCATCGGA TCCTTTGGCCCTGGGGAGGACCTTCTATACCTGCGGGCA TCTGAGATGGCCCGGGCAGAGGGCATTCCCAAAATCTAC GTGGCAGCCAACAGTGGCGCCCGTATTGGCATGGCAGA GGAGATCAAACACATGTTCCACGTGGCTTGGGTGGACCC AGAAGACCCCCACAAAGGATTTAAATACCTGTACCTGAC TCCCCAAGACTACACCAGAATCAGCTCCCTGAACTCTGT CCATTGTAAACACATCGAGGAAGGAGGAGAATCCAGAT ACATGATCACGGATATCATCGGGAAAGATGATGGCTTGG GCGTGGAGAATCTGAGGGGCTCAGGCATGATTGCTGGG GAGTCCTCTCTGGCTTACGAAGAGATCGTCACCATTAGC TTGGTGACCTGCCGAGCCCTTGGGATCGGGGCCTACTTG GTGAGGCTGGGCCAGCGAGTGATCCAGGTGGAGAACTC TCACATCATCCTCACGGGAGCAAGTGCTCTCAACAAGGT CCTGGGAAGAGAGGTCTACACATCCAACAACCAGCTGG GTGGCGTTCAGATCATGCATTACAATGGCGTTTCCCACA TCACCGTGCCAGATGACTTTGAGGGGGTTTATACCATTC TGGAGTGGCTGTCCTATATGCCAAAGGATAATCACAGCC CTGTCCCTATCATCACACCCACTGACCCCATTGACAGAG AAATTGAATTCCTCCCATCCAGAGCTCCCTACGACCCCC GGTGGATGCTTGCAGGAAGGCCTCACCCAACGCTGAAG GGAACGTGGCAGAGCGGATTCTTTGACCATGGCAGTTTC AAGGAAATCATGGCACCCTGGGCGCAGACCGTGGTGAC AGGACGAGCAAGGCTTGGGGGGATTCCCGTGGGAGTGA TTGCTGTGGAGACGCGGACTGTGGAGGTGGTGGTCCCTG CAGACCCTGCCAACCTGGATTCTGAGGCCAAGATAATTC AGCAGGCAGGGCAGGTGTGGTTCCCAGACTCAGCCTAC AAAACCGCCCAGGCCATCAAGGACTTCAACCGGGAGAA GTTGCCCCTGATGATCTTTGCCAACTGGAGGGGGTTCTC CGGTGGCATGAAAGACATGTATGATCAGGTGCTGAAGTT TGGAGCCTACATCGTGGATGGCCTCAGACAATACAAACA GCCCATCCTGATCTATATCCCACCCTATGCGGAGCTCCG GGGAGGCTCCTGGGTGGTCATAGATGCCACCATCAACCC GCTGTGCATAGAAATGTATGCGGACAAAGAGAGCAGGG GGGGTGTTCTGGAGCCAGAGGGGACAGTGGAGATTAAG TTCCGAAAGAAAGATCTGATAAAGTCCATGAGAAGGAT CGATCCAGCTTACAAGAAGCTCATGGAACAGCTAGGGG AACCTGATCTCTCAGACAAGGACCGCAGGGACCTGGAG GGCCGGCTGAAGGCTCGCGAGGACCTGCTGCTCCCCATC TACCACCAGGTGGCGGTGCAGTTCGCCGACTTCCACGAC ACGCCCGGCCGGATGCTGGAGAAGGGCGTCATATCTGA CATCCTGGAGTGGAAGACCGCGCGCACCTTCCTGTATTG |
| | | GCGTCTGCGCCGCCTCCTCCTGGAGGACCAGGTCAAGCA GGAGATCCTGCGGGCCAGCGGGGAGCTGAGTCACGTGC ACATCCAGTCCATGCTGCGCCGCTGGTTCGTGGAGACGG AGGGGGCTGTCAAGGCCTACCTGTGGGACAACAACCAG GTGGTTGTGCAGTGGCTAGAACAGCACTGGCAGGTGGA GGACGGCCCGCGCTCCACCATCCGCGAGAACATCACGTA CCTGAAGCACGACTCTGTCCTCAAGACCATCCGAGGCCT GGTTCAAGAAAACCCCGAGGTGGCCGTGGACTGTGTGAT ATACCTGAGCCAGCACATCAGCCCAGCTGAGCGGGCAC AGGTCATCCACCTGCTGTCTACCATGGACAGCCCGGCCT CCACCTGACCCCGGCCTGCCCCGCCATTCCCGGGACCAC AGCAGGAGGAACCACCCACACCCACCATCGGTACGCCC TCAGCAGACCTTGAAGACCTGCTTTTAAAAAAAGAAAAT CCTGGGCACTTCTGCAGGGCTGCTGGTTCCGAGCTGACA CCTGTCTCAATAAAAGGCCCAGGAGTGCCTCTCCCAAAC ACAAACAACCTCCTCTGCATAGCTGGGAAGTTTCTTTTG TTTTGTCTCTGAAGACAGCATTTTTATTGCATCACTAAAT CTAATCAAGCTAAAACATCCCTGTTTCCTTTTGCAAAAC AGTGCCTGGCCTGTGGGATCCAGGCGTTCTTCAGGCTTC TTGGATATCATATCATGAAATCTTTTATTTTTTTACTCTG AGACCAGCACTAGATGTAAGCATCTCATATATTTCAGCC AAATAAATGGGCCAAGGAAAAAAAATATATATATATAG ACAGGACTAGAGAAAAACCTATTTTTGTAATGATGTTTC TTTGGATACTGTCTAGTCACCCAGGAAAATGTATGGATG AAATTTTTTTTTTTTTTTTTTTGAGACAGAGTCTTACTCTG TCACCTAGGCTGGAATGGGGTAGCATGATCTCACTGCAA CCTTCATCTCCCAGGTTCAAGCTGTTCTCCTGTCTCAGCC TCCTAGATAGCTGGGATTACAGGTGCCCATCACCATGTC CAGCTAATTCTTGTATTTTTAGTAGAGACACAGTTTCACC ATGTAGGTCAGGCTGGTCTTGAACCCTTGACTTCAGGTA ATCCACCCACCTTGGCCCCCCAAAGTGCTGGGATTACAG GCGTGAGCCACCATCTTTGGCCAGATGATTTTTTATTGCA AGAATGAAACGGCATTTTGTTCCCCAAATGGCCCTAGTG AATCACTAGGAGGGCTCCACTGGTAGGCCATGTTTAGCA CTGGTTGCCAGGGATTCTCTTTTTGAGAGAGGGAAAGCA AAATGAATGGAAGTGCCTGGCCAGAGGTTTCAGGGCTTC TGGAGGATCCTCTCGCATAGCTCGAGGTCCTCTGCCTGC CTCTTCCCTCCAAGGAAAATGAGGACTGCCCCTTCCCCC TGCAGGATTGGCCCCCAGCCTGTGCAGGCACCCTTCTCT TGCCCAAGTGGGGAGCACAGAGGCGGAGAGGAATCCCT TGCCACACCCATGGCCCAGCTTGCTCACAAGTGTCACGT CTGTGACGGTCACCACTGCTCCCTTGGAGGGCCACTTGA GTCACTGTTGCTCCCTTGCCTGCTGGCTTGATGAGCACA GATGGTGGGATCTGACCGAGGGGCAGAGCTGTCGGTGA CTAAGGACTGGACTGTGGTGACCATACCGATTTGCTCAG GGAGGATGTTGCAATGCATCCAGCAGCTCCTGGCTCTGC AGGCGGCACAGCCCGGGGCCCTGCAATCCTCTGGTTTCT CCCATTGGGGTGGAGTGGGGGAGTGGAGGGAGTTGGCC ACAACCCACTGCTGTGATGGGCGGTTTGTCCAAGGATGC TGTATATGATGCCTGGTCAATGTGGAAGCCCATGAAGTC |
| | | ACCCAGGGAAGCCTCCAAAAGCTGGGATGCTTGAGGGT ATCCAAGTTGAAAAAGACAAAATCTGACCATTAGCCAGT GACGGCCCTGGCAAATGAAGGTGTGGTGGGGCAGTGAG GGATGGGAGAAGGTGAATTATTCCTTATTCCACCCCGAG GTTTGCTGGGGTGAGGGGAAGAATTGATGCTGCTTTGGG AACTGAAGGTTTTTCTGTTGGGAAGGCCCTCTTGGTTTTG GAGAGAAAGGCACGTTATGAGTAGCTGCTACCCTGGAA CGGTGGGCAGAGAGCCTACCAGGAAATGTGCAGAATAA ACTATTTTTTGAAGGAAA |
| 154 | ACACA Mouse (Mm) NM_133360 (GenBank RefSeq #) | CTCTGAGAGCTTATTTTGAAAGAATAATGGATGAACCAT CTCCGTTGGCCAAAACTCTGGAGCTAAACCAGCACTCCC GATTCATAATTGGGTCTGTGTCTGAAGACAACTCAGAAG ATGAGATCAGTAACCTGGTGAAGCTGGACCTAGAAGAG AAGGAGGGCTCCCTGTCACCAGCCTCCGTCAGCTCAGAT ACACTTTCTGATTTGGGGATCTCTGGCTTACAGGATGGTT TGGCCTTTCACATGAGATCCAGCATGTCTGGCTTGCACC TAGTAAAACAAGGTCGAGACAGAAAGAAAATAGACTCA CAACGAGATTTCACTGTGGCTTCTCCAGCAGAATTTGTT ACTCGTTTTGGGGGAAATAAAGTAATTGAGAAGGTTCTT ATCGCCAACAATGGTATTGCAGCAGTGAAATGCATGCGA TCTATCCGTCGGTGGTCTTATGAAATGTTTCGAAATGAA CGTGCAATCCGATTTGTTGTCATGGTTACACCTGAAGAC CTTAAAGCCAATGCAGAATACATTAAGATGGCAGACCA CTATGTTCCAGTGCCTGGAGGACCCAACAACAACAATTA CGCAAATGTGGAGTTGATTCTTGATATTGCTAAAAGGAT ACCTGTACAAGCAGTGTGGGCTGGCTGGGGTCATGCCTC TGAGAACCCGAAACTCCCAGAACTGCTCTTAAAAAATGG CATTGCTTTCATGGGCCCTCCAAGCCAGGCCATGTGGGC TTTGGGGGATAAGATTGCATCTTCTATTGTGGCTCAAAC TGCAGGTATCCCAACTCTTCCCTGGAGTGGCAGTGGTCT TCGAGTGGATTGGCAAGAAAATGATTTTTCGAAACGTAT CTTAAATGTTCCACAGGATCTGTATGAGAAAGGCTATGT GAAGGATGTGGATGATGGTCTGAAGGCAGCTGAGGAAG TTGGCTATCCAGTGATGATCAAGGCCTCAGAGGGAGGA GGAGGGAAAGGGATCAGAAAAGTTAACAATGCAGATGA CTTCCCTAACCTCTTCAGACAGGTTCAAGCTGAAGTTCCT GGATCACCTATATTTGTAATGAGACTAGCAAAACAATCT CGACATCTGGAGGTCCAGATTCTGGCAGATCAGTATGGC AATGCTATTTCTTTGTTTGGTCGTGACTGCTCTGTGCAGC GCAGGCATCAGAAGATCATTGAAGAAGCTCCTGCTGCG ATTGCTACCCCAGCAGTATTTGAACACATGGAACAGTGT GCAGTGAAACTTGCCAAAATGGTTGGTTATGTGAGTGCT GGGACTGTGGAATACTTGTACAGCCAGGATGGAAGCTTC TACTTTTTGGAACTGAACCCTCGGCTACAGGTTGAACAT CCTTGTACAGAGATGGTGGCTGATGTCAATCTTCCTGCA GCACAGCTCCAGATTGCCATGGGGATCCCTCTATTTAGG ATCAAGGATATTCGTATGATGTATGGGGTATCTCCTTGG GGAGATGCTCCCATTGATTTTGAAAATTCTGCTCATGTTC CTTGCCCAAGGGGCCACGTGATTGCTGCTCGGATCACCA GTGAAAACCCAGATGAGGGGTTTAAGCCCAGCTCTGGA |
| | | ACAGTTCAGGAACTTAATTTTCGTAGCAATAAGAACGTT TGGGGTTATTTCAGTGTTGCTGCTGCTGGAGGACTTCAT GAATTTGCTGATTCTCAGTTCGGGCACTGCTTTTCCTGGG GAGAAAACAGGGAGGAAGCAATCTCAAATATGGTGGTG GCACTGAAGGAGCTGTCTATTCGGGGTGACTTTCGAACT ACAGTGGAATACCTCATCAAACTGCTGGAGACAGAAAG CTTTCAGCTTAACAGAATCGACACTGGCTGGCTGGACAG ACTGATCGCAGAGAAAGTGCAGGCAGAGCGACCTGACA CCATGTTGGGAGTTGTGTGTGGGGCTCTCCATGTAGCAG ATGTGAGCCTGAGGAACAGCATCTCTAACTTCCTTCACT CCTTAGAGAGGGGTCAAGTCCTTCCTGCTCACACACTTC TGAACACAGTAGATGTTGAACTTATCTATGAAGGAATCA AATATGTACTTAAGGTGACTCGGCAGTCTCCCAACTCCT ACGTAGTGATAATGAATGGCTCGTGTGTGGAAGTGGATG TGCATCGGCTGAGTGATGGTGGCCTGCTCTTGTCTTATG ACGGCAGCAGTTACACCACATACATGAAGGAAGAGGTG GACAGATATCGAATCACAATTGGCAATAAAACCTGTGTG TTTGAGAAGGAAAATGACCCATCTGTAATGCGCTCACCG TCTGCTGGGAAGTTAATCCAGTATATTGTGGAAGATGGC GGACATGTGTTTGCTGGCCAGTGCTATGCTGAGATTGAG GTAATGAAGATGGTGATGACTTTAACAGCTGTAGAATCT GGCTGCATCCATTATGTCAAACGACCTGGAGCAGCACTT GACCCTGGCTGTGTGATAGCCAAAATGCAACTGGACAAC CCCAGTAAAGTTCAACAGGCTGAGCTTCACACGGGCAGT CTACCACAGATCCAGAGCACAGCTCTCAGAGGCGAGAA GCTCCATCGAGTTTTCCACTATGTCCTGGATAACCTGGTC AATGTGATGAATGGATACTGCCTTCCAGACCCTTTCTTC AGCAGCAGGGTAAAAGACTGGGTAGAAAGATTGATGAA GACTCTGAGAGACCCCTCCTTGCCTCTGCTAGAGCTGCA GGATATCATGACCAGTGTCTCTGGCCGGATCCCCCTCAA TGTGGAGAAGTCTATTAAGAAGGAAATGGCTCAGTATGC TAGCAACATCACATCAGTCCTGTGTCAGTTTCCCAGCCA GCAGATTGCCAACATCCTAGATAGTCATGCAGCTACACT GAACCGGAAATCTGAGCGGGAAGTCTTCTTCATGAACAC CCAGAGCATTGTCCAGCTGGTGCAGAGGTACCGAAGTG GCATCCGTGGCCACATGAAGGCTGTGGTGATGGATCTGC TGCGGCAGTACCTGCGAGTAGAGACACAGTTTCAGAAC GGCCACTACGACAAATGTGTATTCGCCCTTCGGGAAGAG AACAAAAGCGACATGAACACCGTACTGAACTACATCTTC TCCCACGCTCAGGTCACCAAAAAGAATCTCCTGGTGACA ATGCTTATTGATCAGTTATGTGGCCGGGACCCTACACTT ACTGATGAGCTGCTAAATATCCTCACAGAGCTAACCCAA CTCAGCAAGACCACCAACGCTAAAGTGGCGCTGCGCGCT CGTCAGGTTCTTATTGCTTCCCATTTGCCATCATATGAGC TTCGCCATAACCAAGTAGAGTCTATCTTCCTATCAGCCA TTGACATGTATGGACACCAGTTTTGCATTGAGAACCTGC AGAAACTCATCCTCTCGGAAACATCTATTTTCGATGTCCT CCCAAACTTTTTTTACCACAGCAACCAGGTGGTGAGGAT GGCAGCTCTGGAGGTGTATGTTCGAAGGGCTTACATTGC CTATGAACTCAACAGCGTACAACACCGCCAGCTTAAGGA |
| | | CAACACCTGTGTGGTGGAATTTCAGTTCATGCTGCCCAC ATCCCATCCAAACAGAGGGAACATCCCCACGCTAAACA GAATGTCCTTTGCCTCCAACCTCAACCACTATGGCATGA CTCATGTAGCTAGTGTCAGCGATGTTCTGTTGGACAACG CCTTCACGCCACCTTGTCAGCGGATGGGCGGAATGGTCT CTTTCCGGACCTTTGAAGATTTTGTCAGGATCTTTGATGA AATAATGGGCTGCTTCTGTGACTCCCCACCCCAAAGCCC CACATTCCCAGAGTCTGGTCATACTTCGCTCTATGATGA AGACAAGGTCCCCAGGGATGAACCAATACATATTCTGA ATGTGGCTATCAAGACTGATGGCGATATTGAGGATGACA GGCTTGCAGCTATGTTCAGAGAGTTCACCCAGCAGAATA AAGCTACTTTGGTTGAGCATGGCATCCGGCGACTTACGT TCCTAGTTGCACAAAAGGATTTCAGAAAACAAGTCAACT GTGAGGTGGATCAGAGATTTCATAGAGAATTCCCCAAAT TTTTCACATTCCGAGCAAGGGATAAGTTTGAGGAGGACC GCATTTATCGACACCTGGAGCCTGCTCTGGCTTTCCAGTT AGAGTTGAACCGGATGAGAAATTTTGACCTTACTGCCAT CCCATGTGCTAATCACAAGATGCACCTGTACCTTGGGGC TGCTAAGGTGGAAGTAGGCACAGAAGTGACTGACTACA GGTTCTTTGTTCGTGCGATCATCAGGCACTCTGATCTGGT CACAAAGGAAGCTTCTTTCGAATATCTACAAAATGAAGG GGAACGACTGCTCCTGGAAGCTATGGATGAATTGGAAGT TGCTTTTAATAATACAAATGTCCGCACTGACTGTAACCA CATCTTCCTCAACTTTGTGCCCACGGTCATCATGGACCCA TCAAAGATTGAAGAATCTGTGCGGAGCATGGTAATGCGC TATGGAAGTCGGCTATGGAAATTGCGGGTCCTCCAGGCA GAACTGAAAATCAACATTCGCCTGACAACAACTGGAAA AGCAATTCCCATCCGCCTCTTCCTGACAAACGAGTCTGG CTACTACTTGGACATCAGCCTGTATAAGGAAGTGACTGA CTCCAGGACAGCACAGATCATGTTTCAGGCATATGGAGA CAAGCAGGGACCACTGCATGGAATGTTAATTAATACTCC ATATGTGACCAAAGACCTTCTTCAATCAAAGAGGTTCCA GGCACAGTCCTTAGGAACAACATATATATATGATATCCC AGAGATGTTTCGGCAGTCACTCATCAAACTCTGGGAGTC CATGTCCACCCAAGCATTTCTTCCTTCGCCTCCTTTGCCT TCCGACATCCTGACGTATACTGAACTGGTGTTGGATGAT CAAGGCCAGCTGGTCCATATGAACAGACTTCCAGGAGG AAATGAGATTGGCATGGTAGCCTGGAAAATGAGCCTTA AAAGCCCTGAATATCCAGATGGCCGAGATATCATTGTCA TCGGCAATGACATTACATATCGGATCGGTTCCTTTGGGC CTCAGGAGGATTTGCTGTTTCTCAGAGCTTCTGAACTTGC CAGAGCAGAAGGCATCCCACGCATCTACGTGGCAGCGA ACAGTGGAGCTAGAATTGGACTTGCAGAAGAAATACGC CATATGTTCCATGTGGCCTGGGTAGATCCTGAAGATCCC TACAAGGGATACAAGTATTTATATCTGACACCCCAGGAT TATAAGAGAGTCAGTGCCCTCAATTCTGTCCACTGTGAA CATGTGGAGGATGAAGGGGAATCCAGGTACAAGATAAC AGATATTATCGGGAAAGAAGAAGGACTTGGAGCAGAGA ACCTTCGGGGTTCTGGAATGATTGCTGGGGAATCCTCAT TGGCTTACGATGAGGTCATCACCATCAGCCTGGTTACAT |
| | | GCCGGGCCATTGGTATTGGGGCTTACCTTGTCCGGCTGG GACAAAGAACCATCCAGGTTGAGAATTCTCACTTAATTC TGACAGGAGCAGGTGCCCTCAACAAAGTCCTTGGTCGGG AAGTATACACCTCCAACAACCAGCTTGGGGGCATCCAGA TTATGCACAACAATGGGGTTACCCACTCCACTGTTTGTG ATGACTTTGAGGGAGTCTTCACAGTCTTACACTGGCTGT CATACATGCCGAAGAGCGTACACAGTTCAGTTCCTCTCC TGAATTCCAAGGATCCTATAGATAGAATCATCGAGTTTG TTCCCACAAAGGCCCCATATGATCCTCGGTGGATGCTAG CAGGCCGTCCTCACCCAACCCAGAAAGGCCAATGGTTGA GTGGGTTTTTTGACTATGGATCTTTCTCAGAAATCATGCA GCCCTGGGCACAGACCGTGGTAGTTGGCAGAGCCAGGTT AGGGGGAATACCCGTGGGAGTAGTTGCTGTAGAAACCC GAACAGTGGAACTCAGTATCCCAGCTGATCCTGCGAACC TGGATTCTGAAGCCAAGATAATCCAGCAGGCCGGCCAG GTTTGGTTCCCAGACTCTGCATTTAAGACCTATCAAGCT ATCAAGGACTTTAACCGTGAAGGGCTACCTCTAATGGTC TTTGCCAACTGGAGAGGTTTCTCTGGTGGGATGAAAGAT ATGTATGACCAAGTGCTCAAGTTTGGCGCTTACATTGTG GATGGCTTGCGGGAATGTTCCCAGCCTGTAATGGTTTAC ATCCCGCCCCAGGCTGAGCTTCGGGGTGGTTCTTGGGTT GTGATCGACCCCACCATCAACCCTCGGCACATGGAGATG TACGCTGACCGAGAAAGCAGGGGATCTGTTCTGGAGCC AGAAGGGACAGTAGAAATCAAATTCCGTAAAAAGGATC TGGTGAAAACCATGCGTCGGGTAGATCCAGTTTACATCC GCTTGGCTGAGCGATTGGGCACCCCAGAGCTAAGCCCCA CTGAGCGGAAGGAGCTGGAGAGCAAGTTGAAGGAGCGG GAGGAGTTCCTAATTCCCATTTACCATCAGGTAGCTGTG CAGTTTGCTGACTTGCACGACACACCAGGCCGGATGCAG GAGAAGGGTGTCATTAACGATATCTTGGATTGGAAAACA TCCCGCACCTTCTTCTACTGGAGGCTGAGGCGCCTCCTG CTGGAGGACCTGGTCAAGAAGAAAATCCACAATGCCAA CCCTGAGCTGACTGACGGCCAGATCCAGGCCATGTTGAG ACGCTGGTTTGTAGAAGTTGAAGGCACAGTGAAGGCTTA CGTCTGGGACAATAATAAGGACCTGGTGGAGTGGCTGG AGAAGCAACTGACAGAGGAAGATGGCGTCCGCTCTGTG ATAGAGGAGAACATCAAATACATCAGCAGGGACTATGT CCTGAAGCAGATCCGCAGCTTGGTCCAGGCCAACCCAGA AGTCGCCATGGACTCCATCGTCCACATGACCCAGCACAT CTCACCCACCCAGCGAGCAGAAGTTGTAAGGATCCTCTC CACTATGGATTCCCCTTCCACGTAGGAGGAGCTTCCCGC CCACCCCTGCCCTGTCTCTGGAGAAGAGAGTCGGGCTGC CTCTCCCATCTGAGACCACTGTAATGAGAAGGCACCGGA GGCCTGAGACTGGATCAGTGGCATTTGCTTCCCTTGAGT GTTTCAGGCTCTGCATGACATCCTGGGCTATAGGATCAC ACAGCCCAGTCACACATACCCGATTCAGTATTTATTAGC CCAGCTATGATGACAGTCCTCTTCCCATGCACAGGACTG AGAAGGCAATGAAAGGTACTTGCCTGTACCATGAGGTCT TACTAGACTAAAGCAGGACTGCCCTCCGTCCTGCCTGCC CCAGCATAGGGTCGTGTGAACAGTGTCCAAGTGTCTGCA |
| | | GCCCCTGCCCCATGAGCCACAGCCAACAGGGGAGGGCT GGGGCTGCCAGGAGACGCAAGTCCATCGATCACTACCCC ACATTGCAACCAAGCACATGCCAGGCACTGGAGAGAGA CAGTCCCATCTAACCACAGCAAGATAACTGAGAAATGTC AGAGCAGAAAATCTGTCGCAAGCCCGTGAGAACACAGA GATAAAGGCAAAGCCATGATCTAACAGGCAGGCTCAAA ACCAGATCATGTCATTGTTCCTGGTCAAACACACTCAAC CACCTCAGTGGCGTCTCAGTATCCCTGGGGAGACGGTCA CCCTGCCACCACCTTATCACTATGTAATCACATAGTGAC TATACCTAGGAGCTAAAGGCCTGTGCATAAGGGCCCTAG CCTCTGCGGGTGGATGGAGACAGGCAGGCAGTGCAGGA GCCTGAGGTGGCTAAGAGGAGGCTCTTTTCTGTTGGTGA TAGGGGAAGAGTACTTCAAAACAGGAGAAGTCTCTTCTG GGGACAGGTTGTCACTATGGGGCCATGCCTATTCCTGGC AGAAGGTATGGCGGGTCACCCACCTGATGACACCTGTGC TTTCAGCACCAGAAACTGGCCTTCTTGATGTTAGGAGGC TTCCAGGGAACAAGAATTCACCTGCCCTTAAGATACCTT ACAGTTCAGTCTCGGTGCTGGTGTTTCCAGACTTCTGGG CCCTATGTGGCCAGCAACACGGCTTCCTCAGGGGTGGCC TGAAAAGTTTAACTACCCATTTGTCCAAGAAAGCAGCTG ATGGTTCAGCATTTGTGTTCATAAAGCAAGAATGAAGAT GATCATAACTCGTATAACATCCAGTTTCAACTTTATGACT AATACATCTGTCGCTGAAGATCGAGAAGGAAAAAGATA CCTGCCACAGAAACCATTCAAGTGAGTACTTCAGTCTGA GCCTTAACCTGGATTCCACGAAAAGAGCTGACCTCCCCT TCAGGAGCACCTCAAGCAGATATTCCAGAGGACCTTCTC AATGGTGCCACATTGCAAGTGGACATCCAAGGGACTATA GATGCAAGTTGCCCCACCCCCACAAGCCCTGGTGGAAGG AACAGGAGGGCCAGAGCTGGATCAGGTGGGGAGCATCC TTAGTCCAGAAGGAAGCTCCCTGGCCCCTGGTTCCCTGC TTACCTGGTAGGTAAGGATGGGATTTATCTCTGGCCTCC ACTTTTGCTACAGCACGTCCAATCCCACACCCCCGGTGG TATGAAAGCTGCTTTCCTGGAGAGGTGGAGTGGGCTGGG CTTGTATAAGTCCCTTTTCCCTGCTGCCCCATCCCCGGGA CCGGGCCGTCTCAATACGGAGAAGTCAGAGCCACGGCA CATGGGCAGTGTGATGGTACCACAGCCCATTACACATGC AGGGTTACTGAGGAGGAGGGTGAATGATGTATTGACCC AGACTGGCTTGAACTGAGATTGCCATATTATCTGTCCAC TTGTTCACAAAGCAGCCTTCACACGTGTCAGTGAGAGTT CGATGGACACACACGCCAGCGAGCAGGGGCTTCAGTCC AATGCACTCTTCTCACGTTTTGTTGAAATAAACCTCCACA TTTGTAGAAGAAAAAAAAAAAAAAAAAAAAAAA |
| 155 | ACACB Mouse (Mm) NM_133904 (GenBank RefSeq #) | ATGGTCTTGCTTCTCTTTCTGACTTGCCTGGTTTTCTCCTG CCTGACCTTTTCCTGGCTAAAAATCTGGGGGAAGATGAC AGACTCGAAGCCGCTCACCAACAGTAAGGTGGAAGCCA ATCTCCTTTCAAGCGAGGAGTCCCTTTCAGCCTCAGAGC TGTCAGGGGAACAGCTGCAGGAACATGGCGACCACAGC TGTCTGTCCTACAGAGGTCCCAGAGACGCCAGTCAGCAG AGGAACTCCCTGCCAAGCTCATGCCAGAGGCCTCCGAGG AACCCACTGTCTTCCAATGACACCTGGCCCTCCCCAGAA |
| | | CTCCAAACCAACTGGACAGCCGCCCCGGGCCCAGAGGTT CCAGATGCTAATGGGTTGTCCTTCCCAGCCAGGCCCCCG AGCCAGAGGACAGTATCCCCCTCCAGAGAGGACAGGAA GCAGGCACACATCAAGAGACAGCTGATGACCAGCTTTAT CCTGGGCTCCCTGGATGACAACTCCTCTGATGAGGACCC TAGTGCCGGCTCCTTCCAGAACTCCTCCCGGAAGAGCAG CAGGGCTAGCCTGGGCACTCTGTCCCAGGAGGCTGCATT GAACACAAGTGATCCTGAATCTCACGCGCCTACTATGAG GCCCAGCATGTCTGGACTCCATCTGGTGAAGAGAGGCCG AGAACACAAGAAACTGGACCTGCACAGAGATTTCACCG TCGCGTCCCCAGCCGAGTTTGTCACTCGGTTTGGAGGCA ACAGGGTCATAGAGAAGGTGCTCATCGCCAACAATGGC ATCGCTGCGGTCAAGTGTATGCGCTCCATCCGCCGCTGG GCCTACGAGATGTTCCGTAATGAACGTGCCATCCGGTTC GTGGTCATGGTGACGCCCGAGGATCTGAAGGCCAACGC AGAGTACATCAAGATGGCCGATCAGTACGTCCCTGTCCC AGGAGGACCCAATAACAACAACTATGCCAATGTTGAGC TGATCATAGACATTGCCAAGAGAATCCCTGTACAGGCCG TGTGGGCTGGCTGGGGCCACGCTTCGGAAAACCCCAAAC TTCCAGAGCTGCTGTGTAAACACGAGATTGCTTTCCTAG GTCCCCCCAGTGAGGCCATGTGGGCCCTGGGAGACAAG ATCGCCTCCACCATCGTAGCGCAGACACTGCAAATCCCA ACCTTACCCTGGAGTGGAAGCGGTCTCACAGTGGAGTGG ACGGAGGACAGCCGGCACCAGGGCAAATGCATCAGTGT CCCCGAAGACGTTTACGAGCAGGGCTGTGTGAAGGATGT GGACGAAGGCTTGCAGGCAGCAGAAAAAATAGGATTTC CTTTGATGATCAAAGCCTCCGAAGGCGGAGGCGGGAAA GGTATCCGCAAGGCTGAGAGTGCAGAGGACTTCCCGAT GCTTTTCCGACAAGTGCAGAGTGAGATTCCGGGCTCGCC GATCTTTCTAATGAAGCTGGCCCAGAACGCACGGCACCT GGAGGTCCAGGTCTTGGCAGATCAGTATGGAAACGCGG TGTCACTGTTCGGACGGGACTGTTCCATTCAGAGGCGGC ACCAGAAGATCATTGAGGAGGCTCCGGCCACCATCGCC GCGCCTGCCGTGTTTGAGTTCATGGAACAGTGTGCTGTC CTCCTGGCCAAGATGGTGGGTTATGTGAGCGCGGGGACT GTGGAGTACTTATACAGCCAGGATGGCAGCTTCCACTTC CTGGAGCTGAACCCACGCCTGCAGGTGGAGCATCCGTGC ACTGAAATGATCGCAGATGTCAACCTGCCTGCTGCGCAG TTGCAGATCGCCATGGGCGTGCCCCTGCACCGACTGAAG GACATACGGCTTCTGTACGGAGAGTCCCCCTGGGGAGTG ACCCCCATTCCTTTTGAAACCCCCTTGAGCCCTCCCATTG CCCGAGGCCACGTCATTGCAGCCAGGATCACCAGCGAA AACCCAGATGAGGGCTTTAAGCCAAGCTCGGGGACAGT GCAGGAGCTGAACTTCCGCAGCAACAAGAACGTGTGGG GTTACTTCAGCGTGGCCGCTGCTGGGGGCTTGCACGAGT TCGCCGATTCCCAGTTTGGGCACTGCTTCTCCTGGGGCG AGAACCGTGAAGAGGCCATTTCGAACATGGTAGTGGCTT TGAAGGAACTGTCCATCCGGGGTGACTTCCGGACCACTG TGGAGTATCTCGTTAACCTTCTGGAGACCGAGAGCTTCC AGAACAACGATATCGACACGGGGTGGCTGGACCACCTC |
| | | ATCGCTCAGCGGGTGCAGGCAGAGAAGCCGGACATCAT GCTTGGGGTGGTGTGTGGGGCCTTGAATGTGGCAGACGC GATGTTCAGAACCTGCATGACGGAATTCTTGCACTCCCT GGAAAGGGGTCAGGTCCTCCCTGCTGATTCTCTGCTGAA CATTGTGGACGTTGAATTGATTTACGGCGGCATCAAGTA TGCTCTCAAGGTAGCCCGACAGTCCCTGACTATGTTTGT CCTCATCATGAACGGCTGCCACATCGAGATCGATGCCCA CCGGCTGAACGATGGGGGGCTGCTCCTGTCCTACAATGG CAGCAGTTACACTACGTACATGAAGGAAGAGGTTGACA GTTACCGGATCACTATCGGCAATAAGACTTGCGTGTTTG AAAAGGAAAACGACCCCACTGTCCTGAGATCCCCCTCGG CTGGGAAGCTGATGCAGTATACGGTGGAGGACGGAGAC CACGTGGAAGCTGGGAGCAGCTACGCGGAGATGGAGGT GATGAAGATGATCATGACCCTGAACGTGCAGGAGAGCG GCCGGGTGAAGTACATCAAGCGGCCAGGGGTTATACTG GAGGCTGGCTGCGTGGTGGCGAGGCTAGAACTCGATGA CCCTTCAAAAGTGCACGCGGCCCAGCCGTTTACTGGGGA GCTCCCTGCCCAGCAGACTCTGCCCATCCTCGGGGAGAA GCTACACCAAGTGTTCCACGGCGTCTTGGAAAATCTGAC CAATGTCATGAGTGGCTACTGCCTGCCCGAGCCCTTCTT CAGCATGAAGCTGAAGGACTGGGTCCAGAAGCTCATGA TGACGCTCCGGCATCCCTCCCTACCTCTGCTGGAGCTAC AGGAGATCATGACCAGTGTGGCCGGCCGCATCCCCGCCC CGGTGGAGAAGGCAGTCCGCAGGGTGATGGCCCAGTAC GCCAGTAACATCACCTCCGTGCTATGCCAGTTCCCCAGC CAGCAGATAGCCACCATCCTGGACTGCCATGCCGCCACC CTGCAGCGTAAGGCGGACCGAGAGGTCTTCTTCATGAAC ACACAGAGCATCGTGCAGCTGGTCCAGAGATACCGCAG CGGGACCCGTGGCTACATGAAGGCTGTGGTGCTGGACCT CCTGAGGAAATATCTAAACGTGGAGCACCATTTTCAGCA AGCCCACTATGACAAGTGTGTGATCAACCTGAGGGAGC AGTTCAAGCCGGACATGACCCAGGTGCTGGACTGCATCT TCTCACACTCCCAGGTGGCCAAGAAGAACCAGCTGGTGA CCATGCTGATAGATGAGCTGTGCGGCCCAGACCCCACCC TGTCAGACGAGCTGACCTCCATCCTCTGTGAGCTCACGC AGCTGAGCAGAAGCGAGCACTGCAAGGTTGCCCTCAGA GCCAGACAGGTCCTGATCGCCTCCCACCTCCCCTCCTAC GAGCTGCGGCACAACCAGGTGGAGTCCATCTTCCTGTCC GCCATCGACATGTACGGCCACCAGTTCTGCCCAGAAAAT CTCAAGAAACTAATACTTTCAGAAACGACCATATTCGAC GTCCTGCCCACTTTCTTCTATCACGAGAACAAGGTTGTCT GTATGGCGTCCCTGGAGGTTTACGTGCGGAGAGGCTACA TCGCCTACGAGCTAAACAGCCTGCAGCACCGGGAGCTCC CAGATGGCACCTGTGTGGTGGAGTTCCAGTTCATGCTGC CCTCTTCCCACCCCAACCGGATGGCCGTGCCCATCAGTG TCTCCAACCCCGACCTGCTGAGGCACAGCACAGAACTCT TCATGGACAGTGGCTTCTCCCCACTGTGCCAGCGGATGG GGGCCATGGTGGCCTTCAGGAGATTTGAAGAATTCACCA GGAACTTTGATGAAGTCATCTCCTGCTTTGCCAACGTGC AGACAGACACTCTTCTCTTCAGTAAGGCGTGCACTTCCC |
| | | TGTACTCAGAGGAAGACAGCAAGAGCCTCCGAGAGGAA CCCATCCACATCCTGAATGTGGCCATCCAGTGTGCCGAC CACATGGAGGACGAGGCGCTGGTGCCAGTCTTCCGTGCC TTTGTACAGTCCAAGAAGCACATCCTTGTGGATTACGGA CTCCGAAGAATCACATTCCTTGTTGCCCAAGAGAGAGAA TTCCCCAAGTTCTTCACGTTCAGAGCGAGGGATGAGTTT GCAGAAGACCGGATTTATCGCCACTTGGAGCCTGCCCTG GCCTTCCAGCTGGAGCTGAGCCGGATGCGCAACTTTGAC CTGACCGCTGTGCCCTGTGCCAACCACAAGATGCATCTT TACCTGGGAGCCGCTAAGGTGAAGGAAGGGTTGGAGGT GACCGACCACAGGTTCTTTATCCGAGCCATTATCCGGCA CTCAGACCTGATCACCAAGGAAGCCTCCTTCGAGTACCT GCAGAACGAAGGTGAGCGGCTGCTGCTGGAAGCCATGG ACGAGCTGGAGGTGGCGTTCAACAACACCAGTGTGCGC ACTGACTGCAACCACATCTTCCTCAACTTTGTGCCCACA GTCATCATGGACCCGCTCAAGATCGAGGAGTCGGTGCGT GACATGGTCATGCGCTATGGCAGCCGTCTGTGGAAGCTC CGAGTGCTGCAGGCCGAGGTTAAGATCAACATCCGTCAG ACAACCTCAGATAGTGCCATCCCCATCCGCCTCTTCATC ACCAACGAGTCTGGCTACTACCTGGACATCAGCCTCTAT AGAGAAGTGACGGACTCCAGATCCGGAAATATCATGTTT CATTCCTTTGGCAACAAGCAAGGTAGCCTGCATGGGATG CTGATCAATACACCCTATGTCACCAAGGACCTGCTCCAG GCTAAGCGATTCCAGGCACAGTCCCTCGGGACCACCTAT GTGTACGACTTCCCAGAGATGTTCAGGCAGGCTCTCTTT AAACTGTGGGGCTCCCCAGAGAAGTACCCCAAAGATAT CCTGACATACACAGAGCTGGTGTTGGACTCTCAAGGTCA GCTGGTGGAGATGAACCGGCTTCCTGGTTGTAACGAGGT GGGCATGGTGGCCTTCAAAATGAGGTTCAAGACCCCGG AGTATCCAGAAGGCCGGGACGCCGTTGTCATCGGCAATG ACATCACCTTCCAAATCGGCTCTTTCGGCATAGGGGAGG ACTTCCTGTACCTGCGAGCGTCTGAGATGGCCCGGACAG AGGGCATCCCCCAAATCTACCTGGCAGCTAACAGCGGG GCCCGTATGGGCCTGGCTGAGGAGATCAAACAGATATTC CAAGTGGCTTGGGTGGACCCAGAGGATCCCCACAAAGG ATTTAGATACCTGTACCTGACGCCCCAAGACTACACCCA GATCAGTTCCCAGAACTCAGTGCACTGCAAGCACATCGA AGATGAAGGCGAATCCAGATACGTCATCGTGGATGTCAT CGGGAAGGATGCCAACCTGGGTGTGGAGAACCTGAGGG GCTCGGGCATGATTGCAGGAGAAGCTTCCCTGGCTTATG AGAAAACGGTCACCATCAGCATGGTGACCTGCCGCGCCC TTGGAATCGGGGCTTACCTGGTGAGGCTTGGCCAGCGGG TGATCCAGGTGGAAAACTCCCACATCATCCTCACCGGAG CCGGTGCTCTCAACAAGGTCCTGGGAAGAGAGGTCTACA CGTCCAACAACCAGCTAGGTGGCGTGCAGATCATGCATA CCAACGGGGTCTCCCACGTCACCGTGCCAGATGACTTCG AGGGGGTCTGCACCATTCTGGAATGGCTGTCATTTATAC CGAAGGACAATCGCAGCCCGGTCCCCATCACCACTCCTT CTGACCCCATCGACAGGGAAATTGAATTCACCCCAACCA AAGCTCCCTACGACCCCAGGTGGATGCTTGCAGGCAGGC |
| | | CTCACCCAACTCTGAAGGGGACCTGGCAGAGTGGATTCT TCGACCATGGCAGTTTCAAGGAAATCATGGCACCCTGGG CCCAGACTGTGGTGACGGGACGAGCAAGGCTGGGGGGC ATCCCTGTGGGAGTGATTGCTGTGGAGACTCGGACTGTG GAGGTGGCTGTCCCCGCTGACCCTGCCAACTTGGATTCT GAGGCCAAGATCATCCAGCAGGCGGGCCAGGTGTGGTT CCCGGACTCGGCCTACAAGACGGCGCAGGTCATCAGGG ACTTCAACAAGGAGCGCCTGCCTCTCATGATCTTCGCCA ACTGGAGGGGCTTCTCCGGAGGCATGAAGGACATGTAT GAGCAGATGCTGAAGTTTGGCGCCTACATCGTGGATGGT CTCCGCCTGTACGAGCAGCCCATTCTCATCTACATCCCTC CCTGTGCGGAGCTCCGTGGGGGCTCCTGGGTTGTCCTGG ACTCCACCATCAACCCCCTGTGCATAGAGATGTATGCAG ACAAAGAGAGCAGGGGGGGTGTTCTGGAGCCGGAGGGC ACGGTGGAGATTAAGTTCCGGAAGAAAGATCTGGTGAA AACCATAAGGAGGATAGACCCAGTGTGTAAGAAGCTCG TGGGACAGCTGGGGAAAGCCCAGCTTCCCGACAAGGAC CGGAAGGAGCTGGAGGGCCAGCTGAAGGCCCGGGAGGA ACTGCTGCTTCCCATCTACCACCAGGTGGCAGTGCAGTT CGCAGACCTGCACGACACCCCGGGCCACATGCTGGAGA AAGGAATCATTTCTGATGTGCTGGAATGGAAGACTGCCC GCACCTTCTTCTACTGGAGGCTGCGCCGGCTGCTGCTGG AGGCCCAGGTGAAGCAGGAGATACTCCGAGCCAGCCCC GAGCTGAACCACGAGCACACACAGTCCATGCTGCGACG CTGGTTCGTGGAGACTGAGGGCGCTGTCAAGGCCTACCT GTGGGACAGCAACCAGGTGGTGGTCCAGTGGCTGGAAC AGCACTGGTCAGCCAAGGACGGCTTGCGCTCCACCATCC GTGAAAACATCAATTATCTGAAGCGGGACTCTGTCCTCA AGACCATCCAAAGTCTGGTCCAAGAACATCCAGAGGTC ATCATGGACTGCGTGGCCTACCTGAGCCAGCACCTCACG CCAGCCGAGCGGATACAGGTGGCTCAGCTGCTGTCTACC ACGGAGAGCCCAGCTTCCTCCTGATGAGCAACTCTGGCC ATCCCCAGGACCCTGGACGGTGGGATGGCCATGCGGCA GGCCTTCGAGGGACACGGGCTTATTTTGGGAAAACTCAC GGAGCCACTCCAAATGTAGCTGCGGTTCCTGACCAGCGA CTTCTGGACAGAAGGCCCCTGGGCCCCTGCTAAGGCGGG CCCAGCCTCTCCTCACCTGGGGACTTCAGGGTTTGTCCC AGTGGATAATTCTGCCACTGCCTCTCTGAGGCCAACTGA ACCAGGCACACCGAGTTCCTTGTGGTTTTTGCCTGAGGT GGGGATCCGGTGTCTGATCACAAGACACTTGTTCTACCA CGTTGGTTTGCACTCAGCTCCTTGTGTCCCTCAGCCGAGT AAATGGGCCAAAGAGAACGCCAGATGAGACCCAAGAAA ACGTCTTTTTGTGATGAGGCTTATTAGACACTTAGCAAG AGCAAAAATGATTTCCTTTTTTGTTGTTGTTGTTGTTTTTC ATTTAAGAGAATAAAGTGACATTTTATTCTCCAAGTGGC CCTAGTGAGTCAGGACAACCATCTGTCCCATTAGAGAGG TTCTCCAGAGATAAAGGACAAACAGACAGGACACCTAG CATTCAGCATGGCTGGAGGAGAGTTACTGCCTCGGTTCC CAGGCTCTGTAAGCCCTCTCTTCCCAAGTGGAGCAAGGA CTGGACCTCCGTGCCTCCTGCAGATTGGCCACCCACAGG |
| | | GTACTGAGTGCCCTACTGCCTGAGTGCAGAGAGGCTGTC CCTCACCCCTCCCAGAACCTCTGGCCAATATACTTCCTCA CCTTGTGCCTTGATACCACGGCAGGAGCCAACAGCGCTG GACCAGTGACCCAGGGCTGTGCCTCGAACACTCTGGATC CGCTTACAGAGACTTTCCCAGCACTGCCAGGCACCGCAG CATCAGGACCCGAATGAGATGCTCCGGCCAGGGGTGGG GTGGGGATGTTGGGGTAGTGGAGAACCCCACTCTGTCGC TGCCCTTCTTGGGGGATAAAGGGTTCTTCTGGGCAAGGT CCTCTTGATGTGGAGGAAAGCCCAGACCGACACTGGCTG CCACCCTGACCACTTGGGTGCGCTGAAGAGGCTGCTGAA AGAGCTGCAAAATAAACTATTTTTTTTGAAGAAAAAAAA AAAA |
| 156 | DGAT2 (variant 1) Human NM_032564.5 (GenBank RefSeq #) | GCCGCGGCTGCCGCCTCTGCTGGGGTCTAGGCTGTTTCT CTCGCGCCACCACTGGCCGCCGGCCGCAGCTCCAGGTGT CCTAGCCGCCCAGCCTCGACGCCGTCCCGGGACCCCTGT GCTCTGCGCGAAGCCCTGGCCCCGGGGGCCGGGGCATG GGCCAGGGGCGCGGGGTGAAGCGGCTTCCCGCGGGGCC GTGACTGGGCGGGCTTCAGCCATGAAGACCCTCATAGCC GCCTACTCCGGGGTCCTGCGCGGCGAGCGTCAGGCCGAG GCTGACCGGAGCCAGCGCTCTCACGGAGGACCTGCGCTG TCGCGCGAGGGGTCTGGGAGATGGGGCACTGGATCCAG CATCCTCTCCGCCCTCCAGGACCTCTTCTCTGTCACCTGG CTCAATAGGTCCAAGGTGGAAAAGCAGCTACAGGTCAT CTCAGTGCTCCAGTGGGTCCTGTCCTTCCTTGTACTGGGA GTGGCCTGCAGTGCCATCCTCATGTACATATTCTGCACT GATTGCTGGCTCATCGCTGTGCTCTACTTCACTTGGCTGG TGTTTGACTGGAACACACCCAAGAAAGGTGGCAGGAGG TCACAGTGGGTCCGAAACTGGGCTGTGTGGCGCTACTTT CGAGACTACTTTCCCATCCAGCTGGTGAAGACACACAAC CTGCTGACCACCAGGAACTATATCTTTGGATACCACCCC CATGGTATCATGGGCCTGGGTGCCTTCTGCAACTTCAGC ACAGAGGCCACAGAAGTGAGCAAGAAGTTCCCAGGCAT ACGGCCTTACCTGGCTACACTGGCAGGCAACTTCCGAAT GCCTGTGTTGAGGGAGTACCTGATGTCTGGAGGTATCTG CCCTGTCAGCCGGGACACCATAGACTATTTGCTTTCAAA GAATGGGAGTGGCAATGCTATCATCATCGTGGTCGGGGG TGCGGCTGAGTCTCTGAGCTCCATGCCTGGCAAGAATGC AGTCACCCTGCGGAACCGCAAGGGCTTTGTGAAACTGGC CCTGCGTCATGGAGCTGACCTGGTTCCCATCTACTCCTTT GGAGAGAATGAAGTGTACAAGCAGGTGATCTTCGAGGA GGGCTCCTGGGGCCGATGGGTCCAGAAGAAGTTCCAGA AATACATTGGTTTCGCCCCATGCATCTTCCATGGTCGAG GCCTCTTCTCCTCCGACACCTGGGGGCTGGTGCCCTACTC CAAGCCCATCACCACTGTTGTGGGAGAGCCCATCACCAT CCCCAAGCTGGAGCACCCAACCCAGCAAGACATCGACC TGTACCACACCATGTACATGGAGGCCCTGGTGAAGCTCT TCGACAAGCACAAGACCAAGTTCGGCCTCCCGGAGACT GAGGTCCTGGAGGTGAACTGAGCCAGCCTTCGGGGCCA ATTCCCTGGAGGAACCAGCTGCAAATCACTTTTTTGCTCT GTAAATTTGGAAGTGTCATGGGTGTCTGTGGGTTATTTA |
| | | AAAGAAATTATAACAATTTTGCTAAACCATTACAATGTT AGGTCTTTTTTAAGAAGGAAAAAGTCAGTATTTCAAGTT CTTTCACTTCCAGCTTGCCCTGTTCTAGGTGGTGGCTAAA TCTGGGCCTAATCTGGGTGGCTCAGCTAACCTCTCTTCTT CCCTTCCTGAAGTGACAAAGGAAACTCAGTCTTCTTGGG GAAGAAGGATTGCCATTAGTGACTTGGACCAGTTAGATG ATTCACTTTTTGCCCCTAGGGATGAGAGGCGAAAGCCAC TTCTCATACAAGCCCCTTTATTGCCACTACCCCACGCTCG TCTAGTCCTGAAACTGCAGGACCAGTTTCTCTGCCAAGG GGAGGAGTTGGAGAGCACAGTTGCCCCGTTGTGTGAGG GCAGTAGTAGGCATCTGGAATGCTCCAGTTTGATCTCCC TTCTGCCACCCCTACCTCACCCCTAGTCACTCATATCGGA GCCTGGACTGGCCTCCAGGATGAGGATGGGGGTGGCAA TGACACCCTGCAGGGGAAAGGACTGCCCCCCATGCACC ATTGCAGGGAGGATGCCGCCACCATGAGCTAGGTGGAG TAACTGGTTTTTCTTGGGTGGCTGATGACATGGATGCAG CACAGACTCAGCCTTGGCCTGGAGCACATGCTTACTGGT GGCCTCAGTTTACCTTCCCCAGATCCTAGATTCTGGATGT GAGGAAGAGATCCCTCTTCAGAAGGGGCCTGGCCTTCTG AGCAGCAGATTAGTTCCAAAGCAGGTGGCCCCCGAACC CAAGCCTCACTTTTCTGTGCCTTCCTGAGGGGGTTGGGC CGGGGAGGAAACCCAACCCTCTCCTGTGTGTTCTGTTAT CTCTTGATGAGATCATTGCACCATGTCAGACTTTTGTATA TGCCTTGAAAATAAATGAAAGTGAGAATCCTC |
| 157 | DGAT2 (variant 2) Human NM_00125389 1.1 (GenBank RefSeq #) | TGCCCCGTTGTGAGGTGATAAAGTGTTGCGCTCCGGGAC GCCAGCGCCGCGGCTGCCGCCTCTGCTGGGGTCTAGGCT GTTTCTCTCGCGCCACCACTGGCCGCCGGCCGCAGCTCC AGGTGTCCTAGCCGCCCAGCCTCGACGCCGTCCCGGGAC CCCTGTGCTCTGCGCGAAGCCCTGGCCCCGGGGGCCGGG GCATGGGCCAGGGGCGCGGGGTGAAGCGGCTTCCCGCG GGGCCGTGACTGGGCGGGCTTCAGCCATGAAGACCCTCA TAGCCGCCTACTCCGGGGTCCTGCGCGGCGAGCGTCAGG CCGAGGCTGACCGGAGCCAGCGCTCTCACGGAGGACCT GCGCTGTCGCGCGAGGGGTCTGGGAGATGGGGAGTGGC CTGCAGTGCCATCCTCATGTACATATTCTGCACTGATTGC TGGCTCATCGCTGTGCTCTACTTCACTTGGCTGGTGTTTG ACTGGAACACACCCAAGAAAGGTGGCAGGAGGTCACAG TGGGTCCGAAACTGGGCTGTGTGGCGCTACTTTCGAGAC TACTTTCCCATCCAGCTGGTGAAGACACACAACCTGCTG ACCACCAGGAACTATATCTTTGGATACCACCCCCATGGT ATCATGGGCCTGGGTGCCTTCTGCAACTTCAGCACAGAG GCCACAGAAGTGAGCAAGAAGTTCCCAGGCATACGGCC TTACCTGGCTACACTGGCAGGCAACTTCCGAATGCCTGT GTTGAGGGAGTACCTGATGTCTGGAGGTATCTGCCCTGT CAGCCGGGACACCATAGACTATTTGCTTTCAAAGAATGG GAGTGGCAATGCTATCATCATCGTGGTCGGGGGTGCGGC TGAGTCTCTGAGCTCCATGCCTGGCAAGAATGCAGTCAC CCTGCGGAACCGCAAGGGCTTTGTGAAACTGGCCCTGCG TCATGGAGCTGACCTGGTTCCCATCTACTCCTTTGGAGA GAATGAAGTGTACAAGCAGGTGATCTTCGAGGAGGGCT |
| | | CCTGGGGCCGATGGGTCCAGAAGAAGTTCCAGAAATAC ATTGGTTTCGCCCCATGCATCTTCCATGGTCGAGGCCTCT TCTCCTCCGACACCTGGGGGCTGGTGCCCTACTCCAAGC CCATCACCACTGTTGTGGGAGAGCCCATCACCATCCCCA AGCTGGAGCACCCAACCCAGCAAGACATCGACCTGTAC CACACCATGTACATGGAGGCCCTGGTGAAGCTCTTCGAC AAGCACAAGACCAAGTTCGGCCTCCCGGAGACTGAGGT CCTGGAGGTGAACTGAGCCAGCCTTCGGGGCCAATTCCC TGGAGGAACCAGCTGCAAATCACTTTTTTGCTCTGTAAA TTTGGAAGTGTCATGGGTGTCTGTGGGTTATTTAAAAGA AATTATAACAATTTTGCTAAACCATTACAATGTTAGGTC TTTTTTAAGAAGGAAAAAGTCAGTATTTCAAGTTCTTTC ACTTCCAGCTTGCCCTGTTCTAGGTGGTGGCTAAATCTG GGCCTAATCTGGGTGGCTCAGCTAACCTCTCTTCTTCCCT TCCTGAAGTGACAAAGGAAACTCAGTCTTCTTGGGGAAG AAGGATTGCCATTAGTGACTTGGACCAGTTAGATGATTC ACTTTTTGCCCCTAGGGATGAGAGGCGAAAGCCACTTCT CATACAAGCCCCTTTATTGCCACTACCCCACGCTCGTCTA GTCCTGAAACTGCAGGACCAGTTTCTCTGCCAAGGGGAG GAGTTGGAGAGCACAGTTGCCCCGTTGTGTGAGGGCAGT AGTAGGCATCTGGAATGCTCCAGTTTGATCTCCCTTCTGC CACCCCTACCTCACCCCTAGTCACTCATATCGGAGCCTG GACTGGCCTCCAGGATGAGGATGGGGGTGGCAATGACA CCCTGCAGGGGAAAGGACTGCCCCCCATGCACCATTGCA GGGAGGATGCCGCCACCATGAGCTAGGTGGAGTAACTG GTTTTTCTTGGGTGGCTGATGACATGGATGCAGCACAGA CTCAGCCTTGGCCTGGAGCACATGCTTACTGGTGGCCTC AGTTTACCTTCCCCAGATCCTAGATTCTGGATGTGAGGA AGAGATCCCTCTTCAGAAGGGGCCTGGCCTTCTGAGCAG CAGATTAGTTCCAAAGCAGGTGGCCCCCGAACCCAAGCC TCACTTTTCTGTGCCTTCCTGAGGGGGTTGGGCCGGGGA GGAAACCCAACCCTCTCCTGTGTGTTCTGTTATCTCTTGA TGAGATCATTGCACCATGTCAGACTTTTGTATATGCCTTG AAAATAAATGAAAGTGAGAATCCTCTAAAAAAAAAAAA |
| 158 | DGAT2 Cynomolgus monkey (Mf) XM_00557911 8.2 (GenBank RefSeq #) | GCCCAGTTGTGAGGTGATAAAGTGCCGCGCTCCCGGACG CCAGCACCGCCGTCGCAGTCGCTGCTGCGCCCTAGGTTG TTTCTCCCGCGCCGCCTCTGGCCGACTGGCCGACCGGCC GCAGCTCCAGGTGTCCTAGCCACCCAACCTCGGCGCCGT CCCGGGGCCCCCGTGCTCTGCGCGAAGCCCTGGCCCCGG CGGCCGGGGCATGGGCCAGGGGCTCGGGGTGAGGCGGC TTCCCGCGGGGCCGTGACTGGGCGGGCTTCATATTCAGC CATGAAGACCCTCATAGCCGCCTACTCCGGGGTCCTGCG CGGAGAGCGTCAGGCCGAGGCTGACCGGAGCCAGCGCT CTCACGGAGGACCTGCGCTGTCGCGCGAGGGGTCTGGG AGATGGGGCACTGGATCCAGCATCCTCTCCGCCCTCCAG GACCTCTTCTCCGTCACCTGGCTCAATAGGTCCAAGGTG GAAAAGCAGCTACAGGTCATCTCAGTGCTCCAGTGGGTC CTGTCCTTCCTTGTACTGGGAGTGGCCTGCAGTGCCATCC TCATGTACATATTCTGCACCGATTGCTGGCTCATCGCTGT GCTCTACTTCACTTGGCTGGTGTTTGACTGGAACACACC |
| | | CAAGAAGGGTGGCAGGAGGTCACAGTGGGTCCGAAACT GGGCTGTGTGGCGCTACTTTCGAGACTACTTTCCCATCC AGCTGGTGAAGACACACAACCTGCTGACCACCAGGAAC TATATCTTTGGATACCACCCCCATGGCATCATGGGCCTG GGTGCCTTCTGCAACTTCAGCACAGAGGCCACAGAAGTG AGCAAGAAGTTCCCAGGCATACGGCCTTACCTGGCTACA CTGGCGGGCAACTTCCGATTGCCTGTGTTGAGGGAGTAC CTGATGTCTGGAGGTATCTGCCCTGTCAACCGGGACACC ATAGACTATTTGCTTTCAAAGAATGGGAGTGGCAATGCT ATCATCATCGTGGTGGGGGGTGCGGCTGAGTCTCTGAGC TCCATGCCTGGCAAGAATGCAGTCACCCTGCGGAACCGC AAGGGCTTTGTGAAACTGGCCCTGCGCCATGGAGCCGAC CTGGTTCCCATGTACTCCTTTGGAGAGAATGAAGTGTAC AAGCAGGTGATCTTCGAGGAGGGCTCCTGGGGCCGATG GGTCCAGAAGAAGTTCCAGAAATACATTGGTTTCGCCCC ATGCATCTTCCATGGCCGAGGCCTCTTCTCCTCCGACACC TGGGGGCTGGTGCCCTACTCCAAGCCCATCACCACTGTT GTGGGAGAGCCCATCACCATCCCCAAGCTGGAGCACCC AACCCAGCAAGATATCGACCTGTACCACACCATGTACAT GGAGGCCCTGGTGAAGCTCTTCGACAAGCACAAGACCA AGTTCGGCCTCCTGGAGACTGAGGTCCTGGAGGTGAACT GAGCCAGGCTTCGGGGCCAACTCCTTGGAGGAACCAGTT GCAAATTGCTTTTATGCTCTGTAAATTTGGAAGCGTCAT GGGTGTCTGTGGGTTATTTAAAAGAAATTATAATAATTT TGCTAAACCATTACAATGTTAGGTCTTTTTTAAGAAGGA AAAAGTCAGTAATTCAAGCTCTTTTACTTCCAGTTTGCCC TGTTCTAGGTGGTGGCTAAATCTGGGCCTAATCTGGGTG GCTCAGCTAACCTCTATTCTTCCCTTCCTGAAGTGACAGA GGAAACTCAGTCTTGGCCTGGGGAAGAAGGATCGCCATT AGTGACTTGGGCCAGTTAGATGATTCACTTTTTGCCCCC AGGGATGAGAGGCAAAAGCCACTTCTCATACAAGCCCC TCTATTGCCACTACCCCACACTTGTCTAGTCCTGAAACTG CAGGACCAGTTTCTCTGCCAAGGGGAGGAGTTGGAGAG CACGGTTGACCTGTTGTGTGAGGACAGTAGTAGGCATCT GGAATGCTCCTGTTTGATCTCCCTTCTGTCACCCACTCCT CACCCCCAGTCACTCACATCAGAGCCTGGACTGGCCTTC AGGATGAGGATGGGGGTGGCAATGACACCGTGCAGGGG AAAGGACTGCCCCCCATGCACCATTGCAGAGAGGATGC CGCCACCATGAGCTAGGTGGAGTAACTGGTTTTTCTTGG GTGGCTGATGACATGGATGCAGCACAGACTCAGCCTTGG CCTGGAGCACATACTTGCTGGTGGCCTCAGTTTACCTTCC CCACATCACAGATTCTGGATGTGAGGAAGAGGTCCCTCT TCAGAAGGGGCCTGGCCTTCTGAGCAGCAGATTAGTTCC AAAGCAGGTGGCCCCCGAACCCAAGCCTCACTTTTCTGT GCCTTCCTGAGGGGGTTGGGCCAGGGAGGAAACCCAAC CCTCTCCTGTGTATTCTGTTATCTCTTGATGAGATCATAG CACCATGTCAGACTTTTGTATATGCCTAGAAAATAAATG AAAGTGAGAATCCTCTA |
| 159 | Stem loop sequence | GCAGCCGAAAGGCUGC |
| 160 | GalXC-ACAC-1919 (36mer sense strand) | GUUAUGUGAGUGCUGGGACA GCAGCCGAAAGGCUGC |
| 161 | GalXC-ACAC-2384 (36mer sense strand) | UUUCAAACAUGGUGGUGGCA GCAGCCGAAAGGCUGC |
| 162 | GalXC-ACAC-2389 (36mer sense strand) | AACAUGGUGGUGGCUUUGAA GCAGCCGAAAGGCUGC |
| 163 | GalXC-ACAC-2392 (36mer sense strand) | AUGGUGGUGGCUUUGAAGGA GCAGCCGAAAGGCUGC |
| 164 | GalXC-ACAC-2896 (36mer sense strand) | ACCUGUGUGUUUGAGAAGGA GCAGCCGAAAGGCUGC |
| 165 | GalXC-ACAC-2897 (36mer sense strand) | CCUGUGUGUUUGAGAAGGAA GCAGCCGAAAGGCUGC |
| 166 | GalXC-ACAC-3442 (36mer sense strand) | CAGUAUGCUAGCAACAUCAA GCAGCCGAAAGGCUGC |
| 167 | GalXC-ACAC-3443 (36mer sense strand) | AGUAUGCUAGCAACAUCACA GCAGCCGAAAGGCUGC |
| 168 | GalXC-ACAC-4201 (36mer sense strand) | GUGGUGGAAUUCCAGUUCAA GCAGCCGAAAGGCUGC |
| 169 | GalXC-ACAC-4204 (36mer sense strand) | GUGGAAUUCCAGUUCAUGCA GCAGCCGAAAGGCUGC |
| 170 | GalXC-ACAC-4205 (36mer sense strand) | UGGAAUUCCAGUUCAUGCUA GCAGCCGAAAGGCUGC |
| 171 | GalXC-ACAC-4206 (36mer sense strand) | GGAAUUCCAGUUCAUGCUGA GCAGCCGAAAGGCUGC |
| 172 | GalXC-ACAC-4207 (36mer sense strand) | GAAUUCCAGUUCAUGCUGCA GCAGCCGAAAGGCUGC |
| 173 | GalXC-ACAC-4208 (36mer sense strand) | AAUUCCAGUUCAUGCUGCCA GCAGCCGAAAGGCUGC |
| 174 | GalXC-ACAC-5082 (36mer sense strand) | UAACCACAUCUUCCUCAACA GCAGCCGAAAGGCUGC |
| 175 | GalXC-ACAC-5083 (36mer sense strand) | AACCACAUCUUCCUCAACUA GCAGCCGAAAGGCUGC |
| 176 | GalXC-ACAC-5084 (36mer sense strand) | ACCACAUCUUCCUCAACUUA GCAGCCGAAAGGCUGC |
| 177 | GalXC-ACAC-5305 (36mer sense strand) | UACAAGGAAGUGACUGACUA GCAGCCGAAAGGCUGC |
| 178 | GalXC-ACAC-5307 (36mer sense strand) | CAAGGAAGUGACUGACUCCA GCAGCCGAAAGGCUGC |
| 179 | GalXC-ACAC-5309 (36mer sense strand) | AGGAAGUGACUGACUCCAGA GCAGCCGAAAGGCUGC |
| 180 | GalXC-ACAC-6331 (36mer sense strand) | GAUGACUUUGAAGGGGUUUA GCAGCCGAAAGGCUGC |
| 181 | GalXC-ACAC-6683 (36mer sense strand) | UGGAUUCUGAAGCCAAGAUA GCAGCCGAAAGGCUGC |
| 182 | GalXC-ACAC-6684 (36mer sense strand) | GGAUUCUGAAGCCAAGAUAA GCAGCCGAAAGGCUGC |
| 183 | GalXC-ACAC-6685 (36mer sense strand) | GAUUCUGAAGCCAAGAUAAA GCAGCCGAAAGGCUGC |
| 184 | GalXC-ACAC-6686 (36mer sense strand) | AUUCUGAAGCCAAGAUAAUA GCAGCCGAAAGGCUGC |
| 185 | GalXC-ACAC-6755 (36mer sense strand) | AGGCCAUCAAGGACUUCAAA GCAGCCGAAAGGCUGC |
| 186 | GalXC-ACAC-6759 (36mer sense strand) | CAUCAAGGACUUCAACCGGA GCAGCCGAAAGGCUGC |
| 187 | GalXC-ACAC-6761 (36mer sense strand) | UCAAGGACUUCAACCGGGAA GCAGCCGAAAGGCUGC |
| 188 | GalXC-ACAC-6793 (36mer sense strand) | AUGGUCUUUGCCAACUGGAA GCAGCCGAAAGGCUGC |
| 189 | GalXC-ACAC-6845(36mer sense strand) | ACCAAGUGCUGAAGUUUGGA GCAGCCGAAAGGCUGC |
| 190 | GalXC-DGAT2-1139 (36mer sense strand) | AAGAAGUUCCAGAAAUACAA GCAGCCGAAAGGCUGC |
| 191 | GalXC-DGAT2-0899 | AUAGACUAUUUGCUUUCAAA GCAGCCGAAAGGCUGC |
| | (36mer sense strand) | |
| 192 | GalXC-DGAT2-1080(36mer sense strand) | AGAAUGAAGUGUACAAGCAA GCAGCCGAAAGGCUGC |
| 193 | GalXC-DGAT2-1086 (36mer sense strand) | AAGUGUACAAGCAGGUGAUA GCAGCCGAAAGGCUGC |
| 194 | GalXC-DGAT2-0898 (36mer sense strand) | CAUAGACUAUUUGCUUUCAA GCAGCCGAAAGGCUGC |
| 195 | GalXC-DGAT2-0894 (36mer sense strand) | ACACCAUAGACUAUUUGCUA GCAGCCGAAAGGCUGC |
| 196 | GalXC-DGAT2-1074 (36mer sense strand) | UUGGAGAGAAUGAAGUGUAA GCAGCCGAAAGGCUGC |
| 197 | GalXC-DGAT2-1083 (36mer sense strand) | AUGAAGUGUACAAGCAGGUA GCAGCCGAAAGGCUGC |
| 198 | GalXC-DGAT2-0895 (36mer sense strand) | CACCAUAGACUAUUUGCUUA GCAGCCGAAAGGCUGC |
| 199 | GalXC-DGAT2-0897 (36mer sense strand) | CCAUAGACUAUUUGCUUUCA GCAGCCGAAAGGCUGC |
| 200 | GalXC-DGAT2-0509 (36mer sense strand) | CUCAUGUACAUAUUCUGCAA GCAGCCGAAAGGCUGC |
| 201 | GalXC-DGAT2-0893 (36mer sense strand) | GACACCAUAGACUAUUUGCA GCAGCCGAAAGGCUGC |
| 202 | GalXC-DGAT2-1138 (36mer sense strand) | GAAGAAGUUCCAGAAAUACA GCAGCCGAAAGGCUGC |
| 203 | GalXC-DGAT2-0896 (36mer sense strand) | ACCAUAGACUAUUUGCUUUA GCAGCCGAAAGGCUGC |
| 204 | GalXC-DGAT2-0506 (36mer sense strand) | AUCCUCAUGUACAUAUUCUA GCAGCCGAAAGGCUGC |
| 205 | GalXC-DGAT2-1079 (36mer sense strand) | GAGAAUGAAGUGUACAAGCA GCAGCCGAAAGGCUGC |
| 206 | GalXC ACAC-1919 (Modified 36mer sense strand) | [mGs][mU][mU][mA][mU][mG][mU][fG][fA][fG][fU][mG][mC] [mU][mG][mG][mG][mA][mC][mA][mG][mC][mA][mG][mC][ mC][mG][ademA-GalNAc][ademA-GalNAc][ademA-GalNAc][mG][mG][mC][mU][mG][mC] |
| 207 | GalXC ACAC-2384 (Modified 36mer sense strand) | [mUs][mU][mU][mC][mA][mA][mA][fC][fA][fU][fG][mG][mU] [mG][mG][mU][mG][mG][mC][mA][mG][mC][mA][mG][mC][ mC][mG][ademA-GalNAc][ademA-GalNAc][ademA-GalNAc][mG][mG][mC][mU][mG][mC] |
| 208 | GalXC ACAC-2389 (Modified 36mer sense strand) | [mAs][mA][mC][mA][mU][mG][mG][fU][fG][fG][fU][mG][mG] [mC][mU][mU][mU][mG][mA][mA][mG][mC][mA][mG][mC][ mC][mG][ademA-GalNAc][ademA-GalNAc][ademA-GalNAc][mG][mG][mC][mU][mG][mC] |
| 209 | GalXC ACAC-2392 (Modified 36mer sense strand) | [mAs][mU][mG][mG][mU][mG][mG][fU][fG][fG][fC][mU][mU] [mU][mG][mA][mA][mG][mG][mA][mG][mC][mA][mG][mC][ mC][mG][ademA-GalNAc][ademA-GalNAc][ademA-GalNAc][mG][mG][mC][mU][mG][mC] |
| 210 | GalXC ACAC-2896 (Modified 36mer sense strand) | [mAs][mC][mC][mU][mG][mU][mG][fU][fG][fU][fU][mU][mG] [mA][mG][mA][mA][mG][mG][mA][mG][mC][mA][mG][mC][ mC][mG][ademA-GalNAc][ademA-GalNAc][ademA-GalNAc][mG][mG][mC][mU][mG][mC] |
| 211 | GalXC ACAC-2897 (Modified 36mer sense strand) | [mCs][mC][mU][mG][mU][mG][mU][fG][fU][fU][fU][mG][mA] [mG][mA][mA][mG][mG][mA][mA][mG][mC][mA][mG][mC][ mC][mG][ademA-GalNAc][ademA-GalNAc][ademA-GalNAc][mG][mG][mC][mU][mG][mC] |
| 212 | GalXC ACAC-3442 (Modified 36mer sense strand) | [mCs][mA][mG][mU][mA][mU][mG][fC][fU][fA][fG][mC][mA] [mA][mC][mA][mU][mC][mA][mA][mG][mC][mA][mG][mC][ mC][mG][ademA-GalNAc][ademA-GalNAc][ademA-GalNAc][mG][mG][mC][mU][mG][mC] |
| 213 | GalXC ACAC-3443 (Modified 36mer sense strand) | [mAs][mG][mU][mA][mU][mG][mC][fU][fA][fG][fC][mA][mA] [mC][mA][mU][mC][mA][mC][mA][mG][mC][mA][mG][mC][m C][mG][ademA-GalNAc][ademA-GalNAc][ademA-GalNAc][mG][mG][mC][mU][mG][mC] |
| 214 | GalXC ACAC-4201 (Modified | [mGs][mU][mG][mG][mU][mG][mG][fA][fA][fU][fU][mC][mC] [mA][mG][mU][mU][mC][mA][mA][mG][mC][mA][mG][mC][ |
| | 36mer sense strand) | mC][mG][ademA-GalNAc][ademA-GalNAc][ademA-GalNAc][mG][mG][mC][mU][mG][mC] |
| 215 | GalXC ACAC-4204 (Modified 36mer sense strand) | [mGs][mU][mG][mG][mA][mA][mU][fU][fC][fC][fA][mG][mU] [mU][mC][mA][mU][mG][mC][mA][mG][mC][mA][mG][mC][ mC][mG][ademA-GalNAc][ademA-GalNAc][ademA-GalNAc][mG][mG][mC][mU][mG][mC] |
| 216 | GalXC ACAC-4205 (Modified 36mer sense strand) | [mUs][mG][mG][mA][mA][mU][mU][fC][fC][fA][fG][mU][mU] [mC][mA][mU][mG][mC][mU][mA][mG][mC][mA][mG][mC][ mC][mG][ademA-GalNAc][ademA-GalNAc][ademA-GalNAc][mG][mG][mC][mU][mG][mC] |
| 217 | GalXC ACAC-4206 (Modified 36mer sense strand) | [mGs][mG][mA][mA][mU][mU][mC][fC][fA][fG][fU][mU][mC] [mA][mU][mG][mC][mU][mG][mA][mG][mC][mA][mG][mC][ mC][mG][ademA-GalNAc][ademA-GalNAc][ademA-GalNAc][mG][mG][mC][mU][mG][mC] |
| 218 | GalXC ACAC-4207 (Modified 36mer sense strand) | [mGs][mA][mA][mU][mU][mC][mC][fA][fG][fU][fU][mC][mA] [mU][mG][mC][mU][mG][mC][mA][mG][mC][mA][mG][mC][ mC][mG][ademA-GalNAc][ademA-GalNAc][ademA-GalNAc][mG][mG][mC][mU][mG][mC] |
| 219 | GalXC ACAC-4208 (Modified 36mer sense strand) | [mAs][mA][mU][mU][mC][mC][mA][fG][fU][fU][fC][mA][mU] [mG][mC][mU][mG][mC][mC][mA][mG][mC][mA][mG][mC][m C][mG][ademA-GalNAc][ademA-GalNAc][ademA-GalNAc][mG][mG][mC][mU][mG][mC] |
| 220 | GalXC ACAC-5082 (Modified 36mer sense strand) | [mUs][mA][mA][mC][mC][mA][mC][fA][fU][fC][fU][mU][mC][ mC][mU][mC][mA][mA][mC][mA][mG][mC][mA][mG][mC][m C][mG][ademA-GalNAc][ademA-GalNAc][ademA-GalNAc][mG][mG][mC][mU][mG][mC] |
| 221 | GalXC ACAC-5083 (Modified 36mer sense strand) | [mAs][mA][mC][mC][mA][mC][mA][fU][fC][fU][fU][mC][mC][ mU][mC][mA][mA][mC][mU][mA][mG][mC][mA][mG][mC][m C][mG][ademA-GalNAc][ademA-GalNAc][ademA-GalNAc][mG][mG][mC][mU][mG][mC] |
| 222 | GalXC ACAC-5084 (Modified 36mer sense strand) | [mAs][mC][mC][mA][mC][mA][mU][fC][fU][fU][fC][mC][mU][ mC][mA][mA][mC][mU][mU][mA][mG][mC][mA][mG][mC][m C][mG][ademA-GalNAc][ademA-GalNAc][ademA-GalNAc][mG][mG][mC][mU][mG][mC] |
| 223 | GalXC ACAC-5305 (Modified 36mer sense strand) | [mUs][mA][mC][mA][mA][mG][mG][fA][fA][fG][fU][mG][mA] [mC][mU][mG][mA][mC][mU][mA][mG][mC][mA][mG][mC][ mC][mG][ademA-GalNAc][ademA-GalNAc][ademA-GalNAc][mG][mG][mC][mU][mG][mC] |
| 224 | GalXC ACAC-5307 (Modified | [mCs][mA][mA][mG][mG][mA][mA][fG][fU][fG][fA][mC][mU] [mG][mA][mC][mU][mC][mC][mA][mG][mC][mA][mG][mC][m |
| | 36mer sense strand) | C][mG][ademA-GalNAc][ademA-GalNAc][ademA-GalNAc][mG][mG][mC][mU][mG][mC] |
| 225 | GalXC ACAC-5309 (Modified 36mer sense strand) | [mAs][mG][mG][mA][mA][mG][mU][fG][fA][fC][fU][mG][mA] [mC][mU][mC][mC][mA][mG][mA][mG][mC][mA][mG][mC][m C][mG][ademA-GalNAc][ademA-GalNAc][ademA-GalNAc][mG][mG][mC][mU][mG][mC] |
| 226 | GalXC ACAC-6331 (Modified 36mer sense strand) | [mGs][mA][mU][mG][mA][mC][mU][fU][fU][fG][fA][mA][mG] [mG][mG][mG][mU][mU][mU][mA][mG][mC][mA][mG][mC][ mC][mG][ademA-GalNAc][ademA-GalNAc][ademA-GalNAc][mG][mG][mC][mU][mG][mC] |
| 227 | GalXC ACAC-6683 (Modified 36mer sense strand) | [mUs][mG][mG][mA][mU][mU][mC][fU][fG][fA][fA][mG][mC] [mC][mA][mA][mG][mA][mU][mA][mG][mC][mA][mG][mC][ mC][mG][ademA-GalNAc][ademA-GalNAc][ademA-GalNAc][mG][mG][mC][mU][mG][mC] |
| 228 | GalXC ACAC-6684 (Modified 36mer sense strand) | [mGs][mG][mA][mU][mU][mC][mU][fG][fA][fA][fG][mC][mC] [mA][mA][mG][mA][mU][mA][mA][mG][mC][mA][mG][mC][ mC][mG][ademA-GalNAc][ademA-GalNAc][ademA-GalNAc][mG][mG][mC][mU][mG][mC] |
| 229 | GalXC ACAC-6685 (Modified 36mer sense strand) | [mGs][mA][mU][mU][mC][mU][mG][fA][fA][fG][fC][mC][mA] [mA][mG][mA][mU][mA][mA][mA][mG][mC][mA][mG][mC][ mC][mG][ademA-GalNAc][ademA-GalNAc][ademA-GalNAc][mG][mG][mC][mU][mG][mC] |
| 230 | GalXC ACAC-6686 (Modified 36mer sense strand) | [mAs][mU][mU][mC][mU][mG][mA][fA][fG][fC][fC][mA][mA] [mG][mA][mU][mA][mA][mU][mA][mG][mC][mA][mG][mC][ mC][mG][ademA-GalNAc][ademA-GalNAc][ademA-GalNAc][mG][mG][mC][mU][mG][mC] |
| 231 | GalXC ACAC-6755 (Modified 36mer sense strand) | [mAs][mG][mG][mC][mC][mA][mU][fC][fA][fA][fG][mG][mA] [mC][mU][mU][mC][mA][mA][mA][mG][mC][mA][mG][mC][ mC][mG][ademA-GalNAc][ademA-GalNAc][ademA-GalNAc][mG][mG][mC][mU][mG][mC] |
| 232 | GalXC ACAC-6759 (Modified 36mer sense strand) | [mCs][mA][mU][mC][mA][mA][mG][fG][fA][fC][fU][mU][mC] [mA][mA][mC][mC][mG][mG][mA][mG][mC][mA][mG][mC][ mC][mG][ademA-GalNAc][ademA-GalNAc][ademA-GalNAc][mG][mG][mC][mU][mG][mC] |
| 233 | GalXC ACAC-6761 (Modified 36mer sense strand) | [mUs][mC][mA][mA][mG][mG][mA][fC][fU][fU][fC][mA][mA] [mC][mC][mG][mG][mG][mA][mA][mG][mC][mA][mG][mC][ mC][mG][ademA-GalNAc][ademA-GalNAc][ademA-GalNAc][mG][mG][mC][mU][mG][mC] |
| 234 | GalXC ACAC-6793 (Modified | [mAs][mU][mG][mG][mU][mC][mU][fU][fU][fG][fC][mC][mA] [mA][mC][mU][mG][mG][mA][mA][mG][mC][mA][mG][mC][ |
| | 36mer sense strand) | mC][mG][ademA-GalNAc][ademA-GalNAc][ademA-GalNAc][mG][mG][mC][mU][mG][mC] |
| 235 | GalXC ACAC-6845 (Modified 36mer sense strand) | [mAs][mC][mC][mA][mA][mG][mU][fG][fC][fU][fG][mA][mA] [mG][mU][mU][mU][mG][mG][mA][mG][mC][mA][mG][mC][ mC][mG][ademA-GalNAc][ademA-GalNAc][ademA-GalNAc][mG][mG][mC][mU][mG][mC] |
| 236 | GalXC Dgat2-1139 (Modified 36mer sense strand) | [mAs][mA][mG][mA][mA][mG][mU][fU][fC][fC][fA][mG][mA] [mA][mA][mU][mA][mC][mA][mA][mG][mC][mA][mG][mC][ mC][mG][ademA-GalNAc][ademA-GalNAc][ademA-GalNAc][mG][mG][mC][mU][mG][mC] |
| 237 | GalXC Dgat2-0899 (Modified 36mer sense strand) | [mAs][mU][mA][mG][mA][mC][mU][fA][fU][fU][fU][mG][mC] [mU][mU][mU][mC][mA][mA][mA][mG][mC][mA][mG][mC][ mC][mG][ademA-GalNAc][ademA-GalNAc][ademA-GalNAc][mG][mG][mC][mU][mG][mC] |
| 238 | GalXC Dgat2-1080 (Modified 36mer sense strand) | [mAs][mG][mA][mA][mU][mG][mA][fA][fG][fU][fG][mU][mA] [mC][mA][mA][mG][mC][mA][mA][mG][mC][mA][mG][mC][ mC][mG][ademA-GalNAc][ademA-GalNAc][ademA-GalNAc][mG][mG][mC][mU][mG][mC] |
| 239 | GalXC Dgat2-1086 (Modified 36mer sense strand) | [mAs][mA][mG][mU][mG][mU][mA][fC][fA][fA][fG][mC][mA] [mG][mG][mU][mG][mA][mU][mA][mG][mC][mA][mG][mC][ mC][mG][ademA-GalNAc][ademA-GalNAc][ademA-GalNAc][mG][mG][mC][mU][mG][mC] |
| 240 | GalXC Dgat2-0898 (Modified 36mer sense strand) | [mCs][mA][mU][mA][mG][mA][mC][fU][fA][fU][fU][mU][mG] [mC][mU][mU][mU][mC][mA][mA][mG][mC][mA][mG][mC][ mC][mG][ademA-GalNAc][ademA-GalNAc][ademA-GalNAc][mG][mG][mC][mU][mG][mC] |
| 241 | GalXC Dgat2-0894 (Modified 36mer sense strand) | [mAs][mC][mA][mC][mC][mA][mU][fA][fG][fA][fC][mU][mA] [mU][mU][mU][mG][mC][mU][mA][mG][mC][mA][mG][mC][ mC][mG][ademA-GalNAc][ademA-GalNAc][ademA-GalNAc][mG][mG][mC][mU][mG][mC] |
| 242 | GalXC Dgat2-1074 (Modified 36mer sense strand) | [mUs][mU][mG][mG][mA][mG][mA][fG][fA][fA][fU][mG][mA] [mA][mG][mU][mG][mU][mA][mA][mG][mC][mA][mG][mC][ mC][mG][ademA-GalNAc][ademA-GalNAc][ademA-GalNAc][mG][mG][mC][mU][mG][mC] |
| 243 | GalXC Dgat2-1083 (Modified 36mer sense strand) | [mAs][mU][mG][mA][mA][mG][mU][fG][fU][fA][fC][mA][mA] [mG][mC][mA][mG][mG][mU][mA][mG][mC][mA][mG][mC][ mC][mG][ademA-GalNAc][ademA-GalNAc][ademA-GalNAc][mG][mG][mC][mU][mG][mC] |
| 244 | GalXC Dgat2-0895 (Modified | [mCs][mA][mC][mC][mA][mU][mA][fG][fA][fC][fU][mA][mU] [mU][mU][mG][mC][mU][mU][mA][mG][mC][mA][mG][mC][ |
| | 36mer sense strand) | mC][mG][ademA-GalNAc][ademA-GalNAc][ademA-GalNAc][mG][mG][mC][mU][mG][mC] |
| 245 | GalXC Dgat2-0897 (Modified 36mer sense strand) | [mCs][mC][mA][mU][mA][mG][mA][fC][fU][fA][fU][mU][mU] [mG][mC][mU][mU][mU][mC][mA][mG][mC][mA][mG][mC][ mC][mG][ademA-GalNAc][ademA-GalNAc][ademA-GalNAc][mG][mG][mC][mU][mG][mC] |
| 246 | GalXC Dgat2-0509 (Modified 36mer sense strand) | [mCs][mU][mC][mA][mU][mG][mU][fA][fC][fA][fU][mA][mU] [mU][mC][mU][mG][mC][mA][mA][mG][mC][mA][mG][mC][ mC][mG][ademA-GalNAc][ademA-GalNAc][ademA-GalNAc][mG][mG][mC][mU][mG][mC] |
| 247 | GalXC Dgat2-0893 (Modified 36mer sense strand) | [mGs][mA][mC][mA][mC][mC][mA][fU][fA][fG][fA][mC][mU] [mA][mU][mU][mU][mG][mC][mA][mG][mC][mA][mG][mC][ mC][mG][ademA-GalNAc][ademA-GalNAc][ademA-GalNAc][mG][mG][mC][mU][mG][mC] |
| 248 | GalXC Dgat2-1138 (Modified 36mer sense strand) | [mGs][mA][mA][mG][mA][mA][mG][fU][fU][fC][fC][mA][mG] [mA][mA][mA][mU][mA][mC][mA][mG][mC][mA][mG][mC][ mC][mG][ademA-GalNAc][ademA-GalNAc][ademA-GalNAc][mG][mG][mC][mU][mG][mC] |
| 249 | GalXC Dgat2-0896 (Modified 36mer sense strand) | [mAs][mC][mC][mA][mU][mA][mG][fA][fC][fU][fA][mU][mU] [mU][mG][mC][mU][mU][mU][mA][mG][mC][mA][mG][mC][ mC][mG][ademA-GalNAc][ademA-GalNAc][ademA-GalNAc][mG][mG][mC][mU][mG][mC] |
| 250 | GalXC Dgat2-0506 (Modified 36mer sense strand) | [mAs][mU][mC][mC][mU][mC][mA][fU][fG][fU][fA][mC][mA] [mU][mA][mU][mU][mC][mU][mA][mG][mC][mA][mG][mC][ mC][mG][ademA-GalNAc][ademA-GalNAc][ademA-GalNAc][mG][mG][mC][mU][mG][mC] |
| 251 | GalXC Dgat2-1079 (Modified 36mer sense strand) | [mGs][mA][mG][mA][mA][mU][mG][fA][fA][fG][fU][mG][mU] [mA][mC][mA][mA][mG][mC][mA][mG][mC][mA][mG][mC][ mC][mG][ademA-GalNAc][ademA-GalNAc][ademA-GalNAc][mG][mG][mC][mU][mG][mC] |
| 252 | GalXC ACAC-1919 (Modified 22mer antisense strand) | [Phosphonate-40-mUs][fGs][fU][mC][fC][mC][fA][mG][mC][fA][mC][mU][mC][f A][mC][mA][mU][mA][mA][mCs][mGs][mG] |
| 253 | GalXC ACAC-2384 (Modified 22mer antisense strand) | [Phosphonate-40-mUs][fGs][fC][mC][fA][mC][fC][mA][mC][fC][mA][mU][mG][f U][mU][mU][mG][mA][mA][mAs][mGs][mG] |
| 254 | GalXC ACAC-2389 (Modified 22mer antisense strand) | [Phosphonate-40-mUs][fUs][fC][mA][fA][mA][fG][mC][mC][fA][mC][mC][mA][f C][mC][mA][mU][mG][mU][mUs][mGs][mG] |
| 255 | GalXC ACAC-2392 (Modified 22mer antisense strand) | [Phosphonate-40-mUs][fCs][fC][mU][fU][mC][fA][mA][mA][fG][mC][mC][mA][f C][mC][mA][mC][mC][mA][mUs][mGs][mG] |
| 256 | GalXC ACAC-2896 (Modified 22mer antisense strand) | [Phosphonate-40-mUs][fCs][fC][mU][fU][mC][fU][mC][mA][fA][mA][mC][mA][f C][mA][mC][mA][mG][mG][mUs][mGs][mG] |
| 257 | GalXC ACAC-2897 (Modified 22mer antisense strand) | [Phosphonate-40-mUs][fUs][fC][mC][fU][mU][fC][mU][mC][fA][mA][mA][mC][f A][mC][mA][mC][mA][mG][mGs][mGs][mG] |
| 258 | GalXC ACAC-3442 (Modified 22mer antisense strand) | [Phosphonate-40-mUs][fUs][fG] [mA] [fU][mG] [fU][mU] [mG] [fC] [mU] [mA] [mG] [ fC][mA][mU][mA][mC][mU][mGs][mGs][mG] |
| 259 | GalXC ACAC-3443 (Modified 22mer antisense strand) | [Phosphonate-40-mUs][fGs][fU][mG][fA][mU][fG][mU][mU][fG][mC][mU][mA][ fG][mC][mA][mU][mA][mC][mUs][mGs][mG] |
| 260 | GalXC ACAC-4201 (Modified 22mer antisense strand) | [Phosphonate-40-mUs][fUs][fG][mA][fA][mC][fU][mG][mG][fA][mA][mU][mU][ fC][mC][mA][mC][mC][mA][mCs][mGs][mG] |
| 261 | GalXC ACAC-4204 (Modified 22mer antisense strand) | [Phosphonate-40-mUs][fGs][fC][mA][fU][mG][fA][mA][mC][fU][mG][mG][mA][ fA][mU][mU][mC][mC][mA][mCs][mGs][mG] |
| 262 | GalXC ACAC-4205 (Modified 22mer antisense strand) | [Phosphonate-40-mUs][fAs][fG][mC][fA][mU][fG][mA][mA][fC][mU][mG][mG][ fA][mA][mU][mU][mC][mC][mAs][mGs][mG] |
| 263 | GalXC ACAC-4206 (Modified 22mer antisense strand) | [Phosphonate-40-mUs][fCs][fA][mG][fC][mA][fU][mG][mA][fA][mC][mU][mG][ fG][mA][mA][mU][mU][mC][mCs][mGs][mG] |
| 264 | GalXC ACAC-4207 (Modified 22mer antisense strand) | [Phosphonate-40-mUs][fGs][fC][mA][fG][mC][fA][mU][mG][fA][mA][mC][mU][ fG][mG][mA][mA][mU][mU][mCs][mGs][mG] |
| 265 | GalXC ACAC-4208 (Modified 22mer antisense strand) | [Phosphonate-40-mUs][fGs][fG][mC][fA][mG][fC][mA][mU][fG][mA][mA][mC][ fU][mG][mG][mA][mA][mU][mUs][mGs][mG] |
| 266 | GalXC ACAC-5082 (Modified 22mer antisense strand) | [Phosphonate-40-mUs][fGs][fU][mU][fG][mA][fG][mG][mA][fA][mG][mA][mU][ fG][mU][mG][mG][mU][mU][mAs][mGs][mG] |
| 267 | GalXC ACAC-5083 (Modified 22mer antisense strand) | [Phosphonate-40-mUs][fAs][fG][mU][fU][mG][fA][mG][mG][fA][mA][mG][mA][ fU][mG][mU][mG][mG][mU][mUs][mGs][mG] |
| 268 | GalXC ACAC-5084 (Modified 22mer antisense strand) | [Phosphonate-40-mUs][fAs][fA][mG][fU][mU][fG][mA][mG][fG][mA][mA][mG][ fA][mU][mG][mU][mG][mG][mUs][mGs][mG] |
| 269 | GalXC ACAC-5305 (Modified 22mer antisense strand) | [Phosphonate-40-mUs][fAs][fG][mU][fC][mA][fG][mU][mC][fA][mC][mU][mU][ fC][mC][mU][mU][mG][mU][mAs][mGs][mG] |
| 270 | GalXC ACAC-5307 (Modified 22mer antisense strand) | [Phosphonate-40-mUs][fGs][fG][mA][fG][mU][fC][mA][mG][fU][mC][mA][mC][ fU][mU][mC][mC][mU][mU][mGs][mGs][mG] |
| 271 | GalXC ACAC-5309 (Modified 22mer antisense strand) | [Phosphonate-40-mUs][fCs][fU][mG][fG][mA][fG][mU][mC][fA][mG][mU][mC][ fA][mC][mU][mU][mC][mC][mUs][mGs][mG] |
| 272 | GalXC ACAC-6331 (Modified 22mer antisense strand) | [Phosphonate-40-mUs][fAs][fA][mA][fC][mC][fC][mC][mU][fU][mC][mA][mA][f A][mG][mU][mC][mA][mU][mCs][mGs][mG] |
| 273 | GalXC ACAC-6683 (Modified 22mer antisense strand) | [Phosphonate-40-mUs][fAs][fU][mC][fU][mU][fG][mG][mC][fU][mU][mC][mA][ fG][mA][mA][mU][mC][mC][mAs][mGs][mG] |
| 274 | GalXC ACAC-6684 (Modified 22mer antisense strand) | [Phosphonate-40-mUs][fUs][fA][mU][fC][mU][fU][mG][mG][fC][mU][mU][mC][ fA][mG][mA][mA][mU][mC][mCs][mGs][mG] |
| 275 | GalXC ACAC-6685 (Modified 22mer antisense strand) | [Phosphonate-40-mUs][fUs][fU][mA] [fU][mC] [fU][mU] [mG] [fG] [mC] [mU] [mU] [ fC][mA][mG][mA][mA][mU][mCs][mGs][mG] |
| 276 | GalXC ACAC-6686 (Modified 22mer antisense strand) | [Phosphonate-40-mUs][fAs][fU][mU][fA][mU][fC][mU][mU][fG][mG][mC][mU][ fU][mC][mA][mG][mA][mA][mUs][mGs][mG] |
| 277 | GalXC ACAC-6755 (Modified 22mer antisense strand) | [Phosphonate-40-mUs][fUs][fU][mG][fA][mA][fG][mU][mC][fC][mU][mU][mG][ fA][mU][mG][mG][mC][mC][mUs][mGs][mG] |
| 278 | GalXC ACAC-6759 (Modified 22mer antisense strand) | [Phosphonate-40-mUs][fCs][fC][mG][fG][mU][fU][mG][mA][fA][mG][mU][mC][ fC][mU][mU][mG][mA][mU][mGs][mGs][mG] |
| 279 | GalXC ACAC-6761 (Modified 22mer antisense strand) | [Phosphonate-40-mUs][fUs][fC][mC][fC][mG][fG][mU][mU][fG][mA][mA][mG][ fU][mC][mC][mU][mU][mG][mAs][mGs][mG] |
| 280 | GalXC ACAC-6793 (Modified 22mer antisense strand) | [Phosphonate-40-mUs][fUs][fC][mC][fA][mG][fU][mU][mG][fG][mC][mA][mA][ fA][mG][mA][mC][mC][mA][mUs][mGs][mG] |
| 281 | GalXC ACAC-6845 (Modified 22mer antisense strand) | [Phosphonate-40-mUs][fCs][fC][mA][fA][mA][fC][mU][mU][fC][mA][mG][mC][f A][mC][mU][mU][mG][mG][mUs][mGs][mG] |
| 282 | GalXC Dgat2-1139 (Modified 22mer antisense strand) | [MePhosphonate-40-mUs][fUs][fG][fU][fA][mU][fU][mU][mC][fU][mG][mG][mA][f A][mC][mU][mU][mC][mU][mUs][mGs][mG] |
| 283 | GalXC Dgat2-0899 (Modified 22mer antisense strand) | [MePhosphonate-40-mUs][fUs][fU][fG][fA][mA][fA][mG][mC][fA][mA][mA][mU][f A][mG][mU][mC][mU][mA][mUs][mGs][mG] |
| 284 | GalXC Dgat2-1080 (Modified 22mer antisense strand) | [MePhosphonate-40-mUs][fUs][fG][fC][fU][mU][fG][mU][mA][fC][mA][mC][mU][f U][mC][mA][mU][mU][mC][mUs][mGs][mG] |
| 285 | GalXC Dgat2-1086 (Modified 22mer antisense strand) | [MePhosphonate-40-mUs][fAs][fU][fC][fA][mC][fC][mU][mG][fC][mU][mU][mG][f U][mA][mC][mA][mC][mU][mUs][mGs][mG] |
| 286 | GalXC Dgat2-0898 (Modified 22mer antisense strand) | [MePhosphonate-40-mUs][fUs][fG][fA][fA][mA][fG][mC][mA][fA][mA][mU][mA][f G][mU][mC][mU][mA][mU][mGs][mGs][mG] |
| 287 | GalXC Dgat2-0894 (Modified 22mer antisense strand) | [MePhosphonate-40-mUs][fAs][fG][fC][fA][mA][fA][mU][mA][fG][mU][mC][mU][f A][mU][mG][mG][mU][mG][mUs][mGs][mG] |
| 288 | GalXC Dgat2-1074 (Modified 22mer antisense strand) | [MePhosphonate-40-mUs][fUs][fA][fC][fA][mC][fU][mU][mC][fA][mU][mU][mC][f U][mC][mU][mC][mC][mA][mAs][mGs][mG] |
| 289 | GalXC Dgat2-1083 (Modified 22mer antisense strand) | [MePhosphonate-40-mUs][fAs][fC][fC][fU][mG][fC][mU][mU][fG][mU][mA][mC][f A][mC][mU][mU][mC][mA][mUs][mGs][mG] |
| 290 | GalXC Dgat2-0895 (Modified 22mer antisense strand) | [MePhosphonate-40-mUs][fAs][fA][fG][fC][mA][fA][mA][mU][fA][mG][mU][mC][f U][mA][mU][mG][mG][mU][mGs][mGs][mG] |
| 291 | GalXC Dgat2-0897 (Modified 22mer antisense strand) | [MePhosphonate-40-mUs][fGs][fA][fA][fA][mG][fC][mA][mA][fA][mU][mA][mG][f U][mC][mU][mA][mU][mG][mGs][mGs][mG] |
| 292 | GalXC Dgat2-0509 (Modified 22mer antisense strand) | [MePhosphonate-40-mUs][fUs][fG][fC][fA][mG][fA][mA][mU][fA][mU][mG][mU][f A][mC][mA][mU][mG][mA][mGs][mGs][mG] |
| 293 | GalXC Dgat2-0893 (Modified 22mer antisense strand) | [MePhosphonate-40-mUs][fGs][fC][fA][fA][mA][fU][mA][mG][fU][mC][mU][mA][f U][mG][mG][mU][mG][mU][mCs][mGs][mG] |
| 294 | GalXC Dgat2-1138 (Modified 22mer antisense strand) | [MePhosphonate-40-mUs][fGs][fU][fA][fU][mU][fU][mC][mU][fG][mG][mA][mA][f C][mU][mU][mC][mU][mU][mCs][mGs][mG] |
| 295 | GalXC Dgat2-0896 (Modified 22mer antisense strand) | [MePhosphonate-40-mUs][fAs][fA][fA][fG][mC][fA][mA][mA][fU][mA][mG][mU][f C][mU][mA][mU][mG][mG][mUs][mGs][mG] |
| 296 | GalXC Dgat2-0506 (Modified 22mer antisense strand) | [MePhosphonate-40-mUs][fAs][fG][fA][fA][mU][fA][mU][mG][fU][mA][mC][mA][f U][mG][mA][mG][mG][mA][mUs][mGs][mG] |
| 297 | GalXC Dgat2-1079 (Modified 22mer antisense strand) | [MePhosphonate-40-mUs] [fGs] [fC] [fU][fU][mG] [fU][mA] [mC] [fA] [mC] [mU] [mU] [f C][mA][mU][mU][mC][mU][mCs][mGs][mG] |

1. An oligonucleotide comprising:
(i) an antisense strand comprising a region of complementarity to a target sequence of ACC, wherein the antisense strand comprises a nucleotide sequence selected from SEQ ID Nos: 2, 30, 32, 44 and 56; or
(ii) an antisense strand comprising a region of complementarity to a target sequence of DGAT2, wherein the antisense strand comprises a nucleotide sequence selected from SEQ ID Nos:118, 120, 126, 130, 138 and 144.

2. The oligonucleotide of claim 1, wherein the oligonucleotide comprises the antisense strand of (i) and a sense strand comprising a nucleotide sequence selected from SEQ ID Nos: 1, 29, 31, 43 and 55; or comprises the antisense strand of (ii) and a sense strand comprising a nucleotide sequence selected from SEQ ID Nos: 117, 119, 125, 129, 137 and 143.

3. An oligonucleotide comprising:
(i) an antisense strand of 15-30 nucleotides in length and a sense strand of 15-40 nucleotides in length, wherein the antisense strand has a region of complementarity to a target sequence of ACC set for in SEQ ID Nos: 150 or 151, and wherein the region of complementarity is at least 15 contiguous nucleotides in length; or
(ii) an antisense strand of 15-30 nucleotides in length and a sense strand of 15-40 nucleotides in length, wherein the antisense strand has a region of complementarity to a target sequence of DGAT2 set forth in SEQ ID Nos: 156 or 157, and wherein the region of complementarity is at least 15 contiguous nucleotides in length.

4. The oligonucleotide of claim 3, wherein the sense strand is 36 nucleotides in length and/or the antisense strand is 22 nucleotides in length.

5. The oligonucleotide of claim 3 or 4, wherein the oligonucleotide comprises the antisense and sense strands of (i) and wherein the antisense strand comprises a nucleotide sequence selected from SEQ ID Nos: 2, 30, 32, 44 and 56, and optionally the sense strand comprises a nucleotide sequence selected from SEQ ID Nos: 1, 29, 31, 43 and 55; or comprises the antisense and sense strands of (ii) and wherein the antisense strand comprises a nucleotide sequence selected from SEQ ID Nos: 118, 120, 126, 130, 138 and 144, and optionally the sense strand comprises a nucleotide sequence selected from SEQ ID Nos: 117, 119, 125, 129, 137 and 143.

6. The oligonucleotide of any one of claims 1-5, wherein the region of complementarity to the target sequence of ACC or DGAT2 is at least 19 contiguous nucleotides in length.

7. The oligonucleotide of any one of claims 2-6, wherein the antisense strand and the sense strand form a duplex region, optionally wherein the duplex region is at least 19 nucleotides in length.

8. The oligonucleotide of any one of claims 1-7, wherein the region of complementarity (i) differs by no more than 3 nucleotides in length to the ACC or DGAT2 target sequence, or (ii) is fully complementary to the ACC or DGAT2 target sequence.

9. The oligonucleotide of any one of claims 2-8, wherein the 3′ end of the sense strand comprises a stem-loop set forth as S1-L-S2, wherein S1 is complementary to S2, and wherein L forms a loop between S1 and S2 of 3-5 nucleotides in length, and optionally wherein L is a tetraloop.

10. The oligonucleotide of claim 9, wherein the tetraloop comprises the sequence 5′-GAAA-3′.

11. The oligonucleotide of any one of claims 1-10, wherein the antisense strand comprises a 3′ overhang sequence of one or more nucleotides in length, optionally wherein the 3′ overhang sequence is 2 nucleotides in length, and further optionally wherein the 3′ overhang sequence is GG.

12. The oligonucleotide of any one of claims 1-11, wherein the oligonucleotide comprises at least one modified nucleotide.

13. The oligonucleotide of claim 12, wherein the modified nucleotide comprises a 2′-modification, optionally wherein the 2′-modification is a modification selected from 2′-aminoethyl, 2′-fluoro, 2′—O—methyl, 2′—O—methoxyethyl, and 2′-deoxy-2′-fluoro- β -d-arabinonucleic acid.

14. The oligonucleotide of claim 13, wherein (i) about 10-15%, 10%, 11%, 12%, 13%, 14% or 15% of the nucleotides of the sense strand comprise a 2′-fluoro modification; (ii) about 25-35%, 25%, 26%, 27%, 28%, 29%, 30%, 31%, 32%, 33%, 34% or 35% of the nucleotides of the antisense strand comprise a 2′-fluoro modification; and/or (iii) about 15-25%, 15%, 16%, 17%, 18%, 19%, 20%, 21%, 22%, 23%, 24%, or 25% of the nucleotides of the oligonucleotide comprise a 2′-fluoro modification.

15. The oligonucleotide of any one of claims 13-14, wherein (i) the sense strand comprises 36 nucleotides with positions numbered 1-36 from 5′ to 3′, wherein positions 8-11 comprise a 2′-fluoro modification; and/or (ii) the antisense strand comprises 22 nucleotides with positions numbered 1-22 from 5′ to 3′, and wherein positions 2, 3, 5, 7, 10 and 14, and optionally position 4, comprise a 2′-fluoro modification.

16. The oligonucleotide of any one of claims 1-15, wherein the oligonucleotide comprises at least one modified internucleotide linkage, optionally wherein the at least one modified internucleotide linkage is a phosphorothioate linkage.

17. The oligonucleotide of any one of claims 1-16, wherein the 4′-carbon of the sugar of the 5′-nucleotide of the antisense strand comprises a phosphate analog, optionally wherein the phosphate analog is oxymethylphosphonate, vinylphosphonate or malonylphosphonate, and further optionally wherein the phosphate analog is a 4′-phosphate analog comprising 4′-oxymethylphosphonate.

18. The oligonucleotide of any one of claims 1-17, wherein at least one nucleotide of the oligonucleotide is conjugated to one or more targeting ligands, wherein the one or more targeting ligands is selected from a carbohydrate, amino sugar, cholesterol, polypeptide, lipid and a N-acetylgalactosamine (GalNAc) moiety.

19. The oligonucleotide of claim 18, wherein the GalNac moiety is a monovalent GalNAc moiety, a bivalent GalNAc moiety, a trivalent GalNAc moiety or a tetravalent GalNAc moiety.

20. The oligonucleotide of any one of claims 2-19, wherein the sense and antisense strands comprise nucleotide sequences selected from the group consisting of:
(a) SEQ ID NOs: 162 and 30, respectively;
(b) SEQ ID NOs: 163 and 32, respectively;
(c) SEQ ID NOs: 169 and 44, respectively;
(d) SEQ ID NOs: 200 and 138, respectively;
(e) SEQ ID NOs: 203 and 144, respectively;
(f) SEQ ID NOs: 194 and 126, respectively;
(g) SEQ ID NOs: 191 and 120, respectively;
(h) SEQ ID NOs: 196 and 130, respectively; and
(i) SEQ ID NOs: 190 and 118, respectively.

21. The oligonucleotide of any one of claims 2-19, wherein the sense and antisense strands comprise nucleotide sequences selected from the group consisting of:
(a) SEQ ID NOs: 208 and 254, respectively;
(b) SEQ ID NOs: 209 and 255, respectively;
(c) SEQ ID NOs: 215 and 261, respectively;
(d) SEQ ID NOs: 246 and 292, respectively;
(e) SEQ ID NOs: 249 and 295, respectively;
(f) SEQ ID NOs: 240 and 286, respectively;
(g) SEQ ID NOs: 237 and 283, respectively;
(h) SEQ ID NOs: 242 and 288, respectively; and
(i) SEQ ID NOs: 236 and 282, respectively.

22. A pharmaceutical composition comprising the oligonucleotide of any one of claims 1-21, and a pharmaceutically acceptable carrier, delivery agent or excipient.

23. A composition comprising an inhibitor of ACC expression and an inhibitor of DGAT2 expression.

24. The composition of claim 23, wherein the inhibitor of ACC expression and the inhibitor of DGAT2 expression are each oligonucleotides.

25. The composition of claim 24, wherein the inhibitor of ACC expression comprises:
(a) an antisense strand of 15-30 nucleotides in length and a sense strand of 15-40 nucleotides in length, wherein the antisense strand has a region of complementarity to a target sequence of ACC set for in SEQ ID Nos: 150 or 151, and wherein the region of complementarity is at least 15 contiguous nucleotides in length;
(b) an antisense strand comprising a region of complementarity to a target sequence of ACC, wherein the antisense strand comprises a nucleotide sequence selected from SEQ ID Nos: 2, 30, 32, 44 and 56, and optionally a sense strand comprising a nucleotide sequence selected from SEQ ID Nos: 1, 29, 31, 43 and 55; or
(c) an antisense strand and a sense strand comprising the nucleotide sequences selected from:
(i) SEQ ID NOs: 162 and 30, respectively;
(ii) SEQ ID NOs: 163 and 32, respectively;
(iii) SEQ ID NOs: 169 and 44, respectively;
(iv) SEQ ID NOs: 208 and 254, respectively;
(v) SEQ ID NOs: 209 and 255, respectively; and
(vi) SEQ ID NOs: 215 and 261, respectively.

26. The composition of claim 24 or 25, wherein the inhibitor of DGAT2 expression comprises:
(a) an antisense strand of 15-30 nucleotides in length and a sense strand of 15-40 nucleotides in length, wherein the antisense strand has a region of complementarity to a target sequence of DGAT2 set for in SEQ ID Nos: 156 or 157, and wherein the region of complementarity is at least 15 contiguous nucleotides in length;
(b) an antisense strand comprising a region of complementarity to a target sequence of DGAT2, wherein the antisense strand comprises a nucleotide sequence selected from SEQ ID Nos: 118, 120, 126, 130, 138 and 144, and optionally a sense strand comprising a nucleotide sequence selected from SEQ ID Nos: 117, 119, 125, 129, 137 and 143; or
(c) an antisense strand and a sense strand comprising the nucleotide sequences selected from:
(i) SEQ ID NOs: 200 and 138, respectively;
(ii) SEQ ID NOs: 203 and 144, respectively;
(iii) SEQ ID NOs: 194 and 126, respectively;
(iv) SEQ ID NOs: 191 and 120, respectively;
(v) SEQ ID NOs: 196 and 130, respectively;
(vi) SEQ ID NOs: 190 and 118, respectively;
(vii) SEQ ID NOs: 246 and 292, respectively;
(viii) SEQ ID NOs: 249 and 295, respectively;
(ix) SEQ ID NOs: 240 and 286, respectively;
(x) SEQ ID NOs: 237 and 283, respectively;
(xi) SEQ ID NOs: 242 and 288, respectively; and
(xii) SEQ ID NOs: 236 and 282, respectively.

27. A method of treating an inflammatory, metabolic, fibrotic or cholestatic disease in a subject in need thereof, comprising administering the oligonucleotide of any one of claims 1-21, the pharmaceutical composition of claim 22, or the composition of any one of claims 23-26.

28. A method of treating an inflammatory, metabolic, fibrotic or cholestatic disease in a subject in need thereof that has received or is receiving an ACC inhibitor, comprising administering a DGAT2 inhibitor.

29. A method of treating an inflammatory, metabolic, fibrotic or cholestatic disease in a subject in need thereof that has received or is receiving a DGAT2 inhibitor, comprising administering an ACC inhibitor.

30. The method of any one of claims 27-29, wherein the disease is selected from the group consisting of, the disease is selected in the group consisting of metabolic liver diseases, non-alcoholic fatty liver disease (NAFLD), non-alcoholic steatohepatitis (NASH), drug-induced liver diseases, alcohol-induced liver diseases, infectious agent induced liver diseases, inflammatory liver diseases, immune system dysfunction-mediated liver diseases, dyslipidemia, cardiovascular diseases, restenosis, syndrome X, metabolic syndrome, diabetes, obesity, hypertension, chronic cholangiopathies, Primary Sclerosing Cholangitis (PSC), Primary Biliary Cholangitis (PBC), biliary atresia, progressive familial intrahepatic cholestasis type 3 (PFIC3), inflammatory bowel diseases, Crohn’s disease, ulcerative colitis, liver cancer, hepatocellular carcinoma, gastrointestinal cancer, gastric cancer, colorectal cancer, metabolic disease-induced liver fibrosis or cirrhosis, NAFLD induced fibrosis or cirrhosis, NASH-induced fibrosis or cirrhosis, alcohol-induced liver fibrosis or cirrhosis, drug-induced liver fibrosis or cirrhosis, infectious agent-induced liver fibrosis or cirrhosis, parasite infection-induced liver fibrosis or cirrhosis, bacterial infection-induced liver fibrosis or cirrhosis, viral infection-induced fibrosis or cirrhosis, HBV-infection induced liver fibrosis or cirrhosis, HCV-infection induced liver fibrosis or cirrhosis, HIV-infection induced liver fibrosis or cirrhosis, dual HCV and HIV-infection induced liver fibrosis or cirrhosis, radiation or chemotherapy-induced fibrosis or cirrhosis, biliary tract fibrosis, liver fibrosis or cirrhosis due to any chronic cholestatic disease, gut fibrosis of any etiology, Crohn’s disease induced fibrosis, ulcerative colitis-induced fibrosis, intestine (e.g. small intestine) fibrosis, colon fibrosis, stomach fibrosis, lung fibrosis, lung fibrosis consecutive to chronic inflammatory airway diseases, COPD, asthma, emphysema, smoker’s lung, tuberculosis, pulmonary fibrosis, and idiopathic pulmonary fibrosis (IPF).

31. The method of claim 30, wherein the disease is metabolic liver disease, non-alcoholic fatty liver disease (NAFLD) or non-alcoholic steatohepatitis (NASH).

32. A method of reducing an amount of liver fibrosis in a subject in need thereof, comprising administering the oligonucleotide of any one of claims 1-21, the pharmaceutical composition of claim 22, or the composition of any one of claims 23-26.

33. A method of treating a disease, disorder or condition associated with ACC and/or DGAT expression, comprising administering to a subject in need thereof the oligonucleotide of any one of claims 1-21, the pharmaceutical composition of claim 22, or the composition of any one of claims 23-26.

34. Use of the oligonucleotide of any one of claims 1-21, the pharmaceutical composition of claim 22, or the composition of any one of claims 23-26, in the manufacture of a medicament for treating a disease, disorder or condition associated with ACC and/or DGAT expression.

35. A kit comprising the oligonucleotide of any one of claims 1-21, the pharmaceutical composition of claim 22, or the composition of any one of claims 23-26, and a package insert comprising instructions for administration to a subject having a disease, disorder or condition associated with ACC and/or DGAT expression.
